(19) United States
(12) Patent Application Publication
(10) Pub. No.: US 2023/0227924 A1
(43) Pub. Date: Jul. 20, 2023

(54) COMPOSITIONS AND METHODS FOR DETECTING PICOBIRNAVIRUS

(71) Applicant: Abbott Laboratories, Abbott Park, IL (US)

(72) Inventors: Michael G. BERG, Abbott Park, IL (US); Kenn FORBERG, Abbott Park, IL (US); Todd V. MEYER, Abbott Park, IL (US); Ka-Cheung LUK, Abbott Park, IL (US)

(21) Appl. No.: 17/784,212

(22) PCT Filed: Dec. 23, 2020
(86) PCT No.: PCT/US2020/066858

§ 371 Date: Jun. 10, 2022

Related U.S. Application Data
(60) Provisional application No. 62/975,419, filed on Feb. 12, 2020.
(60) Provisional application No. 62/952,956, filed on Dec. 23, 2019.

(51) Int. Cl. C12Q 1/70; C12Q 1/686; C12N 15/63; C07K 14/085

(52) U.S. Cl. CPC C12Q 1/701; C12Q 1/686; C12N 15/63; C07K 14/085; C12N 2710/00021; C12Q 2600/16

(57) ABSTRACT

Provided herein are compositions, methods, and kits for detecting human picobirnavims (PBV). In certain embodiments, provided herein are PBV specific nucleic acid probes and primers, and methods for detecting PBV nucleic acid.

### CROSS REFERENCE TO RELATED APPLICATIONS

This application is a national stage application of PCT/US2020/066858 filed Dec. 23, 2020, which claims priority to U.S. Provisional Application No. 62/975,419 filed Feb. 12, 2020 and U.S. Provisional Application No. 62/952,956 filed Dec. 23, 2019, each of which are hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 137,768 Byte ASCII (Text) file named “38035-253_ST25.TXT,” created on Jun. 9, 2022.

### TECHNICAL FIELD

Provided herein are compositions, methods, and kits for detecting human picobirnavirus (PBV). In certain embodiments, provided herein are PBV specific nucleic acid probes and primers, and methods for detecting PBV nucleic acid.

### BACKGROUND

Picobirnaviruses (PBV) are segmented, double stranded RNA viruses found in a range of hosts and are primarily known to be associated with gastroenteritis and diarrhea. The Picobirnavirus name is derived from Latin being small (pico), having two segments (bi), and viral nucleic made up of RNA, which is double stranded in this case. The virus is non-enveloped and the 2 RNA bands can be larger in size (Genogroup I: 2.3-2.6 kb and 1.5-1.9 kb) or smaller (Genogroup II: 1.75 and 1.55 kb). It was initially discovered in fecal samples from both humans and pigmy rats in Brazil.

PBV's have been found in humans as the ‘sole’ pathogen in cases of watery diarrhea and gastroenteritis, often in immunocompromised patients. However, they have also been found in a wide range of animal species worldwide, whether they have diarrhea or not. Indeed, these are genetically distinct viruses that appear to be rapidly evolving via reassortment, due to their segmented nature. For example, the close relatedness of porcine and human strains points to the likelihood of a crossover events or circulation between these hosts, much like influenza. Indeed, unlike other viruses that have co-evolved with their host, PBV strains do not segregate into distinct clades by host. Rather, the simple capsid appears to have obtained a generalized means of infecting animal cells and there does not appear to be a species restriction. Thus again, detection of PBVs in farm animals, birds, reptiles, domestic pets, wild birds, and in sewage in every part of the world, coupled with the documented examples of interspecies transmission (Argentina, Hungary, Venezuela, India) suggests PBVs have zoonotic potential and may present a public health threat (1-4). Accordingly, what is needed are compositions, methods, and kits for diagnosing PBVs, particularly in human subjects.

### SUMMARY

Provided herein are materials and methods for detecting PBV in a sample. In some aspects, provided herein are primers for amplifying PBVin a sample. In some embodiments, the primer comprises a sequence with 80% or more sequence identity to SEQ ID NO: 4, SEQ ID NO: 5, SEQ ID NO: 7, SEQ ID NO: 8, or complements thereof.

In some aspects, provided herein are probes for detecting PBVin a sample. In some embodiments, the probe comprises a sequence with 80% or more sequence identity to SEQ ID NO: 6, SEQ ID NO: 9, or complements thereof.

In some aspects, provided herein are compositions for amplifying PBVin a sample. In some embodiments, the composition comprises at least one forward primer comprising a sequence with 80% or more sequence identity to SEQ ID NO: 4 or a complement thereof and at least one reverse primer comprising a sequence with 80% or more sequence identity to SEQ ID NO: 5 or a complement thereof. In some embodiments, the composition comprises at least one forward primer comprising a sequence with 80% or more sequence identity to SEQ ID NO: 7 or a complement thereof and at least one reverse primer comprising a sequence with 80% or more sequence identity to SEQ ID NO: 8 or a complement thereof.

In some embodiments, the composition comprises at least one forward primer comprising a sequence with 80% or more sequence identity to SEQ ID NO: 4 or a complement thereof, at least one reverse primer comprising a sequence with 80% or more sequence identity to SEQ ID NO: 5 or a complement thereof, and a probe comprising a sequence with 80% or more sequence identity to SEQ ID NO: 6 or a complement thereof. In some embodiments, the composition comprises at least one forward primer comprising a sequence with 80% or more sequence identity to SEQ ID NO: 7 or a complement thereof, at least one reverse primer comprising a sequence with 80% or more sequence identity to SEQ ID NO: 8 or a complement thereof, and a probe comprising a sequence with 80% or more sequence identity to SEQ ID NO: 9 or a complement thereof.

In some aspects, provided herein are methods for detecting PBV in a sample. In some embodiments, the methods comprise contacting the sample with at least one primer and/or at least one probe. In some embodiments, the PBV comprises at least one sequence selected from SEQ ID NO: 1, SEQ ID NO: 6, SEQ ID NO: 7, SEQ ID NO: 9, SEQ ID NO: 10, and SEQ ID NO: 11.

In some aspects, provided herein are kits for detecting PBV in a sample. In some embodiments, the kit comprises at least one forward primer comprising a sequence with 80% or more sequence identity to SEQ ID NO: 4 or a complement thereof, at least one reverse primer comprising a sequence with 80% or more sequence identity to SEQ ID NO: 5 or a complement thereof, and a probe comprising a sequence with 80% or more sequence identity to SEQ ID NO: 6 or a complement thereof. In some embodiments, the kit comprises at least one forward primer comprising a sequence with 80% or more sequence identity to SEQ ID NO: 7 or a complement thereof, at least one reverse primer comprising a sequence with 80% or more sequence identity to SEQ ID NO: 8 or a complement thereof, and a probe comprising a sequence with 80% or more sequence identity to SEQ ID NO: 9 or a complement thereof.

In some aspects, provided herein are isolated polynucleotides having 50% or more sequence identity to SEQ ID NO: 1, SEQ ID NO: 6, SEQ ID NO: 9, SEQ ID NO: 10, or fragments thereof. In some aspects, provided herein are vectors and host cells comprising the same.

In some aspects, provided herein are isolated polypeptides having 80% or more sequence identity to SEQ ID NO: 7, SEQ ID NO: 11, or fragments thereof. In some aspects, provided herein are host cells comprising the same.

### BRIEF DESCRIPTION OF THE DRAWINGS

FIGS. 1A-1B show representative drawings of the structure of PBV. Picobirnaviruses (PBV) are segmented, double stranded RNA viruses consisting of two segments and a capsid (FIG. 1A). Segment 1 is approximately 2.5 kb long and encodes a hypothetical, hydrophilic protein (ORF1) of ˜200 aa in one reading frame, and the capsid protein in another (˜500 aa). Segment 2 is approximately 1.7 kb long and encodes only the RDRP (FIG. 1B).

FIG. 2A shows a coverage plot for segment 1 (capsid) of the novel PBV described herein obtained by next-generation sequencing of the index case (MRN3406) sputum sample.

FIG. 2B shows a coverage plot for segment 2 (RDRP) of of the novel PBV described herein (e.g. ABT-PBV) obtained by next-generation sequencing of the index case (MRN3406) sputum sample.

FIG. 3 shows the pairwise alignment of the amino acid sequence of the capsid for the novel PBV strain (MRN3406) described herein with the capsid from various other strains.

FIG. 4 shows the pairwise alignment of the amino acid sequence of the RDRP for the novel PBV strain (MRN3406) described herein with the RDRP sequence from various other strains.

FIG. 5A-5B show neighbor-joining radial trees of the capsid protein determined from a 521 amino acid gapped alignment (FIG. 3A) and a 156 amino acid gap-stripped alignment (FIG. 3B).

FIG. 6 shows an example of an RDRP tree from Smits, et al which is based on the typical, conserved 165 nt (55 aa) segment interrogated to infer phylogenetic relationships among strains. This tree highlights pig and human sequences obtained from respiratory tracts, such as VS2000252/2005 shown in red (5).

FIG. 7A shows a partial-length RDRP neighbor-joining tree of the same 55 aa region in FIG. 6, rooted on human Genotype II strain, AF246940 (4-GA-91) and includes the ABT-PBV strain. RDRP sequences retrieved from GenBank (n=841) were reduced to n=215, to include a diversity of strains and those with implications for respiratory disease. The ABT-PBV branch has been expanded to show it groups with strains KM285233 & KM285234, each obtained in 2009 from swabs of upper respiratory tracts from two patients in Cambodia. GU968930 branches with 99% bootstrap support with VS2000252/2005 shown in FIG. 6. FIG. 7B shows linear and radial trees from an alignment of 132 sequences spanning 348 aa (ABT coordinates: 126-473). The ABT-PBV sequence continues to branch with Cambodian respiratory strains over the longer region analyzed.

FIG. 8A shows an amino acid alignment of the RDRP qPCR target region. Note the identity (*) of the ABT-PBV RDRP protein with Cambodian proteins (AK92636.1 & AKG92637.1). FIG. 8B shows the nucleotide alignment of the RDRP qPCR target region and relative position of primers and probes within the amplicon. MRN3406=Novel ABT-PBV strain; KM285233 & KM285234 are respiratory strains.

FIG. 9 outlines the scheme and expected results for two independent, quantitative RT-PCR reactions detecting infections of six different picobirnavirus strains. Column 1 depicts amplification curves of serially diluted positive controls detecting capsid with a single FAM-labeled probe. Only the novel ABT-PBV or highly identical strains will be detected. Columns 2-4 depict curves for a 2^{nd} multiplex PCR reaction detecting the RDRP segment. Universal primers generate an amplicon for which a universal probe (FAM; column 2) detects all 6 PBV strains, a Cy5 probe detects only ABT PBV, and a Cy3 probe detects only the respiratory PBV strains from Cambodia.

FIG. 10 shows an ethidium bromide stained agarose gel of in vitro transcripts (IVT). Lanes 1-3 are aichivirus VP0 sequences, lanes 4-8 & 10 are RDRP sequences derived from 6 different PBV strains, and lane 9 is the capsid sequence derived from the ABT-PBV strain. IVTs serve as positive controls in the qPCR assay.

FIG. 11A-B shows actual rtPCR results for 10-fold serial dilutions of the ABT-PBV capsid IVT (9: PVABTCA) using the capsid primers and probes, as depicted in FIG. 9, column 1. Amplification curves are shown in FIG. 11A. The linear regression plot is shown in FIG. 11B.

FIG. 12 shows actual rtPCR results for 10-fold serial dilutions of RDRP IVT for various in vitro transcripts, as depicted in FIG. 9, columns 2-4. RDRP from all 6 strains are detected by FAM (column 1), whereas only those similar to ABT-PBV (8: MRN3406) are detected by Cy5 and to the Cambodian (6: KM285233) strain are detected by Cy3.

FIG. 13 summarizes the hits detected in a screen of n=130 sputum samples obtained from US and Colombian individuals hospitalized with severe respiratory illness.

FIG. 14 shows a linear tree for capsid (as in FIG. 5A) from an alignment of 147 sequences spanning 242 aa (ABT coordinates: 91-333), and includes the newly sequenced respiratory strains identified by the qPCR assay. The new respiratory sequences cluster into distinct groups but are distant from with Cambodian respiratory strains and branch with GI tract-derived strains.

FIG. 15 shows a linear tree for RDRP (as in FIG. 7B) from an alignment of 143 sequences spanning 348 aa (ABT coordinates: 126-473), and includes the newly sequenced respiratory strains identified by the qPCR assay. The new respiratory sequences cluster into distinct groups and are found on the same branch with Cambodian respiratory strains without any GI tract-derived strains.

### DETAILED DESCRIPTION

In some aspects, provided herein are provided herein are materials and methods for detecting any picobirnavirus infection in a subject. For example, provided herein are materials and methods for detecting picobirnaviruses associated with gastroenterirtis, diarrhea, or respiratory illness. In other embodiments, provided herein are materials and methods for detecting specific picobirnaviruses associated with respiratory illness in a subject.

PBVs have recently been detected in respiratory secretions, both in pigs and in humans (5). For example, novel PBV strains were detected in 2 patients with severe, acute respiratory illness in a surveillance study conducted in Uganda (6). It is possible that the significance of these viruses' role in respiratory disease is just beginning to be appreciated. One question raised is whether these viruses actually infect animals or are found in intestinal bacteria or other eukaryotic parasites. Their ability to auto-proteolyze their capsid and invade liposomes suggests they are in fact vertebrate viruses, unlike the related partitiviruses that infect unicellular organisms and fungi. Studies in pigs and chickens suggest the virus can persist chronically, with periods of large shedding interspersed by periods of silence, and that some hosts can serve as asymptomatic reservoirs. This implies the virus is adapted to the host and may underscore why pathogenicity (e.g. diarrhea) is seen often in the immunocompromised or those co-infected with other enteric viruses like rotavirus, calicivirus, and astrovirus, and thus PBVs may be opportunistic pathogens.

Diagnosis has been previously made by PAGE and silver stain detection of the two RNA segments, although PCR is now a simpler approach in widespread use. Segment 1 is approximately 2.5 kb long and encodes a hypothetical, hydrophilic protein (ORF1) of ˜200 aa in one reading frame, and the capsid protein in another (˜500 aa). Segment 2 is approximately 1.7 kb long and encodes only the RDRP. Given the high genetic diversity of PBVs, even degenerate primer sets in the conserved RDRP region (280 bp) yield limited success. Phylogenetic analyses are often on the basis of only 168 nt/55 aa in the RDRP7. Their heterologous nature is further pronounced by the documented detection of multiple PBV strains in individuals. Unbiased NGS is now the preferred means of detection and sequencing. At present there are only 6 complete PBV genomes in NCBI (e.g. both segments). All of these are from enteric-derived samples; ours would be the first from respiratory specimens.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “amplicon” refers to a nucleic acid generated via an amplification reaction. The amplicon is typically double stranded DNA; however, it may be RNA and/or a DNA:RNA hybrid. The amplicon comprises DNA complementary to a sample nucleic acid. In some embodiments, primer pairs are configured to generate amplicons from a sample nucleic acid. As such, the base composition of any given amplicon may include the primer pair, the complement of the primer pair, and the region of a sample nucleic acid that was amplified to generate the amplicon. In one embodiment, the incorporation of the designed primer pair sequences into an amplicon may replace the native sequences at the primer binding site, and complement thereof. In certain embodiments, after amplification of the target region using the primers, the resultant amplicons having the primer sequences are used for subsequent analysis (e.g. base composition determination, for example, via direct sequencing). In some embodiments, the amplicon further comprises a length that is compatible with subsequent analysis. An example of an amplicon is a DNA or an RNA product (usually a segment of a gene, DNA or RNA) produced as a result of PCR, real-time PCR, RT-PCR, competitive RT-PCR, ligase chain reaction (LCR), gap LCR, strand displacement amplification (SDA), nucleic acid sequence-based amplification (NASBA), transcription-mediated amplification (TMA), or the like.

As used herein, the phrases “amplification,” “amplification method,” or “amplification reaction,” are used interchangeably and refer to a method or process that increases the representation of a population of specific nucleic acid (all types of DNA or RNA) sequences (such as a target sequence or a target nucleic acid) in a sample. Examples of amplification methods that can be used in the present disclosure include, but are not limited to, PCR, real-time PCR, RT-PCR, competitive RT-PCR, and the like, all of which are known to one skilled in the art.

As used herein, the phrase “amplification conditions” refers to conditions that promote annealing and/or extension of primer sequences. Such conditions are well-known in the art and depend on the amplification method selected. For example, PCR amplification conditions generally comprise thermal cycling, e.g., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, pH, and the like.

As used herein, the phrase “amplification reagents” refers to reagents used in amplification reactions and may include, but is not limited to, buffers, reagents, enzymes having reverse transcriptase, and/or polymerase, or exonuclease activities; enzyme cofactors such as magnesium or manganese; salts; and deoxynucleotide triphosphates (dNTPs), such as deoxyadenosine triphosphate (dATP), deoxyguanosine triphosphate (dGTP), deoxycytidine triphosphate (dCTP), deoxythymidine triphosphate (dTTP), and deoxyuridine triphosphate (dUTP). Amplification reagents may readily be selected by one skilled in the art depending on the amplification method employed.

A “coding sequence” is a polynucleotide sequence which is transcribed into mRNA and translated into a polypeptide when placed under the control of appropriate regulatory sequences. The boundaries of the coding sequence are determined by and include a translation start codon at the 5′-terminus and one or more translation stop codons at the 3′-terminus. A coding sequence can include, but is not limited to, mRNA, cDNA, and recombinant polynucleotide sequences.

The term “control sequence” refers to polynucleotide sequences which are necessary to effect the expression of coding sequences to which they are ligated. The nature of such control sequences differs depending upon the host organism. In prokaryotes, such control sequences generally include promoter, ribosomal binding site and terminators; in eukaryotes, such control sequences generally include promoters, terminators and, in some instances, enhancers. The term “control sequence” thus is intended to include at a minimum all components whose presence is necessary for expression, and also may include additional components whose presence is advantageous, for example, leader sequences.

A “conformational epitope” is an epitope that is comprised of specific juxtaposition of amino acids in an immunologically recognizable structure, such amino acids being present on the same polypeptide in a contiguous or non-contiguous order or present on different polypeptides.

As used herein, the phrase, “directly detectable,” when used in reference to a detectable label or detectable moiety, means that the detectable label or detectable moiety does not require further reaction or manipulation to be detectable. For example, a fluorescent moiety is directly detectable by fluorescence spectroscopy methods. In contrast, the phrase “indirectly detectable,” when used herein in reference to a detectable label or detectable moiety, means that the detectable label or detectable moiety becomes detectable after further reaction or manipulation. For example, a hapten becomes detectable after reaction with an appropriate antibody attached to a reporter, such as a fluorescent dye.

“Encoded by” refers to a nucleic acid sequence which codes for a polypeptide sequence. Also encompassed are polypeptide sequences which are immnunologically identifiable with a polypeptide encoded by the sequence. Thus, a “polypeptide,” “protein,” or “amino acid” sequence as claimed herein may have at least 60% similarity, more preferably at least about 70% similarity, and most preferably about 80% similarity to a particular polypeptide or amino acid sequence specified below.

As used herein, “epitope” means an antigenic determinant of a polypeptide. Conceivably, an epitope can comprise three amino acids in a spatial conformation which is unique to the epitope. Generally, an epitope consists of at least five such amino acids, and more usually, it consists of at least eight to ten amino acids. Methods of examining spatial conformation are known in the art and include, for example, x-ray crystallography and two-dimensional nuclear magnetic resonance.

The terms, “fluorophore,” “fluorescent moiety,” “fluorescent label,” and “fluorescent dye” are used interchangeably herein and refer to a molecule that absorbs a quantum of electromagnetic radiation at one wavelength, and emits one or more photons at a different, typically longer, wavelength in response thereto. Numerous fluorescent dyes of a wide variety of structures and characteristics are suitable for use in the practice of the present disclosure. Methods and materials are known for fluorescently labeling nucleic acid molecules (See, R. P. Haugland, “Molecular Probes: Handbook of Fluorescent Probes and Research Chemicals 1992-1994,” 5th Ed., 1994, Molecular Probes, Inc.). Preferably, a fluorescent label or moiety absorbs and emits light with high efficiency (e.g., has a high molar absorption coefficient at the excitation wavelength used, and a high fluorescence quantum yield), and is photostable (e.g., does not undergo significant degradation upon light excitation within the time necessary to perform the analysis). Rather than being directly detectable themselves, some fluorescent dyes transfer energy to another fluorescent dye in a process called fluorescence resonance energy transfer (FRET), and the second dye produces the detected signal. Such FRET fluorescent dye pairs are also encompassed by the term “fluorescent moiety.” The use of physically- linked fluorescent reporters/quencher moieties is also within the scope of the present disclosure. In these aspects, when the fluorescent reporter and quencher moiety are held in close proximity, such as at the ends of a probe, the quencher moiety prevents detection of a fluorescent signal from the reporter moiety. When the two moieties are physically separated, such as after cleavage by a DNA polymerase, the fluorescent signal from the reporter moiety becomes detectable.

A “fragment” of a specified polypeptide refers to an amino acid sequence which comprises at least about 3-5 amino acids, more preferably at least about 8-10 amino acids, and even more preferably at least about 15-20 amino acids, derived from the specified polypeptide. A “fragment” of a specified polynucleotide refers to a nucleotide sequence which comprises at least 10 base pairs. For example, a fragment may comprise at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 base pairs.

As used herein, the term “hybridization” refers to the formation of complexes between nucleic acid sequences which are sufficiently complementary to form complexes via Watson-Crick base pairing or non-canonical base pairing. For example, when a primer “hybridizes” with a target sequence (template), such complexes (or hybrids) are sufficiently stable to serve the priming function required by, e.g., the DNA polymerase, to initiate DNA synthesis. It will be appreciated by one skilled in the art that hybridizing sequences need not have perfect complementarity to provide stable hybrids. In many situations, stable hybrids will form where fewer than about 10% of the bases are mismatches. Accordingly, as used herein, the term “complementary” refers to an oligonucleotide that forms a stable duplex with its complement under assay conditions, generally where there is about 80%, about 81%, about 82%, about 83%, about 84%, about 85%, about 86%, about 87%, about 88%, about 89%, about 90%, about 91%, about 92%, about 93%, about 94% about 95%, about 96%, about 97%, about 98%, or about 99% greater homology. Those skilled in the art understand how to estimate and adjust the stringency of hybridization conditions such that sequences having at least a desired level of complementarity will stably hybridize, while those having lower complementarity will not. Examples of hybridization conditions and parameters can be found, for example in, Sambrook et al., “Molecular Cloning: A Laboratory Manual” 1989, Second Edition, Cold Spring Harbor Press: Plainview, N.Y.; F. M. Ausubel, “Current Protocols in Molecular Biology” 1994, John Wiley & Sons: Secaucus, N.J.

The term “immunologically identifiable with/as” refers to the presence of epitope(s) and polypeptide(s) which also are present in and are unique to the designated polypeptide(s). Immunological identity may be determined by antibody binding and/or competition in binding. These techniques are known to the skilled artisan and also are described herein. The uniqueness of an epitope also can be determined by computer searches of known data banks, such as GenBank, for the polynucleotide sequences which encode the epitope, and by amino acid sequence comparisons with other known proteins.

A polypeptide is “immunologically reactive” with an antibody when it binds to an antibody due to antibody recognition of a specific epitope contained within the polypeptide. Immunological reactivity may be determined by antibody binding, more particularly by the kinetics of antibody binding, and/or by competition in binding using as competitor(s) a known polypeptide(s) containing an epitope against which the antibody is directed. The methods for determining whether a polypeptide is immunologically reactive with an antibody are known in the art.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

As used herein, the terms “labeled” and “labeled with a detectable label (or agent or moiety)” are used interchangeably herein and specify that an entity (e.g., a primer or a probe) can be visualized, for example following binding to another entity (e.g., an amplification product or amplicon). Preferably, the detectable label is selected such that it generates a signal which can be measured and whose intensity is related to (e.g., proportional to) the amount of bound entity. A wide variety of systems for labeling and/or detecting nucleic acid molecules, such as primer and probes, are well-known in the art. Labeled nucleic acids can be prepared by incorporation of, or conjugation to, a label that is directly or indirectly detectable by spectroscopic, photochemical, biochemical, immunochemical, electrical, optical, chemical, or other means. Suitable detectable agents include, but are not limited to, radionuclides, fluorophores, chemiluminescent agents, microparticles, enzymes, colorimetric labels, magnetic labels, haptens, Molecular Beacons, aptamer beacons, and the like.

As used herein, the terms “nucleic acid,” “nucleic acid sequence,” “oligonucleotide,” and “polynucleotide” refer to at least two nucleotides covalently linked together. The depiction of a single strand also defines the sequence of the complementary strand. Thus, an oligonucleotide also encompasses the complementary strand of a depicted single strand. An oligonucleotide also encompasses substantially identical nucleic acids and complements thereof. Oligonucleotides can be single-stranded or double-stranded, or can contain portions of both double-stranded and single-stranded sequences. The oligonucleotide can be DNA, both genomic and complimentary DNA (cDNA), RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Oligonucleotides can be obtained by chemical synthesis methods or by recombinant methods. A particular oligonucleotide sequence can encompass conservatively modified variants thereof (e.g., codon substitutions), alleles, orthologs, single nucleotide polymorphisms (SNPs), and complementary sequences as well as the sequence explicitly indicated.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequences.

“Polypeptide” and “protein” are used interchangeably herein and indicate a molecular chain of amino acids linked through covalent and/or noncovalent bonds. The terms do not refer to a specific length of the product. Thus, peptides, oligopeptides and proteins are included within the definition of polypeptide. The terms include post-expression modifications of the polypeptide, for example, glycosylations, acetylations, phosphorylations and the like. In addition, protein fragments, analogs, mutated or variant proteins, fusion proteins and the like are included within the meaning of polypeptide.

The term “primer” or “oligonucleotide primer” as used interchangeably herein as used herein, refers to an oligonucleotide capable of acting as a point of initiation for DNA synthesis under suitable conditions. Suitable conditions include those in which hybridization of the oligonucleotide to a template nucleic acid occurs, and synthesis or amplification of the target sequence occurs, in the presence of four different nucleoside triphosphates and an agent for extension (e.g., a DNA polymerase) in an appropriate buffer and at a suitable temperature. A “forward oligonucleotide primer” or “sense primer,” as used herein, refers to an oligonucleotide capable of acting as a point of initiation for DNA synthesis at the 5′ end of a target nucleic acid sequence. A “reverse oligonucleotide primer” or “anti-sense primer,” as used herein, refers to an oligonucleotide capable of acting as a point of initiation for DNA synthesis at the 3′ end of a target nucleic acid sequence.The phrase “forward primer” refers to a primer that hybridizes (or anneals) with the target sequence (e.g., template strand). The phrase “reverse primer” refers to a primer that hybridizes (or anneals) to the complementary strand of the target sequence. The forward primer hybridizes with the target sequence 5′ with respect to the reverse primerThe phrase “forward primer” refers to a primer that hybridizes (or anneals) with the target sequence (e.g., template strand). The phrase “reverse primer” refers to a primer that hybridizes (or anneals) to the complementary strand of the target sequence. The forward primer hybridizes with the target sequence 5′ with respect to the reverse primer.

As used herein, the phrase “primer set” refers to two or more primers which together are capable of priming the amplification of a target sequence or target nucleic acid of interest (e.g., a target sequence within the PBV). In certain embodiments, the term “primer set” refers to a pair of primers including a 5′ (upstream) primer (or forward primer) that hybridizes with the 5′-end of the target sequence or target nucleic acid to be amplified and a 3′ (downstream) primer (or reverse primer) that hybridizes with the complement of the target sequence or target nucleic acid to be amplified. Such primer sets or primer pairs are particularly useful in PCR amplification reactions.

The term “probe” or “oligonucleotide primer” as used interchangeably herein refers to an oligonucleotide that hybridizes specifically to a target sequence in a nucleic acid, preferably in an amplified nucleic acid, under conditions that promote hybridization, to form a detectable hybrid. A probe may contain a detectable moiety (e.g., a label) which either may be attached to the end(s) of the probe or may be internal. The nucleotides of the probe which hybridize to the target nucleic acid sequence need not be strictly contiguous, as may be the case with a detectable moiety internal to the sequence of the probe. Detection may either be direct (i.e., resulting from a probe hybridizing directly to the target sequence or amplified nucleic acid) or indirect (i.e., resulting from a probe hybridizing to an intermediate molecular structure that links the probe to the target sequence or amplified nucleic acid). An oligonucleotide probe may comprise target-specific sequences and other sequences that contribute to three-dimensional conformation of the probe (e.g., as described in, e.g., U.S. Pat. Nos. 5,118,801 and 5,312,728).

As used herein, the phrase “primer and probe set” refers to a combination including two or more primers which together are capable of priming the amplification of a target sequence or target nucleic acid, and least one probe which can detect the target sequence or target nucleic acid. The probe generally hybridizes to a strand of an amplification product (or amplicon) to form an amplification product/probe hybrid, which can be detected using routine techniques known to those skilled in the art.

“Purified polypeptide” or “purified polynucleotide” refers to a polypeptide or polynucleotide of interest or fragment thereof which contains less than about 50%, preferably less than about 70%, and more preferably, less than about 90% of cellular components with which the polypeptide or polynucleotide of interest or fragment thereof is naturally associated. Methods for purifying are known in the art.

The terms “recombinant polypeptide” or “recombinant protein”, used interchangeably herein, describe a polypeptide which by virtue of its origin or manipulation is not associated with all or a portion of the polypeptide with which it is associated in nature and/or is linked to a polypeptide other than that to which it is linked in nature. A recombinant or encoded polypeptide or protein is not necessarily translated from a designated nucleic acid sequence. It also may be generated in any manner, including chemical synthesis or expression of a recombinant expression system.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

As used herein “replicon” means any genetic element, such as a plasmid, a chromosome or a virus, that behaves as an autonomous unit of polynucleotide replication within a cell.

As used herein, the term “sample” generally refers to a biological material being tested for and/or suspected of containing an analyte of interest, such as an PBV sequence. The sample may be derived from any biological source, such as, a cervical, vaginal or anal swab or brush, or a physiological fluid including, but not limited to, whole blood, serum, plasma, interstitial fluid, saliva, ocular lens fluid, cerebral spinal fluid, sweat, urine, milk, ascites fluid, mucus, nasal fluid, sputum, synovial fluid, peritoneal fluid, vaginal fluid, menses, amniotic fluid, semen, and so forth. The sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, lyophilization, inactivation of interfering components, the addition of reagents, lysing, etc. Moreover, it may also be beneficial to modify a solid sample to form a liquid medium or to release the analyte. Preferably, the sample may be plasma.

The term “sequence identity” refers to the degree of similarity between two sequences (e.g., nucleic acid (e.g., oligonucleotide or polynucleotide sequences) or amino acid sequences). To determine the percent identity of two nucleic acid or amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistically significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment. In some embodiments, the subject is suspected of having a respiratory illness.

The term “synthetic peptide” as used herein means a polymeric form of amino acids of any length, which may be chemically synthesized by methods well-known to those skilled in the art. These synthetic peptides are useful in various applications.

The phrases “target sequence” and “target nucleic acid” are used interchangeably herein and refer to that which the presence or absence of which is desired to be detected. In the context of the present disclosure, a target sequence preferably includes a nucleic acid sequence to which one or more primers will complex. The target sequence can also include a probe-hybridizing region with which a probe will form a stable hybrid under appropriate amplification conditions. As will be recognized by one of ordinary skill in the art, a target sequence may be single-stranded or double-stranded.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Variant” is used herein to describe a peptide or polypeptide that differs in sequence by the insertion, deletion, or conservative substitution of amino acids, but retain at least one biological activity. Representative examples of “biological activity” include the ability to be bound by a specific antibody or to promote an immune response. Variant is also used herein to describe a protein with a sequence that is substantially identical to a referenced protein with a sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to describe a polypeptide or a fragment thereof that has been differentially processed, such as by proteolysis, phosphorylation, or other post-translational modification, yet retains its antigen reactivity.

A “vector” is a replicon to which another polynucleotide segment is attached, such as to bring about the replication and/or expression of the attached segment.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Novel Picobirnavirus

In some aspects, provided herein is a novel strain of picobirnavirus. The novel picobirnavirus strain described herein is referred to interchangeably herein as ABT-PBV, the inde. In some embodiments, the strain may be present in respiratory specimens. In some embodiments, the strain may cause respiratory illness.

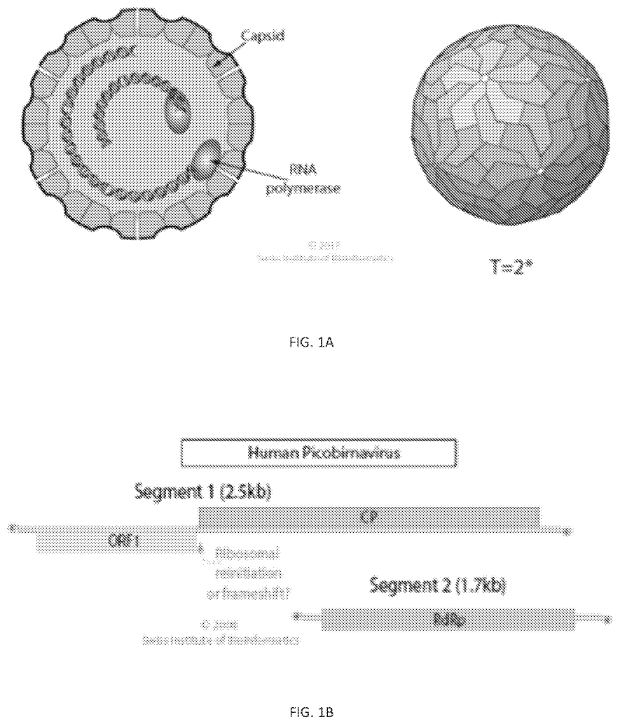

PBV comprises two segments (FIG. 1A-1B). Segment 1 is approximately 2.5 kb long and encodes a hypothetical, hydrophilic protein (ORF1) of ˜200 aa in one reading frame, and the capsid protein in another (˜500 aa). Segment 2 is approximately 1.7 kb long and encodes the RDRP.

In some aspects, the present disclosure provides polynucleotide sequences derived from PBV and polypeptides encoded thereby. The polynucleotide(s) may be in the form of mRNA or DNA. Polynucleotides in the form of DNA, cDNA, genomic DNA, and synthetic DNA are within the scope of the present disclosure. In some aspects, the polynucleotide is in the form of DNA. In other aspects, the polynucleotide is in the form of cDNA. In yet other aspects, the polynucleotide is in the form of genomic DNA. In still yet further aspect, the polynucleotide is in the form of synthetic DNA.

The DNA may be double-stranded or single-stranded, and if single stranded may be the coding (sense) strand or non-coding (anti-sense) strand. The coding sequence which encodes the polypeptide may be identical to the coding sequence provided herein or may be a different coding sequence which coding sequence, as a result of the redundancy or degeneracy of the genetic code, encodes the same polypeptide as the DNA provided herein.

The polynucleotides provided herein may include only the coding sequence for the polypeptide, or the coding sequence for the polypeptide and additional coding sequence such as a leader or secretory sequence or a proprotein sequence, or the coding sequence for the polypeptide (and optionally additional coding sequence) and non-coding sequence, such as a non-coding sequence 5′ and/or 3′ of the coding sequence for the polypeptide.

In addition, the disclosure includes variant polynucleotides containing modifications such as polynucleotide deletions, substitutions or additions; and any polypeptide modification resulting from the variant polynucleotide sequence. A polynucleotide of the present disclosure also may have a coding sequence which is a naturally-occurring variant of the coding sequence provided herein.

In addition, the coding sequence for the polypeptide may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the polypeptide. The polynucleotides may also encode for a proprotein which is the protein plus additional 5′ amino acid residues. A protein having a prosequence is a proprotein and may in some cases be an inactive form of the protein. Once the prosequence is cleaved an active protein remains. Thus, the polynucleotide of the present disclosure may encode for a protein, or for a protein having a prosequence or for a protein having both a presequence (leader sequence) and a prosequence.

The polynucleotides of the present disclosure may also have the coding sequence fused in frame to a marker sequence which allows for purification of the polypeptide of the present disclosure. The marker sequence may be a hexa-histidine tag supplied by a pQE-9 vector to provide for purification of the polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein. See, for example, I. Wilson et al., Cell 37:767 (1984).

For the novel PBV described herein, the complete sequence of segment is provided in SEQ ID NO: 1. In some embodiments, provided herein are isolated polynucleotides having 50% or more sequence identity (e.g. at least 50%, 55%, 60%, 65%, 70%, 75%, 80%, 85%, 90%, 91%, 92%, 93%, 94%, 95%, 96%, 97%, 98%, 99%, or 100%) to SEQ ID NO: 1 or a fragment thereof.

For the novel PBV described herein, the nucleotide sequence of the capsid is provided in SEQ ID NO: 6. In some embodiments, provided herein are isolated polynucleotides having 50% or more sequence identity (e.g. at least 50%, 55%, 60%, 65%, 70%, 75%, 80%, 85%, 90%, 91%, 92%, 93%, 94%, 95%, 96%, 97%, 98%, 99%, or 100%) to SEQ ID NO: 6 or a fragment thereof. For example, provided herein are isolated polynucleotides of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45.

The complete sequence of segment 2 is provided in SEQ ID NO: 9. In some embodiments, provided herein are isolated polynucleotides having 50% or more sequence identity (e.g. at least 50%, 55%, 60%, 65%, 70%, 75%, 80%, 85%, 90%, 91%, 92%, 93%, 94%, 95%, 96%, 97%, 98%, 99%, or 100%) to SEQ ID NO: 9 or a fragment thereof.

The nucleotide sequence of the RNA-dependent RNA polymerase (RDRP) is provided in SEQ ID NO: 10. In some embodiments, provided herein are isolated polynucleotides having 50% or more sequence identity (e.g. at least 50%, 55%, 60%, 65%, 70%, 75%, 80%, 85%, 90%, 91%, 92%, 93%, 94%, 95%, 96%, 97%, 98%, 99%, or 100%) to SEQ ID NO: 10 or a fragment thereof. For example, provided herein are isolated polynucleotides of SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

The present disclosure further relates to PBV polypeptides. The PBV polypeptides may be encoded by any one of the polynucleotides provided herein. The PBV polypeptides may have the deduced amino acid sequence as provided herein, as well as fragments, analogs and derivatives of such polypeptides. The polypeptides of the present disclosure may be recombinant polypeptides, natural purified polypeptides or synthetic polypeptides. The fragment, derivative or analog of such a polypeptide may be one in which one or more of the amino acid residues is substituted with a conserved or non-conserved amino acid residue (preferably a conserved amino acid residue) and such substituted amino acid residue may or may not be one encoded by the genetic code; or it may be one in which one or more of the amino acid residues includes a substituent group; or it may be one in which the polypeptide is fused with another compound, such as a compound to increase the half-life of the polypeptide (for example, polyethylene glycol); or it may be one in which the additional amino acids are fused to the polypeptide, such as a leader or secretory sequence or a sequence which is employed for purification of the polypeptide or a proprotein sequence. Such fragments, derivatives and analogs are within the scope of the present disclosure. The polypeptides and polynucleotides of the present disclosure are provided in an isolated form, are purified or are in isolated form and purified.

Thus, a polypeptide of the present disclosure may have an amino acid sequence that is identical to that of the naturally-occurring polypeptide or that is different by minor variations due to one or more amino acid substitutions. The variation may be a “conservative change” typically in the range of about 1 to 5 amino acids, wherein the substituted amino acid has similar structural or chemical properties, e.g., replacement of leucine with isoleucine or threonine with serine. In contrast, variations may include nonconservative changes, e.g., replacement of a glycine with a tryptophan. Similar minor variations may also include amino acid deletions or insertions, or both. Guidance in determining which and how many amino acid residues may be substituted, inserted or deleted without changing biological or immunological activity may be found using computer programs well known in the art, for example, DNASTAR software (DNASTAR Inc., Madison Wis.).

The amino acid sequence of the capsid is provided in SEQ ID NO: 7. Accodingly, further provided herein are isolated polypeptides having an amino acid sequence with 80% or more sequence identity (e.g. at least 80%, 85%, 90%, 91%, 92%, 93%, 94%, 95%, 96%, 97%, 98%, 99%, or 100%) to SEQ ID NO: 7 or a fragment thereof.

The amino acid sequence of the RNA-dependent RNA polymerase (RDRP) is provided in SEQ ID NO: 11. Accodingly, further provided herein are isolated polypeptides having an amino acid sequence with 80% or more sequence identity (e.g. at least 80%, 85%, 90%, 91%, 92%, 93%, 94%, 95%, 96%, 97%, 98%, 99%, or 100%) to SEQ ID NO: 11 or a fragment thereof.

Further provided herein are isolated polypeptides having 80% or more sequence identity (e.g. at least 80%, 85%, 90%, 91%, 92%, 93%, 94%, 95%, 96%, 97%, 98%, 99%, or 100%) to the polypeptide encoded by SEQ ID NO: 1 or a fragment thereof.

Further provided herein are isolated polypeptides having 80% or more sequence identity (e.g. at least 80%, 85%, 90%, 91%, 92%, 93%, 94%, 95%, 96%, 97%, 98%, 99%, or 100%) to the polypeptide encoded by SEQ ID NO: 9 or a fragment thereof.

In some aspects, further provided herein are vectors comprising a polynucleotide as disclosed herein. Any suitable vector may be used so long as it is replicable and viable in a host. For example, in some embodiments provided herein are vectors comprising a polynucleotide having at least 50% sequence identity (e.g. 50%, 55%, 60%, 65%, 70%, 75%, 80%, 85%, 90%, 91%, 92%, 93%, 94%, 95%, 96%, 97%, 98%, 99%, or 100%) to SEQ ID NO: 1, SEQ ID NO: 6, SEQ ID NO: 9, SEQ ID NO: 10, or fragments thereof. The polynucleotides of the present disclosure may be included in any one of a variety of expression vehicles, in particular vectors or plasmids for expressing a polypeptide.

In some embodiments, the vector further comprises one or more regulatory sequences, such as a promoter. The promoer may be operably linked to the polynucleotide sequence. Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, SP6, T7, gpt, lambda P sub R, P sub L and trp. Eukaryotic promoters include cytomegalovirus (CMV) immediate early, herpes simplex virus (HSV) thymidine kinase, early and late SV40, LTRs from retrovirus, and mouse metallothionein-I. Selection of the appropriate vector and promoter is well within the level of ordinary skill in the art.

Generally, vectors will include origins of replication and selectable markers permitting transformation of a host cell, e.g., the ampicillin resistance gene of E. coli and the S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a routine matter of choice.

Useful expression vectors for bacterial use comprise a selectable marker and bacterial origin of replication derived from plasmids comprising genetic elements of the well-known cloning vector pBR322 (ATCC 37017). Other vectors include but are not limited to PKK223-3 (Pharmacia Fine Chemicals, Uppsala, Sweden) and GEM1 (Promega Biotec, Madison, Wis.). These pBR322 “backbone” sections are combined with an appropriate promoter and the structural sequence to be expressed. Such vectors include chromosomal, nonchromosomal and synthetic DNA sequences, e.g., derivatives of SV40; bacterial plasmids; phage DNA; yeast plasmids; vectors derived from combinations of plasmids and phage DNA, viral DNA such as vaccinia, adenovirus, fowl pox virus, and pseudorabies. The following vectors are provided by way of example. Bacterial: pINCY (Incyte Pharmaceuticals Inc., Palo Alto, Calif.), pSPORT1 (Life Technologies, Gaithersburg, Md.), pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK2330-3, pDR540, pRIT5 (Pharmacia). Eukaryotic: pWLneo, pSV2cat, pOG44, pXT1, pSG (Stratagene) pSVK3, pBPV, pMSG, pSVL (Pharmacia).

In some embodiments, the vector is a mammalian vector. Mammalian expression vectors will comprise an origin of replication, a suitable promoter and enhancer, and also any necessary ribosome binding sites, polyadenylation site, splice donor and acceptor sites, transcriptional termination sequences, 5′ flanking nontranscribed sequences, and selectable markers such as the neomycin phosphotransferase gene. DNA sequences derived from the SV40 viral genome, for example, SV40 origin, early promoter, enhancer, splice, and polyadenylation sites may be used to provide the required nontranscribed genetic elements. Representative, useful vectors include pRc/CMV and pcDNA3 (available from Invitrogen, San Diego, Calif.).

The desired polynucleotide may be inserted into the vector by a variety of procedures. In general, the polynucleotide is inserted into appropriate restriction endonuclease sites by procedures known in the art. Such procedures and others are deemed to be within the scope of those skilled in the art. The polynucleotide in the expression vector may be operatively linked to an appropriate expression control sequence(s) (promoter) to direct mRNA synthesis. The expression vector may also contain a ribosome binding site for translation initiation and a transcription terminator. The vector may also include appropriate sequences for amplifying expression. In addition, the expression vectors preferably contain a gene to provide a phenotypic trait for selection of transformed host cells such as dihydrofolate reductase or neomycin resistance for eukaryotic cell culture, or such as tetracycline or ampicillin resistance in E. coli.

Transcription may be increased by inserting an enhancer sequence into the vector. Enhancers are cis-acting elements of DNA, usually about from 10 to 300 bp, that acts on a protmoter increase its transcription. Examples include the SV40 enhancer on the late side of the replication origin (bp 100 to 270), a cytomegalovirus early promoter enhancer, a polyoma enhancer on the late side of the replication origin, and adenovirus enhancers.

In some embodiments, further provided herein are host cells comprising a polynucleotide or a polypeptide as described herein. In some embodiments, provided herein are host cells comprising a vector as described herein. For example, provided herein are host cells that have been transformed with a vector comprising a polynucleotide having at least 50% sequence identity (e.g. 50%, 55%, 60%, 65%, 70%, 75%, 80%, 85%, 90%, 91%, 92%, 93%, 94%, 95%, 96%, 97%, 98%, 99%, or 100%) to SEQ ID NO: 1, SEQ ID NO: 6, SEQ ID NO: 9, SEQ ID NO: 10, or fragments thereof. The vector containing the appropriate polynucleotide sequence, as well as an appropriate promoter or control sequences, may be employed to transform an appropriate host to permit the host to express a polypeptide as described herein.

In some embodiments provided herein are host cells comprising a polypeptide as described herein. For example, in some embodiments provided herein are host cells expressing a polypeptide having at least 80% sequence identity (e.g. 80%, 85%, 90%, 91%, 92%, 93%, 94%, 95%, 96%, 97%, 98%, 99%, or 100%) to SEQ ID NO: 7, SEQ ID NO: 11, or fragments thereof. In yet other embodiments provided herein are host cells expressing a polypeptide having at least 80% sequence identity to the polypeptide sequence encoded by SEQ ID NO: 1, SEQ ID NO: 9, or fragments thereof.

The host cell used herein can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (L. Davis et al., “Basic Methods in Molecular Biology”, 2nd edition, Appleton and Lang, Paramount Publishing, East Norwalk, Conn. [1994]). As representative examples of appropriate hosts, there may be mentioned: bacterial cells, such as E. coli, Salmonella typhimurium; Streptomyces sp.; fungal cells, such as yeast; insect cells such as Drosophila and Sf9; animal cells such as chinese hamster ovary (CHO), COS or Bowes melanoma; plant cells, etc. In some embodiments, the host cells is a mammalian host cell. Various mammalian cell culture systems can also be employed to express recombinant protein. Examples of mammalian expression systems include the COS-7 lines of monkey kidney fibroblasts described by Gluzman, Cell 23:175 (1981), and other cell lines capable of expressing a compatible vector, such as the C127, 3T3, CHO, HeLa and BHK cell lines. The selection of an appropriate host is deemed to be within the scope of those skilled in the art from the teachings provided herein.

The vectors in host cells can be used in a conventional manner to produce the gene product encoded by the polynucleotide sequence. Alternatively, the polypeptides of the disclosure can be synthetically produced by conventional peptide synthesizers.

Polypeptides can be expressed in mammalian cells, yeast, bacteria, or other cells under the control of appropriate promoters. Cell-free translation systems also can be employed to produce such proteins using RNAs derived from the DNA constructs of the present disclosure. Appropriate cloning and expression vectors for use with prokaryotic and eukaryotic hosts are described by Sambrook et al., Molecular Cloning: A Laboratory Manual, Second Edition, (Cold Spring Harbor, N.Y., 1989), which is hereby incorporated by reference.

Following transformation of a suitable host strain and growth of the host strain to an appropriate cell density, the selected promoter is derepressed by appropriate means (e.g., temperature shift or chemical induction), and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents; such methods are well-known to the ordinary artisan.

The PBV-derived polypeptides may be recovered and purified from cell cultures by known methods including ammonium sulfate or ethanol precipitation, acid extraction, anion or cation exchange chromatography, phosphocellulose chromatography, hydrophobic interaction chromatography, hydroxyapatite chromatography or lectin chromatography. It is preferred to have low concentrations (approximately 0.1-5 mM) of calcium ion present during purification (Price et al., J. Biol. Chem. 244:917 [1969]). Protein refolding steps can be used, as necessary, in completing configuration of the protein. Finally, high performance liquid chromatography (HPLC) can be employed for final purification steps.

The polypeptides of the present disclosure may be naturally purified products expressed from a high expressing cell line, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present disclosure may be glycosylated with mammalian or other eukaryotic carbohydrates or may be non-glycosylated. The polypeptides of the disclosure may also include an initial methionine amino acid residue.

The present disclosure further includes modified versions of the polypeptides described herein, such polypeptides comprising inactivated glycosylation sites, removal of sequences such as cysteine residues, removal of the site for proteolytic processing, and the like.

### 3. Primers and Probes

In some aspects, provided herein are primers, probes, and sets comprising the same for detecting human picobirnavirus (PBV) in a subject.

In some embodiments, provided herein are primers for amplifying PBV in a sample. In some embodiments, the primer is any suitable primer derived from SEQ ID NO: 1, SEQ ID NO: 6, SEQ ID NO: 9, SEQ ID NO: 10, or fragments thereof. In some embodiments, the primer is any suitable primer that is a complement derived from SEQ ID NO: 1, SEQ ID NO: 6, SEQ ID NO: 9, SEQ ID NO: 10, or fragments thereof. In some embodiments, the primer has 80% or more sequence identity (e.g. 80%, 85%, 90%, 91%, 92%, 93%, 94%, 95%, 96%, 97%, 98%, 99%, or 100%) to the sequence of SEQ ID NO: 13, SEQ ID NO: 14, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or complements thereof.

In some embodiments, the primer has a sequence of SEQ ID NO: 13, SEQ ID NO: 14, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or complements thereof. In some embodiments, the primer has a sequence of SEQ ID NO: 13 or a complement thereof. In some embodiments, the primer has a sequence of SEQ ID NO: 14 or a complement thereof. In some embodiments, the primer has a sequence of SEQ ID NO: 16 or a complement thereof. In some embodiments, the primer has a sequence of SEQ ID NO: 17 or a complement thereof. In some embodiments, the primer has a sequence of SEQ ID NO: 18 or a complement thereof. In some embodiments, the primer has a sequence of SEQ ID NO: 19 or a complement thereof. In some embodiments, the primer has a sequence of SEQ ID NO: 20 or a complement thereof. In some embodiments, the primer has a sequence of SEQ ID NO: 21 or a complement thereof. In some embodiments, the primer has a sequence of SEQ ID NO: 22 or a complement thereof. In some embodiments, the primer has a sequence of SEQ ID NO: 23 or a complement thereof.

In some embodiments, the primer is labeled with a detectable label. One or more primers (e.g., the one or more primers can be: (i) derived from SEQ ID NO: 1, SEQ ID NO: 6, SEQ ID NO: 9, SEQ ID NO: 10, or fragments thereof; (ii) a complement derived from SEQ ID NO: 1, SEQ ID NO: 6, SEQ ID NO: 9, SEQ ID NO: 10, or fragments thereof; or (iii) SEQ ID NO: 13, SEQ ID NO: 14, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, complements thereof) may be labeled with a detectable label.

In some aspects, provided herein are probes for detecting PBV in a sample. In some embodiments, the probe is any suitable probe derived from SEQ ID NO: 1, SEQ ID NO: 6, SEQ ID NO: 9, SEQ ID NO: 10, or fragments thereof. In some embodiments, the probe is a complement derived from SEQ ID NO: 1, SEQ ID NO: 6, SEQ ID NO: 9, SEQ ID NO: 10, or fragments thereof. In some embodiments, provided herein is a probe for detecting PBV in a sample, the probe has a sequence having 80% or more sequence identity to a sequence of SEQ ID NO: 15, SEQ ID NO: 24, SEQ ID NO: 25, SEQ ID NO: 26, SEQ ID NO: 27, SEQ ID NO: 28 or complements thereof. For example, the probe may have 80%, 85%, 90%, 91%, 92%, 93%, 94%, 95%, 96%, 97%, 98%, 99%, or 100% sequence identity to SEQ ID NO: 15, SEQ ID NO: 24, SEQ ID NO: 25, SEQ ID NO: 26, SEQ ID NO: 27, SEQ ID NO: 28 or complements thereof. In some embodiments, the probe has a sequence of SEQ ID NO: 15, SEQ ID NO: 24, SEQ ID NO: 25, SEQ ID NO: 26, SEQ ID NO: 27, SEQ ID NO: 28 or complements thereof. In some embodiments, the probe has a sequence of SEQ ID NO: 15 or a complement thereof. In some embodiments, the probe has a sequence of SEQ ID NO: 24 or a complement thereof. In some embodiments, the probe has a sequence of SEQ ID NO: 25 or a complement thereof. In some embodiments, the probe has a sequence of SEQ ID NO: 26 or a complement thereof. In some embodiments, the probe has a sequence of SEQ ID NO: 27 or a complement thereof. In some embodiments, the probe has a sequence of SEQ ID NO: 28 or a complement thereof.

In some embodiments, the probe is labeled with a detectable label. In some aspects, one or more probes can be: (i) derived from SEQ ID NO: 1, SEQ ID NO: 6, SEQ ID NO: 9, SEQ ID NO: 10, or fragments thereof; (ii) a complement derived from SEQ ID NO: 1, SEQ ID NO: 6, SEQ ID NO: 9, SEQ ID NO: 10, or fragments thereof; or (iii) SEQ ID NO: 15, SEQ ID NO: 24, SEQ ID NO: 25, SEQ ID NO: 26, SEQ ID NO: 27, SEQ ID NO: 28, or complements thereof) are labeled with a detectable label.

In some aspects, provided herein are compositions for amplifying PBV in a sample. The composition may comprise any two or more primers as disclosed herein (e.g. a primer set). In some embodiments, the composition comprises at least one forward primer having a sequence with at least 80% sequence identity (e.g., 80%, 85%, 90%, 91%, 92%, 93%, 93%, 95%, 96%, 97%, 98%, 99%, or 100%) to the sequence of SEQ ID NO: 13, SEQ ID NO: 16, SEQ ID NO: 17, SEQ ID NO: 18 or a complement thereof, and at least one reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 14, SEQ ID NO: 19, SEQ ID NO: 20, SEQ ID NO: 21, SEQ ID NO: 22, SEQ ID NO: 23 or a complement thereof.

In some embodiments, the composition comprises at least one forward primer having a sequence with at least 80% sequence identity (e.g., 80%, 85%, 90%, 91%, 92%, 93%, 93%, 95%, 96%, 97%, 98%, 99%, or 100%) to SEQ ID NO: 13 or a complement thererof and at least one reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 14 or a complement thereof.

In some embodiments, the composition comprises at least one forward primer having a sequence with at least 80% sequence identity (e.g., 80%, 85%, 90%, 91%, 92%, 93%, 93%, 95%, 96%, 97%, 98%, 99%, or 100%) to SEQ ID NO: 16, SEQ ID NO: 17, SEQ ID NO: 18, or a complement thererof and at least one reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 19, SEQ ID NO: 20, SEQ ID NO: 21, SEQ ID NO: 22, SEQ ID NO: 23, or complements thereof. In some embodiments, the composition comprises one forward primer and one reverse primer. In some embodiments, the composition comprises two or more forward primers (e.g. 2, 3, 4, 5, or more) and two or more reverse primers (e.g. 2, 3, 4, 5, or more).

In some embodiments, the composition further comprises at least one probe. The composition may further comprise any probe described herein. In some embodiments, the composition further comprises a probe having a sequence with at least 80% sequence identity (e.g., 80%, 85%, 90%, 91%, 92%, 93%, 93%, 95%, 96%, 97%, 98%, 99%, or 100%) to SEQ ID NO: 15 or a complement thereof. In some embodiments, the composition further comprises a probe having a sequence with at least 80% sequence identity (e.g., 80%, 85%, 90%, 91%, 92%, 93%, 93%, 95%, 96%, 97%, 98%, 99%, or 100%) to SEQ ID NO: 24, SEQ ID NO: 25, SEQ ID NO: 26, SEQ ID NO: 27, SEQ ID NO: 28, or complements thereof. In some embodiments, the composition comprises one probe. In some embodiments, the composition comprises two or more probes (e.g. 2, 3, 4, 5, or more).

In some aspects, provided herein are compositions for amplifying and detecting PBV in a sample. The composition may comprise any suitable combination of primers and probes described herein (e.g. a primer and probe set). In some embodiments, the composition comprises at least one forward primer, at least one reverse primer and at least one probe can be: (i) derived from SEQ ID NO: 1, SEQ ID NO: 6, SEQ ID NO: 9, SEQ ID NO: 10, or fragments thereof; or (ii) a complement derived from SEQ ID NO: 1, SEQ ID NO: 6, SEQ ID NO: 9, SEQ ID NO: 10, or fragments thereof. The composition may comprise one forward primer or more than one (e.g. 2, 3, 4, or more) forward primers. The composition may comprise one reverse primer or more than one (e.g. 2, 3, 4, 5, or more) reverse primers. The composition may comprise one probe or more than one (e.g. 2, 3, 4, 5, ore more) probes. Any or all of the at least one forward primer, at least one reverse primer and at least one probe may be labeled with one or more detectable labels.

In some embodiments, the composition comprises at least one forward primer having a sequence with at least 80% sequence identity (e.g., 80%, 85%, 90%, 91%, 92%, 93%, 93%, 95%, 96%, 97%, 98%, 99%, or 100%) to SEQ ID NO: 13 or a complement thereof, at least one reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 14 or a complement thereof, and a probe having a sequence with at least 80% sequence identity (e.g., 80%, 85%, 90%, 91%, 92%, 93%, 93%, 95%, 96%, 97%, 98%, 99%, or 100%) to SEQ ID NO: 15 or a complement thereof. For example, the composition may comprise a forward primer having the sequence of SEQ ID NO: 13 or a complement thereof, the reverse primer having the sequence of SEQ ID NO: 14 or a complement thereof, and the probe having the sequence of SEQ ID NO: 15 or a complement thereof. Such compositions would be useful for detecting the capsid of PBV. The primers and/or probes can be labeled with one or more detectable labels.

In some embodiments, the composition comprises at least one forward primer having a sequence with at least 80% sequence identity (e.g., 80%, 85%, 90%, 91%, 92%, 93%, 93%, 95%, 96%, 97%, 98%, 99%, or 100%) to SEQ ID NO: 16, SEQ ID NO: 17, SEQ ID NO: 18, or complements thereof, at least one reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 19, SEQ ID NO: 20, SEQ ID NO: 21, SEQ ID NO: 22, SEQ ID NO: 23, or complements thereof, and a probe having a sequence with at least 80% sequence identity (e.g., 80%, 85%, 90%, 91%, 92%, 93%, 93%, 95%, 96%, 97%, 98%, 99%, or 100%) to SEQ ID NO: 24, SEQ ID NO: 25, SEQ ID NO: 26, SEQ ID NO: 27, SEQ ID NO: 28, or complements thereof. The composition may comprise one forward primer or more than one (e.g. 2, 3, 4, or more) forward primers. The composition may comprise one reverse primer or more than one (e.g. 2, 3, 4, 5, or more) reverse primers. The composition may comprise one probe or more than one (e.g. 2, 3, 4, 5, ore more) probes. Such a composition would be useful for detecting the RDRP of PBV.

One or more oligonucleotide analogues can be prepared based on the primers and probes of the present disclosure. Such analogues may contain alternative structures such as peptide nucleic acids or “PNAs” (e.g., molecules with a peptide-like backbone instead of the phosphate sugar backbone of naturally occurring nucleic acids) and the like. These alternative structures are also encompassed by the primers and probes of the present disclosure. Similarly, it is understood that the primers and probes of the present disclosure may contain deletions, additions and/or substitutions of nucleic acid bases, to the extent that such alterations do not negatively affect the properties of these sequences. In particular, the alterations should not result in a significant decrease of the hybridizing properties of the primers and probes described herein. The primers and probes of the present disclosure may be prepared by any of a variety of methods known in the art (See, for example, Sambrook et al., “Molecular Cloning. A Laboratory Manual,” 1989, 2. Supp. Ed., Cold Spring Harbour Laboratory Press: New York, N.Y.; “PCR Protocols. A Guide to Methods and Applications ,” 1990, M. A. Innis (Ed.), Academic Press: New York, N.Y.; P. Tijssen “Hybridization with Nucleic Acid Probes—Laboratory Techniques in Biochemistry and Molecular Biology (Parts I and II),” 1993, Elsevier Science; “PCR Strategies,” 1995, M. A. Innis (Ed.), Academic Press: New York, N.Y.; and “Short Protocols in Molecular Biology,” 2002, F. M. Ausubel (Ed.), 5. Supp. Ed., John Wiley & Sons: Secaucus, N.J.). For example, primers and probes described herein may be prepared by chemical synthesis and polymerization based on a template as described, for example, in Narang et al., Meth. Enzymol, 1979, 68: 90-98; Brown et al., Meth. Enzymol., 1979, 68: 109-151 and Belousov et al., Nucleic Acids Res., 1997, 25: 3440-3444).

Syntheses may be performed on oligo synthesizers, such as those commercially available from Perkin Elmer/Applied Biosystems, Inc. (Foster City, Calif.), DuPont (Wilmington, Del.) or Milligen (Bedford, Mass.). Alternatively, the primers and probes of the present disclosure may be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), BioSearch Technologies, Inc. (Novato, Calif.), and many others.

Purification of the primers and probes of the present disclosure, where necessary or desired, may be carried out by any of a variety of methods well-known in the art. Purification of primers and probes can be performed either by native acrylamide gel electrophoresis, by anion-exchange HPLC as described, for example, by Pearson et al., J. Chrom., 1983, 255: 137-149 or by reverse phase HPLC (See, McFarland et al., Nucleic Acids Res., 1979, 7: 1067-1080).

As previously mentioned, modified primers and probes may be prepared using any of several means known in the art. Non-limiting examples of such modifications include methylation, substitution of one or more of the naturally occurring nucleotides with an analog, and internucleotide modifications such as, for example, those with uncharged linkages (e.g., methyl phosphonates, phosphotriesters, phosphoroamidates, carbamates, etc.), or charged linkages (e.g., phosphorothioates, phosphorodithioates, etc). Primers and probes may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., to chelate metals, radioactive metals, oxidative metals, etc), and alkylators. Primers and probes may also be derivatized by formation of a methyl or ethyl phosphotriester or an alkyl phosphoramidate linkage. Furthermore, primers and/or probes of the present disclosure may be modified with a detectable label.

As discussed briefly previously herein, in some embodiments, the primers and/or the probes may be labeled with a detectable label or moiety before being used in one or more amplification/detection methods. Preferably, for use in the methods described herein, one or more probes are labeled with a detectable label or moiety. The role of a detectable label is to allow visualization and/or detection of amplified target sequences (e.g., amplicons). Preferably, the detectable label is selected such that it generates a signal which can be measured and whose intensity is related (e.g., proportionally) to the amount of amplification product in the test sample being analyzed.

The association between one or more labeled probes and the detectable label can be covalent or non-covalent. Labeled probes can be prepared by incorporation of, or conjugation to, a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (See, for example, Mansfield et al., Mol. Cell. Probes, 1995, 9: 145-156).

Methods for labeling oligonucleotides, such as primers and/or probes, are well-known to those skilled in the art. Reviews of labeling protocols and label detection techniques can be found in, for example, L. J. Kricka, Ann. Clin. Biochem., 2002, 39: 114-129; van Gijlswijk et al, Expert Rev. Mol. Diagn., 2001, 1: 81-91; and Joos et al, J. Biotechnol., 1994, 35: 135-153. Standard nucleic acid labeling methods include: incorporation of radioactive agents, direct attachments of fluorescent dyes (See, Smith et al., Nucl. Acids Res., 1985, 13: 2399-2412) or enzymes (See, Connoly et al., Nucl. Acids. Res., 1985, 13: 4485-4502); chemical modifications of nucleic acid molecules rendering them detectable immunochemically or by other affinity reactions (See, Broker et al., Nucl. Acids Res., 1978, 5: 363-384; Bayer et al., Methods of Biochem. Analysis, 1980, 26: 1-45; Langer et al., Proc. Natl. Acad. Sci. USA, 1981, 78: 6633-6637; Richardson et al., Nucl. Acids Res., 1983, 11: 6167-6184; Brigati et al., Virol., 1983, 126: 32-50; Tchen et al., Proc. Natl. Acad. Sci. USA, 1984, 81: 3466-3470; Landegent et al., Exp. Cell Res., 1984, 15: 61-72; and A. H. Hopman et al., Exp. Cell Res., 1987, 169: 357-368); and enzyme-mediated labeling methods, such as random priming, nick translation, PCR, and tailing with terminal transferase (For a review on enzymatic labeling, see, for example, Temsamani et al., Mol. Biotechnol., 1996, 5: 223-232). Any of a wide variety of detectable labels can be used in the present disclosure.

Suitable detectable labels include, but are not limited to, various ligands, radionuclides or radioisotopes (e.g., ³²P, ³⁵S, ³H, ¹⁴C, ¹²⁵I, ¹³¹I, and the like); fluorescent dyes; chemiluminescent agents (e.g., acridinium esters, stabilized dioxetanes, and the like); spectrally resolvable inorganic fluorescent semiconductor nanocrystals (e.g., quantum dots), metal nanoparticles (e.g., gold, silver, copper and platinum) or nanoclusters; enzymes (e.g., horseradish peroxidase, beta-galactosidase, luciferase, alkaline phosphatase); colorimetric labels (e.g., dyes, colloidal gold, and the like); magnetic labels (e.g., Dynabeads™); and biotin and dioxigenin, or other haptens and proteins for antisera or monoclonal antibodies are available. In certain embodiments, the contemplated probes are fluorescently labeled.

Numerous known fluorescent labeling moieties of a wide variety of chemical structures and physical characteristics are suitable for use in the practice of this disclosure. Suitable fluorescent dyes include, but are not limited to, Quasar® dyes available from Biosearch Technologies, Novato, Calif.), fluorescein and fluorescein dyes (e.g., fluorescein isothiocyanine (FITC), naphthofluorescein, 4′,5′-dichloro-2′,7′-dimethoxy-fluorescein, 6-carboxyfluoresceins (e.g., FAM), VIC, NED, carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. Examples of other suitable fluorescent dyes that can be used and methods for linking or incorporating fluorescent dyes to oligonucleotides, such as probes, can be found in RP Haugland, “The Handbook of Fluorescent Probes and Research Chemicals”, Publisher, Molecular Probes, Inc., Eugene, Oreg. (June 1992)). Fluorescent dyes, as well as labeling kits, are commercially available from, for example, Amersham Biosciences, Inc. (Piscataway, N.J.), Molecular Probes Inc. (Eugene, Oreg.), and New England Biolabs Inc. (Beverly, Mass.). Rather than being directly detectable themselves, some fluorescent groups (donors) transfer energy to another fluorescent group (acceptor) in a process of fluorescence resonance energy transfer (FRET), and the second group produces the detectable fluorescent signal. In these embodiments, the probe may, for example, become detectable when hybridized to an amplified target sequence. Examples of FRET acceptor/donor pairs suitable for use in the present disclosure include, for example, fluorescein/tetramethylrhodamine, IAEDANS/FITC, IAEDANS/5-(iodoacetomido)fluorescein, B-phycoerythrin/Cy-5, and EDANS/Dabcyl, among others.

FRET pairs also include the use of physically-linked fluorescent reporter/quencher pairs. For example, a detectable label and a quencher moiety may be individually attached to either the 5′ end or the 3′ end of a probe, therefore placing the detectable label and the quencher moiety at opposite ends of the probe, or apart from one another along the length of the probe. During such time as the probe is not bound to its target sequence, the detectable label and quencher moiety are reversibly maintained within such proximity that the quencher blocks the detection of the detectable label. Upon binding of the probe to a target sequence, the detectable label and quencher moiety are separated thus permitting detection of the detectable label under appropriate conditions.

The use of such systems in TaqMan® assays (as described, for example, in U.S. Pat. Nos. 5,210,015, 5,804,375, 5,487,792, and 6,214,979) or as Molecular Beacons (as described, for example in, Tyagi et al, Nature Biotechnol., 1996, 14: 303-308; Tyagi et al, Nature Biotechnol, 1998, 16: 49-53; Kostrikis et al., Science, 1998, 279: 1228-1229; Sokol et al., Proc. Natl Acad. Sci. USA, 1998, 95: 11538-11543; Marras et al., Genet. Anal, 1999, 14: 151-156; and U.S. Pat. Nos. 5,846,726, 5,925,517, 6,277,581 and 6,235,504) is well-known to those skilled in the art. With the TaqMan® assay format, products of the amplification reaction can be detected as they are formed in a “real-time” manner: amplification product/probe hybrids are formed and detected while the reaction mixture is under amplification conditions.

In some embodiments of the present disclosure, the PCR detection probes are TaqMan®-like probes that are labeled at the 5′-end with a fluorescent moiety and at the 3′-end with a quencher moiety or alternatively the fluorescent moiety and quencher moiety are in reverse order, or further they may be placed along the length of the sequence to provide adequate separation when the probe hybridizes to a target sequence to allow satisfactory detection of the fluorescent moiety. Suitable fluorophores and quenchers for use with TaqMan®-like probes are disclosed in U.S. Pat. Nos. 5,210,015, 5,804,375, 5,487,792, and 6,214,979, and WO 01/86001. Examples of quenchers include, but are not limited, to DABCYL (e.g., 4-(4′-dimethylaminophenylazo)-benzoic acid) succinimidyl ester, diarylrhodamine carboxylic acid, succinimidyl ester (or QSY-7), and 4′,5′-dinitrofluorescein carboxylic acid, succinimidyl ester (or QSY-33) (all of which are available from Molecular Probes (which is part of Invitrogen, Carlsbad, Calif.)), quencher 1 (Q1; available from Epoch Biosciences, Bothell, Wash.), or “Black hole quenchers” BHQ-I, BHQ-2, and BHQ-3 (available from BioSearch Technologies, Inc., Novato, Calif.). In certain embodiments, the PCR detection probes are TaqMan®-like probes that are labeled at the 5′ end with FAM and at the 3′ end with a Black Hole Quencher® or Black Hole Quencher® plus (Biosearch Technologies, Novato, Calif.).

A “tail” of normal or modified nucleotides can also be added to probes for detectability purposes. A second hybridization with nucleic acid complementary to the tail and containing one or more detectable labels (such as, for example, fluorophores, enzymes, or bases that have been radioactively labeled) allows visualization of the amplicon/probe hybrids.

The selection of a particular labeling technique may depend on the situation and may be governed by several factors, such as the ease and cost of the labeling method, spectral spacing between different detectable labels used, the quality of sample labeling desired, the effects of the detectable moiety on the hybridization reaction (e.g., on the rate and/or efficiency of the hybridization process), the nature of the amplification method used, the nature of the detection system, the nature and intensity of the signal generated by the detectable label, and the like.

### 4. Methods of Detecting PBV

In some aspects, provided herein are methods of detecting PBV in a sample.

In some embodiments, provided herein are methods of detecting PBV in a sample, comprising contacting the sample with at least one primer and/or at least one probe. In some embodiments, the methods are performed using PCR. In some embodiments, the methods are performed using fluorescence in-situ hybridization (FISH). For example, the primer(s) and/or probe(s) may be suitable for PCR or FISH techniques. The at least one primer and/or the at least one probe may be labeled with at least one detectable label. In some embodiments, the PBV comprises the sequence of SEQ ID NO: 1, SEQ ID NO: 6, SEQ ID NO: 7, SEQ ID NO: 9, SEQ ID NO: 10, SEQ ID NO: 11, or a combination thereof.

The methods comprise contacting the sample with any suitable combination of primers and probes as described herein. The present disclosure provides methods for detecting the presence of PBV in a test sample. Further, PBV levels may be quantified per test sample by comparing test sample detection values against standard curves generated using serial dilutions of previously quantified suspensions of one or more PBV sequences or other standardized PBV profiles.

In some embodiments, the method comprises contacting the sample with a composition described herein. For example, the method may comprise contacting the sample with a primer and probe set described herein. For example, the method may comprise contacting the sample with at least one forward primer, at least one reverse primer, and at least one probe can be: (i) derived from SEQ ID NO: 1, SEQ ID NO: 6, SEQ ID NO: 9, SEQ ID NO: 10, or fragments thereof; or (ii) a complement derived from SEQ ID NO: 1, SEQ ID NO: 6, SEQ ID NO: 9, SEQ ID NO: 10, or fragments thereof. Any or all of the at least one forward primer, at least one reverse primer and at least one probe may be labeled with one or more detectable labels.

In some embodiments, the method may comprise contacting the sample with a primer and probe set suitable for detecting the capsid of PBV. For example, the method may comprise contacting the sample with a forward primer having a sequence with at least 80% sequence identity (e.g., 80%, 85%, 90%, 91%, 92%, 93%, 93%, 95%, 96%, 97%, 98%, 99%, or 100%) to SEQ ID NO: 13 or a complement thereof, a reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 14 or a complement thereof, and a probe having a sequence with at least 80% sequence identity (e.g., 80%, 85%, 90%, 91%, 92%, 93%, 93%, 95%, 96%, 97%, 98%, 99%, or 100%) to SEQ ID NO: 15 or a complement thereof.

In some embodiments, the method comprises contacting the sample with a primer and probe set suitable for detecting the RDRP of PBV. For example, the method may comprise contacting the sample with at least one forward primer having a sequence with at least 80% sequence identity (e.g., 80%, 85%, 90%, 91%, 92%, 93%, 93%, 95%, 96%, 97%, 98%, 99%, or 100%) to SEQ ID NO: 16, SEQ ID NO: 17, SEQ ID NO: 18, or complements thereof, at least one reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 19, SEQ ID NO: 20, SEQ ID NO: 21, SEQ ID NO: 22, SEQ ID NO: 23, or complements thereof, and a probe having a sequence with at least 80% sequence identity (e.g., 80%, 85%, 90%, 91%, 92%, 93%, 93%, 95%, 96%, 97%, 98%, 99%, or 100%) to SEQ ID NO: 24, SEQ ID NO: 25, SEQ ID NO: 26, SEQ ID NO: 27, SEQ ID NO: 28, or complements thereof. The method may comprise contacting the sample with one forward primer or more than one (e.g. 2, 3, 4, or more) forward primers. The method may comprise contacting the sample with one reverse primer or more than one (e.g. 2, 3, 4, 5, or more) reverse primers. The method may comprise contacting the sample with one probe or more than one (e.g. 2, 3, 4, 5, ore more) probes.

In some embodiments, methods for detecting PBV in a sample comprise contacting the sample with at least one forward primer and at least one reverse primer under amplification conditions to generate a first target sequence, and detecting hybridization between the first target sequence and fat least one probe as an indication of the presence of PBV in the sample. The amplification conditions may comprise submitting the sample to an amplification reaction carried out in the presence of suitable amplification reagents. In some embodiments, the amplification reaction comprises PCR, real-time PCR, or reverse-transcriptase PCR.

The use of primers or primer sets of the present disclosure to amplify PBV target sequences in test samples is not limited to any particular nucleic acid amplification technique or any particular modification thereof. In fact, the primers and primer sets of the present disclosure can be employed in any of a variety of nucleic acid amplification methods that are known in the art (See, for example, Kimmel et al., Methods Enzymol., 1987, 152: 307-316; Sambrook et al., “Molecular Cloning. A Laboratory Manual”, 1989, 2.Supp. Ed., Cold Spring Harbour Laboratory Press: New York, N.Y.; “Short Protocols in Molecular Biology”, F. M. Ausubel (Ed.), 2002, 5. Supp. Ed., John Wiley & Sons: Secaucus, N.J.).

Such nucleic acid amplification methods include, but are not limited to, the Polymerase Chain Reaction (PCR). PCR is described in a number of references, such as, but not limited to, “PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR. A Practical Approach”, McPherson et al. (Eds.), 1991, IRL Press: Oxford; Saiki et al., Nature, 1986, 324: 163; and U.S. Pat. Nos. 4,683,195, 4,683,202 and 4,889,818. Variations of PCR including, TaqMan®-based assays (See, Holland et al., Proc. Natl. Acad. Sci., 1991, 88: 7276-7280), and reverse transcriptase polymerase chain reaction (or RT-PCR, described in, for example, U.S. Pat. Nos. 5,322,770 and 5,310,652) are also included.

Generally, in PCR, a pair of primers is added to a test sample obtained from a subject (and thus contacted with the test sample) in excess to hybridize to the complementary strands of the target nucleic acid. The primers are each extended by a DNA polymerase using the target sequence as a template. The extension products become targets themselves after dissociation (denaturation) from the original target strand. New primers are then hybridized and extended by the polymerase, and the cycle is repeated to exponentially increase the number of amplicons. Examples of DNA polymerases capable of producing primer extension products in PCR reactions include, but are not limited to, E. coli DNA polymerase I, Klenow fragment of DNA polymerase I, T4 DNA polymerase, thermostable DNA polymerases isolated from Thermus aquaticus (Taq), available from a variety of sources (e.g., Perkin Elmer, Waltham, Mass.), Thermus thermophilus (USB Corporation, Cleveland, Ohio), Bacillus stereothermophilus (Bio-Rad Laboratories, Hercules, Calif.), AmpliTaq Gold® Enzyme (Applied Biosystems, Foster City, Calif.), recombinant Thermus thermophilus (rTth) DNA polymerase (Applied Biosystems, Foster City, Calif.) or Thermococcus litoralis (“Vent” polymerase, New England Biolabs, Ipswich, Mass.). RNA target sequences may be amplified by first reverse transcribing (RT) the mRNA into cDNA, and then performing PCR (RT-PCR), as described above. Alternatively, a single enzyme may be used for both steps as described in U.S. Pat. No. 5,322,770.

In addition to the enzymatic thermal amplification methods described above, isothermal enzymatic amplification reactions can be employed to amplify PBV sequences using primers and primer sets of the present disclosure (Andras et al., Mol. Biotechnol., 2001, 19: 29-44). These methods include, but are not limited to, Transcription-Mediated Amplification (TMA; TMA is described in Kwoh et al., Proc. Natl. Acad. ScL USA, 1989, 86: 1173-1177; Giachetti et al., J. Clin. Microbiol, 2002, 40: 2408-2419; and U.S. Pat. No. 5,399,491); Self-Sustained Sequence Replication (3SR; 3SR is described in Guatelli et al., Proc. Natl. Acad. Sci. USA, 1990, 87: 1874-1848; and Fahy et al., PCR Methods and Applications, 1991, 1: 25-33); Nucleic Acid Sequence Based Amplification (NASBA; NASBA is described in, Kievits et al., J. Virol. Methods, 1991, 35: 273-286; and U.S. Pat. No. 5,130,238) and Strand Displacement Amplification (SDA; SDA is described in Walker et al., PNAS, 1992, 89: 392-396; EP 0 500 224 A2).

In certain embodiments of the present disclosure, the probes described herein are used to detect amplification products generated by the amplification reaction. The probes described herein may be employed using a variety of well-known homogeneous or heterogeneous methodologies.

Homogeneous detection methods include, but are not limited to, the use of FRET labels that are attached to the probes and that emit a signal in the presence of the target sequence, Molecular Beacons (See, Tyagi et al., Nature Biotechnol., 1996, 14: 303-308; Tyagi et al., Nature Biotechnol, 1998, 16: 49-53; Kostrikis et al., Science, 1998, 279: 1228-1229; Sokol et al., Proc. Natl. Acad. Sci. USA, 1998, 95: 11538-11543; Marras et al., Genet. Anal, 1999, 14: 151-156; and U.S. Pat. Nos. 5,846,726, 5,925,517, 6,277,581 and 6,235,504), and the TaqMan® assays (See, U.S. Pat. Nos. 5,210,015; 5,804,375; 5,487,792 and 6,214,979 and WO 01/86001). Using these detection techniques, products of the amplification reaction can be detected as they are formed, namely, in a real time manner. As a result, amplification product/probe hybrids are formed and detected while the reaction mixture is under amplification conditions.

In certain embodiments, the probes of the present disclosure are used in a TaqMan® assay. In a TaqMan® assay, analysis is performed in conjunction with thermal cycling by monitoring the generation of fluorescence signals. The assay system has the capability of generating quantitative data allowing the determination of target copy numbers. For example, standard curves can be generated using serial dilutions of previously quantified suspensions of one or more PBV sequences, against which unknown samples can be compared. The TaqMan® assay is conveniently performed using, for example, AmpliTaq Gold™ DNA polymerase, which has endogenous 5′ nuclease activity, to digest a probe labeled with both a fluorescent reporter dye and a quencher moiety, as described above. Assay results are obtained by measuring changes in fluorescence that occur during the amplification cycle as the probe is digested, uncoupling the fluorescent and quencher moieties and causing an increase in the fluorescence signal that is proportional to the amplification of the target sequence.

Other examples of homogeneous detection methods include hybridization protection assays (HPA). In such assays, the probes are labeled with acridinium ester (AE), a highly chemiluminescent molecule (See, Weeks et al, Clin. Chem., 1983, 29: 1474-1479; Berry et al., Clin. Chem., 1988, 34: 2087-2090), using a non-nucleotide-based linker arm chemistry (See, U.S. Pat. Nos. 5,585,481 and 5,185,439). Chemiluminescence is triggered by AE hydrolysis with alkaline hydrogen peroxide, which yields an excited N-methyl acridone that subsequently deactivates with emission of a photon. In the absence of a target sequence, AE hydrolysis is rapid. However, the rate of AE hydrolysis is greatly reduced when the probe is bound to the target sequence. Thus, hybridized and un-hybridized AE-labeled probes can be detected directly in solution without the need for physical separation.

Heterogeneous detection systems are also well-known in the art and generally employ a capture agent to separate amplified sequences from other materials in the reaction mixture. Capture agents typically comprise a solid support material (e.g., microtiter wells, beads, chips, and the like) coated with one or more specific binding sequences. A binding sequence may be complementary to a tail sequence added to oligonucleotide probes of the disclosure. Alternatively, a binding sequence may be complementary to a sequence of a capture oligonucleotide, itself comprising a sequence complementary to a tail sequence of a probe. After separation of the amplification product/probe hybrids bound to the capture agents from the remaining reaction mixture, the amplification product/probe hybrids can be detected using any detection methods, such as those described herein.

In some embodiments, the methods further comprise administering an appropriate therapy to the subject if PBV is detected in the sample. For example, the method may further comprise administering an appropriate anti-viral agent to the subject if PBV is detected in the sample.

### 5. Kits

In another embodiment, the present disclosure provides kits including materials and reagents useful for the detection of PBV according to methods described herein. The description of the primers, probes, and compositions herein are also applicable to those same aspects of the methods for detecting PBV described herein. The kits can be used by diagnostic laboratories, experimental laboratories, or practitioners. In certain embodiments, the kits comprise at least one of the primer sets or primer and probe sets described in herein and optionally, amplification reagents. Each kit preferably comprises amplification reagents for a specific amplification method. Thus, a kit adapted for use with NASBA preferably contains primers with an RNA polymerase promoter linked to the target binding sequence, while a kit adapted for use with SDA preferably contains primers including a restriction endonuclease recognition site 5′ to the target binding sequence. Similarly, when the kit is adapted for use in a 5′ nuclease assay, such as the TaqMan® assay, the probes of the present disclosure can contain at least one fluorescent reporter moiety and at least one quencher moiety.

In some embodiments, the kit comprises at least one forward primer, at least one reverse primer, at least one probe, and amplification reagents and instructions for amplifying and detecting PBV in a sample. Any of the primers and/or probe contained in kit may comprise a detectable label.

In some embodiments, the kit comprises at least one forward primer, at least one reverse primer, and at least one probe can be: (i) derived from SEQ ID NO: 1, SEQ ID NO: 6, SEQ ID NO: 9, SEQ ID NO: 10, or fragments thereof; or (ii) a complement derived from SEQ ID NO: 1, SEQ ID NO: 6, SEQ ID NO: 9, SEQ ID NO: 10, or fragments thereof. The kit may comprise one forward primer or more than one (e.g. 2, 3, 4, or more) forward primers. The kit may comprise one reverse primer or more than one (e.g. 2, 3, 4, 5, or more) reverse primers. The kit may comprise one probe or more than one (e.g. 2, 3, 4, 5, or more) probes. Any one or more primers and/or probes may be labeled with a detectable label.

In some embodiments, the kit comprises at least one forward primer having 80% or more sequence identity (e.g. 80%, 85%, 90%, 91%, 92%, 93%, 94%, 95%, 96%, 97%, 98%, 99%, or 100%) to SEQ ID NO: 13 or a complement thereof, and at least one reverse primer having 80% or more (e.g. a reverse primer (i) derived from SEQ ID NO: 1, SEQ ID NO: 6, SEQ ID NO: 9, SEQ ID NO: 10, or fragments thereof; (ii) complement derived from from SEQ ID NO: 1, SEQ ID NO: 6, SEQ ID NO: 9, SEQ ID NO: 10, or fragments thereof; or (iii) having 80%, 85%, 90%, 91%, 92%, 93%, 94%, 95%, 96%, 97%, 98%, 99%, or 100%) sequence identity to SEQ ID NO: 14 or a complement thereof. In some embodiments, the kit may further comprise at least one probe having 80% or more sequence identity (e.g. a probe (i) derived from SEQ ID NO: 1, SEQ ID NO: 6, SEQ ID NO: 9, SEQ ID NO: 10, or fragments thereof; (ii) complement derived from from SEQ ID NO: 1, SEQ ID NO: 6, SEQ ID NO: 9, SEQ ID NO: 10, or fragments thereof; or (iii) having 80%, 85%, 90%, 91%, 92%, 93%, 94%, 95%, 96%, 97%, 98%, 99%, or 100%) to SEQ ID NO: 15 or a complement thereof. Any one or more primers and/or probe may be labeled with a detectable label. The kit may comprise one forward primer or more than one (e.g. 2, 3, 4, or more) forward primers. The kit may comprise one reverse primer or more than one (e.g. 2, 3, 4, 5, or more) reverse primers. The kit may comprise one probe or more than one (e.g. 2, 3, 4, 5, or more) probes. Any one or more primers and/or probes may be labeled with a detectable label.

In some embodiments, the kit comprises at least one forward primer having a sequence with at least 80% sequence identity (e.g., 80%, 85%, 90%, 91%, 92%, 93%, 93%, 95%, 96%, 97%, 98%, 99%, or 100%) to SEQ ID NO: 16, SEQ ID NO: 17, SEQ ID NO: 18, or complements thereof, at least one reverse primer having a sequence with at least 80% sequence identity (e.g., 80%, 85%, 90%, 91%, 92%, 93%, 93%, 95%, 96%, 97%, 98%, 99%, or 100%) to SEQ ID NO: 19, SEQ ID NO: 20, SEQ ID NO: 21, SEQ ID NO: 22, SEQ ID NO: 23, or complements thereof. The kit may further comprise at least one probe having a sequence with at least 80% sequence identity (e.g., 80%, 85%, 90%, 91%, 92%, 93%, 93%, 95%, 96%, 97%, 98%, 99%, or 100%) to SEQ ID NO: 24, SEQ ID NO: 25, SEQ ID NO: 26, SEQ ID NO: 27, SEQ ID NO: 28, or complements thereof. The kit may comprise one forward primer or more than one (e.g. 2, 3, 4, or more) forward primers. The kit may comprise one reverse primer or more than one (e.g. 2, 3, 4, 5, or more) reverse primers. The kit may comprise one probe or more than one (e.g. 2, 3, 4, 5, ore more) probes. Any one or more primers and/or probes may be labeled with a detectable label.

Suitable amplification reagents additionally include, for example, one or more of: buffers, reagents, enzymes having reverse transcriptase and/or polymerase activity or exonuclease activity, enzyme cofactors such as magnesium or manganese; salts; deoxynucleotide triphosphates (dNTPs) suitable for carrying out the amplification reaction. Depending on the procedure, kits may further comprise one or more of: wash buffers, hybridization buffers, labeling buffers, detection means, and other reagents. The buffers and/or reagents are preferably optimized for the particular amplification/detection technique for which the kit is intended. Protocols for using these buffers and reagents for performing different steps of the procedure may also be included in the kit. Furthermore, kits may be provided with an internal control as a check on the amplification efficiency, to prevent occurrence of false negative test results due to failures in the amplification, to check on cell adequacy, sample extraction, etc. An optimal internal control sequence is selected in such a way that it will not compete with the target nucleic acid sequence in the amplification reaction. Such internal control sequences are known in the art. Kits may also contain reagents for the isolation of nucleic acids from test samples prior to amplification before nucleic acid extraction.

The reagents may be supplied in a solid (e.g., lyophilized) or liquid form. Kits of the present disclosure may optionally comprise different containers (e.g., vial, ampoule, test tube, flask, or bottle) for each individual buffer and/or reagent. Each component will generally be suitable as aliquoted in its respective container or provided in a concentrated form. Other containers suitable for conducting certain steps of the amplification/detection assay may also be provided. The individual containers are preferably maintained in close confinement for commercial sale.

Kits may also comprise instructions for using the amplification reagents and primer sets or primer and probe described herein: for processing the test sample, extracting nucleic acid molecules, and/or performing the test; and for interpreting the results obtained as well as a notice in the form prescribed by a governmental agency. Such instructions optionally may be in printed form or on CD, DVD, or other format of recorded media. By way of example, and not of limitation, examples of the present disclosures shall now be given.

The present disclosure has multiple aspects, illustrated by the following non-limiting examples.

### EXAMPLE 1

### Discovery of a Novel Picobirnavirus

Samples: A panel of 24 samples were sourced from MRN Diagnostics, consisting of sputum, bronchial alveolar lavages (BAL), and endotracheal aspirates (ETA). Patients providing sputum were confirmed to be hospitalized and ill with respiratory symptoms. The study participants were enrolled at a site in Colombia, South America drawing from individuals in 4 different cities as shown in Table 1.

TABLE 1

| | | | | | | | | STORAGE | | | | | |
|---|---|---|---|---|---|---|---|---|---|---|---|---|---|
| | | | | | | | | TEMP | | | | | SHIPPED |
| SAMPLE | DONOR | | GEN- | COUNTRY | | COLLECTION | SAMPLE | DEGREES | NUMBER OF | | | | VOLUME |
| ID | ID | AGE | DER | OF ORIGIN | CITY | DATE | TYPE | CELCIUS | ALIQUOTS | | | | (ML) |
| 0 [symbol] | 5 [symbol] 18 | [symbol] | F | COLOMBIA | BARRANQUILLA | Nov. 16, 2016 | BAL | [symbol] 20 C. | 1 | x | 1 | ml | 1 |
| 0 [symbol] 0 [symbol] 3 [symbol] | 8140307 | 50 | M | COLOMBIA | CUCUTA | Aug. 14, 2016 | Sputum | [symbol] 20 C. | 1 | x | 1 | ml | 1 |
| 0 [symbol] 0 [symbol] 340 [symbol] | [symbol] 78 [symbol] | 74 | M | COLOMBIA | BARRANQUILLA | Oct. 11, 2016 | Sputum | [symbol] 20 C. | 1 | x | 2 | ml | 2 |
| 0 [symbol] 0 [symbol] 3406 | 578 [symbol] | [symbol] | M | COLOMBIA | BARRANQUILLA | Oct. 11, 2016 | Sputum | [symbol] 20 C. | 1 | x | 1 | ml | 1 |
| 0 [symbol] 4 [symbol] | 51673 | 51 | F | COLOMBIA | BARRANQUILLA | Jan. 10, 2017 | BAL | [symbol] 20 C. | 1 | x | 1 | ml | 1 |
| 08 [symbol] 64337 | 51674 | 6 [symbol] | F | COLOMBIA | BARRANQUILLA | Jan. 8, 2017 | BAL | [symbol] 20 C. | 1 | x | 3 | ml | 3 |
| 0 [symbol] | 518 [symbol] 8 | [symbol] 1 | M | COLOMBIA | BARRANQUILLA | Feb. 9, 2017 | Sputum | [symbol] 20 C. | 1 | x | 1 | ml | 1 |
| 08 [symbol] 4 [symbol] 44 | 51873 | [symbol] | M | COLOMBIA | BARRANQUILLA | Feb. 9, 2017 | Sputum | [symbol] 20 C. | 1 | x | 1 | ml | 1 |
| 0 [symbol] 064241 | 51888 | 46 | F | COLOMBIA | BARRANQUILLA | Feb. 9, 2017 | Sputum | [symbol] 20 C. | 1 | x | 2 | ml | 2 |
| 088 [symbol] 4 [symbol] 42 | 423 | 91 | M | COLOMBIA | Medellin | Dec, 2016 | Sputum | [symbol] 20 C. | 1 | x | 1 | ml | 1 |
| 08 [symbol] 64 [symbol] 40 | [symbol] 1 | 77 | F | COLOMBIA | Medellin | Jan, 2017 | Sputum | [symbol] 20 C. | 1 | x | 1 | ml | 1 |
| 08 [symbol] 4338 | [symbol] 4 | [symbol] | F | COLOMBIA | Medellin | Jan, 2017 | endotracheal | [symbol] 20 C. | 1 | x | 2 | ml | 2 |
| | | | | | | | aspirate | | | | | | |
| 08 [symbol] 64 [symbol] | 545 | [symbol] | F | COLOMBIA | Medellin | Jan, 2017 | endotracheal | [symbol] 20 C. | 1 | x | 5 | ml | 5 |
| | | | | | | | aspirate | | | | | | |
| 0 [symbol] 64 [symbol] 42 | 550 | 72 | M | COLOMBIA | Medellin | Jan, 2017 | endotracheal | [symbol] 20 C. | 1 | x | 2 | ml | 2 |
| | | | | | | | aspirate | | | | | | |
| 08 [symbol] 64242 | 51941 | 48 | M | COLOMBIA | BARRANQUILLA | Feb. 16, 2017 | Sputum | [symbol] 20 C. | 1 | x | 1 | ml | 1 |
| 08 [symbol] 6424 [symbol] | 51945 | [symbol] | M | COLOMBIA | BARRANQUILLA | Feb. 16, 2017 | Sputum | [symbol] 20 C. | 1 | x | 1 | ml | 1 |
| 08 [symbol] 42 [symbol] | 5195 [symbol] | 48 | M | COLOMBIA | BARRANQUILLA | Feb. 16, 2017 | Sputum | [symbol] 20 C. | 1 | x | 1 | ml | 1 |
| 08 [symbol] 6427 [symbol] | 51978 | 4 [symbol] | F | COLOMBIA | BARRANQUILLA | Feb. 16, 2017 | Sputum | [symbol] 20 C. | 1 | x | 2 | ml | 2 |
| 08 [symbol] 43 [symbol] 2 | 52051 | 23 | M | COLOMBIA | BARRANQUILLA | Feb. 20, 2017 | Sputum | [symbol] 20 C. | 1 | x | 1 | ml | 1 |
| 0886436 [symbol] | 52064 | 74 | M | COLOMBIA | VALLEDUPAR | Feb. 16, 2017 | Sputum | [symbol] 20 C. | 1 | x | 3 | ml | 3 |
| 088 [symbol] 436 [symbol] | 52065 | 74 | M | COLOMBIA | VALLEDUPAR | Feb. 16, 2017 | Sputum | [symbol] 20 C. | 1 | x | 3 | ml | 3 |
| 0886439 [symbol] | 52094 | 4 [symbol] | F | COLOMBIA | BARRANQUILLA | Feb. 21, 2017 | BAL | [symbol] 20 C. | 1 | x | 3 | ml | 3 |
| 0886441 [symbol] | 52117 | [symbol] 4 | M | COLOMBIA | BARRANQUILLA | Feb. 19, 2017 | BAL | [symbol] 20 C. | 1 | x | 2.5 | ml | 2.5 |
| 0886441 [symbol] | 52118 | 73 | M | COLOMBIA | BARRANQUILLA | Feb. 19, 2017 | BAL | [symbol] 20 C. | 1 | x | 3.5 | ml | 3.5 |

[symbol]

indicates data missing or illegible when filed

Extraction: Sputum samples (n=15) were pre-treated with a cocktail of nucleases and physically disrupted using disposable pestles. Total nucleic acid was extracted on the automated m2000sp (Abbott Molecular).

Library prep: Nucleic acid was converted to cDNA and barcoded Nextera libraries.

mNGS sequencing: Two sets of libraries were sequenced. Library concentrations and MiSeq run metrics were as follows:

### Run 1:

MLP 0% Phix

| | | Calibrated | Peak | | | |
|---|---|---|---|---|---|---|
| Size | Conversion | Conc. | Molarity | | | |
| [bp] | factor | [ng/μl] | [nmol/l] | Loaded [ ] | Cluster/PF | Flow Cell |
| 230 | 0.1518 | 0.197 | 1.30 | 14 pM | 607 K/82% | C3DTN |

### Run 2:

| MLP 0% | | Calibrated | Peak | | | |
|---|---|---|---|---|---|---|
| PhixSize | Conversion | Conc. | Molarity | | | |
| [bp] | factor | [ng/μl] | [nmol/l] | Loaded [ ] | Cluster/PF | Flow Cell |
| 220 | 0.1452 | 0.23 | 1.58 | 14 pM | 703 K/86.4% | C3DTJ |

Summary of mNGS results: Below is a brief summary of the pathogens that were found to be enriched/present in the samples and suspected to play a role in the respiratory illness. NGS reads were analyzed by SURPI (Naccache, et al 2104) and an Abbott data analysis pipeline named DiVir. Notable and perhaps expected of gram negative enterobacteria with known roles in nosocomial infections, including respiratory infections, there were >10K reads found in ˜20% (3/14) patients. Rather surprising, however, was the presence of Aichivirus A in sample #9-4352: this is a picornavirus causing gastroenteritis, for which 80% of the genome by was obtained by mNGS. HHV-1 has been observed in respiratory infections, particularly in the immunocompromised. Other viruses were detected at low levels making it difficult to argue for causality, but they are noted below, with read numbers in parenthesis.

| Sample | Pathogen(s) detected (# of viral reads) |
|---|---|
| 1-Sputum-4279 | Burkholderia cenocepacia |
| 2-Sputum-3406 | Porcine picbirnavirus-3 (2) |
| 3-Sputum-4940 | Influenza A (8) |
| 4-Sputum-4942 | — |
| 5-Sputum-4242 | Citrobacter sp, Klebsiella sp |
| 6-Sputum-4246 | — |
| 7-Sputum-4256 | Citrobacter sp, Klebsiella sp, Acinetobacter sp |
| 8-Sputum-3405_CtRL9 | Klebsiella sp, Acinetobacter sp, HHV-4 (15) |
| 8-Sputum-4366 | HHV-4 (79) |
| 9-Sputum-4352 | Aichivirus A (65), HHV-4 (42), HHV-7 (61) |
| 10-Sputum-4946 | — |
| 11-Sputum-4938 | Stenotrophomonas sp, HHV-1 (6045) |
| 12-Sputum-4947 | HHV-1 (30) |
| 13-Sputum-4395 | — |
| 14-Sputum-4418 | — |
| 15-Sputum-4419 | — |

MRN3406: Sample #2 was enriched for Pasteurellaceae family bacteria, such as Haemophilus parainfluenzae and Haemophilus influenzae, but <10K reads were observed for other bacteria in other patients. H. parainfluenzae is normal flora of the respiratory tract, but is an opportunistic pathogen that has been associated with endocarditis, bronchitis, otitis, conjunctivitis, pneumonia, abscesses and genital tract infections.

Divergent picobirnavirus reads were identified among reads without a match in NT in sample MRN3406.

| | | | | | | | | | | | | |
|---|---|---|---|---|---|---|---|---|---|---|---|---|
| | | | | | | | [symbol] query | | | | | |
| bit | | align | [symbol] | [symbol] | | gap | coverage | subject | | | subject | subject |
| score | evalue | length | id | pos | [symbol] | opens | per hsp | tax ids | subject titles | virus | start | end |

[symbol]

Query: M00641:16:000000000-C3DIN:1:2107:23091:15563 [symbol] 2/2:F1

| | | | | | | | | | | | | |
|---|---|---|---|---|---|---|---|---|---|---|---|---|
| 107 | 9.88E−29 | 50 | 70 | 88 | 15 | 0 | 100 | 145856 | RNA-dependent | Human | 422 | 471 |
| | | | | | | | | | RNA polymerase | picobirnavirus | | |
| 105 | 4.01E−28 | 50 | 70 | 80 | 15 | 0 | 100 | 442302 | RNA-dependent | Porcine | 428 | 477 |
| | | | | | | | | | RNA polymerase | picobirnavirus | | |

[symbol]

Query: contig_4566_161_394:F1

| | | | | | | | | | | | | |
|---|---|---|---|---|---|---|---|---|---|---|---|---|
| 106 | 3. [symbol] 4E−28 | 53 | 37.7 | 64.2 | 33 | 0 | 100 | 442302 | capsid protein | Porcine | 183 | 235 |
| | | | | | | | | | | picobirnavirus | | |
| 104 | 9.16 [symbol] E−28 | 53 | 43.4 | 58.5 | 30 | 0 | 100 | 145856 | hypothetical protein | Human | 181 | 233 |
| | | | | | | | | | HPV_s1gp2 | picobirnavirus | | |

[symbol]

indicates data missing or illegible when filed

There were 2 porcine picobirnavirus-3 reads detected by SNAP to nt (SURPI). This was investigated further since there were also related PBV reads detected in RAPsearch (SURPI) and DiVir 2.0 data.

| SAMPLE | DONOR | | | COUNTRY | | COLLECTION | SAMPLE |
|---|---|---|---|---|---|---|---|
| ID | ID | AGE | GENDER | OF ORIGIN | CITY | DATE | TYPE |
| 08853406 | S789 | 29 | M | COLUMBIA | BARRANQUILLA | Oct. 11, 2016 | Sputum |

This sample was obtained from a 24-year-old male hospitalized in Colombia in October of 2016 for respiratory illness. The summary table below illustrates that hits to picobirnavirus were detected in all of our divergent virus prediction algorithms. Notable is that contigs were formed that produced extended reads. After this first MiSeq run, >50% of the sequence was assembled, with reads mapping throughout the genome and to each protein. Only 462,336 total reads were obtained for the MRN3406 sample in this initial run.

| | Haemophilus parainfluenzae, Neisseria |
|---|---|
| | meningitidis, Haemophilus |
| Bacteria | influenzae, Neisseria gonorrhoeae |
| SNAP to virus | Porcine picobirnavirus 3 |
| RAPsearch viral in nr | Human picobirnavirus, Fox picobirnavirus, |
| | Otarine picobirnavirus |
| RAPsearch not in nr | Human picobirnavirus, Fox picobirnavirus, |
| | Otarine picobirnavirus |
| ARM1 virus to nt virus | Human picobirnavirus, Fox picobirnavirus, |
| | Otarine picobirnavirus |
| ARM2 psiBLAST 49dn | Human picobirnavirus, Fox picobirnavirus, |
| | Otarine picobirnavirus |
| ARM2 psiBLAST 50-84 | Human picobirnavirus, Fox picobirnavirus, |
| | Otarine picobirnavirus |
| Extended reads | Human picobirnavirus, Fox picobirnavirus, |
| | Otarine picobirnavirus |

Examples of hits detected by RAPsearch: The very low (negative) expect (e) values and high Bit scores indicate high confidence protein matches to the virus species listed.

| | | | E- | | |
|---|---|---|---|---|---|
| Qlen | Mismatch | Gaps | value | Bit score | Species |
| 45 | 9 | 0 | −12.8 | 81.2629 | Fox picobirnavirus |
| 50 | 16 | 0 | −11.86 | 78.1814 | Human picobirnavirus |
| 43 | 14 | 0 | −7.34 | 63.1586 | Otarine picobirnavirus |

Examples of hits detected by DiVir: The very low e-values and long query lengths indicate high confidence protein matches to the virus species listed.

### ARM1 (BLASTn):

| Qlen | E-value | Species |
|---|---|---|
| 178 | 8.88E−43 | Human picobirnavirus RNA segment 2, complete |
| | | sequence |
| 59 | 1.61E−07 | Porcine picobirnavirus strain segment S, complete |
| | | sequence |

ARM2 (psiBLAST): Bit scores were >100 for most hits, with e-values<10-24. Note that strong hits to both the capsid and the RDRP are detected.

| bit | | | |
|---|---|---|---|
| score | evalue | Species Association | Species |
| 101 | 3.61E−26 | capsid protein | Porcine picobirnavirus] |
| 128 | 1.24E−35 | RNA-dependent RNA | Human picobirnavirus] |
| | | polymerase | |

Resequencing: The MRN3406 library was re-sequenced on 2 separate runs and each time fewer than expected reads were obtained. Regardless, these additional datasets allowed 95% of the genome to be completed. The final gap in RDRP was filled by RT-PCR, which upon lowering mapping stringency, was found to have been present in the NGS data all along. An accounting of PBV reads versus the total reads for each run yielded consistent results:

Run 1 (C3DTN) 140 of 462,336 (0.03%)=302 reads/million

Run 2 (C5968) 420 of 1,408,024 (0.03%)=298 reads/million

Run 3 (C7DWY) 116 of 456,878 (0.025%)=253 reads/million

Combined runs 1-3: 676 of 2,327,238 (0.03%)=290 reads/million

Generally speaking, these reads per million (rpm) are rather high values for viruses, especially from sputum, so it is conceivable the titers are well in excess of 10⁵ copies/ml.

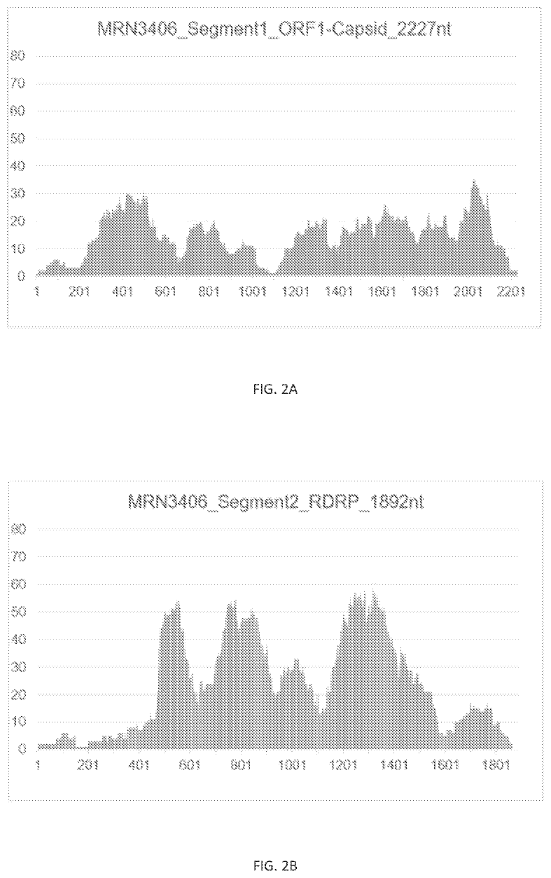

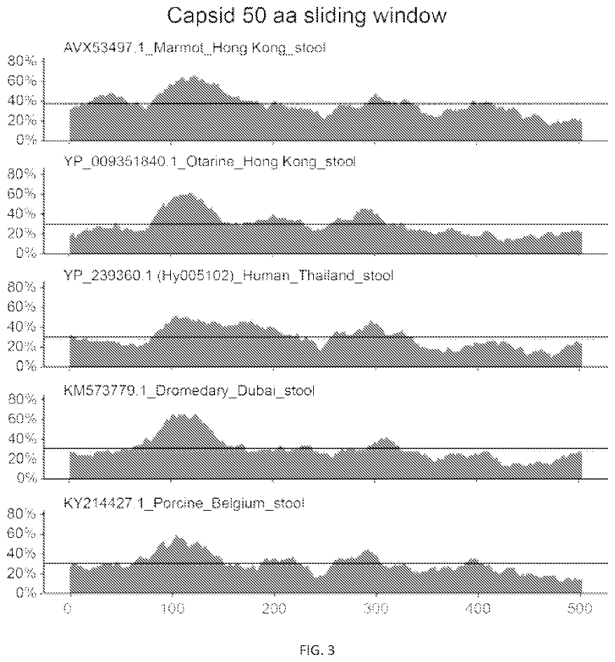

The complete genome was assembled. The total reference length is 4119 nt and the average coverage depth is 19×. A linear coverage plot of segments 1 and 2 are shown in FIG. 2A and FIG. 2B, respectively.

Using the complete genome sequences as references, the number of reads mapped and the percent genome coverage in CLC Bio Genomics Workbench software from those predicted by RAPsearch and DiVir 2.0 was assessed.

| ORF1- | RAPsearch | RAPsearch | RAPsearch | DiVir | DiVir | DiVir | Raw |
|---|---|---|---|---|---|---|---|
| Cap | viral in nr | not in nr | combined | ARM1 | ARM2 | combined | Data |
| Run #1 | 6/37% | 11/31% | 17/47% | 0/0% | 15/44% | 15/44% | 66/83% |
| Run #2 | 11/48% | 29/45% | 40/66% | 3/1% | 25/59% | 28/65% | 170/96% |
| Run #3 | 1/7% | 7/22% | 8/23% | 0/0% | 2/14% | 2/14% | 53/82% |

| | RAPsearch | RAPsearch | RAPsearch | DiVir | DiVir | DiVir | Raw |
|---|---|---|---|---|---|---|---|
| RDRP | viral in nr | not in nr | combined | ARM1 | ARM2 | combined | Data |
| Run #1 | 51/71% | 13/28% | 64/82% | 22/45% | 29/68% | 51/76% | 74/82% |
| Run #2 | 156/85% | 31/55% | 187/88% | 64/77% | 111/77% | 175/79% | 250/90% |
| Run #3 | 50/71% | 5/19% | 55/78% | 22/58% | 28/56% | 50/66% | 63/78% |

Both divergent virus prediction tools worked well to identify comparable numbers of reads and genome coverage. Indeed, most of the available RDRP reads were found by both, whereas fewer capsid reds were found since this is less conserved. Note that DiVir removes reverse complements and reads with stop codons, so it is expected to have fewer total reads.

Nucleotide and Sequences

The complete nucleotide sequence of segment 1 (2251 nt) was identified as:

(SEQ ID NO: 1)
AATTTGTACTTTAATGGTTTACAAGAGTTTAAAACCATACAACACTTTC
TACACTCTAAGAACACCAGCTACCGCACATAGTTTAGTGCAAATAGCTA
GGATCAGAGATAGTAAAGTGGGATTATCTGAAAGGAGGTTAAATTAATG
ACAGGTAATCAAATTAAATATGGTGAATTACAAGAAAATATTCGCCATA
ACACTACAACAGAAGTTGAAACCAATAGACACAACGTCGTGACTGAAGG
TGAAACCAACAGACATAACGTTGTTACAGAGGTTGAAACTAATCGACAC
AATACTGTGACTGAAAGTATTGGATGGTACGATGCTGTATCAAAACGAA
TCTCAGCAAATGCTTCAATGAGTCAAGCGGGTGCAGCTTGGGCTAATGT
TGCAATTAATCAACAAAATGCAGATACAAAGCGATTTGAAGCTGAACGC
AATGCTGAAATAAATCAGCAAAATGCGGACACTAGAACATTTAGTGCAC
GTAGTGAGGATGCAGCTAGATATGCTCATTCTTACAATGAAGATCGTAA
AACTACAGCTGAAATTGAGCGAATGAACACACAAAATTCGCAAGGATGG
GTGAAATCAATCACTGATGCAATCAGCTCACCTATCAAAGCATTACCAT
TATTAGGAGGATAAATTTTATGGTAAAGAATAACAACAAAAAGCGTTTT
CAGGATAAAAGTGATAAGTATTCTAGAAAACCTAAGTTCAAGGTTGAAA
AGAAAGATATCTTGGACGATGACAAATTGGAAGGATCTAAGTTTGGCAA
AGTTAATGACATATCCTGGTATCAGAAGAATGCTGATTTACTCAGAGCT
GCTGGTAACTTGTCTTTTGCTAATGCGTTGGGATCTGGAATTGATCTAT
CTAACGCAAACTTTAACGTTAAGCTTGCTGCTGATGAGCAACGTGTTCC
TGGTATTGCAACTATACATACTATTACAGGACCTGGACTCAGTCGCGAC
GCACACTCTGGTGTCAACGTGGCAATGCGTAACTTATATTCTTTTGTTC
GTCATGCAAATAGTGGTCATAGTAACTATGATCCTGTAGATCTAATGTT
ATATCTACCTGCTATGGATGCAGCATACATGCTCTACTACCGTGCTGTT
CGTGCATATGGCGCAATGTTCACATTTAATACTGTGAATCGCTATGCTC
CAAAAGCTCTTGTGGAAGCGTTAGGTTTTGATTATGAAGATGTCAACTC
AAACCTTGCTACATTCAGATATGCAATTAACGCATACGCTGCAAGAATC
AACGCATACGCTGTGCCTACGAATATGCCTATCTTCAAACGACATGCAT
GGCTCTTTTCATCTATCTATACAGATGAAAACGTATCTAAAGCTCAGAT
TTATGCATTTACTTCTGATCATTATAGAGTATTTGATGAGAAGTATTCT
AAAGGTGGACGCCTTGTGGCTAAAGCCTGGAAAACAAAGTTAACTGTTA
AAGATTGGATTACAGTAGCAAATGAGGTTGCTGATCCAATTACAGTTTC
AGAAGATTTAGGTATTATCTCAGGTGACTTAATTAAAGCATTTGGTAAG
GAAAACTTACACATGTTAGCTACCTTGGCTGATAACTACGTTGTATTAC
CAACATATGTACCTGAAGTTATGGATCAAATTCATAACTTGCAAGCAGT
AGGTCAGATTGATCTAGAAAGTAACAATATTGAACAAGATCCAAACATT
GGTAAGGGTAACTTGATTTACAACCCAGTTGTAACTGTCAATAATAATC
CAATGGCTTACGCAAATCGTATTATGGATTTCAAAATTGATACACCTAC
TCCAGATGATGTCGTTGTAGCTTCACGATTAGCTGTGGCATTAGAACCA
GGCGCTACAACCGGTAAGGCAGTATTCACTGCTATGGGTACAGAATTTG
TGACTAAAGTTGGTATTCACACATTCTACAAGGGAAATAATGGATTACT
TAAGTCTATTGAACAGACTTTCAATACTTTTGATTCTACTGAAGGTGGT
CTCACTGACGCCGCATCAGTTAGTTTGCACATGTCTGCCTACACAAAGG
CCTCTAAGTTTGTACACTTTCCAATTCAATATATGTGTATGGGTAGCCC
TACTCAACCTGACAAACGTGAAGTCAGAATCTTTGGCGAATTGGGCACG
TACACTATTATTAATGGGGTCACTCTTAATAAGTTACACGACGTGTGTG
TATTAAGTTTATTTGATGTACCTATTAAGCTTTAGATGCATTAGGG.

The sequence of the 5′ UTR for segment 1 (length 144 nt, coordinates 1 . . . 144) was identified as:

(SEQ ID NO: 2)
AATTTGTACTTTAATGGTTTACAAGAGTTTAAAACCATACAACACTTTC
TACACTCTAAGAACACCAGCTACCGCACATAGTTTAGTGCAAATAGCTA
GGATCAGAGATAGTAAAGTGGGATTATCTGAAAGGAGGTTAAATTA.

The 5′UTR length (144 nt) and base composition (66% AT-rich) are consistent with other reports describing 44-169 base 5′UTRs and sequences with only 22-38% G+C content.

In Woo P C Y et al., the authors describe a short open reading frame (ORF1) in a subset of the otarine PBVs sequenced, which precedes what all others are calling ORF1 and ORF2 (capsid)⁸. This is the only known publication that asserts there are 3 ORFs on segment 1. The sequence disclosed herein also possesses a methionine start codon at nt 14 in the presumed 5′UTR that yields a 61 aa protein (SEQ ID NO: 3). It bears minimal aa identity to the otarine PBV sequence and the human PBV in Wakuda, et al⁹.

The sequence of ORF1 (length 132 nt, coordinates 14 . . . 145), 61 aa (+2 frame) was identified as: MVYKSLKPYNTFYTLRTPATAHSLVQIARIRDSKVGLSERRLN (SEQ ID NO: 3).

The nucleotide sequence of ORF1 (length 507 nt, coordinates 145 . . . 651), 169 aa (+1 frame) was identified as:

(SEQ ID NO: 4)
ATGACAGGTAATCAAATTAAATATGGTGAATTACAAGAAAATATTCGCC
ATAACACTACAACAGAAGTTGAAACCAATAGACACAACGTCGTGACTGA
AGGTGAAACCAACAGACATAACGTTGTTACAGAGGTTGAAACTAATCGA
CACAATACTGTGACTGAAAGTATTGGATGGTACGATGCTGTATCAAAAC
GAATCTCAGCAAATGCTTCAATGAGTCAAGCGGGTGCAGCTTGGGCTAA
TGTTGCAATTAATCAACAAAATGCAGATACAAAGCGATTTGAAGCTGAA
CGCAATGCTGAAATAAATCAGCAAAATGCGGACACTAGAACATTTAGTG
CACGTAGTGAGGATGCAGCTAGATATGCTCATTCTTACAATGAAGATCG
TAAAACTACAGCTGAAATTGAGCGAATGAACACACAAAATTCGCAAGGA
TGGGTGAAATCAATCACTGATGCAATCAGCTCACCTATCAAAGCATTAC
CATTATTAGGAGGATAA.

The ORF1 protein has a predicted molecular weight of 18.7 kDa and an acidic pI of 5.93

ORF1_aa Sequence

(SEQ ID NO: 5)
MTGNQIKYGELQENIRHNTTTEVETNRHNVVTEGETNRHNVVTEVETNR
HNTVTESIGWYDAVSKRISANASMSQAGAAWANVAINQQNADTKRFEAE
RNAEINQQNADTRTFSARSEDAARYAHSYNEDRKTTAEIERMNTQNSQG
WVKSITDAISSPIKALPLLGG

The ExxRxNxxxE repeated motif underlined above has been observed in other picobirnaviruses (Da Costa, et al)¹⁰.

The top hit (BLASTp vs vvrsaa) shows porcine PBV 33% identity, 47% positive (partial: 132/168 aa aligned).

The sequence of the capsid (ORF2), length of 1563 nt, coordinates (657 . . . 2219), 521 aa (+3 frame) was identified as:

>2_PBV-MRN3406 Capsid V2 Positions 703 to 2304

(SEQ ID NO: 6)
ATGGTAAAGAATAACAACAAAAAGCGTTTTCAGGATAAAAGTGATAAGT
ATTCTAGAAAACCTAAGTTCAAGGTTGAAAAGAAAGATATCTTGGACGA
TGACAAATTGGAAGGATCTAAGTTTGGCAAAGTTAATGACATATCCTGG
TATCAGAAGAATGCTGATTTACTCAGAGCTGCTGGTAACTTGTCTTTTG
CTAATGCGTTGGGATCTGGAATTGATCTATCTAACGCAAACTTTAACGT
TAAGCTTGCTGCTGATGAGCAACGTGTTCCTGGTATTGCAACTATACAT
ACTATTACAGGACCTGGACTCAGTCGCGACGCACACTCTGGTGTCAACG
TGGCAATGCGTAACTTATATTCTTTTGTTCGTCATGCAAATAGTGGTCA
TAGTAACTATGATCCTGTAGATCTAATGTTATATCTACCTGCTATGGAT
GCAGCATACATGCTCTACTACCGTGCTGTTCGTGCATATGGCGCAATGT
TCACATTTAATACTGTGAATCGCTATGCTCCAAAAGCTCTTGTGGAAGC
GTTAGGTTTTGATTATGAAGATGTCAACTCAAACCTTGCTACATTCAGA
TATGCAATTAACGCATACGCTGCAAGAATCAACGCATACGCTGTGCCTA
CGAATATGCCTATCTTCAAACGACATGCATGGCTCTTTTCATCTATCTA
TACAGATGAAAACGTATCTAAAGCTCAGATTTATGCATTTACTTCTGAT
CATTATAGAGTATTTGATGAGAAGTATTCTAAAGGTGGACGCCTTGTGG
CTAAAGCCTGGAAAACAAAGTTAACTGTTAAAGATTGGATTACAGTAGC
AAATGAGGTTGCTGATCCAATTACAGTTTCAGAAGATTTAGGTATTATC
TCAGGTGACTTAATTAAAGCATTTGGTAAGGAAAACTTACACATGTTAG
CTACCTTGGCTGATAACTACGTTGTATTACCAACATATGTACCTGAAGT
TATGGATCAAATTCATAACTTGCAAGCAGTAGGTCAGATTGATCTAGAA
AGTAACAATATTGAACAAGATCCAAACATTGGTAAGGGTAACTTGATTT
ACAACCCAGTTGTAACTGTCAATAATAATCCAATGGCTTACGCAAATCG
TATTATGGATTTCAAAATTGATACACCTACTCCAGATGATGTCGTTGTA
GCTTCACGATTAGCTGTGGCATTAGAACCAGGCGCTACAACCGGTAAGG
CAGTATTCACTGCTATGGGTACAGAATTTGTGACTAAAGTTGGTATTCA
CACATTCTACAAGGGAAATAATGGATTACTTAAGTCTATTGAACAGACT
TTCAATACTTTTGATTCTACTGAAGGTGGTCTCACTGACGCCGCATCAG
TTAGTTTGCACATGTCTGCCTACACAAAGGCCTCTAAGTTTGTACACTT
TCCAATTCAATATATGTGTATGGGTAGCCCTACTCAACCTGACAAACGT
GAAGTCAGAATCTTTGGCGAATTGGGCACGTACACTATTATTAATGGGG
TCACTCTTAATAAGTTACACGACGTGTGTGTATTAAGTTTATTTGATGT
ACCTATTAAGCTT.

The capsid protein has a predicted molecular weight of 57.8 kDa and a basic pI of 8.42.

The capsid sequence was identified as:

>2_PBV-MRN3406 Capsid V2 Positions 703 to 2304

(SEQ ID NO: 7)
MVKNNKKRFQDKSKYSRKPKSREKKDILDDDKLEGSKFGKVNDISWYQK
NADLLRAAGNLSFANALGSGIDLSNANFNVKLAADEQRVPGIATIHTIT
GPGLSRDAHSGVNVAMRNLYSFVRHANSGHSNYDPVDLMLYLPAMDAAY
MLYYRAVRAYGAMFTFNTVNRYAPKALVEALGFDYEDVNSNLATFRYAI
NAYAARINAYAVPTNMPIFKRHAWLFSSIYTDENVSKAQIYAFTSDHYR
VFDEKYSKGGRLVAKAWKTKLTVKDWITVANEVADPITVSEDLGIISGD
LIKAFGKENLHMLATLADNYVVLPTYVPEVMDQIHNLQAVGQIDLESNN
IEQDPNIGKGNLIYNPVVTVNNNPMAYANRIMDFKIDTPTPDDVVVASR
LAVALEPGATTGKAVFTAMGTEFVTKVGIHTFYKGNNGLLKSIEQTFNT
FDSTEGGLTDAASVSLHMSAYTKASKFVHFPIQYMCMGSPTQPDKREVR
IFGELGTYTIINGVTLNKLHDVCVLSLFDVPIKL.

The top hit (BLASTp vs nrVirusX) showed Marmot PBV at 37% identity, 55% positive (entire). This low degree of amino acid identity compared to other capsid proteins is expected given the observed diversity reported in the literature.

The sequence of the 3′UTR (length 8 nt, coordinates 2220 . . . 2227) was identified as: TGATGCGG (SEQ ID NO: 8).

The complete nucleotide sequence (1892 nt) for segment 2 was identified as:

(SEQ ID NO: 9)
CTAAATGAATAGAAAAGTAGTCAAGTTAGGTAATTATTTTAAATTACCG
AATCCCGGATTGAAGACCTATCTATTGAAAACTAAGAGAGGTAACGATG
AAGAGTATCGTACTCCATTTTTCAAAGATAAATCTTTGTCCGATGTATT
ACAAGGCTGGTTAGTGCACCTAGCCCCTCTCAAGAGTGAGTGGCCTGGT
TTACACCAGTTTGAATTAGACCTAGCGGAAAAGGTCGGGCCTTTAAGCA
TCCAGAAACCTTTAGATGAGCGGTTTAAGGATATTGAGGCTTATTACAA
AGGTATTCTCCTACCTTCCAAACCAATCAGTGAAACAGCAATCCGATCT
GTTTTAACTGAATGGAATAGGGCACGTGGCTTGTCGGTACGCAGTGTCT
CCAAAACGTGGGATAACATGAAGAAATCTACATCTTCAGGTTCTCCATT
CTTTACTAAACGTAAAGCAGTCGGAAAATATACGATGTATATGGAGCCA
TGTTTTGACAAAAGAACGCAAGAAGTTCATTTTAAGAACTCAAACCGTT
GGGATCCAATTGCGGTCTTAGGTTGGCGTGGACAAGAAGGTGGACCTGA
TTTTGAGGATGTAAAGCAAAGGGTTGTATGGATGTTCCCTGCTTCGGTA
AACCTACAAGAGTTACGTGTTTACCAACCTCTAATCGAAACAGCGCAAC
GTTTCAACTTAGTTCCTGCTTGGGTTGGCATGGATAGTGTTGATTTGCA
CATCACACGTATGTTTGATACGAAAGGCGAAGACGATGTCGTAATATGT
ACAGATTTCTCAAAATTTGACCAACATTTTAATGCTGATATGGCTCGCG
GTGCATCCGAAATATTGGATGGCCTCTTTAACGGGAGCAGAGATTTTGT
ACAATGGATGTGGGATATATATCACATCAAATACACGATACCTCTATTA
GACTCAGAAGATCATGCCTGGTTTGGCAGACATGGTATGGGCTCTGGTT
CAGGTGGAACCAATGCCGATGAAACATTAGCTCATAGAGCTTTGCAGTA
CGAAGCTGCTTTATCACAGAACCAAACATTAAACCCTTATTCACAATGT
CTAGGTGATGATGGAGTACTAACATATCCTGGAATTAAAGTGGATGATG
TAATGCGATCATATACTGCACATGGTCAAGAGATGAATGAGTCAAAACA
GTATGTGAGCAAACATGAATGCATATATCTTCGTAGATGGCATCATATT
AATTATCGTGTCGATGATGTATGTGTCGGAGTTTACGCAACAACTCGTG
CTTTGGGTAGATTGTGTGAACAAGAGAGATATTTTGACCCAGAGATATG
GTCAAAAGAAATGGTAGCTTTACGTCAGCTATCGATACTTGAGAATGTG
AAATACCACCCTCTCAAGGAAGAATTTGTTAAATATTGCATGAAAGGGG
ATAAGTACAGACTGGGACTGGACTTACCAGGCTTCTTGGAGAACATAGA
TGGACTCGCAAAGCAAGCTACTGATCTAATGCCGGACTTTTTAGGTTAC
GTTAAATCACAACAGAAATCTGTCGGTGGTATATCAGAATGGTGGATAG
TAAAATATCTACGTAGTCTAAAGTAAAGATTGGGATGGTGCAGTAAACC
ATTAGAATTCTAACGAATTCTAACTGCACCATCCCAATCTTTACTTTAG
ACTACGTAGATATTTTACTATCCACCACTCTGATATACCACCGACAGAT
TTCTGTTGTGATTTAACGTAACCTAAAAAGTCCGGCATCAGATCAGTAG
CTTGCTTTGCGAGTCCATCTATGTTCTCCAAGAAGCCTGGTAAGTCCAG
TCCCAGTCTGTACTTATCCCCTTTCATGCAATATTTAACAAATTCTTCC
TTGAGAGGGTGGTATTTCACATTCTCAAGT.

FIG. 3 shows a pairwise amino acid alignment (50 aa sliding window) of the ABT PBV capsid coding sequence to representative picobirnavirus strains. The mean (solid line) and median (dotted line) identities overall are approximately 35%.

The nucleotide sequence of the RNA-dependent RNA polymerase (RDRP), length 1587 nt, coordinates (5 . . . 1591), 529 aa was identified as:

>RDRP_nt Sequence

(SEQ ID NO: 10)
ATGAATAGAAAAGTAGTCAAGTTAGGTAATTATTTTAAATTACCGAATC
CCGGATTGAAGACCTATCTATTGAAAACTAAGAGAGGTAACGATGAAGA
GTATCGTACTCCATTTTTCAAAGATAAATCTTTGTCCGATGTATTACAA
GGCTGGTTAGTGCACCTAGCCCCTCTCAAGAGTGAGTGGCCTGGTTTAC
ACCAGTTTGAATTAGACCTAGCGGAAAAGGTCGGGCCTTTAAGCATCCA
GAAACCTTTAGATGAGCGGTTTAAGGATATTGAGGCTTATTACAAAGGT
ATTCTCCTACCTTCCAAACCAATCAGTGAAACAGCAATCCGATCTGTTT
TAACTGAATGGAATAGGGCACGTGGCTTGTCGGTACGCAGTGTCTCCAA
AACGTGGGATAACATGAAGAAATCTACATCTTCAGGTTCTCCATTCTTT
ACTAAACGTAAAGCAGTCGGAAAATATACGATGTATATGGAGCCATGTT
TTGACAAAAGAACGCAAGAAGTTCATTTTAAGAACTCAAACCGTTGGGA
TCCAATTGCGGTCTTAGGTTGGCGTGGACAAGAAGGTGGACCTGATTTT
GAGGATGTAAAGCAAAGGGTTGTATGGATGTTCCCTGCTTCGGTAAACC
TACAAGAGTTACGTGTTTACCAACCTCTAATCGAAACAGCGCAACGTTT
CAACTTAGTTCCTGCTTGGGTTGGCATGGATAGTGTTGATTTGCACATC
ACACGTATGTTTGATACGAAAGGCGAAGACGATGTCGTAATATGTACAG
ATTTCTCAAAATTTGACCAACATTTTAATGCTGATATGGCTCGCGGTGC
ATCCGAAATATTGGATGGCCTCTTTAACGGGAGCAGAGATTTTGTACAA
TGGATGTGGGATATATATCACATCAAATACACGATACCTCTATTAGACT
CAGAAGATCATGCCTGGTTTGGCAGACATGGTATGGGCTCTGGTTCAGG
TGGAACCAATGCCGATGAAACATTAGCTCATAGAGCTTTGCAGTACGAA
GCTGCTTTATCACAGAACCAAACATTAAACCCTTATTCACAATGTCTAG
GTGATGATGGAGTACTAACATATCCTGGAATTAAAGTGGATGATGTAAT
GCGATCATATACTGCACATGGTCAAGAGATGAATGAGTCAAAACAGTAT
GTGAGCAAACATGAATGCATATATCTTCGTAGATGGCATCATATTAATT
ATCGTGTCGATGATGTATGTGTCGGAGTTTACGCAACAACTCGTGCTTT
GGGTAGATTGTGTGAACAAGAGAGATATTTTGACCCAGAGATATGGTCA
AAAGAAATGGTAGCTTTACGTCAGCTATCGATACTTGAGAATGTGAAAT
ACCACCCTCTCAAGGAAGAATTTGTTAAATATTGCATGAAAGGGGATAA
GTACAGACTGGGACTGGACTTACCAGGCTTCTTGGAGAACATAGATGGA
CTCGCAAAGCAAGCTACTGATCTAATGCCGGACTTTTTAGGTTACGTTA
AATCACAACAGAAATCTGTCGGTGGTATATCAGAATGGTGGATAGTAAA
ATATCTACGTAGTCTAAAG.

The RDRP protein has a predicted molecular weight of 61.1 kDa and a pI of 7.69

The RDRP sequence was identified as:

(SEQ ID NO: 11)
MNRKVVKLGNYFKLPNPGLKTYLLKTKRGNDEEYRTPFFKDKSLSDVLQ
GWLVHLAPLKSEWPGLHQFELDLAEKVGPLSIQKPLDERFKDIEAYYKG
ILLPSKPISETAIRSVLTEWNRARGLSVRSVSKTWDNMKKSTSSGSPFF
TKRKAVGKYTMYMEPCFDKRTQEVHFKNSNRWDPIAVLGWRGQEGGPDF
EDVKQRVVWMFPASVNLQELRVYQPLIETAQRFNLVPAWVGMDSVDLHI
TRMFDTKGEDDVVICTDFSKFDQHFNADMARGASEILDGLFNGSRDFVQ
WMWDIYHIKYTIPLLDSEDHAWFGRHGMGSGSGGTNADETLAHRALQYE
AALSQNQTLNPYSQCLGDDGVLTYPGIKVDDVMRSYTAHGQEMNESKQY
VSKHECIYLRRWHHINYRVDDVCVGVYATTRALGRLCEQERYFDPEIWS
KEMVALRQLSILENVKYHPLKEEFVKYCMKGDKYRLGLDLPGFLENIDG
LAKQATDLMPDFLGYVKSQQKSVGGISEWWIVKYLRSLK.

Top Blast hits shows otarine/skink/Dromedary PBV at 64% identity, 75% positive (entire).

The RDRP length is consistent with other reports (529-539 aa), as is the amino acid identity to other group I PBVs (44-70%).

The nucleotide sequence of the 3′UTR (length 301 nt, coordinates 1592 . . . 1892) was identified as:

(SEQ ID NO: 12)
TAAAGATTGGGATGGTGCAGTAAACCATTAGAATTCTAACGAATTCTAA
CTGCACCATCCCAATCTTTACTTTAGACTACGTAGATATTTTACTATCC
ACCACTCTGATATACCACCGACAGATTTCTGTTGTGATTTAACGTAACC
TAAAAAGTCCGGCATCAGATCAGTAGCTTGCTTTGCGAGTCCATCTATG
TTCTCCAAGAAGCCTGGTAAGTCCAGTCCCAGTCTGTACTTATCCCCTT
TCATGCAATATTTAACAAATTCTTCCTTGAGAGGGTGGTATTTCACATT
CTCAAGT.

This 3′UTR sequence is much longer than other reports (30-50 nts) and likely represents a more complete sequence than others have been able to obtain.

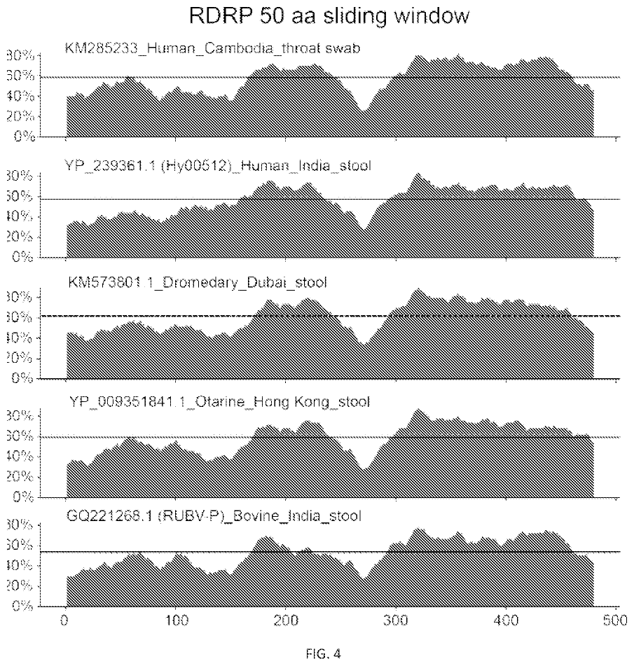

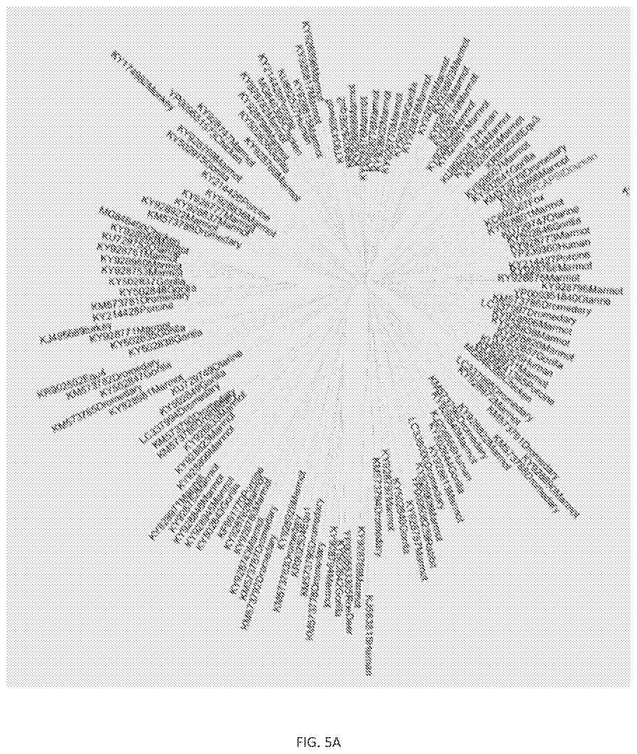

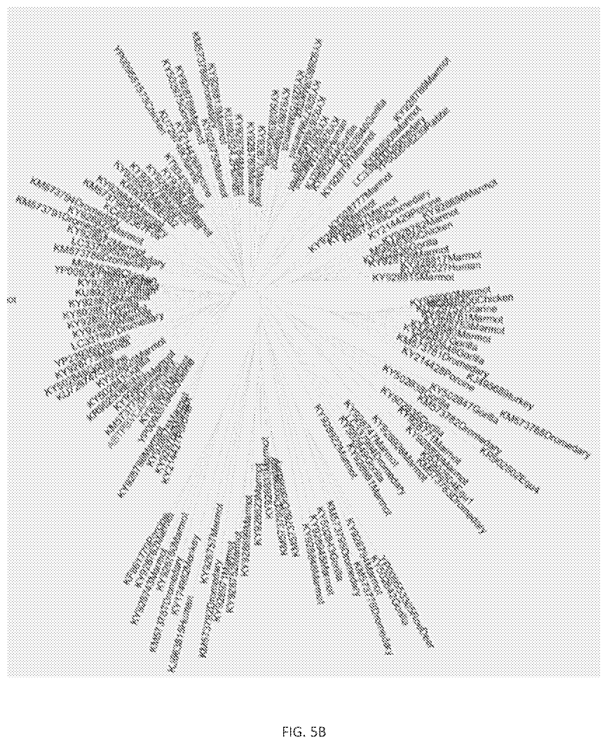

FIG. 4 shows a pairwise amino acid alignment (50 aa sliding window) of the ABT_PBV RDRP coding sequence to representative picobirnavirus strains. The mean (solid line) and median (dotted line) identities overall are approximately 60%.

Phylogenetic Analysis

Phylogenetic analysis was performed on the capsid and RDRP proteins. All available picobirnavirus sequences deposited in GenBank were retrieved. 1566 sequences were downloaded and parsed to separate files by annotation. There were 814 RDRP sequences, 427 capsid, and 325 ORF1 sequences. ABT_PBV sequences were added to each file and a multiple sequence alignment was performed with CLUSTAL-W in BioEdit. Alignments for capsid and RDRP were reduced to the ABT_PBV sequence set as the mask; ORF1 is highly divergent and was not analyzed. Duplicate accessions, those from the same study/location/host that were highly identical, and those without coverage in the desired alignment region were removed through an iterative process to create trees of manageable size.

Capsid: For capsid, the number of references were reduced from 427 to 132 full-length (521 aa) sequences (mostly marmot PBV were removed). Protdist neighbor-joining trees were rooted on the midpoint in Tree Explorer. Two trees were produced, the first in which gaps were not stripped (521 aa alignment) and another in which gaps were stripped (156 aa). Consistent with Knox, et al, branching patterns for picobirnaviruses strains were maintained when comparing these ‘complete’ trees¹¹. The ABT-PBV capsid (red) consistently branched with marmot (KY928866, KY928801; Himalayas), and Dromedary camel (KM573779; United Arab Emirates) PBV sequences (blue). Other sequences consistently on this branch were PBVs of California sea lions (Otarine), gorillas, and humans (blue), as well as horses, pigs and chickens (green). As noted before, capsid sequences are much less conserved and there is not a standard analysis region for the protein reported in the literature.

The strains branching with ABT_PBV capsid are listed below with reported information of the source and any disease association.

| Acession | Type | Source | Disease | Host | Isolate |
|---|---|---|---|---|---|
| KY214427 | porcine | feces | | Sus scro [symbol] | BEL/15V010/2605 |
| KY928766 | marmot | fecal sample | | Marmota himalayana | c223431_ [symbol] 1_i1_libraryA_2618 |
| KY928875 | marmot | fecal sample | | Marmota himalayana | c326 [symbol] 15_ [symbol] 1_i1_libraryA_2610 |
| KY928796 | marmot | fecal sample | | Marmota himalayana | c290190_ [symbol] 1_i1_libraryA_2387 |
| YP009351840 | otarine | fecal swab | | Zalophus californianus | HKG-PF080915 [symbol] Ref Seq NC_034160.1 |
| KY928801 | marmot | fecal sample | | Marmota himalayana | c295860_ [symbol] 1_i1_libraryA_2470 |
| KM573779 | camel | fecal sample | | Camelus dromedarius | c15 [symbol] 1 |
| KY928866 | marmot | fecal sample | | Marmota himalayana | c324215_ [symbol] 1_i1_libraryA_2566 |
| KU729747 | otarine | fecal swab | | Zalophus californianus | PF090303 |
| KY502846 | gorilla | feces | | Gorilla gorilla | IHU-Con-GP [symbol] vs-V12-ContigS6 |
| KY928773 | marmot | fecal sample | | Marmota himalayana | c254432_ [symbol] 1_i1_libraryA_2526 |
| YP239360 | human | stool [symbol] | acute | Homo sapiens | Hy005102 [symbol] Ref Seq NC_007026.1 |
| | | | gastroenteritis | | |
| KR902508 | equine | plasma [symbol] | febrile | Equus cabalius | horse 3 |
| KY502841 | gorilla | feces | | Gorilla gorilla | IHU-Con-GP [symbol] vs-V7-ContigS3 |
| KY928820 | marmot | fecal sample | | Marmota himalayana | c307746_ [symbol] 1_i1_libraryA_2693 |
| LC337997 | camel | fecal sample | | Camelus dromedaruis | 101C/Gp [symbol] |
| KU892526 | human | diarrh [symbol] ic feces | ulcerative | Homo sapiens | human/BEL/HPBV945/2010 |
| | | | colitis | | |
| YP009241385 | porcine | diarrh [symbol] ic feces | diarrhea | Sus scro [symbol] | 221/04−16/ITA/2004 |
| MG846398 | chicken | feces | malabsorption | Gallus gallus | RS/BR/15/4 [symbol] −1 |
| | | | syndrome | | |

| Acession | Country | Collection date |
|---|---|---|
| KY214427 | Belgium | Jan-15 |
| KY928766 | China | 2013 |
| KY928875 | China | 2013 |
| KY928796 | China | 2013 |
| YP009351840 | Hong Kong | Sep. 12, 2008 |
| KY928801 | China | 2013 |
| KM573779 | United Arab Emirates | 2013 |
| KY928866 | China | 2013 |
| KU729747 | Hong Kong | 4 Mar. 2009 |
| KY502846 | Republic of the Congo | Aug-15 |
| KY928773 | China | 2013 |
| YP239360 | Thailand | 2004 |
| KR902508 | USA | 10 Oct. 2012 |
| KY502841 | Republic of the Congo | Aug-15 |
| KY928820 | China | 2013 |
| LC337997 | United Arab Emirates | 2013 |
| KU892526 | Belgium | 2010 |
| YP009241385 | Italy | 2004 |
| MG846398 | Brazil | 2015 |

[symbol]

indicates data missing or illegible when filed

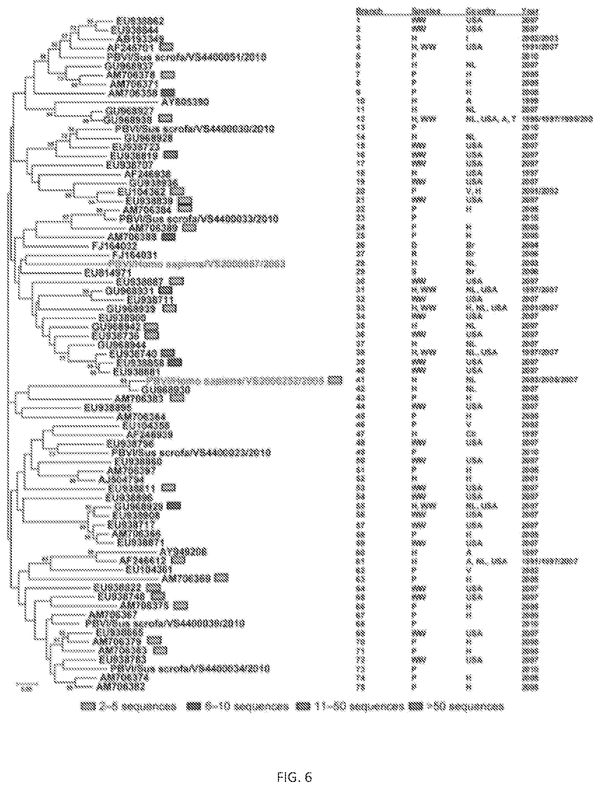

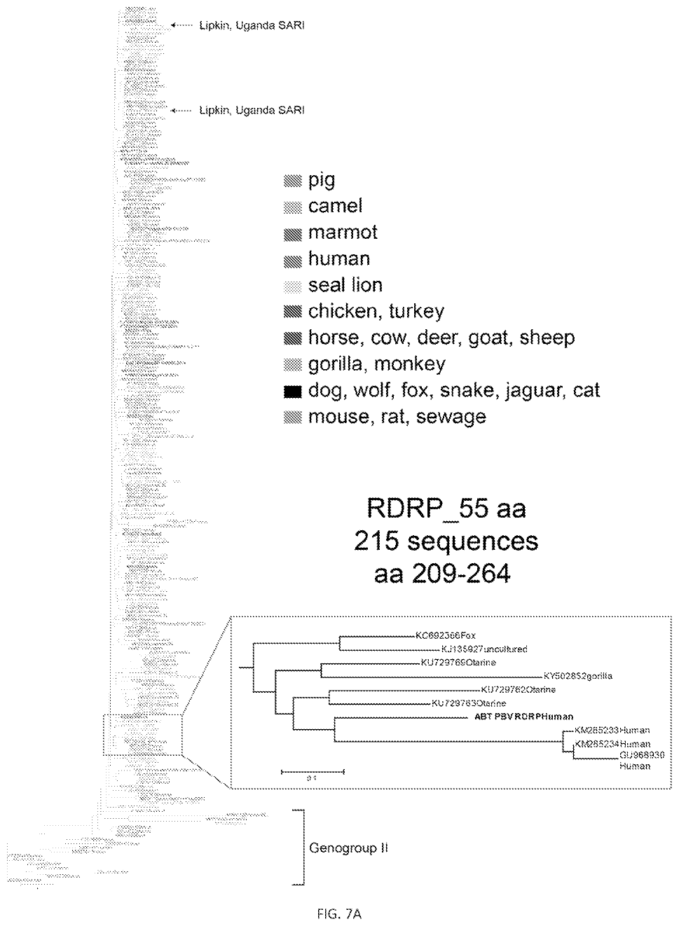

Radial trees of the same alignments more clearly demonstrate genetic distance between strains (e.g. long branch lengths) and just how interchangeable hosts are (FIGS. 5A and 5B). While no clear delineation between species or location is apparent, there do appear to be distinct groupings for capsid. Since there are fewer capsid entries and many are from the same host, it is very likely these presumed relationships are biased.

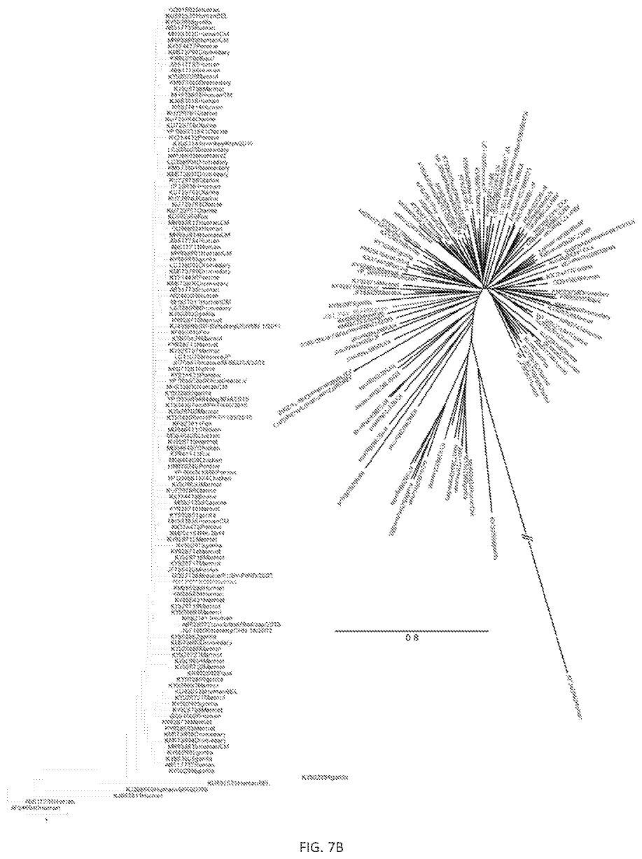

RDRP: RDRP sequences are more conserved than capsid and segregate into Genogroups I and II. Whether due to RDRP being used for classification of strains or since this gene is easier to detect in samples by similarity, there are consequently many more sequences in the database compared to capsid. There is a standard 55 aa region of the protein reported in the literature for phylogenetic analysis which corresponds to amino acids 209-264 in the ABT_RDRP. FIG. 6 shows an example of an RDRP tree on this 165 nt segment from Smits, et al which highlights pig and human sequences obtained from respiratory tracts⁵.

The tree shown in FIG. 7A contains the novel PBV strain identified herein. 841 RDRP sequences in this 55 aa region were reduced to 215, including a diversity of strains and those with implications for respiratory disease. Protdist neighbor-joining trees were rooted on human Genotype II strain, AF246940 (4-GA-91)⁷. Note as above with capsid, beside the delineation of GI and GII, there is no branching along host lines for RDRP.

The branch with the ABT_RDRP sequence was magnified and includes 3 notable sequences of interest. First, the two highly similar references, KM285233 & KM285234, were obtained in 2009 from upper respiratory swabs of two patients in Cambodia. These sequences were never part of a publication, but were deposited in GenBank by Mishra, N. and Lipkin, W. I.

The other strain it branches with, GU968930, originates from diarrhea samples obtained in the Netherlands. What is intriguing is that this sequence found in the above figure from Smits, et al, branches with 99% bootstrap value to the human respiratory strain, VS2000252/20055,12.

Also, on this same branch were several otarine (sea lion) sequences, gorilla, fox and uncultured raw sewage which are related to stool samples.

Indeed, the overwhelming majority of the >800 RDRP sequences in GenBank are derived from stool samples, but the novel sequence identified herein branches with the handful of deposited sequences related to respiratory illness.

Unfortunately, the Osterhaus group did not deposit the porcine or human respiratory sequences in GenBank⁵. Similarly, the sequences from Cummings, et al describing an association of PBV with severe acute respiratory illness (SARI) in Uganda were also not deposited⁶. However, strains branching with these sequences or those indicated to be most similar were included in the table below.

| | | | | | | | |
|---|---|---|---|---|---|---|---|
| Acession | Type | Source | Disease | Host | Isolate | Country | Collection date |

RDRP 55 aa: 209-264

| | | | | | | |
|---|---|---|---|---|---|---|
| KC692366 | fox | feces | Vulpes vulpes | Fox 5 | Netherlands | 15 Mar. 2012 |
| KJ135927 | uncultured | wastewater | n/a | Hunan_137 | China | 2012 |
| KU729769 | California sea lion | rectal swabs, feces | Zalophus californianus | PF100408 | Hong Kong | 25 Apr. 2010 |
| KY502852 | gorilla | feces | Gorilla gorilla | IHU-Con- | Republic of | Aug-15 |
| | | | | GPbvs-V18 | the Congo | |
| KU729762 | California sea lion | rectal swabs, feces | Zalophus californianus | PF090206 | Hong Kong | 17 Feb. 2009 |
| KU729763 | California sea lion | rectal swabs, feces | Zalophus californianus | PF090302 | Hong Kong | 4 Mar. 2009 |
| KM285233 | human | upper respiratory swabs | Homo sapiens | KS02-CSP-202 | Cambodia | 5 Oct. 2009 |
| KM285234 | human | upper respiratory swabs | Homo sapiens | KS02-CSP-203 | Cambodia | 5 Oct. 2009 |

| | | | | | | |
|---|---|---|---|---|---|---|
| GU96 [symbol] 930 | human | stool *branch with human respiratory | Homo sapiens | VS22 | Netherlands | 11 Feb. 2007 |

RDRP 348 aa: 126-473

| | | | | | | |
|---|---|---|---|---|---|---|
| GQ221268 | cow | stool | Bos indicus | RUBV-P | India | Jan-05 |
| KM285233 | human | upper respiratory swabs | Homo sapiens | KS02-CSP-2002 | Cambodia | 5 Oct. 2009 |
| KM285234 | human | upper respiratory swabs | Homo sapiens | KS02-CSP-203 | Cambodia | 5 Oct. 2009 |

[symbol]

indicates data missing or illegible when filed

It has been shown that the trees derived from the 55 aa sequence can reliably predict the branching pattern of the full length RDRP¹³. Nevertheless, a much longer alignment of 132 sequences covering 348 aa (coordinates 126-473) was created to further explore phylogenetic relationships to the novel strain. Phylogenic trees were developed (FIG. 7B), and the results are summarized in the table below. The table below shows that the novel sequence continues to branch with Cambodian respiratory strains (KM28523X.1), in addition to a bovine sequence from India (RUBV-P). These strains along with a select few full-length reference strains were aligned by CLUSTAL-W in two different software programs and yielded similar results. The novel strain only has 57% amino acid identity with the Cambodian respiratory strains. Note that as expected by the branching patterns above, the Cambodian strains are 97% identical, as are the cow (AB828072.1) and monkey (JQ710506.1) strains.

| | ABT_PBV_RDRP | (KM285233.1) | (KM285234.1) | (G [symbol] 221268.1) |
|---|---|---|---|---|
| ABT PBV RDRP | ID | 0.573 | 0.5 [symbol] 9 | 0.509 |
| AKG92636.1(KM285233.1)[Human picobirnavirus] | 0.573 | ID | 0.975 | 0.519 |
| AKG92637.1(KM2 [symbol] 5234.1)[Human picobirnavirus] | 0.569 | 0.975 | ID | 0.517 |
| ACT [symbol] 4131.1( [symbol] 2212 [symbol] .1)[Picobirnavirus | 0.509 | 0.519 | 0.517 | ID |
| bovine/RU [symbol] V [symbol] P [symbol] /2005] | | | | |
| YP_239361.1 [Human picobirnavirus Hy00512] | 0.544 | 0.546 | 0.546 | 0.503 |
| AA [symbol] 53583.1(AF246939.1)[Human picobirnavirus | 0.612 | 0.585 | 0.586 | 0.536 |
| 1-CHN-97] | | | | |
| YP_009351841.1[Otarine picobirnavirus] | 0.567 | 0.571 | 0.570 | 0.551 |
| AHZ246150.1(KJ4 [symbol] 56 [symbol] 0.1)[Picobirnavirus | 0.533 | 0.514 | 0.516 | 0.509 |
| [symbol] PBV/turkey/USA/MN [symbol] 1/2011] | | | | |
| YP_009551574.1[Chicken picobirnavirus] | 0.551 | 0.524 | 0.525 | 0.519 |
| YP_00924138 [symbol] .1[Porcine picobirnavirus] | 0.506 | 0.494 | 0.497 | 0.489 |
| BANS8175.1(A [symbol] 82 [symbol] 072.1)[Picobirnavirus | 0.412 | 0.396 | 0.396 | 0.4 [symbol] 2 |
| cow [symbol] 7944Jap/2013] | | | | |
| AI [symbol] 06802.1(KF823811.1)[Fox fecal picobirnavirus] | 0.517 | 0.527 | 0.531 | 0.487 |
| AIY312 [symbol] 7.1(KMS73801.1[Dromedary picobirnavirus] | 0.564 | 0.520 | 0.523 | 0.514 |
| AFK81927.1(JQ710506.1)[Picobirnavirus | 0.412 | 0.401 | 0.397 | 0.435 |
| monkey/C [symbol] N−14/2002] | | | | |

| | YP_239361.1 | (AF246939.1) | YP_009351841.1 | (KJ495690.1) |
|---|---|---|---|---|
| ABT PBV RDRP | 0.544 | 0.612 | 0.5 [symbol] 7 | 0.533 |
| AKG92636.1(KM285233.1)[Human picobirnavirus] | 0.546 | 0.585 | 0.571 | 0.514 |
| AKG92637.1(KM2 [symbol] 5234.1)[Human picobirnavirus] | 0.546 | 0.586 | 0.570 | 0.516 |
| ACT [symbol] 4131.1( [symbol] 2212 [symbol] .1)[Picobirnavirus | 0.503 | 0.53 [symbol] | 0.551 | 0.509 |
| bovine/RU [symbol] V [symbol] P [symbol] /2005] | | | | |
| YP_239361.1 [Human picobirnavirus Hy00512] | ID | 0.594 | 0.604 | 0.589 |
| AA [symbol] 53583.1(AF246939.1)[Human picobirnavirus | 0.594 | ID | 0.621 | 0.570 |
| 1-CHN-97] | | | | |
| YP_009351841.1[Otarine picobirnavirus] | 0.604 | 0.621 | ID | 0.608 |
| AHZ246150.1(KJ4 [symbol] 56 [symbol] 0.1)[Picobirnavirus | 0.589 | 0.570 | 0.60 [symbol] | ID |
| [symbol] PBV/turkey/USA/MN [symbol] 1/2011] | | | | |
| YP_009551574.1[Chicken picobirnavirus] | 0.580 | 0.604 | 0.621 | 0.582 |
| YP_00924138 [symbol] .1[Porcine picobirnavirus] | 0.545 | 0.550 | 0.58 [symbol] | 0.550 |
| BANS8175.1(A [symbol] 82 [symbol] 072.1)[Picobirnavirus | 0.443 | 0.452 | 0.448 | 0.448 |
| cow [symbol] 7944Jap/2013] | | | | |
| AI [symbol] 06802.1(KF823811.1)[Fox fecal picobirnavirus] | 0.553 | 0.544 | 0.591 | 0.570 |
| AIY312 [symbol] 7.1(KMS73801.1[Dromedary picobirnavirus] | 0.5 [symbol] 4 | 0.591 | 0.668 | 0.557 |
| AFK81927.1(JQ710506.1)[Picobirnavirus | 0.444 | 0.456 | 0.450 | 0.450 |
| monkey/C [symbol] N−14/2002] | | | | |

| | YP_009551574.1 | YP_009241386.1 | (AB828072.1) | KF823811.1) |
|---|---|---|---|---|
| ABT PBV RDRP | 0.551 | 0.506 | 0.412 | 0.517 |
| AKG92636.1(KM285233.1)[Human picobirnavirus] | 0.524 | 0.494 | 0.396 | 0.527 |
| AKG92637.1(KM2 [symbol] 5234.1)[Human picobirnavirus] | 0.525 | 0.497 | 0.396 | 0.531 |
| ACT [symbol] 4131.1( [symbol] 2212 [symbol] .1)[Picobirnavirus | 0.519 | 0.489 | 0.432 | 0.487 |
| bovine/RU [symbol] V [symbol] P [symbol] /2005] | | | | |
| YP_239361.1 [Human picobirnavirus Hy00512] | 0.580 | 0.545 | 0.433 | 0.533 |
| AA [symbol] 53583.1(AF246939.1)[Human picobirnavirus | 0.604 | 0.550 | 0.452 | 0.544 |
| 1-CHN-97] | | | | |
| YP_009351841.1[Otarine picobirnavirus] | 0.621 | 0.586 | 0.448 | 0.591 |
| AHZ246150.1(KJ4 [symbol] 56 [symbol] 0.1)[Picobirnavirus | 0.582 | 0. [symbol] | 0.448 | 0.570 |
| [symbol] PBV/turkey/USA/MN [symbol] 1/2011] | | | | |
| YP_009551574.1[Chicken picobirnavirus] | ID | 0.632 | 0.444 | 0.617 |
| YP_00924138 [symbol] .1[Porcine picobirnavirus] | 0.632 | ID | 0.412 | 0.633 |
| BANS8175.1(A [symbol] 82 [symbol] 072.1)[Picobirnavirus | 0.444 | 0.412 | ID | 0.406 |
| cow [symbol] 7944Jap/2013] | | | | |
| AI [symbol] 06802.1(KF823811.1)[Fox fecal picobirnavirus] | 0.617 | 0.633 | 0.406 | ID |
| AIY312 [symbol] 7.1(KMS73801.1[Dromedary picobirnavirus] | 0.588 | 0.551 | 0.427 | 0.571 |
| AFK81927.1(JQ710506.1)[Picobirnavirus | 0.449 | 0.410 | 0.971 | 0.407 |
| monkey/C [symbol] N−14/2002] | | | | |

| | (KM573801.1) | (J [symbol] 710506.1) |
|---|---|---|
| ABT PBV RDRP | 0.5 [symbol] 4 | 0.412 |
| AKG92636.1(KM285233.1)[Human picobirnavirus] | 0. [symbol] 2 | 0.401 |
| AKG92637.1(KM2 [symbol] 5234.1)[Human picobirnavirus] | 0.523 | 0.397 |
| ACT [symbol] 4131.1( [symbol] 2212 [symbol] .1)[Picobirnavirus | 0.514 | 0.43 [symbol] |
| bovine/RU [symbol] V [symbol] P [symbol] /2005] | | |
| YP_239361.1 [Human picobirnavirus Hy00512] | 0.564 | 0.444 |
| AA [symbol] 53583.1(AF246939.1)[Human picobirnavirus | 0.591 | 0.456 |
| 1-CHN-97] | | |
| YP_009351841.1[Otarine picobirnavirus] | 0.6 [symbol] | 0.450 |
| AHZ246150.1(KJ4 [symbol] 56 [symbol] 0.1)[Picobirnavirus | 0.557 | 0.450 |
| [symbol] PBV/turkey/USA/MN [symbol] 1/2011] | | |
| YP_009551574.1[Chicken picobirnavirus] | 0.588 | 0.449 |
| YP_00924138 [symbol] .1[Porcine picobirnavirus] | 0.551 | 0.410 |
| BANS8175.1(A [symbol] 82 [symbol] 072.1)[Picobirnavirus | 0.427 | 0.971 |
| cow [symbol] 7944Jap/2013] | | |
| AI [symbol] 06802.1(KF823811.1)[Fox fecal picobirnavirus] | 0.571 | 0.407 |
| AIY312 [symbol] 7.1(KMS73801.1[Dromedary picobirnavirus] | ID | 0.428 |
| AFK81927.1(JQ710506.1)[Picobirnavirus | 0.428 | ID |
| monkey/C [symbol] N−14/2002] | | |

[symbol]

indicates data missing or illegible when filed

BioEdit sequence identity matrix results.

Percent Identity

| | 1 | 2 | 3 | 4 | 5 | 6 | 7 | 8 | 9 | 10 | 11 | 12 | 13 | 14 | | |
|---|---|---|---|---|---|---|---|---|---|---|---|---|---|---|---|---|
| 1 | | 57.8 | 57.4 | 54.3 | 55.8 | 61.2 | 59.1 | 55.4 | 57.0 | 53.0 | 43.9 | 54.5 | 61.9 | 43.9 | 1 | ABT_PBV_RDRP |
| 2 | 61.3 | | 97.7 | 54.7 | 55.6 | 59.2 | 59.5 | 53.3 | 53.9 | 51.6 | 42.4 | 55.7 | 56.9 | 42.9 | 2 | AKG92636.1 |
| 3 | 62.1 | 2.3 | | 54.5 | 55.3 | 59.2 | 59.7 | 53.6 | 53.9 | 51.8 | 42.1 | 56.2 | 57.1 | 42.3 | 3 | AKG92637.1 |
| 4 | 69.2 | 68.1 | 68.6 | | 53.3 | 57.5 | 58.8 | 54.1 | 55.3 | 52.1 | 46.6 | 53.1 | 57.7 | 47.0 | 4 | ACT64131.1 |
| 5 | 65.6 | 66.1 | 66.8 | 71.5 | | 61.3 | 62.6 | 60.1 | 59.3 | 55.2 | 46.6 | 57.5 | 61.6 | 46.8 | 5 | YP_239361.1 |
| 6 | 54.2 | 58.3 | 58.3 | 61.7 | 54.0 | | 65.1 | 59.4 | 62.6 | 57.3 | 48. [symbol] | 57.8 | 65.3 | 48.4 | 6 | AAG53 [symbol] 83.1 |
| 7 | 58.4 | 57.5 | 57.1 | 59.1 | 51.4 | 46.8 | | 63.3 | 63.6 | 60.0 | 47.9 | 61.5 | 72.1 | 48.1 | 7 | YP_009351841.1 |
| 8 | 66.5 | 71.6 | 70.6 | 69.6 | 56.3 | 57.7 | 50.1 | | 59.6 | 56.7 | 48.2 | 60.2 | 60.9 | 48.4 | 8 | AHZ46150.1 |
| 9 | 63.0 | 70.1 | 70.1 | 66.8 | 58.0 | 51.4 | 49.6 | 57.3 | | 63.8 | 47.2 | 63.9 | 63.5 | 47.7 | 9 | YP_009551574.1 |
| 10 | 72.3 | 75.7 | 75.2 | 74.5 | 67.0 | 62.3 | 56.6 | 63.6 | 49.1 | | 44.1 | 65.2 | 59.9 | 43.9 | 10 | YP_009241386.1 |
| 11 | 97.7 | 102.9 | 103.8 | 89.3 | 89.4 | 85.3 | 85.6 | 84.8 | 87.7 | 97.2 | | 44.3 | 48.1 | 97.1 | 11 | BAN58175.1 |
| 12 | 68.7 | 65.9 | 64.6 | 72.0 | 61.7 | 61.3 | 53.3 | 56.2 | 48.9 | 46.5 | 96.5 | | 60.0 | 44.5 | 12 | AIB06802.1 |
| 13 | 52.7 | 63.2 | 62.8 | 61.3 | 53.3 | 46.4 | 34.8 | 54.7 | 49.7 | 56.7 | 85.2 | 56.5 | | 48.3 | 13 | AIY31287.1 |
| 14 | 97.7 | 101.0 | 103.2 | 88.2 | 88.8 | 84.2 | 85.0 | 84.3 | 86.1 | 97.8 | 2.9 | 95.9 | 84.6 | | 14 | AFK81927.1 |
| | 1 | 2 | 3 | 4 | 5 | 6 | 7 | 8 | 9 | 10 | 11 | 12 | 13 | 14 | | |

DNAstar MegAlign results for sequence identity and divergence (top = identity [symbol] bottom = divergence) [symbol]
indicates data missing or illegible when filed

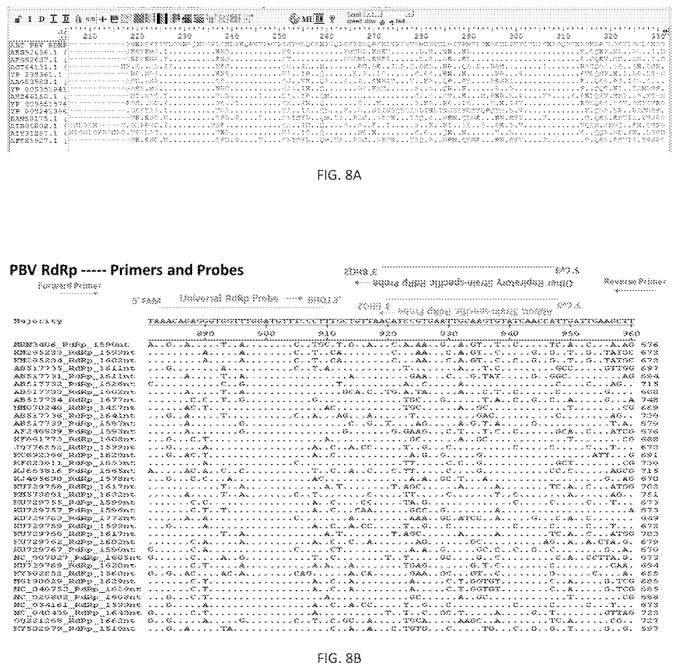

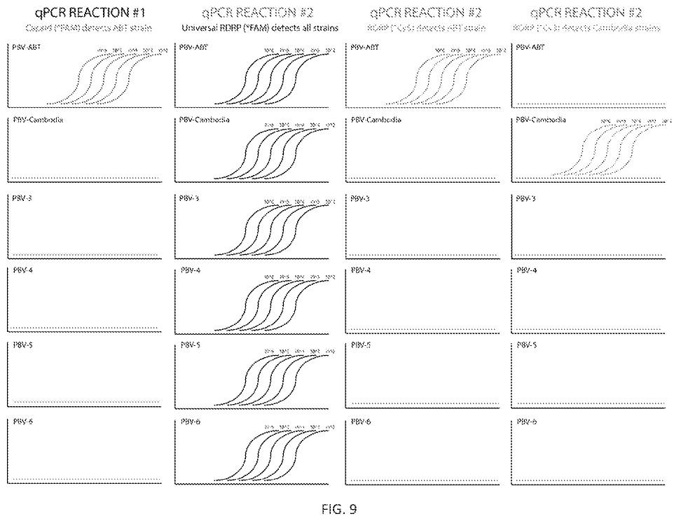

Scanning across the alignment it is clear that considerable identity resides in the portion used for the 55 aa tree (e.g. aa 209-264) (FIG. 8A). Investigating whether conserved RDRP motifs in other viruses resemble this sequence, and which ones, will be of interest to understand if these residues confer respiratory tropism.

In keeping with current established nomenclature, the novel strain described herein is referred to and deposited in GenBank as follows: GI/PBV/human/Colombia/ABT3406/2018.

### EXAMPLE 2

### PCR Detection of Picobirnavirus

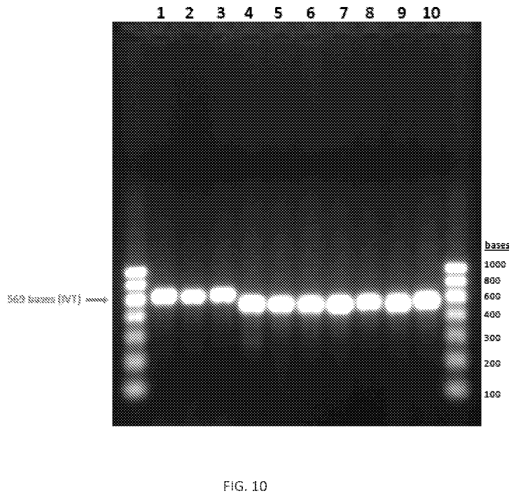

Methods for molecular detection of the novel picobirnavirus described herein (e.g. ABT-PBV) were designed to include the means to detect all picobirnaviruses, as well as the ability to discriminate the novel picobirnavirus described herein from other strains and confirm that both genomic segments are present in a sample. For this reason, the PCR assays described herein use one set of primers to amplify a ‘unique’ target on segment 1 to only detect the capsid sequence present in highly similar strains. In a separate reaction, another set of primers amplifies a ‘common’ target on segment 2 for detection of RDRP. Within this RDRP amplicon, all PBVs can be detected with one ‘general’ probe (FAM) and the novel PBV and highly related respiratory strains can be detected with ‘specific’ probes (Cy5 & Cy3). A nucleotide alignment of the RDRP amplicon region shows the position of these probes and which strains they detect (FIG. 8B.) Note that in FIG. 8B the forward and reverse primers are located outside of the region shown where probes hybridize. The general qPCR scheme and expected results as described above are summarized in FIG. 9.

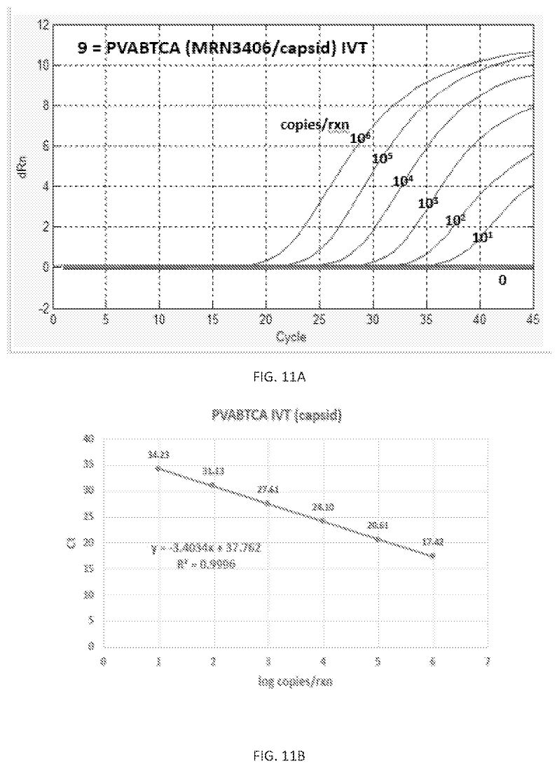

In vitro transcripts of capsid (n=1, lane 9) and RDRP (n=6, lanes 4-8, 10) sequences from ABT-PBV (lanes 9 & 10) and from additional PBV strains (lanes 4-8) were generated as positive controls to demonstrate detection in each qPCR assay (FIG. 10). The transcripts in lanes 1-3 are for aichivirus and are not described in this application.

Transcript of T7 Promoter—Aichi/PBV Insert (512 Bases)—Hind III=569 Bases

1=AVABT (ABT4352)

2=AVDQ (DQ028632)

3=AVNC (NC_001918)

4=PVABRD (AB517739)

5=PVGQRD (GQ221268)

6=PVKMRD (KM285233)

7=PVKURD(KU729763)

8=PVABTRD (MRN3406/RDRP)

9=PVABTCA (MRN3406/capsid)

10=PVNCRD (NC_007027)

The following primers and probes were developed.

(A) Capsid

Forward Primer: CAF1151
(SEQ ID NO: 13)
5′-CACCTACTCCAGATGATGTC-3′
Reverse Primer: CAR1229
(SEQ ID NO: 14)
5′-CTGTACCCATAGCAGTGAATA
Probe: CAP1186
(SEQ ID NO: 15)
5′ FAM-TTAGCTGTGGCATTAGAACCAGGCGC-BHQ1 3′

(B) RDRP

Forward Primers
(1) PVFP1:
(SEQ ID NO: 16)
5′-TGGCGIGGICARGAAGG-3′
(2) PVFP2:
(SEQ ID NO: 17)
5′-TGGAGAGGICAIGARGG-3′
(3) PVFP3:
(SEQ ID NO: 18)
5′-TGGCGIGGICARGAGGG-3′
Reverse Primers:
(1) PVRP1:
(SEQ ID NO: 19)
5′-CCATICIAAYCCAIGCAGG-3′
(2) PVRP2:
(SEQ ID NO: 20)
5′-CIAWGCIAACCCAIGCTGG-3′
(3) KMRP:
(SEQ ID NO: 21)
5′-CAIICCGACCCAWGCTGG-3′
(4) GQRP:
(SEQ ID NO: 22)
5′-ATAAACCAATCCATGGCGCTAT-3′
(5) MGRP:
(SEQ ID NO: 23)
5′-ACCICGTCATTRCIIWCCCA-3′
Probes:
(1) PVPROF1:
(SEQ ID NO: 24)
5′ FAM-CGTIAARCARIGIGTIGTITGGATGTTYCC-BHQ1 3′
(2) PVPROF2:
(SEQ ID NO: 25)
5′ FAM-CGTIAARCARAGIGTIGTITGGATGTTCCC-BHQ1 3′
(3) PVPROF3:
(SEQ ID NO: 26)
5′ FAM-CGTIAARCAGCGIGTIGTITGGATGTTYCC-BHQ1 3′
(4) MRNRPRO:
(SEQ ID NO: 27)
5′ Cy5-CGTTGCGCTGTTTCGATTAGAGGTTGG-BHQ2 3′
(5) KMRPRO:
(SEQ ID NO: 28)
5′ Cy3-TGTAGCATATCCATAAACGGCTGRTAGAC-BHQ2 3′
I = deoxyinosine; R = A + G; W = A + T; Y = C + T

Primers and probe combinations were tested to determine efficacy in detection of PBV.

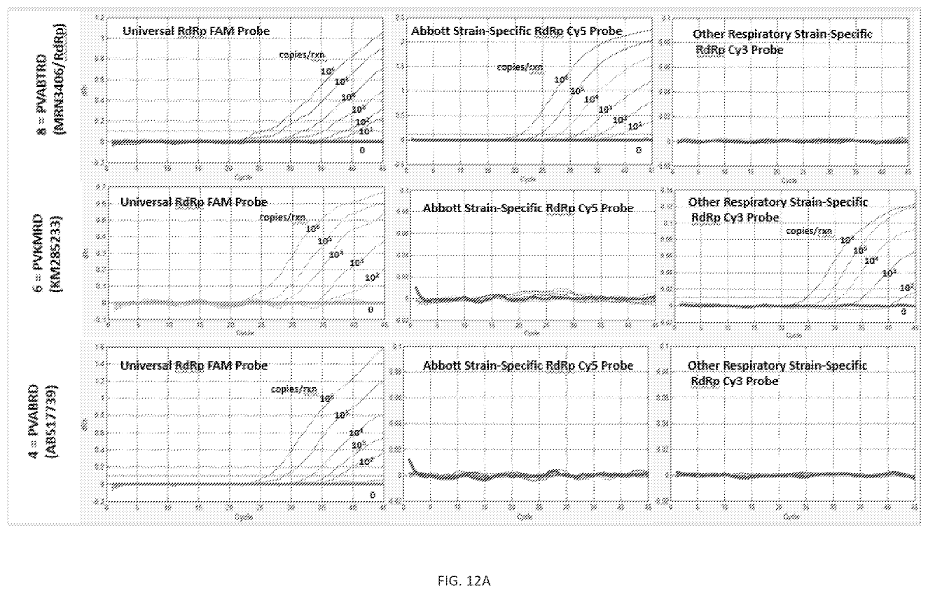

FIG. 11A-B show qPCR results for the serially diluted capsid IVT using the capsid primers and probes expected to detect only the novel PBV strain described herein. The capsid primers and probes described above were used (SEQ ID NO: 13, SEQ ID NO: 14, and SEQ ID NO: 15). Amplification curves are shown in FIG. 11A. The linear regression plot is shown in FIG. 11B. The novel ABT-PBV strain is detected with a limit of detection at or below 10 copies/ml and the response is linear.

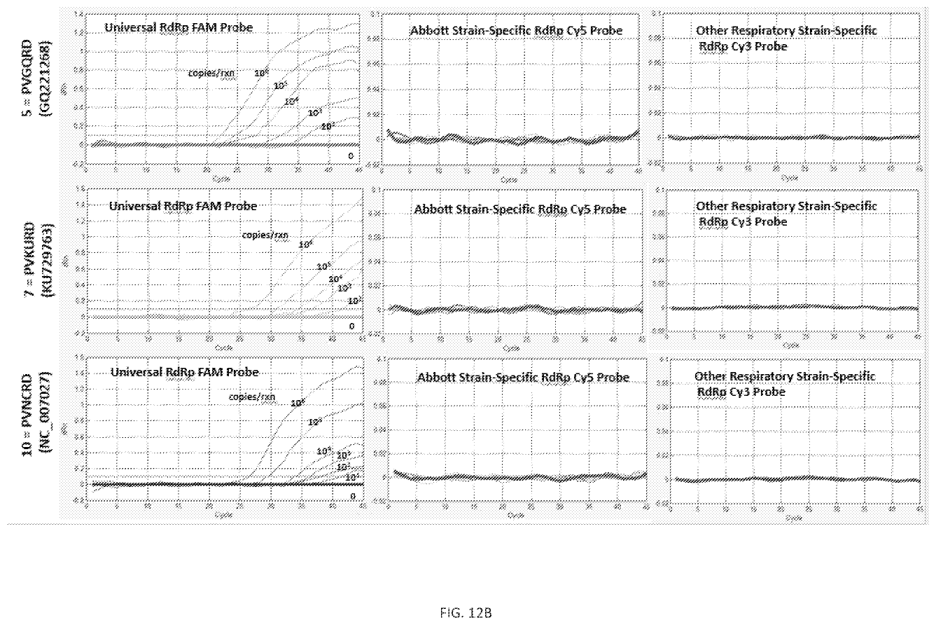

FIG. 12 shows PCR results for RDRP using the following primers and probes:

Forward Primers
(1) PVFP1:
(SEQ ID NO: 16)
5′-TGGCGIGGICARGAAGG-3′
(2) PVFP2:
(SEQ ID NO: 17)
5′-TGGAGAGGICAIGARGG-3′
(3) PVFP3:
(SEQ ID NO: 18)
5′-TGGCGIGGICARGAGGG-3′
Reverse Primers:
(1) PVRP1:
(SEQ ID NO: 19)
5′-CCATICIAAYCCAIGCAGG-3′
(2) PVRP2:
(SEQ ID NO: 20)
5′-CIAWGCIAACCCAIGCTGG-3′
(3) KMRP:
(SEQ ID NO: 21)
5′-CAIICCGACCCAWGCTGG-3′
(4) GQRP:
(SEQ ID NO: 22)
5′-ATAAACCAATCCATGGCGCTAT-3′
(5) MGRP:
(SEQ ID NO: 23)
5′-ACCICGTCATTRCIIWCCCA-3′
Probes:
(1) PVPROF1:
(SEQ ID NO: 24)
5′ FAM-CGTIAARCARIGIGTIGTITGGATGTTYCC-BHQ1 3′
(2) PVPROF2:
(SEQ ID NO: 25)
5′ FAM-CGTIAARCARAGIGTIGTITGGATGTTCCC-BHQ1 3′
(3) PVPROF3:
(SEQ ID NO: 26)
5′ FAM-CGTIAARCAGCGIGTIGTITGGATGTTYCC-BHQ1 3′

These primer and probe sets were first tested for the ability to detect IVT transcripts of sequences derived from multiple PBV strains. Multiple, forward (SEQ IDs 16-18) and reverse (SEQ IDs 19-23) primers located at the same positions and with degenerate bases are included in the reaction to ensure amplification of genetically diverse strains. Likewise, three similar FAM probes were included to accommodate expected mismatches (SEQ IDs 24-26). As shown in column 1 of FIG. 12A and FIG. 12B, the combination was able to detect the IVT of all six strains of PBV that were tested. Accordingly, this combination is referred to herein as a set of “universal primers and probes” that is able to detect all PBV strains, including the novel PBV strain described herein (e.g., ABT-PBV). Amplification curves in the FAM channel illustrate that detection is dose-dependent with LODs between 10-100 copies/ml.

Other probes capable of detecting only the novel PBV strain described herein were subsequently tested. Note that the probes selected for two RDRP sequences reside within the same amplicon described above, and therefore the forward (SEQ IDs 16-18) and reverse (SEQ IDs 19-23) primers are the same. The combination was as follows:

Forward Primers
(1) PVFP1:
(SEQ ID NO: 16)
5′-TGGCGIGGICARGAAGG-3′
(2) PVFP2:
(SEQ ID NO: 17)
5′-TGGAGAGGICAIGARGG-3′
(3) PVFP3:
(SEQ ID NO: 18)
5′-TGGCGIGGICARGAGGG-3′
Reverse Primers:
(1) PVRP1:
(SEQ ID NO: 19)
5′-CCATICIAAYCCAIGCAGG-3′
(2) PVRP2:
(SEQ ID NO: 20)
5′-CIAWGCIAACCCAIGCTGG-3′
(3) KMRP:
(SEQ ID NO: 21)
5′-CAIICCGACCCAWGCTGG-3′
(4) GQRP:
(SEQ ID NO: 22)
5′-ATAAACCAATCCATGGCGCTAT-3′
(5) MGRP:
(SEQ ID NO: 23)
5′-ACCICGTCATTRCIIWCCCA-3′
Probes:
(4) MRNRPRO:
(SEQ ID NO: 27)
5′ Cy5-CGTTGCGCTGTTTCGATTAGAGGTTGG-BHQ2 3′
(5) KMRPRO:
(SEQ ID NO: 28)
5′ Cy3-TGTAGCATATCCATAAACGGCTGRTAGAC-BHQ2 3′

Columns 2 and 3 of FIG. 12A and FIG. 12B show qPCR results from serially diluted IVTs from the same six PBVs strains detected in column 1 in the FAM channel. These primers and the Cy5 probe detected only the novel PBV strain found in sputum and described herein; none of the other strains were detected (FIG. 12A and FIG. 12B, column 2). Similiarly, these primers and the Cy3 probe detected only the respiratory strain from Cambodia; none of the other strains were detected (FIG. 12A and FIG. 12B, column 3).

Below is a detailed description of the PBV Capsid qPCR reaction recipe and cycling conditions:

Prepare a master mix for 1 reaction (final volume 50 μl)

| Water | 11.45 | μl | | | |
|---|---|---|---|---|---|
| 2X RT-PCR Buffer | 25.0 μl (1X) | | | | |
| CAF1151 (100 μM in TE, pH 8.0) | 0.2 | μl | | | (0.4 μM) |
| CAR1229 (100 μM in TE, pH 8.0) | 0.2 | μl | {close oversize brace} | 0.55 μl | (0.4 μM) |
| CAP1186 (FAM) (100 μM in TE, pH 8.0) | 0.15 | μl | | | (0.3 μM) |
| 50 mM MgCl₂ | 1.0 μl (1 mM) | | | | |
| 25 × RT-PCR Enzyme Mix | 2.0 μl (1X) | | | | |
| | 40 | μl | | | |
| RNA | 10 | μl | | | |

50 μl per reaction

Forward primer (CAF1151), reverse primer (CAR1229), and FAM probe (CAP1186) were pre-mixed together in one tube; add 0.55 μl of the premixed primers and probes per 50 μl reaction.

ROX is used a reference dye in the RT-PCR buffer.

The AgPath-ID One-Step RT-PCR Kit (Life Technologies, cat #4387424) includes 2× RT-PCR Buffer, 25× RT-PCR Enzyme Mix, Detection Enhancer (×15) and Nuclease-free Water. The 50 mM MgCl₂ is provided separately.

10 μl Sample RNA (e.g. IVT, patient RNA) is added last, the plate is sealed and placed in the Abbott m2000rt instrument.

Real-Time PCR Cycling Conditions

| Stage | Cycle | Temperature | Time | |
|---|---|---|---|---|
| 1 | 1 | 50° C. | 30 | minutes |
| 2 | 1 | 95° C. | 10 | minutes |
| 3 | 45 | 95° C. | 30 | seconds |
| | | 62° C. | 30 | seconds |

| | |
|---|---|
| 55° C. | 90 seconds (signals read in last 30 seconds) |

Below is a detailed description of the PBV RDRP qPCR reaction recipe and cycling conditions:

Prepare a master mix for 1 reaction (final volume 50 μl)

| Water | 9.65 | μl | | | |
|---|---|---|---|---|---|
| 2 × RT-PCR Buffer | 25.0 μl (1×) | | | | |
| PVFP1 (100 μM in TE, pH 8.0) | 0.2 | μl | | | (0.4 μM) |
| PVFP2 (100 μM in TE, pH 8.0) | 0.2 | μl | | | (0.4 μM) |
| PVFP3 (100 μM in TE, pH 8.0) | 0.2 | μl | | | (0.4 μM) |
| PVRP1 (100 μM in TE, pH 8.0) | 0.2 | μl | | | (0.4 μM) |
| PVRP2 (100 μM in TE, pH 8.0) | 0.2 | μl | | | (0.4 μM) |
| KMRP (100 μM in TE, pH 8.0) | 0.2 | μl | {close oversize brace} | 2.05 μl | (0.4 μM) |
| GQRP (100 μM in TE, pH 8.0) | 0.2 | μl | | | (0.4 μM) |
| MGRP (100 μM in TE, pH 8.0) | 0.2 | μl | | | (0.4 μM) |
| PVPROF1 (FAM) (100 μM in TE, pH 8.0) | 0.15 | μl | | | (0.3 μM) |
| PVPROF2 (FAM) (100 μM in TE, pH 8.0) | 0.15 | μl | | | (0.3 μM) |
| PVPROF3 (FAM) (100 μM in TE, pH 8.0) | 0.15 | μl | | | (0.3 μM) |
| MRNRPRO (Cy5) (100 μM in TE, pH 7.0) | 0.15 | μl | {close oversize brace} | 0.3 μl | (0.3 μM) |
| KMRPRO (Cy3) (100 μM in TE, pH 7.0) | 0.15 | μl | | | (0.3 μM) |
| 50 mM MgCl₂ | 1.0 μl (1 mM) | | | | |
| 25 × RT-PCR Enzyme Mix | 2.0 μl (1×) | | | | |
| | 40 | μl | | | |
| RNA | 10 | μl | | | |

50 μl per reaction

Forward primers (PVFP1/2/3), reverse primers (PVRP1/2, KMRP, GQRP, and MGRP), FAM probes (PVPROF1/2/3 targeting all PBV strains in RdRp) are pre-mixed together in one tube in TE, pH 8.0; add 2.05 μl of the premixed primers and probes for each 50 μl reaction.

MRNRPRO Cy5 probe targeting the novel ABT-PBV strain in RdRp and KMRPRO Cy3 probe targeting other respiratory PBV strains in RdRp are pre-mixed together in one tube in TE, pH 7.0; add 0.3 μl of the premixed Cy5/Cy3 probes for each 50 μl reaction.

ROX is used a reference dye in the RT-PCR buffer.

The AgPath-ID One-Step RT-PCR Kit (Life Technologies, cat #4387424) includes 2× RT-PCR Buffer, 25× RT-PCR Enzyme Mix, Detection Enhancer (×15) and Nuclease-free Water. The 50 mM MgCl₂ is provided separately.

10 μl Sample RNA (e.g. IVT, patient RNA) is added last, the plate is sealed and placed in the Abbott m2000rt instrument.

Real-Time PCR Cycling Conditions

| Stage | Cycle | Temperature | Time | |
|---|---|---|---|---|
| 1 | 1 | 50° C. | 30 | minutes |
| 2 | 1 | 95° C. | 10 | minutes |
| 3 | 45 | 95° C. | 30 | seconds |
| | | 62° C. | 30 | seconds |

| | |
|---|---|
| 55° C. | 90 seconds (read signals in last 30 seconds) |

### EXAMPLE 3

### Detection of Additional Strains in Sputum Samples

To identify additional strains related to the novel PBV described in Example 1 and simultaneously demonstrate the utility of the qPCR assay described in Example 2, sputum specimens from patients ill and/or hospitalized with severe respiratory symptoms were screened. The following 130 sputum samples were obtained from three different commercial vendors:

N=50 from NY Biologics collected at outpatient facility (New York, USA)

N=30 from Boca Biolistics collected from hospitalized patients (USA)

N=50 from MRN Diagnostics newly collected from hospitalized patients (Colombia, South America). Note: The original set had 24 samples, these 50 were collected ˜2 yrs later from the same medical facility.

Selection of these samples from multiple sites were expected to provide an indication of the general prevalence of picobirnaviruses in individuals with respiratory illness. Positive detection of strains highly similar to the novel ABT-PBV (Capsid FAM+; RDRP FAM+, and RDRP Cy5+) will also indicate whether this particular virus is circulating in the population.

Extraction Procedure

Sputum samples were resuspended at 1:1 proportion (e.g. 500 μl of 2× buffer with ˜500 μl of sputum) in 2× pretreatment buffer (below) for 3 hours at 37° C. Forty-eight samples were processed at a time according to the TNA+Proteinase K extraction procedure required of the automated m2000 platform. Therefore, 25 ml of 2× buffer was prepared fresh for each of 3 rounds of samples preparations performed at different time points.

The pre-treatment procedure was performed in a BSL3 facility. All manipulations took place in laminar flow biosafety cabinets and personnel donned full PPE and respirators. All trash (e.g. tips, pestles, etc.) was retained in sealable roller bottles and autoclaved.

2× Pretreatment Buffer (25 ml):
5 ml of 10× Benzonase buffer (2×)
5 ml of 1% DTT in water (0.2%), [1%=0.5 g DTT in 50 ml water]
14.8 ml of water
50 μl of Sigma Benzonase→2 μl/ml=1 μl/sample=250 U/sample
50 μl of Sigma Turbo DNAse→2 μl/ml=1 μl/sample=200 U/sample
100 μl of Roche DNAse 1→4 μl/ml=2 μl/sample=20 U/sample

Nuclease Information

| Sigma ultra-pure benzonase @ 250 U/μl | E8263-5KU | 20 μl/tube (×3 tubes) |
|---|---|---|
| Sigma Turbo DNAse from S. marcescens @ 200 U/μl | T4330-50KU | 250 μl/tube |
| Roche (Sigma) DNAse 1 recombinant @ 10 U/μl | 04716728001 | 1000 μl/tube |

Step by Step Procedure

Step 1. Transfer ˜500 μl of sputum to a labeled 2.0 ml Eppendorf centrifuge tube using either a sterile disposable spatula or wood Q-tip handle. Spin down briefly where needed to line up level of sputum with 500 μl gradation on the tube.

Step 2. Pipette 500 μl of 2× buffer (above) to each sample and vortex. Quick spin to collect.

Step 3. Use a disposable pestle to mechanically disrupt the sputum where necessary. Use ≥10 passes depending on viscosity. Place tubes in 37° C. heat block.

Step 4. At 45 min intervals, repeat vortexing. Return samples to 37° C. heat block and incubate for 3 hr total.

Step 5. Spin samples at 10,000 rpm for 2 min to pellet insoluble debris. Transfer 800 μl of sample to an m2000 sample tube and cap it.

Step 6. Extract material on an m2000 using the TNA+Proteinase K protocol (Abbott Molecular, Des Plaines, Ill.).

Step 7. Freeze deep-well plate of extracted nucleic acid at −80° C. until use.

Patient Specimen Screening by qPCR

Capsid qPCR mastermix (40 μl, as described above) was dispensed to a 96 well PCR plate. 10 μl of each sample RNA was added to mastermix.

In vitro transcript, PVABTCA (novel PBV strain capsid, #9), resuspended in water at 10⁶, 10⁵, and 10⁴ copies/10 ul served as the positive control and water served as the negative control.

RDRP qPCR mastermix (40 μl, as described above) was dispensed to a separate 96 well PCR plate. 10 μl of each sample RNA was added to mastermix.

In vitro transcripts, PVABTRD (novel PBV strain RdRp, #8), PVKMRD (another PBV respiratory strain RdRp, #6), and PVGQRD (a representative non-respiratory PBV strain RdRp, #5), were resuspended in water at 10⁶, 10⁵, and 10⁴ copies/10 ul and served as positive controls. Water served as the negative control.

Reactions were cycled as described above for IVT. Results were analyzed in MultiAnalyze software.

Results:

Separate capsid and RDRP qPCRs were performed and the cycle threshold values are listed below. Positive sample results are highlighted in different colors to represent the different classes of PBVs identified.

The first set of samples screened (column 1, n=48) from NY Biologics (USA) revealed four hits. Two hits were detected by the RDRP qPCR that represent any PBV strain (FAM channel only). Given these are found in the sputum of sick individuals, they are presumably altogether new respiratory PBV strains, but with RDRP sequences (and capsid) not related to the Cambodian (CY3−) or the novel ABT (CY5−) strain from Colombia described herein. In addition two hits were detected that indicate these individuals have PBV strains with an RDRP sequences similar to the novel ABT-PBV strain (FAM+, CY5+).

The second set of samples screened (column 2, n=48) were from all 3 vendors [NY Biologics (USA), Boca Biolistics (USA), and MRN Dx (Colombia)] and revealed six hits. Five hits were detected that indicate these PBV strains have an RDRP similar to the novel ABT-PBV strain (FAM+, CY5+). A single isolate was detected where the RDRP is similar to the respiratory strain from Cambodia (FAM+, CY3+). There were weak signals (italics) that upon further analysis were eliminated as positives.

The third set of samples screened (column 2, n=38) were all from MRN Dx (Colombia) and revealed 15 hits. Three hits were detected that represent any PBV strain (FAM+); four hits with an RDRP similar to the ABT-PBV strain (FAM+, CY5+), and 1 hit where the RDRP is similar to the respiratory strain from Cambodia (FAM+, CY3+); all of these were capsid negative. Additionally, 7 hits were detected that were dually positive for capsid and RDRP (FAM+, FAM+, CY5+). Two of these were also positive in the Cy3 channel (FAM+, FAM+, CY3+, CY5+), which can either represent a mixed infection or cross reactivity with what are indeed highly similar probes.

Set 1: NY Biologics

| | | Capsid [symbol] FAM | | RDRP [symbol] FAM | RDRP [symbol] CY3 | RDRP [symbol] CY5 |
|---|---|---|---|---|---|---|
| | Sample ID | C [symbol] | Sample ID | C [symbol] | C [symbol] | C [symbol] |
| | 564225 | −1 | 564225 | −1 | −1 | −1 |
| | 564226 | −1 | 564226 | −1 | −1 | −1 |
| | 564227 | −1 | 564227 | −1 | −1 | −1 |
| | 564228 | −1 | 564228 | −1 | −1 | −1 |
| | 564229 | −1 | 564229 | −1 | −1 | −1 |
| | 564230 | −1 | 564230 | −1 | −1 | −1 |
| | 564231 | −1 | 564231 | −1 | −1 | −1 |
| | 564232 | −1 | 564232 | −1 | −1 | −1 |
| | 564233 | −1 | 564233 | −1 | −1 | −1 |
| | 564234 | −1 | 564234 | −1 | −1 | −1 |
| | 564235 | −1 | 564235 | −1 | −1 | −1 |
| | 564236 | −1 | 564236 | −1 | −1 | −1 |
| | 564237 | −1 | 564237 | −1 | −1 | −1 |
| | 564238 | −1 | 564238 | −1 | −1 | −1 |
| | 564239 | −1 | 564239 | −1 | −1 | −1 |
| | 564240 | −1 | 564240 | −1 | −1 | −1 |
| [symbol] | 564466 | −1 | 564466 | 29.41 | −1 | −1 |
| | 564467 | −1 | 564467 | −1 | −1 | −1 |
| [symbol] | 564468 | −1 | 564468 | 33.36 | −1 | 30.3 [symbol] |
| | 564469 | −1 | 564469 | −1 | −1 | −1 |
| | 564137 | −1 | 564137 | −1 | −1 | −1 |
| † | 564138 | −1 | 564138 | 26.4 | −1 | [symbol] .87 |
| | 564139 | −1 | 564139 | −1 | −1 | −1 |
| | 564140 | −1 | 564140 | −1 | −1 | −1 |
| | 564141 | −1 | 564141 | −1 | −1 | −1 |
| | 564142 | −1 | 564142 | −1 | −1 | −1 |
| | 564143 | −1 | 564143 | −1 | −1 | −1 |
| | 564144 | −1 | 554144 | −1 | −1 | −1 |
| | 564221 | −1 | 564221 | −1 | −1 | −1 |
| | 564222 | −1 | 564222 | −1 | −1 | −1 |
| | 564223 | −1 | 564223 | −1 | −1 | −1 |
| | 564224 | −1 | 564224 | −1 | −1 | −1 |
| | 564475 | −1 | 564475 | −1 | −1 | −1 |
| | 564476 | −1 | 564476 | −1 | −1 | −1 |
| [symbol] | 564477 | −1 | 564477 | 36. [symbol] 6 | −1 | −1 |
| | 564478 | −1 | 564478 | −1 | −1 | −1 |
| | 564479 | −1 | 564479 | −1 | −1 | −1 |
| | 564480 | −1 | 564480 | −1 | −1 | −1 |
| | 564505 | −1 | 564505 | −1 | −1 | −1 |
| | 564506 | −1 | 564506 | −1 | −1 | −1 |
| | 564507 | −1 | 564507 | −1 | −1 | −1 |
| | 564508 | −1 | 564508 | −1 | −1 | −1 |
| | 564509 | −1 | 564509 | −1 | −1 | −1 |
| | 564510 | −1 | 564510 | −1 | −1 | −1 |
| | 564511 | −1 | 564511 | −1 | −1 | −1 |
| | NHP | −1 | NHP | −1 | −1 | −1 |
| | C [symbol] _9 | −1 | C [symbol] _9 | −1 | −1 | −1 |
| | Sputum C | −1 | Sputum C | −1 | −1 | −1 |
| | 10 [symbol] | 20.1 [symbol] | 10\hat{ }6 [symbol] | 23.9 [symbol] | −1 [symbol] | −1 [symbol] |
| | 10 [symbol] 5 [symbol] | 26. [symbol] | 10\hat{ }5 [symbol] | 26.98 [symbol] | −1 [symbol] | −1 [symbol] |
| | 10 [symbol] 4 [symbol] | [symbol] .8 [symbol] | 10\hat{ }4 [symbol] | [symbol] 0. [symbol] | −1 [symbol] | −1 [symbol] |
| | NTC | −1 | 10\hat{ }6 [symbol] | 27. [symbol] 4 [symbol] | 24.82 [symbol] | −1 [symbol] |
| | | | 10\hat{ } [symbol] | 30.18 [symbol] | 27. [symbol] | −1 [symbol] |
| | | | 10\hat{ }4 [symbol] | 33.19 [symbol] | 30.7 [symbol] | −1 [symbol] |
| | | | 10\hat{ }6 [symbol] | 28.17 [symbol] | −1 [symbol] | 23.43 [symbol] |
| | | | 10\hat{ }5 [symbol] | [symbol] 0.34 [symbol] | −1 [symbol] | 2 [symbol] .75 [symbol] |
| | | | 10\hat{ }4 [symbol] | 35.92 [symbol] | −1 [symbol] | 31. [symbol] 5 [symbol] |
| | | | NTC | −1 | −1 | −1 |
| | | | NTC | −1 | −1 | −1 |
| | | | NTC | −1 | −1 | −1 |

Set 2: NY Biologics Boca Bio MRN Dx

| | | Capsid [symbol] FAM | | RDRP [symbol] FAM | RDRP [symbol] CY [symbol] | RDRP [symbol] CY [symbol] |
|---|---|---|---|---|---|---|
| | Sample ID | C [symbol] | Sample ID | C [symbol] | C [symbol] | C [symbol] |
| † | 564470 | −1 | 564470 | 34.6 | −1 | 32.28 |
| | 564471 | −1 | 564471 | −1 | −1 | −1 |
| | 564472 | −1 | 564472 | [symbol] 3.29 | −1 | −1 |
| | 564473 | −1 | 564473 | −1 | −1 | −1 |
| | 564474 | −1 | 564474 | −1 | −1 | −1 |
| | D000024297 | −1 | D000024297 | −1 | −1 | −1 |
| | D000030 [symbol] 4 | −1 | D000030 [symbol] 4 | −1 | −1 | −1 |
| | D00030560 | −1 | D00030560 | −1 | −1 | −1 |
| | D000030566 | −1 | D000030566 | −1 | −1 | −1 |
| | D000046713 | −1 | D000046713 | −1 | −1 | −1 |
| | D000046716 | −1 | D000046716 | −1 | −1 | −1 |
| | D000047120 | −1 | D000047120 | −1 | −1 | −1 |
| † | D000047122 | −1 | D000047122 | 35.04 | −1 | 33.75 |
| | D000047124 | −1 | D000047124 | −1 | −1 | −1 |
| | D000047127 | −1 | D000047127 | −1 | −1 | −1 |
| | D000047140 | −1 | D000047140 | −1 | −1 | −1 |
| | D000047153 | −1 | D000047153 | −1 | −1 | −1 |
| | D000047154 | −1 | D000047154 | −1 | −1 | −1 |
| | D000047157 | −1 | D000047157 | −1 | −1 | −1 |
| | D000047178 | −1 | D000047178 | −1 | −1 | −1 |
| | D000047179 | −1 | D000047179 | −1 | −1 | −1 |
| | D000047262 | −1 | D000047262 | −1 | −1 | −1 |
| | D0000472 [symbol] 7 | −1 | D0000472 [symbol] 7 | −1 | −1 | −1 |
| | D000047307 | −1 | D000047307 | 34. [symbol] | −1 | −1 |
| | D000052296 | −1 | D000052296 | −1 | −1 | −1 |
| | D000052297 | −1 | D000052297 | −1 | −1 | −1 |
| | D000052300 | −1 | D000052300 | 42.1 | −1 | −1 |
| | D000052301 | −1 | D000052301 | −1 | −1 | −1 |
| | D000052314 | −1 | D000052314 | −1 | −1 | −1 |
| | D000052342 | −1 | D000052342 | −1 | −1 | −1 |
| | D000052346 | −1 | D000052346 | −1 | −1 | −1 |
| | D000052348 | −1 | D000052348 | −1 | −1 | −1 |
| | D000052349 | −1 | D000052349 | −1 | −1 | −1 |
| | D000052350 | −1 | D000052350 | −1 | −1 | −1 |
| | D000052351 | −1 | D000052351 | −1 | −1 | −1 |
| ‡ | Sptm19-001 | 26. [symbol] 3 | Sptm19-001 | 30.78 | −1 | 24.06 |
| | Sptm19-002 | −1 | Sptm19-002 | −1 | −1 | −1 |
| | Sptm19-003 | −1 | Sptm19-003 | −1 | −1 | −1 |
| | Sptm19-004 | −1 | Sptm19-004 | −1 | −1 | −1 |
| | Sptm19-005 | −1 | Sptm19-005 | −1 | −1 | −1 |
| ‡ | Sptm19-006 | 22.1 [symbol] | Sptm19-006 | 2 [symbol] .33 | −1 | 20.42 |
| | Sptm19-007 | −1 | Sptm19-007 | −1 | −1 | −1 |
| | Sptm19-008 | −1 | Sptm19-008 | −1 | −1 | −1 |
| | Sptm19-009 | −1 | Sptm19-009 | −1 | −1 | −1 |
| | Sptm19-010 | −1 | Sptm19-010 | −1 | −1 | −1 |
| | Sptm19-011 | −1 | Sptm19-011 | −1 | −1 | −1 |
| [symbol] | Sptm19-012 | −1 | Sptm19-012 | 23.36 | 22.78 | −1 |
| ‡ | Sput Ctrl | 35.26 | Sput Ctrl | 36.34 | −1 | 33.8 |
| | 10\hat{ }6 [symbol] | 19.85 [symbol] | 10\hat{ }6 [symbol] | [symbol] .09 [symbol] | −1 [symbol] | −1 [symbol] |
| | 10\hat{ }5 [symbol] | 26.21 [symbol] | 10\hat{ }5 [symbol] | 2 [symbol] . [symbol] 2 [symbol] | −1 [symbol] | −1 [symbol] |
| | 10\hat{ }4 [symbol] | 26. [symbol] | 10\hat{ }4 [symbol] | 28.4 [symbol] | −1 [symbol] | −1 [symbol] |
| | NTC | −1 | 10\hat{ }6 [symbol] | 28.31 [symbol] | 27.45 [symbol] | −1 [symbol] |
| | | | 10\hat{ }5 [symbol] | 32.65 [symbol] | [symbol] 2.0 [symbol] | −1 [symbol] |
| | | | 10\hat{ }4 [symbol] | 35.5 [symbol] | 34.98 [symbol] | −1 [symbol] |
| | | | 10\hat{ }6 [symbol] | 27.0 [symbol] | −1 [symbol] | 23.29 [symbol] |
| | | | 10\hat{ }5 [symbol] | 29.51 [symbol] | −1 [symbol] | 26.85 [symbol] |
| | | | 10\hat{ }4 [symbol] | 34.7 [symbol] | −1 [symbol] | 31.3 [symbol] |
| | | | NTC | −1 | −1 | −1 |
| | | | NTC | −1 | −1 | −1 |
| | | | NTC | −1 | −1 | −1 |

Set 3: MRN Dx

| | | Capsid [symbol] FAM | | RDRP [symbol] FAM | RDRP [symbol] CY [symbol] | RDRP [symbol] CY5 |
|---|---|---|---|---|---|---|
| | Well | C [symbol] | Sample ID | C [symbol] | C [symbol] | C [symbol] |
| | Sptm19-013 | −1 | Sptm19-013 | −1 | −1 | −1 |
| | Sptm19-014 | −1 | Sptm19-014 | −1 | −1 | −1 |
| ‡ | Sptm19-015 | 24.69 | Sptm19-015 | 23.1 | 24.15 | 25.5 |
| † | Sptm19-016 | −1 | Sptm19-016 | 28.71 | −1 | 25.56 |
| | Sptm19-017 | −1 | Sptm19-017 | −1 | −1 | −1 |
| | Sptm19-018 | −1 | Sptm19-018 | −1 | −1 | −1 |
| | Sptm19-019 | −1 | Sptm19-019 | −1 | −1 | −1 |
| [symbol] | Sptm19-020 | −1 | Sptm19-020 | 35.51 | −1 | −1 |
| ‡ | Sptm19-021 | 35.59 | Sptm19-021 | 35.51 | −1 | −1 |
| | Sptm19-022 | −1 | Sptm19-022 | −1 | −1 | −1 |
| [symbol] | Sptm19-023 | −1 | Sptm19-023 | 25.12 | 24. [symbol] | −1 |
| | Sptm19-024 | −1 | Sptm19-024 | −1 | −1 | −1 |
| | Sptm19-025 | −1 | Sptm19-025 | −1 | −1 | −1 |
| | Sptm19-026 | −1 | Sptm19-026 | −1 | −1 | −1 |
| | Sptm19-027 | −1 | Sptm19-027 | −1 | −1 | −1 |
| | Sptm19-028 | −1 | Sptm19-028 | −1 | −1 | −1 |
| | Sptm19-029_A | −1 | Sptm19-029_A | −1 | −1 | −1 |
| | Sptm19-029_B | −1 | Sptm19-029_B | −1 | −1 | −1 |
| | Sptm19-030 | −1 | Sptm19-030 | −1 | −1 | −1 |
| † | Sptm19-031 | −1 | Sptm19-031 | 32.26 | −1 | 30.16 |
| † | Sptm19-032 | −1 | Sptm19-032 | 27.69 | −1 | 25.94 |
| [symbol] | Sptm19-033 | −1 | Sptm19-033 | 25.35 | 26.91 | 34.56 |
| [symbol] | Sptm19-034 | −1 | Sptm19-034 | 26.41 | 22.3 [symbol] | 35. [symbol] |
| ‡ | Sptm19-035 | 35.84 | Sptm19-035 | 29.3 | −1 | 2 [symbol] . [symbol] 4 |
| † | Sptm19-036 | −1 | Sptm19-036 | 34.19 | −1 | [symbol] 0.89 |
| | Sptm19-037 | −1 | Sptm19-037 | −1 | −1 | −1 |
| ‡ | Sptm19-038 | 26.57 | Sptm19-038 | 30.67 | −1 | 2 [symbol] .08 |
| ‡ | Sptm19-039 | 34.43 | Sptm19-039 | 21.56 | 25.45 | 2 [symbol] .43 |
| | Sptm19-040 | −1 | Sptm19-040 | −1 | −1 | −1 |
| | Sptm19-041 | −1 | Sptm19-041 | −1 | −1 | −1 |
| | Sptm19-042 | −1 | Sptm19-042 | −1 | −1 | −1 |
| | Sptm19-043 | −1 | Sptm19-043 | −1 | −1 | −1 |
| ‡ | Sptm19-044 | 22.58 | Sptm19-044 | 31.48 | −1 | 26.95 |
| | Sptm19-045 | −1 | −1 | −1 | −1 | −1 |
| † | Sptm19-046 | 19.61 | Sptm19-046 | 27.14 | −1 | 23.29 |
| | Sptm19-047 | −1 | Sptm19-047 | −1 | −1 | −1 |
| | Sptm19-048 | −1 | Sptm19-048 | −1 | −1 | −1 |
| | Sptm19-049 | −1 | Sptm19-049 | −1 | −1 | −1 |
| | 10\hat{ }6 [symbol] | 19.18 [symbol] | 10\hat{ }6 [symbol] | [symbol] 2.96 [symbol] | −1 [symbol] | −1 [symbol] |
| | 10\hat{ }5 [symbol] | 25.81 [symbol] | 10\hat{ }5 [symbol] | 26.57 [symbol] | −1 [symbol] | −1 [symbol] |
| | 10\hat{ }4 [symbol] | 26.52 [symbol] | 10\hat{ }4 [symbol] | 30.18 [symbol] | −1 [symbol] | −1 [symbol] |
| | NTC | −1 | 10\hat{ }6 [symbol] | 28.23 [symbol] | 27.7 [symbol] | −1 [symbol] |
| | | | 10\hat{ }5 [symbol] | 31.18 [symbol] | 31.7 [symbol] | −1 [symbol] |
| | | | 10\hat{ }4 [symbol] | 33.81 [symbol] | 34.75 [symbol] | −1 [symbol] |
| | | | 10\hat{ }6 [symbol] | 28.95 [symbol] | −1 [symbol] | 24.0 [symbol] |
| | | | 10\hat{ }5 | 30.23 [symbol] | −1 [symbol] | 27.67 [symbol] |
| | | | 10\hat{ }4 [symbol] | 36.87 [symbol] | −1 [symbol] | 31.98 [symbol] |
| | | | NTC | −1 | −1 | −1 |
| | | | NTC | 35.2 [symbol] | −1 | −1 |
| | | | NTC | 40.46 | −1 | −1 |

*ABT PBV strain capsid+ ( [symbol] VT #9) [symbol]
Any PBV strain RdRp+ ( [symbol] VT #4 [symbol] 10) [symbol]
Non ABT PBV resp [symbol] ory [symbol] RdRp+ ( [symbol] VT # [symbol] )
† ABT PBV [symbol] in RdRp+ ( [symbol] VT # [symbol] )
‡ ABT PBV [symbol] rain RdRp+ [symbol] capsid+ ( [symbol] VT [symbol] ) [symbol]
indicates data missing or illegible when filed

Genome Characterization and mNGS of qPCR Positives

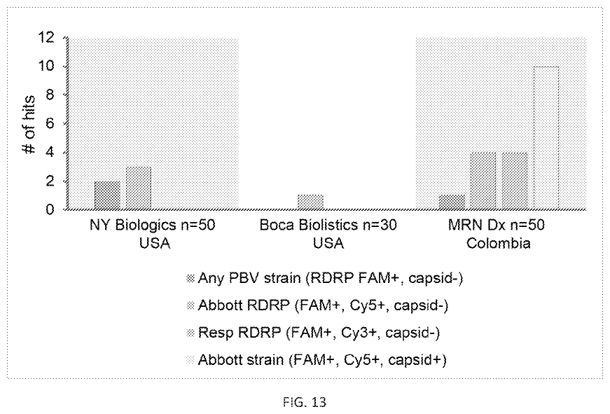

In total, 25 samples (19.2%) were positive for PBV. A summary of the types of hits (qPCR profile) obtained and from which cohort they originate is shown in FIG. 13. Total nucleic acid from the same extraction was converted into cDNA and Nextera libraries (n=25) for mNGS and determination of the full-length sequence of each PBV strain. The number of PBV reads identified in SURPI and DiVir correlated well with the viral loads inferred from the qPCR Ct values (see below). All raw reads were first aligned to the MRN3406 reference sequence as a first attempt to derive each new strain consensus sequence. Most of the Colombian strains (designated in yellow: Cap FAM+/RDRP FAM+/Cy3−/Cy5+) and a few of the US strains bore considerable nucleotide identity to the index which allowed for efficient mapping of reads and genome assembly. To verify the final consensus was not biased by this approach, contigs of PBV reads de novo assembled in both RAPsearch and DiVir pipelines were aligned and the sequences agreed. Similarly, samples like 19-012 (designated in orange: Cap FAM−/RDRP FAM+/Cy3+/Cy5−) were mapped to the RDRP sequence of the Cambodian reference strain, KM285233. This approach also sufficed to compile the RDRP sequences for samples 19-012, 19-023, 19-039, etc., and they were verified by comparison to pipeline-generated de novo contigs. However, capsid sequences for these strains were determined entirely by de novo assembly since there were no accompanying capsid sequences published with the KM285233 strain RDRP. Currently, 12 additional full and 5 partial genomes have been determined from 15 different individuals: 3 are co-infected with PBV similar to the ABT-PBV and Cambodian strains. The majority are from Colombia (n=13) and only 2 are from the US. As expected, samples with high Cts appear to have few PBV reads altogether and will likely not generate considerable genome coverage.

| | | Capsid [symbol] FAM | RDRP [symbol] FAM | RDRP [symbol] CY3 | RDP [symbol] CY5 | PBV Reads RDRP Segment 2 |
|---|---|---|---|---|---|---|
| NGS ID | Sample ID | Ct | Ct | Ct | Ct | Total |
| 1-PBV-4466 | 564466 | −1 | 29.41 | −1 | −1 | 2,647 |
| 2-PBV-4468 | 564468 | −1 | 33.36 | −1 | 30.34 | 10 |
| 3-PBV [symbol] 4138 | 564138 | −1 | 26.4 | −1 | 23. [symbol] 7 | 270 |
| 4-PBV-4477 | 564477 | −1 | 36.26 | −1 | −1 | 0 |
| 8-PBV-4470 | 564470 | −1 | 34.6 | −1 | 32.28 | 3 |
| 9-PBV-6722 | D000046722 | −1 | 35.04 | −1 | 33.75 | 0 |
| 10-PBV-19-001 | Sptm19-001 | 26.53 | 30.78 | −1 | 24.06 | 345 |
| 11-PBV-19-006 | Sptm19-006 | 22.18 | 28.33 | −1 | 20.42 | 2,912 |
| 12-PBV-19-012 | Sptm19-012 | −1 | 23.36 | 22.78 | −1 | 224 |
| 12-PBV-19-012 | Sptm19-012 | −1 | 23.36 | 22.78 | −1 | 3,712 |
| 13-PBV-SPTM- | Sputum Control | 35.26 | 36.34 | −1 | 33. [symbol] | 0 |
| 14-PBV-19-015 | Sptm19-015 | 24.69 | 23.1 | 24.15 | 25.5 | 8,551 |
| 14-PBV-19-015 | Sptm19-015 | 24.69 | 23.1 | 24.15 | 25.5 | 14,004 |
| 15-PBV-19-016 | Sptm19-016 | −1 | 28.71 | −1 | 25.56 | 703,684 |
| 16-PBV-19-020 | Sptm19-020 | −1 | 35.51 | −1 | −1 | 6 |
| 17-PBV-19-021 | Sptm19-021 | 35.59 | 35.25 | −1 | 31.49 | 4 |
| 18-PBV-19-023 | Sptm19-023 | −1 | 25.12 | 24.22 | −1 | 163 |
| 18-PBV-19-023 | Sptm19-023 | −1 | 25.12 | 24.22 | −1 | 4,574 |
| 19-PBV-19-031 | Sptm19-031 | −1 | 32.26 | −1 | 30.16 | 18 |
| 20-PBV-19-032 | Sptm15-032 | −1 | 27.69 | −1 | 25.94 | 54 |
| not uploaded | Sptm19-033 | −1 | 25.35 | 26.91 | 34.56 | 4 |
| not uploaded | Sptm19-033 | −1 | 25.35 | 26.91 | 34.56 | 32 |
| not uploaded | Sptm19-034 | −1 | 26.41 | 27.39 | 35.75 | 1 |
| not uploaded | Sptm19-034 | −1 | 26.41 | 27.39 | 35.75 | 22 |
| 23-PBV-19-035 | Sptm19-035 | 35.84 | 29.3 | −1 | 26.64 | 563 |
| 24-PBV-19-03 [symbol] | Sptm19-036 | −1 | 34.1 [symbol] | −1 | 30.39 | 0 |
| 25-PBV-19-03 [symbol] | Sptm19-03 [symbol] | 26.57 | 30.67 | −1 | 29.08 | 325 |
| 26-PBV-19-039 | Sptm19-039 | 34.43 | 21.56 | 25.45 | 29.49 | 399 |
| 26-PBV-18-039 | Sptm19-039 | 34.43 | 21.56 | 25.45 | 29.43 | 2,803 |
| 27-PBV-19-044 | Sptm19-044 | 22.58 | 31.48 | −1 | 26.95 | 6968 |
| 27-PBV-19-044 | Sptm19-044 | 22.58 | 31.48 | −1 | 26.95 | 340 |
| 28-PBV-19-046 | Sptm19-046 | 19.61 | 27.14 | −1 | 23.29 | 5919 |

| | | | PBV Teads ORF1/Capsid | | | | |
|---|---|---|---|---|---|---|---|
| | PBV Reads RDRP Segment 2 | | Segment 1 | | | Total NGS | |
| NGS ID | Consens | % [symbol] ov | Total | Consens | % [symbol] ov | reads | Reference |
| 1-PBV-4465 | 1,846 | 90% | 3,412 | 2,387 | 100% | [symbol] ,048,648 | ABT |
| 2-PBV-4468 | 572 | 30% | 19 | 759 | 34% | 3,322,400 | ABT |
| 3-PBV [symbol] 4138 | 1,690 | 83% | 210 | 2,246 | 94% | 7,321,340 | ABT |
| 4-PBV-4477 | 0 | 0% | 0 | 0 | 0 | 7,597,178 | ABT |
| 8-PBV-4470 | 43 | 2% | 0 | 0 | 0 | 8,063,558 | ABT |
| 9-PBV-6722 | 0 | 0% | 0 | 0 | 0 | 7,580,048 | ABT |
| 10-PBV-19-001 | 1,689 | [symbol] 3% | 265 | 2,1 [symbol] 7 | 92% | 3,749,428 | ABT |
| 11-PBV-19-006 | 1,925 | 94% | 2,271 | 2,345 | 98% | 3,541,114 | ABT |
| 12-PBV-19-012 | 261 | 14% | 0 | 0 | 0 | 9,517,31 [symbol] | ABT |
| 12-PBV-19-012 | 1,671 | 100% | 3,044 | 2,187 | 83% | 9,517,318 | KM285233 |
| 13-PBV-SPTM- | 0 | 0% | 0 | 0 | 0 | 2,242,462 | ABT |
| 14-PBV-19-015 | 1,990 | 98% | 28,613 | 2,345 | 99% | 4,685,264 | ABT |
| 14-PBV-19-015 | 1,671 | | 15,296 | 2,633 | 100% | 4,685,264 | KM285233 |
| 15-PBV-19-016 | 1,9 [symbol] 6 | 97% | 1,019 | 2,223 | 95% | 14,733,470 | ABT |
| 16-PBV-19-020 | [symbol] 28 | [symbol] 3% | 17 | 7 [symbol] 8 | 34% | 3,791,602 | ABT |
| 17-PBV-19-021 | 401 | 21% | 5 | 381 | 17% | 1,759,914 | ABT |
| 18-PBV-19-023 | 318 | 17% | 0 | 0 | 0 | 8,746, [symbol] 44 | ABT |
| 18-PBV-19-023 | 1,662 | 99% | 3,73 [symbol] | 2,627 | 100% | 8,746,944 | KM2 [symbol] 5233 |
| 19-PBV-19-031 | 751 | 40% | 0 | 0 | 0 | 6,175,024 | ABT |
| 20-PBV-19-032 | 1, [symbol] 21 | 86% | 5 | 164 | 7% | 5, [symbol] 53, [symbol] 46 | ABT |
| not uploaded | 160 | 8% | 0 | 0 | 0 | 374,952 | ABT |
| not uploaded | 1,212 | 64% | 16 | 867 | 33% | 374,952 | |
| not uploaded | 142 | 8% | 0 | 0 | 0 | 207,372 | ABT |
| not uploaded | 957 | 51% | 6 | 448 | 17% | 207,372 | |
| 23-PBV-19-035 | 1,847 | 91% | 407,736 | 2, [symbol] 3 | 99% | [symbol] ,335,93 [symbol] | ABT |
| 24-PBV-19-03 [symbol] | 0 | 0% | 0 | 0 | 0 | 5,209,118 | ABT |
| 25-PBV-19-03 [symbol] | 1, [symbol] 1 | 90% | 398 | 2,226 | 9 [symbol] % | 1,585,992 | ABT |
| 26-PBV-19-039 | 1,406 | 74% | 1 | 199 | 9% | 4,737,986 | ABT |
| 26-PBV-18-039 | 1,656 | 100% | 1355 | 2,565 | 97% | 4,737,986 | KM285233 |
| 27-PBV-19-044 | 2,022 | 99% | 6514 | 2,240 | 94% | [symbol] , [symbol] 91,792 | ABT |
| 27-PBV-19-044 | 1,202 | 73% | 1307 | 1,0 [symbol] 9 | 38% | [symbol] , [symbol] 91,792 | KM285233 |
| 28-PBV-19-046 | 2,0 [symbol] 1 | 98% | 6227 | 2,240 | 94% | 11,745,9 [symbol] 6 | ABT |

[symbol]

indicates data missing or illegible when filed

### Strain Identity

New genomes were aligned with MRN3406 and identity matrices were determined for nucleotide and amino acid sequences in open reading frames of segment 1(ORF1+capsid) and segment 2 (RDRP).

The nucleotide sequences of the new genomes are shown below.

>2_PBV-MRN3406 Capsid
(SEQ ID NO: 64)
AATTTGTACTTTAATGGTTTACAAGAGTTTAAAACCATACAACACTTTCTACACTCTA
AGAACACCAGCTACCGCACATAGTTTAGTGCAAATAGCTAGGATCAGAGATAGTAA
AGTGGGATTATCTGAAAGGAGGTTAAATTAATGACAGGTAATCAAATTAAATATGG
TGAATTACAAGAAAATATTCGCCATAACACTACAACAGAAGTTGAAACCAATAGAC
ACAACGTCGTGACTGAAGGTGAAACCAACAGACATAACGTTGTTACAGAGGTTGAA
ACTAATCGACACAATACTGTGACTGAAAGTATTGGATGGTACGATGCTGTATCAAAA
CGAATCTCAGCAAATGCTTCAATGAGTCAAGCGGGTGCAGCTTGGGCTAATGTTGCA
ATTAATCAACAAAATGCAGATACAAAGCGATTTGAAGCTGAACGCAATGCTGAAAT
AAATCAGCAAAATGCGGACACTAGAACATTTAGTGCACGTAGTGAGGATGCAGCTA
GATATGCTCATTCTTACAATGAAGATCGTAAAACTACAGCTGAAATTGAGCGAATGA
ACACACAAAATTCGCAAGGATGGGTGAAATCAATCACTGATGCAATCAGCTCACCT
ATCAAAGCATTACCATTATTAGGAGGATAAATTTTATGGTAAAGAATAACAACAAA
AAGCGTTTTCAGGATAAAAGTGATAAGTATTCTAGAAAACCTAAGTTCAAGGTTGA
AAAGAAAGATATCTTGGACGATGACAAATTGGAAGGATCTAAGTTTGGCAAAGTTA
ATGACATATCCTGGTATCAGAAGAATGCTGATTTACTCAGAGCTGCTGGTAACTTGT
CTTTTGCTAATGCGTTGGGATCTGGAATTGATCTATCTAACGCAAACTTTAACGTTAA
GCTTGCTGCTGATGAGCAACGTGTTCCTGGTATTGCAACTATACATACTATTACAGG
ACCTGGACTCAGTCGCGACGCACACTCTGGTGTCAACGTGGCAATGCGTAACTTATA
TTCTTTTGTTCGTCATGCAAATAGTGGTCATAGTAACTATGATCCTGTAGATCTAATG
TTATATCTACCTGCTATGGATGCAGCATACATGCTCTACTACCGTGCTGTTCGTGCAT
ATGGCGCAATGTTCACATTTAATACTGTGAATCGCTATGCTCCAAAAGCTCTTGTGG
AAGCGTTAGGTTTTGATTATGAAGATGTCAACTCAAACCTTGCTACATTCAGATATG
CAATTAACGCATACGCTGCAAGAATCAACGCATACGCTGTGCCTACGAATATGCCTA
TCTTCAAACGACATGCATGGCTCTTTTCATCTATCTATACAGATGAAAACGTATCTA
AAGCTCAGATTTATGCATTTACTTCTGATCATTATAGAGTATTTGATGAGAAGTATTC
TAAAGGTGGACGCCTTGTGGCTAAAGCCTGGAAAACAAAGTTAACTGTTAAAGATT
GGATTACAGTAGCAAATGAGGTTGCTGATCCAATTACAGTTTCAGAAGATTTAGGTA
TTATCTCAGGTGACTTAATTAAAGCATTTGGTAAGGAAAACTTACACATGTTAGCTA
CCTTGGCTGATAACTACGTTGTATTACCAACATATGTACCTGAAGTTATGGATCAAA
TTCATAACTTGCAAGCAGTAGGTCAGATTGATCTAGAAAGTAACAATATTGAACAA
GATCCAAACATTGGTAAGGGTAACTTGATTTACAACCCAGTTGTAACTGTCAATAAT
AATCCAATGGCTTACGCAAATCGTATTATGGATTTCAAAATTGATACACCTACTCCA
GATGATGTCGTTGTAGCTTCACGATTAGCTGTGGCATTAGAACCAGGCGCTACAACC
GGTAAGGCAGTATTCACTGCTATGGGTACAGAATTTGTGACTAAAGTTGGTATTCAC
ACATTCTACAAGGGAAATAATGGATTACTTAAGTCTATTGAACAGACTTTCAATACT
TTTGATTCTACTGAAGGTGGTCTCACTGACGCCGCATCAGTTAGTTTGCACATGTCTG
CCTACACAAAGGCCTCTAAGTTTGTACACTTTCCAATTCAATATATGTGTATGGGTA
GCCCTACTCAACCTGACAAACGTGAAGTCAGAATCTTTGGCGAATTGGGCACGTACA
CTATTATTAATGGGGTCACTCTTAATAAGTTACACGACGTGTGTGTATTAAGTTTATT
TGATGTACCTATTAAGCTTTAGATGCATTAGGG.
>1-PBV-4466 Capsid
(SEQ ID NO: 29)
TCTTTAAATAAGTCATTACTAGAAAGGAGAAATTTGTACTTTAATGGTTTACAAGAG
TTTAAAACCATACTTCACTTTCTGCACTCTAAGAACACCAGCTACCGCACATAGTTT
AGTGCAAATAGCTAGGATCAGAGACAGATTTGTGAAATTACCTGAAAGGAGGATAA
AATATGACAGGTAATCAAATTAAATATGGCGAATTACAAGAAAACATTCGTCATAA
TCAAACTACCGAAGTCGAAACCAATCGACATAACGTTGTGACTGAGGGTGAAACAA
ACCGACATAACGTTGTCACTGAGAATGAGACGAATCGGCACAATGTAGTGACAGAG
AGTATTGGATGGTATGATGCAGTATCAAAGAGAATATCTGCTAATGCCTCAATGAGT
CAAGCTGGTGCAGCTTGGGCTAATGTCGCTATAAATCAGCAGAATGCGGATACTCGT
AGATATGAAGCTGAAAGAAATGCTGAGATTAATCAGCAAAATGCCGACACTAAGAG
ATTTAGTGCTGAAAGTGAGGATGCTGCTCGCTATGCGCATTCTTACAATGAAGATCG
TAAAACTACTGCTGAAATTGAGAGAATGCAGAATCAAAATTCTCAGGGATGGGTGA
AAGCTATTACTGATAGTATTAGCGCACCAATTAAAGCTTTACCATTATTAGGAGGAT
AAGATAAAATGGCAAAATTTAAAGATAAAGAAAGTTTCCAGAAAAGAAACAAAAC
AAAGAAATGGGATAAAAAGGATCCTAAGAAGAATCCTAAACATGATGAACCAACTG
AAAAGTTGGACGACGACAAATTGGAAGGATCTAAGTTTGGCAAAGTTAATGACATA
TCCTGGTATCAGAAGAACCCTGATTTACTCAGAGCTGCTGGTAACTTGTCTTTTGCTA
ATGCGTTGGGATCTGGAATTAACCTATCTAACGCTAACTGTAAACTTAGTCTTGCTG
CTGATGAGCAACGTATTCCTGGCATTGCAACTATACATACTATTACAGGACCTGGAC
TTAGCCGATCAGCTAATTCTGGAGTCAATATTGCTATGCGTAATTTATATTCATTTGT
TCGTCATGCTAATAGCGGTCATAGTAACTATGATCCCGTAGATTTAATGTTATATCTC
TTAGCTATGGATGAGGCTTATATGGCCTATTTCCGTGCCGTACGTGCTTATGGCGCTA
TGTTTACTTTTAATACATTAAATCGATACGCACCTAAGGCTCTTGTTGAAGCATTAGG
ATTCGATTATGAAGATATCAACAAGAATCTTGCTACATTCAGATATGCAATTAACGC
ATATGCTGCAAGAATCAATGCTTACGCTGTCCCTACGAATATGCCTTTGTTTAAGAG
ACATGCGTGGCTATTCTCATCTATTTATACAGATGAAAATGTATCTAAAGCTCAGAT
TTATGCATTTACTACTGATCATTATAGAACATATGATGAAAAGTATTCTAAAGGTGG
ACGACTTGTGGCTAAAGCCTGGAAGCCTAAACTAAAGGTAGAGGATTGGATTTCAG
TTGCTAATGAAATTGCGGACCCAATTACTACTTCTGAAGATCTGGGTATTATATCGG
GCGACTTAATTAAAGCGTTCGGTAAAGAAAATTTACACACACTTGCAACATTAGCTG
ATAACTATGTTGTGTTACCAACTTACGTACCTGAAGTTATGGACCAAATTCACAATT
TACAGGCAGTTGGCGATGTTGAATTAGCGAGCAACAACATCGAACAGGACCCTCAA
ATTGGAAAGGGCAACCTAATCTATGATCCAATTCTTAAATCGGGTAAGAATCCGGTA
TTATATGGGGATCGTATTATGGATTTCAAGATTGACACACCAACACCAGAAGATGTA
ATTGTTGCATCACGATTGGCTGTATCACTAGAACCATCACCGGATGGTAATAAGGCT
CATTTTGTAGCCATGGGTACAGAGTTTGTGACACATGTTGGAATTCATACACTTTATC
AGACAACCTCTGGTAATGTTAAATGTCTTGAACAGACTTTTGATACTATTGCAGCTG
TTGAGGGTGGTCTTGCTGATGCCGCATCAGTTAGTTTGTTCCTATCTGGATACACAA
AGGCCTCTAAGTTTGTACATTTTCCTATTCAATATGCTTGTCTGGGTAACGCTAGTGA
CCCTAATGGACAATCAATCAGAATCTTTGGTGAATTGGGGACGTACAGTACTATTAA
CAGCACTACTCTTAATAAATTACACGATGTGTGTGTATTAAGTTTGTTAGATGTACCT
ATCAAATTATAGATACATGGGGGAAGTGAGGAG
>3-PBV-4138 Capsid
(SEQ ID NO: 30)
GGAGAAATTTGTACTTTAATGGTTTACAAGAGTTTAAAACCATACAATGCTTTTCTC
ACTCTAAGAACACCAGCTAACGCACATAGTCTAGTGCAAATAGCTAGGATCAGAGA
TGGAAGAGCAAGATTATCTGAAAGGAGGTTAAATATGAAATGACAGGTAATCAAAT
TAAGTATGGCGAATTACAAGAGAACATACGCCATAACACTACTACCGAAGTTGAGA
CCAACCGTCATAACGTTGTTACAGAAGGCGAAACAAATCGTCACAATGTTGTGACTG
AGGCTGAAACTAATCGGCACAATACTGTAACTGAAAGTATTGGATGGTACGATGCA
GTATCAAAGAGAATCTCAGCTAACGCGTCCATGAGCCAAGCAGGTGCAGCTTGGGC
TAATGTTGCTATCAATCAACAGAACGCAGACACACGTAAATATGAAGTTGAGAAGA
ACGTTGAAATCAATCAACAAAATGCAGATACTAAAGCATTTAGTGCCAGAAGTGAA
GATGCTGCTAGATATGCTCATTCATATAATGAAGATCGCAAAACTACAGCTGAAATT
GAGCGAATGAAGACTCAAAATTCACAAGGATGGGTGAAATCAATTACTGATGCTAT
CAGTGCGCCTATCAAAGCATTACCATTATTAGGAGGATAAATTATATGGTAAAGAA
AAATGATAACAACAAACGTTTTCAGAATAAAAGTGAGAAATATTCTAGAAAACCTA
AATTCAAGATTGAAAAGAAAGATATCTTGGATGATGACAAGCTTGAAGGATCTAAG
TTTGGAAAAGTTAATGACATCAGCTGGTATCAGAAGAATCCTGATTTACTCAGAGCT
GCTGGTAACTTGTCTTTTGCTAACGCTTTGGGATCTGGAATTAACTTATCTNNNNNN
NNNNNNNNNNNNNNNNNNNNNNNNNNNGAACAACGTATTCCTGGTATTGCAACTA
TACATACTATCACAGGACCTGGGCTTAGCAGAGACGCGCACTCTGGTGTTAACGTCG
CAATGCGTAACCTTTATTCTTATGTTCGTCATGCAAATAGCGGTCATAGTAATTATGA
CCCTGTAGATTTGATGCTTTATCTATTGGCAATGGATGAGGCTTATATGGTCTACTAT
CGCGCTGTCCGTGCATATGGAGCAATGTTTACATTTAATACAGTAAATAGATATGCG
CCTAAAGCTCTTGTTGAAGCATTAGGTTTTGATTATGAAGATGTCAACGCAAACCTT
GCTACATTCAGATATGCAATTAACGCATATGCTGCAAGAATCAACGCATACGCTGTT
CCTACGAATATGCCTATCTTCAAACGACACGCATGGCTCTTTTCATCTATCTATACAG
ATGAAAACGTATCTAAGGCTCAGATTTATGCATTTACTTCTGATCATTATAGAATAT
ATGATGAGAAGTATTCTAAAGGTGGACGCCTTGTGGCTAAAGCCTGGAAATCAAAG
TTAACTGTACAAGACTGGATCAATGTAGCAAATCAAGTGGCAGATCCAATTACTGTC
TCAGAGGATTTGGGTATTATATCTGGTGATATAATTAAAGCATTTGGTAAAGAAAAT
CTACATATGCTGGCTACTTTAGCTGATAACTACATAGTTTTACCAACTTATGTTCCTG
AAGTCATGGATCAAATTCATAACTTACAGGCTGTTGGTAACATTACTCTTGAGAGTA
ATAATATTGAACAAGATCCAGCAATTGGTAAAGGTAACTTAATCTATAATCCAATTG
TAACNNNNNNNNNNNNNNNNNNAGCATATGCTGATCGTATTATGGATTTCAAAATT
GACACTCCGACCCCAGACGATGTAGTCATAGCTTCACGTTTAGCTGTGGCACTTGAA
CCTGGATCAACAACCGATAAAGCAGTATTTACTGCAATGGGTACAGAGTTTGTAACA
AAAGTTGGAATTCACACATTATACCGCACATCTGCGGGATCTATTAAGTGTCTTGAA
CAGGACTTCAATACTTTTGAGTCTACTGAAGGTGGTCTTGTTGACGCCTCATCAGTTA
GTTTGCACTTATCTGCATACACGAAGGCCTCTAAATTTGTACACTTTCCAATTCAATA
TATGTGTTTGGGTAGCCCTACTACTCCTGACAAACGTGAAGTCAGAATTTTTGGTGA
GTTGGGCACGTACACTGTTATTAATGGGGTCACTCTTAGTAAGCTTCACGATGTGTG
TGTACTGAGTCTATTTGATGTACCTATCAAATTATAGATACATGGAAAGTGAGGAG
>10-PBV-19-001 Capsid
(SEQ ID NO: 31)
ACACTTTCTACACTCTAAGAACACCAGCTACCGCACATAGTTTAGTGCAAATAGCTA
GGATCAGAGATAGTAAAGTGGGATTATCTGAAAGGAGGTTAAATTTAAATGACAGG
TAATCAGATTAAGTATGGCGAATTACAAGAAAATATTCGTCATAATACAACAACAG
AAGTTGAGACTAACAGACACAACGTTGTTACGGAAGGTGAAACAAATCGTCATAAT
GTTGTAACTGAAGTCGAGACTAATCGACACAATACTGTTACTGAAAGTATTGGATGG
TACGATGCTGTATCAAAACGTATCTCAGCGAATGCTTCAATGAGTCAAGCAGGTGCA
GCTTGGGCTAACGTGGCTATTAATCAGCAAAACGCTGACACTAAGCGCTTTGAAGCC
GAACGCAATGCTGAAATTAATCAGCAGAATGCAGACACTAAAACATTTAGTGCACG
CAGTGAGGATGCCGCTAGATATGCACATTCTTACAATGAAGATCGTAAAACTACAG
CAGAAATTGAGCGAATGAACACACAAAATTCGCAAGGATGGGTGAAATCAATAACT
GATTCAATCAGTGCACCTATCAGAGCATTACCATTATTAGGAGGATAAATTATATGG
TAAAGAATACTAATAAGAAGCGTTTTCAGGATAAAAGTGAGAAATATTCTAGAAAA
CCTAAGTTCAAGGTTGAAAAGAAAGATATCTTGGACGATGACAAACTTGAAGGATC
TAAGTTTGGAAAAGTTAATGACATTTCCTGGTACCAGAAGAACCCTGATTTGCTCAG
AGCTGCTGGTAACTTGTCTTTTGCTAATGCGTTGGGATCTGGAATTGATCTATCTAAC
GCAAACTTTAACGTTAAGCTTGCTGCTGATGAGCAACGTGTTCCTGGTATTGCAACT
ATACATACTATTACAGGACCTGGACTCAGTCGCGACGCACACTCTGGTGTTAACGTG
GCAATGCGTAACTTATATTCTTTTGTTCGTCATGCAAATAGTGGTCATAGTAACTATG
ATCCTGTAGACCTGATGCTATATCTACTAGCCATGGATGAAGCGTATATGGTCTACT
ACCGTGCTGTTCGTGCATATGGCGCAATGTTCACTTTCAACACAGTGAATCGCTATG
CTCCGAAAGCTCTTGTGGAAGCGTTAGGTTTTGATTATGAAGATGTCAACTCAAACC
TTGCTACATTCAGATATGCAATTAACGCATACGCTGCAAGAATCAACGCATACGCTG
TGCCTACGAATATGCCTATCTTCAAACGACATGCATGGCTCTTTTCATCTATCTATAC
AGATGAAAACGTATCTAAAGCTCAGATTTATGCATTTACTTCTGATCATTATAGAGT
ATTTGATGAGAAGTATTCTAAAGGTGGACGCCTTGTGGCTAAAGCCTGGAAATCAA
AATTAACTGTTAAGGATTGGATTACGGTAGCGAATGAAGTTGCAGATCCTATTACAG
TGTCTGAGGATTTAGGTATTATATCAGGTGACTTAATTAAAGCATTTGGTAAGGAAA
ACTTACACATGCTGGCTACTTTAGCCGATAATTATGTCGTATTACCAACATATGTACC
TGAGGTTATGGACCAGATTCATAACTTACAAGCTGTTGGAACAATTGACTTAGAAAG
TAATAATATTGAACAGGATCCAAACATTGGTAAGGGTAATTTAATTTACAATCCTAT
TGTTACTGTCAATAATAATCCAATAGCTTACGCAAATCGTATTATGGATTTCAAGAT
CGAGACACCTACTCCTGAAGATGTAGTTGTTGCATCGAGATTAGCCGTAGCATTAGA
ACCAGGCGCGACAACCGGTAAAGCGGTATTCACTGCTATGGGTACAGAATTTGTGA
CAAAAGTTGGTATTCATACGTTCTATAAAGGAAACAATGGACTACTTACGTCTATTG
AACAGACTTTCAATACTTTTGATTCTACTGAAGGTGGTCTTGCTGACGCCTCATCAGT
TAGTTTGCACATGTCTGCCTACACAAAGGCCTCTAAGTTCTTACACTTTCCTATTCAA
TATATGTGTATGGGTAGCCCTACTCAACCTGACAAACGTGCAGTCAGAATCTTTGGC
GAATTGGGCACTTACACTATTGTTAATGGGGTCACTCTTAGTAAGCTTCACGATGTG
TGTGTATTAAGTCTATTTGATGTACCTATTAAACTTTAGATGCATTAGGGGAAACA
>11-PBV-19-006 Capsid
(SEQ ID NO: 32)
AGTGGAGAAATTTGTACTTTAATGGTTTACAAGAGTTTAAAACCATACAACACTTTC
TACACTCTAAGAACACCAGCTACAGCACATAGTTTAGTGCAAATAGCTAGGATCAG
AGATAGTAAAGTGGGATTATCTGAAAGGAGGTTAAATTAAATGACAGGTAATCAGA
TTAAATATGGCGAGTTACAAGAAAATATTCGTCATAACACAACAACAGAAGTCGAA
ACTAACAGACACAATGTTGTTACGGAAGGTGAAACTAACCGACACAATGTTGCTAC
TGAAGTTGAGACAAATCGACACAATACTGTGACTGAAAGTATTGGATGGTACGATG
CTGTATCAAAACGAATCTCAGCAAATGCTTCAATGAGTCAAGCAGGTGCAGCTTGG
GCAAATGTTGCTATTAATCAGCAAAATGCTGATACAAAACGATTTGAAGCTGAGCGT
AATGCTGAAATTAATCAGCAAAACGCTGACACCAAAAGATTTAGTGCACGTAGTGA
GGATGCCGCTAGATATGCGCACTCCTACAACGAAGATCGTAAAACTACAGCAGAAA
TTGAGCGAATGCACACACAGAATTCGCAAGGATGGGTGAAATCAATTACTGATGCA
ATCAGTGCACCTATCAAAGCATTACCATTATTAGGAGGATAAATTATATGGTAAAGA
ATAACAACAAAAAGCGTTTTCAGAATAAAAGTGAGAAATATTCTCGAAAACCTAAG
TTCAAGGTTGAAAAGAAAGATATCTTGGACGATGACAAACTTGAAGGATCTAAATT
TGGCAAAGTTAATGACATATCGTGGTATCAGAAGAATCCTGATTTACTCAGAGCTGC
TGGTAACTTGTCTTTTGCTAATGCGTTGGGATCTGGAATTGATCTATCTAACGCAAAC
TTTAACGTTAAGCTTGCTGCTGATGAGCAACGTATTCCTGGTATTGCAACTATACAT
ACTATTACAGGACCTGGACTCAGTAGAGACGCTCACTCTGGTGTCAACGTGGCAATG
CGTAACTTATATTCTTTTGTTCGTCATGCAAATAGCGGTCATAGTAATTATGATCCTG
TAGATTTAATGCTTTATCTATTAGCTATGGATGAAGCGTACATGGTCTACTACCGTGC
TGTTCGTGCATATGGCGCAATGTTCACATTTAATACGGTGAACCGCTATGCTCCAAA
GGCTCTTGTTGAAGCGTTAGGTTTCGATTATGAAGATGTCAACTCAAACCTTGCTAC
ATTCAGATATGCAATTAACGCATACGCTGCAAGAATCAACGCATACGCTGTGCCTAC
GAATATGCCTATCTTCAAACGACATGCATGGCTCTTTTCATCTATCTATACAGATGA
AAACGTATCTAAAGCTCAGATTTATGCATTTACTTCTGATCATTATCGAGTATATGAT
GAGAAGTATTCTAAAGGTGGACGCCTTGTGGCTAAAGCCTGGAAAGCAAAATTAAC
AGTACAAGATTGGATAACTGTAGCTAATGAAGTTGCAGATCCTATTACAGTTTCTGA
GGATTTAGGCATCATATCTGGTGACTTAATTAAAGCGTTTGGTAAGGAAAACTTGCA
TATGTTAGCTAATTTAGCTGATAACTACGTTGTATTACCAACTTATGTACCTGAAGTT
ATGGATCAAATTCATAACTTACAATCAGTAGGAACAATCGATCTAGAGAGTAACAA
TATTGAACAAGATCCAAGTATTGGTAAGGGTAATTTAATTTATAACCCAATTGTTAC
TGTAGATAATAATCCAATGGCATTCGCTAATCGTATTATGGATTTTAAGATCGATAC
ACCTACTCCTGATGATGTAGTTGTAGCATCACGATTGGCTGTAGCATTAGAACCAGG
CGCCACGACCGGTAAAGCAGTGTTCACTGCTATGGGTACAGAATTTGTGACCAAAAT
TGGTATTCACACATTCTGCAAAGGAAGTAATGGATTACTTAAGTCTATTGAACAGAC
TTTCAATACTTTTGATTCTGTTGAAGGTGGTCTTGCTGACGCCTCATCAGTTAGTTTG
CACATGTCTGCCTACACAAAGGCCTCTAAGTTTGTACACTTTCCTATTCAATATCTGT
GTATGGGTAGCTCTGCTCAACCTGACAAGCGTGAAGTCAGAGTCTTTGGCGAATTGG
GCACTTACACTATTGTTAGTGGGGTCACTCTTAGTAAGTTACACGATGTGTGTGTATT
AAGTCTATTTGATGTGCCTATTAAACTTTAGATGCATTAGGGGAAGTG
>14_PBV-19-015 Capsid
(SEQ ID NO: 33)
AGAAATTTGTACTTTAATGGTTTACAAGAGTTTAAAACCATACAACACTTTCTACAC
TCTAAGAACACCAGCTACCGCACATAGTTTAGTGCAAATAGCTAGGATCAGAGATA
GTAAAGTGGGATTATCTGAAAGGAGGTTAAATTGAATAAATGACAGGTAATCAGAT
TAAGTATGGCGAATTACAAGAGAGTATTCGTCATAATTCAACGACAGAAGTCGAAA
CCAATAGACACAACGTTGTTACTGAGAATGAAACGAATCGTCACAATGTTGTAACTG
AGGTGGAGACTAATCGACACAATACTGTTACTGAAAGTATTGGATGGTACGATGCT
GTATCAAAACGTATCTCAGCTAATGCTTCAATGAGTCAAGCAGGTGCAGCTTGGGCG
AATGTCGCTATCAATCAGCAAAATGCTGATACCAAACAATTTGAAGCTGAGCGCAA
TGCTGAAATTAATCAGCAAAATGCAGACACTAAAGCGTTTAGTGCACGTAGTGAAG
ATGCTGCGAGATATGCGCATTCCTACAATGAAGATCGTAAAACTACAGCAGAAATC
GAGCGAATGAACGCACAAAATTCGCAAGGATGGGTGAAATCAATTACTGATGCAAT
CAGCGCACCTATCAGAGCATTACCATTATTAGGAGGATAAATTATATGGTAAAGAAT
AACAACAAAAAGCGTTTTCAGGATAAAAGTGATAAGTATTCTAGAAAACCTAAGTT
CAAGGTTGAAAAGAAAGATATCTTGGACGATGACAAATTTGAAGGATCTAAGTTTG
GAAAAGTTAATGACATTAGTTGGTACCAGAAGAATCCTGATTTACTCAGAGCTGCTG
GTAACTTGTCTTTTGCTAATGCGTTGGGATCTGGAATTGATCTATCTAACGCAAACTT
TAACGTTAAGCTTGCTGCTGATGAGCAACGTGTTCCTGGTATTGCAACTATACATAC
TATTACAGGACCTGGACTCAGTCGCGACGCACACTCTGGTGTCAACGTGGCAATGCG
TAACTTATATTCTTTTGTTCGTCATGCAAATAGCGGTCATAGTAACTATGATCCTGTA
GACTTAATGCTATATCTATTAGCCATGGATGAAGCGTACATGGTCTACTACCGTGCT
GTTCGTGCATATGGCGCAATGTTCACATTTAATACAGTGAATCGCTATGCTCCAAAA
GCTCTTGTTGAAGCGTTAGGTTTTGATTATGAAGATGTCAACTCAAACCTTGCTACAT
TCAGATATGCAATTAACGCATACGCTGCAAGAATCAACGCATACGCTGTGCCTACGA
ATATGCCTATCTTCAAACGACATGCATGGCTCTTTTCATCTATCTATACAGATGAAA
ACGTATCTAAAGCTCAGATTTATGCATTTACTTCTGATCATTATAGAGTATTTGATGA
GAAGTATTCTAAAGGTGGACGCCTTGTGGCTAAAGCCTGGAAGTCAAAATTGACTGT
TAAGGATTGGATTACTGTAGCTAATGAAGTTGCAGATCCTATTACAGTGTCTGAGGA
CCTAGGTATTATATCAGGTGACTTAATTAAAGCATTCGGTAAGGAAAACTTACATAT
GTTAGCTACATTAGCTGACAATTATGTTGTGTTACCAACTTATGTACCTGAGGTTATG
GATCAAATCCATAATTTACAAGCAGTTGGAACAATCGATTTGGAAAGTAACAACATT
GAACAGGATCCGACTATCGGTAAGGGTAATTTAATTTATAACCCAATTGCAACTGTC
AATAATAATCCATTGGCGTACGCAAATCGTATCATGGATTTCAAGATCGATACACCT
ACTCCAGATGATGTGGTTGTGGCATCACGATTAGCTGTGGCATTAGAACCAGGCGCT
ACGACCGGTAAAGCAGTATTTACTGCTATGGGTACAGAATTTGTGACAAAGATTGGT
ATTCACACATTCTACAAAGGAAGTAATGGACTTATTAAGTCTATTGAACAGACTTTC
AATACTTTTGATTCTACTGAAGGTGGTCTCACTGACGCCACATCAGTTAGTTTGCAC
ATGTCTGCCTACACAAAGGCCTCTAAGTTTGTACACTTTCCTCTTCAATATATGTGTC
TGGGTAGCCCTACTCAACCTGACAAACGTGAAGTCAGAATCTTTGGTGAATTGGGCA
CTTACACTATTATTAATGGGGTCACTCTTAGTAAGTTACACGACGTGTGTGTATTAA
GCTTATTTGATGTACCTATTAAACTTTAGATGCATTAGGGGAAGTG
>15-PBV-19-016_Capsid
(SEQ ID NO: 34)
TTCAGTCGTCGGCAGCGTCAGATGTGTATAATTTGTACTTTAATGGTTTACAAGAGTT
TAAAACCATACAATGCTTTTCTCACTCTAAGAACACCAGCTACCGCACATAGTTTAG
TGCAAATAGCTAGGATCAGAGACAGAGAAGCAAGATTATCTGAAAGGAGGTTAAAT
ATGAAATGACAGGTAATCAAATTAAGTATGGCGAATTACAAGAGAACATACGCCAT
AACACTACTACTGAGGTTGAAACCAATCGTCACAATGTTGTTACTGAAGGTGAAACT
AATCGCCATAACGTTGTAACTGAGGTTGAGACTAATCGACACAATACTGTAACTGAG
AGTATTGGATGGTACGATGCCGTATCGAAAAGAATTTCTGCGAATGCATCAATGAGT
CAAGCAGGTGCAGCTTGGGCTAATGTTGCAATTAATCAGCAAAATGCGGATACACG
CAGATATGAAGCTGAGAGCAATGTTGCAATTAATCAACAGAACGCAGATACAAAGG
CATTTAGTGCCAGAAGTGAAGATGCTGCTAGATATGCTCATTCATATAACGAAGATC
GCAAAACTACAGCTGAAATTGAGCGAATGAACACTCAAAATTCACAGGGATGGGTG
AAATCAATTACTGATGCAATCAGTGCACCTATCAAAGCATTACCATTATTAGGAGGA
TAAATTATATGGTAAAGAAGAATGACAACAACAAACGTTTTCAGAATAAAAGTGAG
AAATATTCTAGAAAACCTAGATTCAAGATTGAGAAGAAAGATATCTTGGATGATGA
CAAGCTTGAGGGATCTAAGTTTGGAAAAGTTAATGACATCAGCTGGTATCAGAAGA
ACCCTGATTTACTCAGAGCTGCTGGTAACTTGTCTTTTGCTAACGCTTTGGGATCTGG
AATTAACTTATCTAACTCAAACTTTAATATTAAGCTTGCTGCTGATGAACAACGTGTT
CCTGGTATTGCAACTATACATACTATTACAGGACCTGGGCTTAGCAGAGACGCACAC
TCTGGTGTTAACGTCGCAATGCGTAACCTTTATTCTTATGTTCGTCATGCAAATAGTG
GTCATAGTAATTATGATCCTGTAGATCTAATGCTTTATCTCTTAGCCATGGATGAAGC
TTATATGGTCTACTATCGTGCCGTTCGTGCATATGGAGCAATGTTTACATTTAACACA
GTGAATAGATATGCGCCTAAAGCTCTTGTTGAAGCATTAGGTTTTGATTATGAAGAT
GTCAACGCAAACCTTGCTACATTCAGATATGCAATTAACGCATACGCTGCAAGAATC
AACGCATACGCTGTTCCTACGAATATGCCTATCTTCAAACGACACGCATGGCTCTTT
TCATCTATCTATACAGATGAAAACGTATCTAAGGCTCAGATTTATGCATTTACTTCTG
ATCATTATAGAACATATGATGAGAAGTATACTAAAGGTGGACGCCTTGTGGCTAAA
GCCTGGAAACCAAAGTTAACTGTACGAGATTGGATCGCAGTATCAAATGAAGTTGC
GGATCCAATTACTGTTTCTGAAGATTTGGGTATTATATCTGGTGATATAATTAAGGCT
TTTGGTAAAGAAAATCTGCATATGTTAGCTACACTGGCTGACAATTATGTTGTATTA
CCAAGCTATGTGCCTGAAGTTATGGATCAAATTCATAACCTACAAGCAGTAGGTGAT
GTAGCTCTTGAGAGCAATAACATCGAACAAGATCCAACAATTGGTAAGGGCAATTT
AATCTATAACCCAATTGTAACAGTTAACAATAATCCTTTAGCGTACGCTGATCGCAT
TATGGATTTCAAAATTGACACTCCAACTCCGGATGATGTAGTCGTAGCTTCTCGTTTA
GCTGTGGCTCTTGAACCCGGGTCAACAACCGGTAAAGCAGTATTCACTGCTATGGGT
ACAGAATTTGTAACAAAAGTTGGAATTCACACATTATACCGCACAACTGAGGGATCT
ATTAAGTGTATTGAACAGATTTTCAATACTTTTGAGTCTACTGAAGGCGGTCTTGCTG
ACGCCGCATCAGTTAGTCTGCACCTATCTACATACACGAAGGCCTCTAAGTTTGTAC
ACTTTCCAATTCAATATATGTGTCTGGGTAGCCCTACTACTCCTGACAAACGTGAAG
TCAGAATCTTTGGTGAATTGGGCACGTACACTGTTATTAATGGGGTCACTCTTAATA
AGTTACACGATGTGTGTGTATTGAGTCTATTTGATGTACCTATTAAACTTTAGATGCA
TTAGGGGAAGTG
>23-PBV-19-035_Capsid
(SEQ ID NO: 35)
TTAGGAGAAAATTTGTACTTTAATGGTTTACAAGAGTTTAAAACCATACAACACTTT
CTACACTCTAAGAACACCAGCTACAGCACATAGTTTAGTGCAAATAGCTAGGATCA
GAGATAGTAAAGTGGGATTATCTGAAAGGAGGTTAAATTAAATGACAGGTAATCAA
ATTAAGTATGGCGAATTACAAGAAAATATTCGTCATAACACTACAACAGAAGTTGA
AACTAATAGGCACAACGTTGTTACAGAAGGCGAGATTAACCGACACAACATTGTGA
CTGAAGTGGAAACGAATCGACACAATACTGTTACTGAAAGTATTGGATGGTACGAT
GCTGTATCAAAGCGAATCTCAGCAAACGCTTCAATGAGCCAGGCAGGTGCAGCTTG
GGCTAATGTTGCTATTAATCAGCAGAATGCTGATACTAAGCGATTTGAAGCAGAACG
CAATGCTGAAATTAATCAGCAAAATGCAGACACTAAAACATTTAGTGCACGTAGTG
AGGACGCCGCTAGATATGCGCACTCCTACAATGAGGATCGGAAAACTACAGCAGAA
ATTGAGCGAATGAACACACAGAATTCGCAAGGATGGGTGAAATCTATCACTGATGC
AATCGGTGCACCTATCAAAGCATTACCATTATTAGGAGGATAAATTATATGGTAAAG
AATAATAACAAGAAGCGTTTTCAGGATAAAAGTGAGAAATATTCTAGAAAACCTAA
GTTCAAGGTTGAAAAGAAAGATATCTTGGACGATGACAAATTGGAAGGATCTAAGT
TTGGCAAAGTTAATGACATATCATGGTACCAGAAGAATCCTGATTTACTCAGAGCTG
CTGGTAACTTGTCTTTTGCTAATGCGTTGGGATCTGGAATTGATCTATCTAACGCAAA
CTTTAACATTAAGCTTGCTGCTGATGAGCAACGTATTCCTGGTATTGCAACTATACAT
ACTATTACAGGACCTGGACTCAGTAGAGACGCACACTCTGGTGTCAATGTGGCAATG
CGTAACTTATATTCTTTTGTTCGTCATGCAAATAGTGGTCATAGTAACTATGATCCTG
TAGACTTAATGCTTTATCTATTAGCTATGGATGAAGCGTATATGGTTTACTACCGTGC
TGTTCGTGCATATGGCGCAATGTTCACGTTTAATACAGTGAATCGCTATGCTCCAAA
AGCTCTTGTTGAAGCGTTAGGTTTTGATTATGAAGATGTCAACTCAAACCTTGCTAC
ATTCAGATATGCAATTAACGCATACGCTGCAAGAATCAACGCATACGCTGTGCCTAC
GAATATGCCTATCTTCAAACGACATGCATGGCTCTTTTCATCTATCTATACAGATGA
AAACGTATCTAAAGCTCAGATTTATGCATTTACTTCTGATCATTATAGAGTATATGAT
GAGAAGTATTCTAAAGGTGGACGCCTTGTGGCTAAAGCCTGGAAATCAAAATTGAC
TGTAAAAGATTGGATTACTGTAGCAAATGAAGTTGCAGATCCTATTACAGTTTCTGA
AGATTTAGGTATCATTTCAGGTGACTTAATTAAGGCATTTGGTAAAGAGAATTTACA
CATGCTAGCTACACTAGCTGATAACTATGTAGTGCTACCAACATATGTACCTGAAGT
TATGGACCAAATTCATAACTTACAAGCTGTAGGAGCGATTGACCTAGAAAGTAACA
ATATCGAACAGGATCCAAATATTGGTAAGGGTAATTTAATCTATAACCCGATTGTCA
CTGTTAATAATAATCCTATGGCATACGCGAATCGTATTATGGATTTCAAGATTGATA
CACCTACCCCTGATGATGTAGTTGTAGCATCCAGATTAGCTGTAGCATTGGAACCAG
GCGCAACTACCGGTAAAGCAGTATTTACTGCCATGGGTACGGAATTTGTGACAAAA
GTTGGTATTCACACATTCTTCAAAGGAAGTAATGGATTACTTAAAACTATTGAACAG
ACTTTCAACACTTTTGATTCTACTGAAGGTGGTCTCACTGACGCCGCATCAGTTAGTT
TGCACATGTCTGCCTACACAAAGGCCTCTAAGTTTGTACACTTTCCTATTCAATATAT
GTGTATGGGCAGCCCTACTCAACCTGACAAACGTGAAGTCAGAGTCTTTGGCGAATT
GGGCACGTACACTATTGTTAATGGGATCACTCTTAGTAAGTTACACGACGTGTGTGT
ATTAAGTCTATTTGATGTACCTATTAAACTTTAGATGCATTAGGGGAAGTG
>25-PBV-19-038 Capsid
(SEQ ID NO: 36)
AATTTGTACTTTAATGGTTTACAAGAGTTTAAAACCATACAACACTTTCTACACTCTA
AGAACACCAGCTACTGCACATAGTATAGTGCAAATAGCTAGGATCAGAGATAGTGA
AGTGGGATTATCTGAAAGGAGGTTAAATTAATGACAGGTAATCAAATTAAATATGG
TGAATTACAAGAGAATATTCGCCATAACACAACAACAGAAGTTGAAACCAATAGAC
ATAACGTTGTAACTGAAGGTGAAACTAACAGACATAACGTTGTCACAGAGGTTGAG
ACTAATCGACACAATACTGTGACTGAAAGTATTGGATGGTACGATGCTGTATCAAAA
CGTATCTCAGCAAATGCTTCAATGAGCCAAGCCGGTGCAGCTTGGGCTAACGTTGCA
ATCAATCAACAAAATGCAGACACGAAACGATTTGAGGCTGAACGTAATGCTGAAAT
AAATCAGCAAAATGCGGATACTAAAGCATTTAGTGCACGCAGTGAGGATGCAGCTA
GATATGCTCATTCTTACAATGAGGATCGTAAGACTACAGCAGAAATTGAGCGAATG
AACACACAAAATTCGCAAGGATGGGTGAAGTCAATCACTGATGCAATTAGCGCACC
TATCAAAGCATTACCATTATTAGGAGGATAAATTTTATGGTAAAGAATAACAACAA
GAAGCGTTTTCAGGATAAAAGTGAGAAATATTCTAGAAAACCTAAGTTCAAGGTTG
AGAAGAAAGATATCTTGGACGATGACAAATTGGAAGGATCTAAGTTTGGCAAAGTT
AATGACATATCCTGGTATCAGAAGAATCCTGATTTACTCAGAGCTGCTGGTAACTTG
TCTTTTGCTAATGCGTTGGGATCTGGAATTGATCTATCTAACGCAAACTTTAACGTTA
AGCTTGCTGCTGATGAGCAACGTGTTCCTGGTATTGCAACTATACATACTATTACAG
GACCTGGACTCAGTCGCGACGCACACTCTGGTGTCAACGTGGCAATGCGTAACTTAT
ATTCTTTTGTTCGTCATGCAAATAGTGGTCATAGTAACTATGATCCTGTAGATTTAAT
GCTATATCTACTTGCTATGGATGAAGCATATATGGTCTACTACCGTGCTGTTCGTGCA
TATGGCGCAATGTTCACATTTAATACCGTGAACCGCTATGCTCCAAAAGCTCTTGTG
GAAGCGTTAGGTTTTGATTATGAAGATGTCAACTCAAACCTTGCTACATTCAGATAT
GCAATTAACGCATACGCTGCAAGAATCAACGCATACGCTGTGCCTACGAATATGCCT
ATCTTCAAACGACATGCATGGCTCTTTTCATCTATCTATACAGATGAAAACGTATCT
AAAGCTCAGATTTATGCATTTACTTCTGATCATTATAGAGTATATGATGAGAAGTAT
TCTAAAGGTGGACGCCTTGTGGCTAAAGCCTGGAAATCAAAGTTAACTGTCAATGAT
TGGATAACTGTAGCTAATGAAGTTGCGGATCCAATTACAGTCTCTGAGGATCTAGGA
ATCATCTCGGGTGACTTAATCAAGGCATTTGGTAAAGAGAATTTACATATGCTAGCT
ACTTTAGCTGACAATTATGTTGTATTACCAACATATGTACCTGAAGTTATGGATCAG
ATTCATAACTTACAAGCAGTAGGTCAAATTGATCTAGAAAGTAATAATATTGAACAG
GATCCAAATATTGGAAAGGGTAATTTAATTTACAATCCTATTGTAACTGTCAATAAT
AATCCAATGGCATATGCTAACCGCATCATGGATTTCAAGATTGATACACCTACACCA
GATGATGTTGTTGTAGCTTCACGATTAGCTGTGGCATTAGAACCAGGCGCTACAACC
GGTAAAGCAGTATTCACTGCCATGGGTACAGAGTTTGTGACAAATGTTGGTATTCAC
ACATTCTACAAAGGAAGTAATGGATTGCTTAAATCTATTGAACAAACTTTCAACACT
TTTGATTCTACTGAAGGTGGTCTTACTGACGCCGCATCAGTTAGTTTGCACATGTCTG
CCTACACAAAGGCCTCTAAGTTTGTACACTTTCCTATTCAATATATGTGTATGGGTAG
CTCTACTCAACCTGACAAGCGTGAAGTCAGAGTCTTTGGCGAATTGGGCACGTACAC
TATTGTTAATGGGGTCACTCTTAGTAAGTTACACGATGTGTGTGTATTAAGTCTATTT
GATGTACCTATTAAACTTTAGATGCATTAGGGGAAGTG
>27-PBV-19-044_Capsid
(SEQ ID NO: 37)
TTTCTAGTAAGAACTTAAAAGTTATTTACTAGAAAGGAGAAATTTGTACTTTAATGG
TTTACAAGAGTTTAAAACCATACAACACTTTCTACACTCTAAGAACACCAGCTACTG
CACATAGTATAGTGCAAATAGCTAGGATCAGAGATAGTGAAGTGGGATTATCTGAA
AGGAGGTTAAATTAATGACAGGTAATCAAATTAAATATGGTGAATTACAAGAGAAT
ATTCGCCATAACACAACAACAGAAGTTGAAACCAATAGACATAACGTTGTAACTGA
AGGTGAAACTAACAGACATAACGTTGTCACAGAGGTTGAGACTAATCGACACAATA
CTGTGACTGAAAGTATTGGATGGTACGATGCTGTATCAAAACGTATCTCAGCAAATG
CTTCAATGAGCCAAGCCGGTGCAGCTTGGGCTAACGTTGCAATCAATCAACAAAAT
GCAGACACGAAACGATTTGAGGCTGAACGTAATGCTGAAATAAATCAGCAAAATGC
GGATACTAAAGCATTTAGTGCACGCAGTGAGGATGCAGCTAGATATGCTCATTCTTA
CAATGAGGATCGTAAGACTACAGCAGAAATTGAGCGAATGAACACACAAAATTCGC
AAGGATGGGTGAAGTCAATCACTGATGCAATTAGCGCACCTATCAAAGCATTACCA
TTATTAGGAGGATAAATTTTATGGTAAAGAATAACAACAAGAAGCGTTTTCAGGAT
AAAAGTGAGAAATATTCTAGAAAACCTAAGTTCAAGGTTGAGAAGAAAGATATCTT
GGACGATGACAAATTGGAAGGATCTAAGTTTGGCAAAGTTAATGACATATCCTGGT
ATCAGAAGAATCCTGATTTACTCAGAGCTGCTGGTAACTTGTCTTTTGCTAATGCGTT
GGGATCTGGAATTGATCTATCTAACGCAAACTTTAACGTTAAGCTTGCTGCTGATGA
GCAACGTGTTCCTGGTATTGCAACTATACATACTATTACAGGACCTGGACTCAGTCG
CGACGCACACTCTGGTGTCAACGTGGCAATGCGTAACTTATATTCTTTTGTTCGTCAT
GCAAATAGTGGTCATAGTAACTATGATCCTGTAGATTTAATGCTATATCTACTTGCT
ATGGATGAAGCATATATGGTCTACTACCGTGCTGTTCGTGCATATGGCGCAATGTTC
ACATTTAATACCGTGAACCGCTATGCTCCAAAAGCTCTTGTGGAAGCGTTAGGTTTT
GATTATGAAGATGTCAACTCAAACCTTGCTACATTCAGATATGCAATTAACGCATAC
GCTGCAAGAATCAACGCATACGCTGTGCCTACGAATATGCCTATCTTCAAACGACAT
GCATGGCTCTTTTCATCTATCTATACAGATGAAAACGTATCTAAAGCTCAGATTTAT
GCATTTACTTCTGATCATTATAGAGTATATGATGAGAAGTATTCTAAAGGTGGACGC
CTTGTGGCTAAAGCCTGGAAATCAAAGTTAACTGTCAATGATTGGATAACTGTAGCT
AATGAAGTTGCGGATCCAATTACAGTCTCTGAGGATCTAGGAATCATCTCGGGTGAC
TTAATCAAGGCATTTGGTAAAGAGAATTTACATATGCTAGCTACTTTAGCTGACAAT
TATGTTGTATTACCAACATATGTACCTGAAGTTATGGATCAGATTCATAACTTACAA
GCAGTAGGTCAAATTGATCTAGAAAGTAATAATATTGAACAGGATCCAAATATTGG
AAAGGGTAATTTAATTTACAATCCTATTGTAACTGTCAATAATAATCCAATGGCATA
TGCTAACCGCATCATGGATTTCAAGATTGATACACCTACACCAGATGATGTTGTTGT
AGCTTCACGATTAGCTGTGGCATTAGAACCAGGCGCTACAACCGGTAAAGCAGTATT
CACTGCCATGGGTACAGAGTTTGTGACAAATGTTGGTATTCACACATTCTACAAAGG
AAGTAATGGATTGCTTAAATCTATTGAACAAACTTTCAACACTTTTGATTCTACTGA
AGGTGGTCTTACTGACGCCGCATCAGTTAGTTTGCACATGTCTGCCTACACAAAGGC
CTCTAAGTTTGTACACTTTCCTATTCAATATATGTGTATGGGTAGCTCTACTCAACCT
GACAAGCGTGAAGTCAGAGTCTTTGGCGAATTGGGCACGTACACTATTGTTAATGGG
GTCACTCTTAGTAAGTTACACGATGTGTGTGTATTAAGTCTATTTGATGTACCTATTA
AACTTTAGATGCATTAGGGGAAGTG
>28-PBV-19-046_Capsid
(SEQ ID NO: 38)
TGTAAATAACTTTTAAGTTCTTACTAGAAAGGAGAAATTTGTACTTTAATGGTTTAC
AAGAGTTTAAAACCATACAACACTTTCTACACTCTAAGAACACCAGCTACTGCACAT
AGTATAGTGCAAATAGCTAGGATCAGAGATAGTGAAGTGGGATTATCTGAAAGGAG
GTTAAATTAATGACAGGTAATCAAATTAAATATGGTGAATTACAAGAGAATATTCGC
CATAACACAACAACAGAAGTTGAAACCAATAGACATAACGTTGTAACTGAAGGTGA
AACTAACAGACATAACGTTGTCACAGAGGTTGAGACTAATCGACACAATACTGTGA
CTGAAAGTATTGGATGGTACGATGCTGTATCAAAACGTATCTCAGCAAATGCTTCAA
TGAGCCAAGCCGGTGCAGCTTGGGCTAACGTTGCAATCAATCAACAAAATGCAGAC
ACGAAACGATTTGAGGCTGAACGTAATGCTGAAATAAATCAGCAAAATGCGGATAC
TAAAGCATTTAGTGCACGCAGTGAGGATGCAGCTAGATATGCTCATTCTTACAATGA
GGATCGTAAGACTACAGCAGAAATTGAGCGAATGAACACACAAAATTCGCAAGGAT
GGGTGAAGTCAATCACTGATGCAATCAGCGCACCTATCAAAGCATTACCATTATTAG
GAGGATAAATTTTATGGTAAAGAATAACAACAAGAAGCGTTTTCAGGATAAAAGTG
AGAAATATTCTAGAAAACCTAAGTTCAAGGTTGAGAAGAAAGATATCTTGGACGAT
GACAAATTGGAAGGATCTAAGTTTGGCAAAGTTAATGACATATCCTGGTATCAGAA
GAATCCTGATTTACTCAGAGCTGCTGGTAACTTGTCTTTTGCTAATGCGTTGGGATCT
GGAATTGATCTATCTAACGCAAACTTTAACGTTAAGCTTGCTGCTGATGAGCAACGT
GTTCCTGGTATTGCAACTATACATACTATTACAGGACCTGGACTCAGTCGCGACGCA
CACTCTGGTGTCAACGTGGCAATGCGTAACTTATATTCTTTTGTTCGTCATGCAAATA
GTGGTCATAGTAACTATGATCCTGTAGATTTAATGCTATATCTACTTGCTATGGATGA
AGCATATATGGTCTACTACCGTGCTGTTCGTGCATATGGCGCAATGTTCACATTTAAT
ACCGTGAACCGCTATGCTCCAAAAGCTCTTGTGGAAGCGTTAGGTTTTGATTATGAA
GATGTCAACTCAAACCTTGCTACATTCAGATATGCAATTAACGCATACGCTGCAAGA
ATCAACGCATACGCTGTGCCTACGAATATGCCTATCTTCAAACGACATGCATGGCTC
TTTTCATCTATCTATACAGATGAAAACGTATCTAAAGCTCAGATTTATGCATTTACTT
CTGATCATTATAGAGTATATGATGAGAAGTATTCTAAAGGTGGACGCCTTGTGGCTA
AAGCCTGGAAATCAAAGTTAACTGTCAAAGATTGGATAACTGTAGCTAATGAAGTT
GCGGATCCAATTACAGTCTCTGAGGATCTAGGAATCATCTCGGGTGACTTAATCAAG
GCATTTGGTAAAGAGAATTTACATATGCTAGCTACTTTAGCTGACAATTATGTTGTA
TTACCAACATATGTACCTGAAGTTATGGATCAGATTCATAACTTACAAGCAGTAGGT
CAAATTGATCTAGAAAGTAATAATATTGAACAGGATCCAAATATTGGAAAGGGTAA
TTTAATTTACAATCCTATTGTAACTGTCAATAANAATCCAATGGCATATGCTAACCG
CATCATGGATTTCAAGATTGATACACCTACACCAGATGATGTTGTTGTAGCTTCACG
ATTAGCTGTGGCATTAGAACCAGGCGCTACAACCGGTAAAGCAGTATTCACTGCCAT
GGGTACAGAGTTTGTGACAAATGTTGGTATTCACACATTCTACAAAGGAAGTAATGG
ATTGCTTAAATCTATTGAACAAACTTTCAACACTTTTGATTCTACTGAAGGTGGTCTT
ACTGACGCCGCATCAGTTAGTTTGCACATGTCTGCCTACACAAAGGCCTCTAAGTTT
GTACACTTTCCTATTCAATATATGTGTATGGGTAGCTCTACTCAACCTGACAAGCGT
GAAGTCAGAGTCTTTGGCGAATTGGGCACGTACACTATTGTTAATGGGGTCACTCTT
AGTAAGTTACACGATGTGTGTGTATTAAGTCTATTTGATGTACCTATTAAACTTTAGA
TGCATTAGGGAATGTT
>12PBVKM-19-012_Capsid
(SEQ ID NO: 39)
NNNNNNNNNNNTTTATTTTTCTTTTGAGCATTCGCTCATCTAATCCACTATTTTAAAA
TCTTTAATAAGTTAGATTCTAAACAAACTTACACATCTAGAACATTTGTATGATTTAA
CCACAGAAAGGAGGTTAACGCATATGTTTTATGTGATTTACTTTACTCGGAACTGTG
GCACAGATTGGTCATTACATTTGTGTCCTTTGTCCGGGTAGTAACATCCAGAAAGGA
GGTCGATATGACCGAAAACCAATTAAAGTATTGGGATTTGCAAGAAACAAAACGGC
ATAACTTGCGAACAGAAGAACTTGATCAGTATAGAACTGATAAACAATTTGAAGGT
ACTAAGTATAGTGCCGACAGAAACTATGAAGGCGTAGTTTATTCAGCAAATAAGAA
TTATGAAGGAGTAAAGTACTCCGCTGATAGACATTACGCAGCAGCAATTGGATCAG
CTAAAATTCACGCTGGTGCTACGGTTGCAGCAGCTCGTATTGGAGCAGGCGCTGCAA
TAGCTTCAGCAAGAATTGGAGCTAACGCTGCAATAAGTTCTTCACAAATCAACGCAG
CAGCTAATATGTTTAATTCAAATCGAATGGCAGCTGCACAAACTTATAGTGCTGACA
GACATTACCAAGCTCAGATAACGACAACCAAGATGAACAATTACCAATCTTGGAAG
AATACGCGTGATACTAATAACGCAAGTAATTTCAATGCATTAATAGGTGCATTTGGT
AAAGTTGGTGCTGCAGCAGTAGGAAACGGCCTGAGAGGTCGCAGATAAGAAAGGA
GGCCACAAATGGCTAAGACAAACAAAACTAACAAAAGATCCAAGTTTAAGGGAGA
CTCTAAATTTGACAGCAGAGGAGGAAATAGAAATGGTAAAGGTAACAGAAACAAG
CGACCAGATAGTAGAGGCGCTAGCGAACTACCTAAAGANNNNNNNNNNNNNNNNN
NNNNNNNNNNNNACAGTGACGGAACCAACAATGTCAATTGGTATGCTCCTACTGAA
CGCATACTTAATGACACAGCAACGATCCCGTTCAACCACGCGATTGGTAACGTATTT
AACGATAGTATTCCTAACGTTCGTCTTGTTAATGCTATTCCTGGTATTATGGAAGTCA
AGTACATTCCCACAATTGGCATAAGTACTGACTACTCCTCACCTATAAACATTGCAG
CTAAGAATATTTACTCAACGATTCGTCACAGCATATCAGGCACAAGAAAATATGATG
CGAGTGACGTTATGACTTACTTAATTCCAATTACATCTATCTTCAATTATTGGAATTG
GTGCTGCAGGTTATATGGTATTCATAAGTATTTTGTTCTTAGAAACAGATACGTTCCT
GAAGGTATATTCCGTGCAATGCACGTTGACTATGATGATTTTGTTAACAATCTAGCA
GACTTTAGAGCTAAGCTGAATCAGATTGCCTTCAGGTTGTCAAACTACAAATTACCT
AAGGATATACCGCTAATTGATAGACAGACACTCTTAAATGAAGCGGTGTTTAAAGA
TGGTGATAGTGATCTCGATCAGATCTACTTCTTCAATCCAATTGGACACTACAAATA
TCAGCCTGTACTTACACAGACAGGTGGCGCGTGCCAGCTGGTTCCATCTATCACAGA
ACTGTTCACTTATAACAAACCTGCAAAGGTTAAAGATCTAATATCCTATTTTGATAC
ATTATTCTTTGATATCAATACTGACAATGATTTCGATAAGATTGGAGCTGATATCGA
AACTGCATACTCGGACAATGCACTTATGCATATTGCTGAGCTTACTGAGGATTATAT
GATCGAGCCATTCTTCTCAATCGAAATGGCAGAACAGCTAAATAATGCAGATATTGT
GCCTATTAAACCATTCCCTAAGGATATCACAACTAAGGGTTTCACACGTAAAACAGC
TACAGACTTTGATATTATTCAGGATGTAGACAGAAACATTCTGTACAGTGATCCAAA
TACTTGGCTACACTCATCTGTAGATTGGGATAAAGTATTCTACTTACCAACTCTAAG
ACTAATTAATACTAGCGTGGTTAATCCAAATCCAGCTGTAGTCATGGCTTCAACTAG
ATTAAAAGTGGCTATTGNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNAGCAAGAGCGAGGTTTACTACACTTCA
GAATCTAGCAAATTTCACTCCAATCAGTAACTTCAAGTTAGCTGATTTGCATTCTGTT
GCAATAATGGGTGAATTCAATATACCNNNNNNNNNNNNNNNNNNNNNNNNNNNNN
NNNNNNNNNNNNNNNNNNNNNNNNNNNNNNNNNNNNNNNNNNNNNNN
>14PBVKM-19-015_Capsid
(SEQ ID NO: 40)
NNNNNNNNTTTATTTATTTCTTTAGAGCATTCGCTCTTCAAAACAATCCACTTTTAAA
TCTTTAGAAAGTTAGATTCTAAACAAACTTTCATATATCTAGAAAGCATTTGTATGA
TTTAACCACAGAAAGGAGGTTAAACACTTATGTTTTATGTGATTTACAATACTCGAA
ACTGTGGCACAGACTGGTCATTACGTTTGTGTCCTTTAATCGAGTAGTAACATCCAG
AAAGGAGGTCGATATGACCGAGAACCAATTAAAATATTGGGATTTGCAGGAATTAA
AACGGCATAACCTGCGAACAGAAGAATTGGATCAGTACAGAACTGACAAACAATTC
GAAGGAACTAAGTATAGTGCGGATAAGAACTACGAAGGTGTAGTTTATTCAGCTAA
CAAGAATTATGAAGGAGTGAAATACTCCGCTGATCGACACTATGCTGCAGCGATTG
GATCAGCAAGAATACATGCTGGTGCTACAGTAGCAGCAGCGAGAATTGGAGCAGGC
GCAGCAGTCGCTTCTGCTCGTATTAATGCTAATGCTGCAATTAGTTCTGCACAAATT
GGAGCTGCGTCAAACATGTTCAATTCACAGAGAATGGCAGCTGCACAACAGTATAG
TGCTGATAGACATTATCAAGCACAGGTAACAACAACGCGTATGAATAATTACCAGT
CGTGGAAGAACACACGTGACACTAACGACGCTAGCAACTTCAATGCACTTATTGGT
GCATTTGGTAAAGTTGGCGCTGCAGCGGTTGGATCTGGTATGAGACGCGGAAGATA
GAAAGGAGGCCAATAATGGCTAAGAAATCAAACACTAATTCAAGATCCAAGTTTAA
GGGAGAACCAGACTTTAAGTCAAAAGGAGGTAAATTCAATGGTAAAGGTAACAGAA
ACAGGAGATCAGATGGTAGAAGCGCTAACGGCATACCTGAAGAATCAGGAGACAA
ATTCGAAAAGCAGCGCAACAGTGACAGAACCAACTCTGTCAATTGGTATGCTCCTAC
TGAGCGCATACTTACAGATACAGCAACGATCCCGTTCAACCACGCGATTGGTAACGT
ATTTGACGATAGTATTTCTAACGTTCGTCTTGTTAATGCTATTCCTGGTATTATGGAA
GTCAAGTACATACCCACAATTGGCGTAAGCACTGACTATTCATCACCAATTAATATT
GCAGCTAAGAATATATATTCTAATATTAGGCACAGTATCTCAGGTACTAGAAAGTAT
GATGCAAGTGACGTCATGACTTATCTAATACCAATTACATCAATATTCAATTACTGG
GCTTGGTGCTGCAGACTGTACGGAATTCACAAATATTTCGTACTCAGGAATAGATAT
GTACCTGAAGGCATATTCCGTGCTATGCACGTGGATTATGATGATTTTGTCAATCAT
CTAGCTGACTTTAGGGCTAAACTAAATCAGATAGCGTTCCGCCTATCTAATTACAAG
TTACCAAAAGATATTCCACTGATCGATAGACAGATGTTACTTAACGAAGGAATATTT
GCAGACGGCATGAGTGATCTAGACCAGATCTACTTCTATAATACTATAGGCCACTAT
AAATATCAGCCAGCCATGACTGAAACTGGTGGTTCATGCCAACTAATTCCATCTATC
ACGGAATTATTTACTTACAATAAACCAGCAAAAGTTTCTGATTTAATCAGCTACTTT
GATCAGTTATTCTTTGATATTAATACGGATAATGATTTCGATAAGATCGGTGCTGAT
ATTGAAACTGCATATTCCGATGGAGCACTTATGCATATAGCTGAACTTACAGAAGAT
TACAGCATTGCTCCAATCTACTCGTTGGAAATGAATGAACAGCTTAACAACGCTGAC
ATTCTACCAATCAATCCATTCCCTAAGGATATCACAACTAAGGGATTCACTCGTAAA
ACAGCAAGCGTATACGACATAGTGCAGGATGTTGATAGAAACATTCTATACCATGA
TCCAGCTTCGTGGCTATACAATATCAACGGAGATGGAGAAACGTTTGATTTGCCTAC
TTTACGTATTTTAAATACTAAAGTATCTGACCCTAATCCTAGTATTATTATGGCAGCC
ACTCGACTAAAAGTAGCGATTGATGAAACTGGGAAAATACTAGGTTGCGGAACTGA
AATTGTAACAGGTATCACTGTGCATAATATGTCACAAGATATTGATACTAAGGGTAA
GTGGTACACCGTACCACAGGAGTGGTCCATTAAATCCAATATTGTATACACTATTAA
CGGTGAGTTTAAAGTCATTTACTCACGTGACGAAAATAGTGGAGAAATTGGAAACC
TTATGGGTCTGAAGTATCTACTAGAATATTTCAGCAAGTGGGAGTATGCTCCTATGA
TATACACATATGATGTAGCACCTCTATTAGATAACGAGGAAACAGTAGCTCAATTTA
AGAGCAAGAATAAGGAAGCAAGAGGTAGATTTACTACGCTCCAGAATTTGGCCAAT
TTCACACCAATTAGTAACTTCAAACTAAGTGATTTACACTCAGTTGCTATCATGGGT
GAATTTAACATACCTGGCACAATTAGTTATAAAGGTACTAAATAACATTTAGTGTAA
AGGTATGCAGTAGGGAGAACCCTACTGCATACC
>18PBVKM-19-023_Capsid
(SEQ ID NO: 41)
NNNNNNNNNNNNTTTATTTTCTTTAGAGCATTCGCTCTTCAAAACAATCCACTAATA
ATCTTTTAGAAGTTAGATTCAAAACAAACTTTCTATATCTTAAGAACATTTGTATGAT
TTAACCACAGAAAGGAGGTTAAACACTTATGTTTTATGTGATTTACAATACTCGAAA
CTGTGGCACAGACTGGTCATTACGTTTGTGTCCTTTAGTCGAGTAGCAACATCCAGA
AAGGAGGTCGATATGACCGAAAACCAATTAAAGTATTGGGATTTGCAGGAAACAAA
ACGGCATAACCTGCGAACAGAGGAATTGGATCAGTATCGAACTGATAAACAATTCG
AAGGTACTAAGTATAGTGCAGATAAGAACTACGAAGGAGTAGTTTATTCAGCAAAC
AAGAATTACGAAGGAGTCAAGTACTCCGCTGACAGACACTATGCAGCAGCAATTGG
ATCAGCAAGAATACATGCTGGTGCTACCGTAGCAGCAGCAAGAATTGGAGCAGGCG
CAGCAGTCGCTTCTGCCCGTATTAGTGCTAACGCTGCAATTAGTTCCGCACAAATTG
GAGCAGCTTCAAATATGTTTAATTCACAAAGAATGGCTGCCGCACAACAGTATAGTG
CTGATAGACATTATCAAGCACAGATAACAACTACGCGTATGAATAATGCACAATCCT
GGAAGAACACCAGAGATACTAACGATGCAAGTAACTTTAATGCCCTCATTGGAGCA
TTTGGAAAGATAGGAGCATCAGCAGTTAGTTCAGGCATGAGACGCGGAAGATAGAA
AGGAGGCCAAATATGGCTAAGAAAACAAACACTAAATCAAGATCCAAGTTTAAGGG
AGAACCAGACTTTAAGTCAAAAGGAGGTAAATTTAATGGTAAAAGTGACAGAAACA
GGAGATCAAATGGTAGAGGCGCTAACAGCTTACCTGAAGAATCAGGAGACAAATTC
GAAAAGCAGCGCAACAGTGACAGAACCAACTCTGTCAATTGGTATGCTCCTACTGA
GCGCATACTTGCAGATACAGCAACGATCCCGTTCAACCACGCGATTGGTAACGTATT
TGACGATAGTATTTCTAACGTTCGTCTTGTTAATGCTATTCCTGGTATTATGGAAGTC
AAGTACATACCCACAGTTGGCGTAAGCACTGACTATTCATCACCAATTAATATTGCA
GCAAAGAATATTTATTCTACAATTAGACACGAAATTTCAGGTACAAGGAAATATGA
CGCTAGTGACGTCATGACGTACCTAATTCCAATAACATCAATCTTTAATTATTGGGC
TTGGTGCTGCAGACTATATGGAATACATAAATACTTCGTCCTTAGGAATAGATATGT
TCCTGAAGGCATTTTCCGTGCTATGCACGTTGATTATGATGATTTTGTAAATCATCTA
GCTGATTTTAGAGCCAAACTAAATCAGATCGCGTTCAGACTATCAAACTATAAATTA
CCAAAGGATATTCCATTAATTGATCGTCAGATGCTTCTAAACGAGGCAATCTTTGCT
GACGGAATGAGTGATCTTGACCAGATCTATTTTTATAATACTATAGGACATTATAAG
TATCAGCCTGCTATGACAGATACAGGTGGTGCTTGCCAGCTTATTCCGTCCATTACT
GAACTGTTCACCTACAATAAACCAGCTAAGGTATCCGACTTAATTGAATATTTTGAT
AAATTGTTTTTCGATATTAATACAGATAATGATTTTGACAAGATCGGAGCCGATATT
GAAACTGCTTATTCTGAGAATGCTCTAATGCATATAGCTGAATTAACGGAGGACTAT
AACATAGCTCCAATTTATTCTTTAGAAATGAATGAGCAGCTGAATAACGCTGATATT
CTACCAATTAAACCATTTCCTAAAGATATTACAACTAAGGGATTTAATCGTAAAACA
GCTACAGATTTCGACATCATACAGGATGTAAATCGCAATATACTTTATCATGATCCA
AATGCTTGGCTCAAGAACATCAATACTGAGGAAGAAATATTTGATGTGCCTACATTA
AGAATCTTGAATACAAAGGTTTCTGATCCTAATCCTAGCATCGTAATGGCAGCTACA
AGATTGAAAGTAGCCATTGATGAAAAGGGTAGAATTCTAGGCTGTGGCTCAGAGAT
AGTTACAGGCATTACTGTGCATAATATGTCACAGGATCTGGATACAAATGGTAAATG
GTACACAGTACCACAGGAATGGTCCATTAAATCTAACATCGTGTACACGATTAATGG
TGAGTTCAAAGTACTTTACTCAACTGATGAAAACAGTGGAGAAATTGGTAATTTAAT
GGGACTCAAATATTTACTAGAGTACTTTAGTAAATGGGAATATGCCCCAATGATCTA
TACATATGATGTTACACCTCTTCTGGAGAATGAGGAAACAATTGCACAATTTAAGAG
TAAACATAAAGAGGCAAGAGGTAGGTTTACAACACTCCAGAATTTAGCTAACTTCA
CACCAATTAGTAACTTTAAGCTAAGTGATCTACACTCAGTTGCTATAATGGGTGAGT
TTAATATACCTGGTACCATTACATACAAGGGTACTAAATAATTTATCTATAAAGGTA
TGCAGTAGGGAGAACCCTACTGCATACC
>21PBVKM-19-033_Capsid
(SEQ ID NO: 42)
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNACACTACGCTGCAGCTATTGGATCTGCTAAAATTCATGCTGGTGCAACAGTAGCA
GCTGCTCGCATCGGAGCTGGCGCTGCAATCGCTTCAGCAAGAATTGGAGCTAACGCT
GCAATTAGTTCTTCACAAATTNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNT
GACGGAACCAACAATGTCAATTGGTATGCTCCTACTGAACGCATACTTAATGACACA
GCAACGATCCCGTTCAACCACGCGATTGGTAACGTATTTAACGATAGTATTCCTAAC
GTTCGTCTTGTTAATGCTATTCCTGGTATTATGGAAGTCAAGTACATTCCCACAATTG
GCATAAGTACAGACTACTCCTCACCAATAAACATTGCAGCTAAGAATATTTATTCAA
CTATACGTCACAGTATCTCAGGCACAAGAAAATATGATGCTAGTGATGTTATGACTT
ATCTGATNNNNNNNNNNNNNATCTTTAATTATTGGAACTGGTGCTGCAGGTTATATG
GTATTCACAAGTATTTTGTTTTAAGGAACAGATACGTGCCTGAAGGTATATTCCGTG
CTATGCACGTTGATTACGATGATTTCGTTAATAACCTAGCTGATTTCAGAGCTAANN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNTATGCATATCGCTGAGCTCACTGAGGATTAT
ATGATTGAGCCTTTCTTCTCAATTGAGATGGCTGAACAGTTAAATAACGCAGATATT
GTGCCTATTAAACCATTCCCTAAGGATATCACAACTAAGGGATTCACACGTAAAACT
GCNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNGCAAGAGCAAGGTTT
ACCACACTTCAGAATCTAGCGAATTTCACACCAATCAGTAATTTTAAATTAGCTGAT
TTGCATTCTGTTGCAATTATGGGTGAATTTAACATACCTGGTACAATTACTTATAAGG
GTACAAAGTAATATNNNNNNNNNNNNNNNNNNNNNNNNNNNNNNNNNNNNNNNN
NN
>22PBVKM-19-034_Capsid
(SEQ ID NO: 43)
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNTGACGGAACCAACAATGTCAATTGGTATGCTCCTACTGAACGCATACTTA
ATGACACAGCAACGATCCCGTTCAACCACGCGATTGGTAACGTATTTAACGATAGTA
TTCCTAACGTTCGTCTTGTTAATGCTATTCCTGGTATTATGGAAGTCAAGTACATTCC
CACAATTGGCATAAGTACAGACTACTCCTCACCAATAAACATTGCAGCTAAGAATAT
TTATTCAACTATACGTCACAGTATCTCAGGCACAAGAAAATATGATGCTAGTGATGT
TATGACTTATCTGATTCCAATTACATCAATCTTTAATTNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNACTGAACTGTTCACTTACAATAAGCCAGCTAAGGTTAAGGACCTAATATCCTA
TTTTGATACTCTATTCTTTGATATCAATACAGATAATGATTTCGATAAGATTGGAGCT
GATATCGAAACTGCGTACTCNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNN
>26PBVKM-19-039_Capsid
(SEQ ID NO: 44)
NNNNNNNNNNNNNNNNNNNNNNNNNNNNNNNNNNNNNNNNNNNNNNNNNNNNNN
NNNNNNNNNNNNNNNNNNAGATTCTAAACAAACTTACACACCTAGAACATTTGTAT
GATTTAACCACAGAAAGGAGGTTAACGCATATGTTTTATGTGATTTACTTAACTCGG
AACTGTGGCACAGATTGGTCATTACATTTGTGTCCTTTATCCGGGTAGTAACATCCA
GAAAGGAGGTCGATATGACCGAAAACCAATTAAAGTATTGGGATTTGCAAGAAACT
AAACGGCATAACTTGCGAACAGAAGAACTAGATCAGTACAGAACTGACAAACAATT
CGAAGGCACAAAATACAGTGCTGACCGAAATTATGAAGGCGTAGTCTATTCAGCTA
ATAAGAATTATGAAGGAGTGAAATATTCCGCTGATCGACACTACGCTGCAGCTATTG
GATCTGCTAAAATTCATGCTGGTGCAACAGTAGCAGCTGCTCGCATCGGAGCTGGCG
CTGCAATCGCTTCAGCAAGAATTGGAGCTAACGCTGCAATTAGTTCTTCACAAATTA
ACGCAGCGGCTAATATGTTTAATTCTAATAGAATGGCGGCCGCACAGACTTATAGCG
CTGACAGACATTATCAGGCTCAAATTACTACTACCAAGATGAATAATTACCAATCTT
GGAAGAATACGCGTGATACTAATGACGCGAGCAATTTCAATGCACTGATAGGTGCA
TTTGGAAAGGTCGGCGCTGCAGCAGTTGGGAGCGGCTTGAGAGGTCGCAGATAAGA
AAGGAGGCCTACTATGGCTAAGACAAACAAAACTAATAAAAGATCCAGGTCTAAGG
GAGACTTCAAGAATGACACTAGAGGAGGAAACAGAAATGGTAAAGGTAACAGAAA
CAAGCGACCAGATAGTAGAGGCGCTAGCGAACTACCTAAAGACGCCCAGGGAAGA
GGGGACGAGCAATTTAAGAATGACGGAACCAACAATGTCAATTGGTATGCTCCTAC
TGAACGCATACTTAATGACACAGCAACGATCCCGTTCAACCACGCGATTGGTAACGT
ATTTAACGATAGTATTCCTAACGTTCGTCTTGTTAATGCTATTCCTGGTATTATGGAA
GTCAAGTACATTCCCACAATTGGCATAAGTACAGACTACTCCTCACCAATAAACATT
GCAGCTAAGAATATTTATTCAACTATACGTCACAGTATCTCAGGCACAAGAAAATAT
GATGCTAGTGATGTTATGACTTATCTGATTCCAATTACATCAATCTTTAATTATTGGA
ACTGGTGCTGCAGGTTATATGGTATTCACAAGTATTTTGTTTTAAGGAACAGATACG
TGCCTGAAGGTATATTCCGTGCTATGCACGTTGATTACGATGATTTCGTTAATAACCT
AGCTGATTTCAGAGCTAAGCTAAATCAGATTGCTTTCAGGTTGTCAAATTATAAACT
ACCTAAGGATATACCGCTAATAGATAGACAGACTCTATTAAATGAAGCGGTATTCA
AAGATGGTGATAGTGATCTTGACCAGATTTACTTCTTCAATCCAATTGGCCACTATA
AGTATCAGCCTGTACTTACTCAAACAGGTGGTGCTTGCCAGCTGGTTCCATCTATTA
CTGAACTGTTCACTTACAATAAGCCAGCTAAGGTTAAGGACCTAATATCCTATTTTG
ATACTCTATTCTTTGATATCAATACAGATAATGATTTCGATAAGATTGGAGCTGATA
TCGAAACTGCGTACTCAGATAATGCACTTATGCATATCGCTGAGCTCACTGAGGATT
ATATGATTGAGCCTTTCTTCTCAATTGAGATGGCTGAACAGTTAAATAACGCAGATA
TTGTGCCTATTAAACCATTCCCTAAGGATATCACAACTAAGGGATTCACACGTAAAA
CTGCTACGGATTTCGACATTATTCAGGATGTAGATAGAAATATTCTATATAGCGATC
CAAATACTTGGCTACATTCTGATGTTAACTGGGATCAGGTATTTTATTTACCTACACT
GAGATTAATCAACACAAGCGTGGTTAACCCAAACCCAGCTGTAGTAATGGCTTCGA
CAAGATTAAAAGTAGCTATTGATGAAGTTGGTAAAATAGCAGGTTGTGGAACTGAG
ATTGTAACTGGAATTACCATTCATAATATTTCTCAGGAAGTTGATGATAAAGGGAAA
TGGGCTACACTTCCACAAGAGTGGAGTATCAGATCAAACATGGTGTATACATTAAAT
GGTGTATATCAGCATCTATACTCATCTGACGCAGATAGCGGTGAAATAGTTGATACT
ATGACAATGAAGTATTTGCTAGAGTACTTTAGTAAGTGGGAGTATGCTCCAATGGTG
TATACATATGATGTAACACCTCTTTTGAATGAAGACGAAGATATTTCAACGTTTAAA
CGTGGTAATCGTGTTGCAAGAGCAAGGTTTACCACACTTCAGAATCTAGCGAATTTC
ACACCAATCAGTAATTTTAAATTAGCTGATTTGCATTCTGTTGCAATTATGGGTGAAT
TTAACATACCTGGTACAATTACTTATAAGGGTACAAAGTAATATATACCACATTTAG
GTAGCAGTAGGGAGACCCTACTGCTACC
>27PBVKM-19-044_Capsid
(SEQ ID NO: 45)
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNGTCATTACATTTGTGTCCTTTATCCGGGT
AGTAACATCCAGAAAGGAGGTCGATATGACCGAAAACCAATTAAAGTATTGGGATT
TGCAAGAAACTAAACGGCATAACTTGCGAACAGAAGAACTAGATCAGTACAGAACT
GACAAACAATTCGAAGGCACAAAATACAGTGCTGGGCCTATGAGTATTCAGAAACC
TCTTGAAGAGCGTTTCACGGATATTGAGGCTTATTACAAAGGTATTCTCCTACCTTCC
GAACCAATTAGTGATGAAGCAATCCGATCTGTCATCACTGAGTGGAACAGGGCTCG
CGGATTGTCAGTTCGCAGTACTTCCAAAACATGGGACAATATATGTAAATTCTCTTT
ACCAAATGCCTTGATTAAGTCACCCGAGATGATTCCTAGATCCTCAGAGACTGTAAT
TGGATCCGCAACTTCATTAGCTACAGTTATCCAATCATTGACAGTTAACTTTGATTTC
CAGGCTTTAGCCACAAGGCGTCCACCTTTAGAATACTTCTCAATTGAAAACTAAGAG
AGGTAACGATGAAGAGTATCGTACTCCATTTTTCAAAGGTAAATCTTTATCCGATGT
TTTAAAAGGCTGGGAAGTGCACCTCGCCCCTCTCAAAGAGAAGTGGCCTGGTTTACA
CCAGTTTGAATTAGACCTAGCGGAAAAGGTCGGGCCTATGAGTATTNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNACGGAACCAACAATGTCAATTGGTATGC
TCCTACTGAACGCATACTTAATGACACAGCAACGATCCCGTTCAACCACGCGATTGG
TAACGTATTTAACGATAGTATTCCTAACGTTCGTCTTGTTAATGCTATTCCTGGTATT
ATGGAAGTCAAGTACATTCCCACAATTGGCATAAGTACAGACTNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNCTATTTTGATACTCTATTCTTTGATATCA
ATACAGATAATGATTTCGATAAGATTGGAGCTGATATCGAAACTGCGTACTCAGATA
ATGCACTTATGCATATCGCTGAGCTCACTGAGGATTANNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNN
>2-PBV MRN3406_RDRP
(SEQ ID NO: 65)
CTAAATGAATAGAAAAGTAGTCAAGTTAGGTAATTATTTTAAATTACCGAATCCCGG
TTGAAGACCTATCTATTGAAAACTAAGAGAGGTAACGATGAAGAGTATCGTACTCC
ATTTTTCAAAGATAAATCTTTGTCCGATGTATTACAAGGCTGGTTAGTGCACCTAGC
CCCTCTCAAGAGTGAGTGGCCTGGTTTACACCAGTTTGAATTAGACCTAGCGGAAAA
GGTCGGGCCTTTAAGCATCCAGAAACCTTTAGATGAGCGGTTTAAGGATATTGAGGC
TTATTACAAAGGTATTCTCCTACCTTCCAAACCAATCAGTGAAACAGCAATCCGATC
TGTTTTAACTGAATGGAATAGGGCACGTGGCTTGTCGGTACGCAGTGTCTCCAAAAC
GTGGGATAACATGAAGAAATCTACATCTTCAGGTTCTCCATTCTTTACTAAACGTAA
AGCAGTCGGAAAATATACGATGTATATGGAGCCATGTTTTGACAAAAGAACGCAAG
AAGTTCATTTTAAGAACTCAAACCGTTGGGATCCAATTGCGGTCTTAGGTTGGCGTG
GACAAGAAGGTGGACCTGATTTTGAGGATGTAAAGCAAAGGGTTGTATGGATGTTC
CCTGCTTCGGTAAACCTACAAGAGTTACGTGTTTACCAACCTCTAATCGAAACAGCG
CAACGTTTCAACTTAGTTCCTGCTTGGGTTGGCATGGATAGTGTTGATTTGCACATCA
CACGTATGTTTGATACGAAAGGCGAAGACGATGTCGTAATATGTACAGATTTCTCAA
AATTTGACCAACATTTTAATGCTGATATGGCTCGCGGTGCATCCGAAATATTGGATG
GCCTCTTTAACGGGAGCAGAGATTTTGTACAATGGATGTGGGATATATATCACATCA
AATACACGATACCTCTATTAGACTCAGAAGATCATGCCTGGTTTGGCAGACATGGTA
TGGGCTCTGGTTCAGGTGGAACCAATGCCGATGAAACATTAGCTCATAGAGCTTTGC
AGTACGAAGCTGCTTTATCACAGAACCAAACATTAAACCCTTATTCACAATGTCTAG
GTGATGATGGAGTACTAACATATCCTGGAATTAAAGTGGATGATGTAATGCGATCAT
ATACTGCACATGGTCAAGAGATGAATGAGTCAAAACAGTATGTGAGCAAACATGAA
TGCATATATCTTCGTAGATGGCATCATATTAATTATCGTGTCGATGATGTATGTGTCG
GAGTTTACGCAACAACTCGTGCTTTGGGTAGATTGTGTGAACAAGAGAGATATTTTG
ACCCAGAGATATGGTCAAAAGAAATGGTAGCTTTACGTCAGCTATCGATACTTGAG
AATGTGAAATACCACCCTCTCAAGGAAGAATTTGTTAAATATTGCATGAAAGGGGA
TAAGTACAGACTGGGACTGGACTTACCAGGCTTCTTGGAGAACATAGATGGACTCG
CAAAGCAAGCTACTGATCTAATGCCGGACTTTTTAGGTTACGTTAAATCACAACAGA
AATCTGTCGGTGGTATATCAGAATGGTGGATAGTAAAATATCTACGTAGTCTAAAGT
AAAGATTGGGATGGTGCAGTAAACCATTAGAATTCTAACGAATTCTAACTGCACCAT
CCCAATCTTTACTTTAGACTACGTAGATATTTTACTATCCACCACTCTGATATACCAC
CGACAGATTTCTGTTGTGATTTAACGTAACCTAAAAAGTCCGGCATCAGATCAGTAG
CTTGCTTTGCGAGTCCATCTATGTTCTCCAAGAAGCCTGGTAAGTCCAGTCCCAGTCT
GTACTTATCCCCTTTCATGCAATATTTAACAAATTCTTCCTTGAGAGGGTGGTATTTC
ACA
>1-PBV-4466_RDRP
(SEQ ID NO: 46)
CTAGAAAAGGAGGCTACTAATGAATAGAAAAGTAGTCAAGTTAGGTAATTACTTTA
AATTACCAAATCCCGGTTGAAGACCTATCTATTGAAAACAGAGAGAGGTAACGATG
AAGAGTATCGTACTCCATTTTTCAAAGGTAAATCTTTGTCCGAAATATTAGAAGGCT
GGAAAGTGCACCTAGCCCCTCTCGAAGTTGAGTGGCCTGGTTTACACCAGTTTGAAT
TAGACCTAGCGGAAAAGGTCGGGCCATTAAGTATCCAAAAGCCATTAAAAGATAGA
CTTAAGGATATTGAGGCCTATTACAAAGGTATTCTCCTACCTTCCAAACCCATTGAC
TCAGACGCAATCCAAGCGGTTCTTGATGAATGGGAAAAGGCACGCGGTTTGTCACTT
CGATCTACTCCCAAAACGTGGGAAAAGATGAAGAAATCAACTTCATCTGGTAGTCC
ATTATTTACAAAGAGACGCAGTGTAGGTCAATTTACAATGGACTCACAACCGTGTTT
TGACTTAGTTACGCGAGAAGTACATGACGCAAAATATCGTCAGTGGGATCCAATCG
CTATACTAGGTTGGCGAGGACAAGAAGGCGGTCCTGACTTTGAGGATGTAAAACAG
AGGGTTGTATGGATGTTCCCTGCTGCAGTGAACTTGCAAGAATTGCGAGTGTATCAA
CCTCTAGTCGAAGTAGCTCAACGGTTCAACTTAGTTCCTGCTTGGGTTAGCATGGAT
AGTGTTGATTGGCACATCACACGAATGTTTGATACCAAAGGAGCAAATGATGTCGTG
ATTTGTACTGATTTCTCCAAATTTGACCAACATTTTAATGTAGATATGGCGCGCGGC
GCATCCGAAATATTGGATGGCCTCTTTAACGGGCGCGGAGATTTTATTCAGTGGATG
TGGGCAATATTCCACATTAAATACACGATACCTCTATTAGACTCTGAAAATCATGCC
TGGTTTGGCAGACATGGTATGGGTTCCGGATCTGGTGGAACTAACGCTGATGAAACA
TTAGCACATAGAGCGTTACAACATGAAGTAGCGCTATCCCATAACCAAACACTTAAC
CCTTATTCACAATGTCTAGGTGATGATGGAGTACTTACTTACCCTGGAATTAAAGTG
AATGATGTAATGCGATCATATACTGCACATGGTCAAGAAATGAATGAGTCTAAACA
GTATGTGAGCAAACATGAGTGCATATATCTTCGTAGATGGCATCATGAAAATTATCG
TGTCGACGATATATGTGTTGGAGTTTACGCAACCACTAGAGCTTTGGGTAGATTGTG
TGAACAAGAAAGATACTTTGACCCAGGAGTTTGGTCAAAGGAAATGGTAGCTTTAC
GTCAGCTATCGATCCTTGAGAATGTGAAATACCACCCGCTCAAGGAAGAATTTGTTA
AGTATTGCATGAAAGGAGACAAGTATAGACTGGGACTAGACTTGCCAGGCTTCCTC
GAGAACATAGATGGAATCGTTAAGGAAGCTACTGATCTTATGCCGGACTTCTTAGGT
TACGTTAAATCACAACAGAAAAAGGTTGGTGGTGCATCAGAATGGTGGATTGTAAA
ATATCTGCTTAGTCTAAAGTAACAGATCGGGATGGTGCAGTAAACCATTAGAATTCT
TAATGAATTCTAACTGCACCATCCCGATCTGTTACTTTAGACTAAGCAGATATTTTAC
AATCCACCATTCTGATGCACCACCAACCTTTTTCTGTTGTGATTTAACGTAACCTAAG
AAGTCCGGCATAAGATCAGTAGCTTCCTTAACGATTCCATCTATGTTCTCGAGGAAG
CCTGGCAAGTCTAGTCCCAGTCTATACTTGTCN
>3-PBV-4138 RDRP
(SEQ ID NO: 47)
GCAGAAGACGGCATACGAGATGAGCAATCGTCTCGTGGGCTCGGAGATGTGTATAA
GACTTAATGAATAGAAAAGTAGTCAGTTTAGGTAATTACTTTAAATTACCAAATCCC
GGTTGAAGACCTATCTATTGAAAACTAAGAGAGGTAACGATGAAGAGTATCGTACT
CCATTTTTCAAAGGTAAATCTTTATCCGATGTGTTAAAAGGCTGGGAAGTGCACCTT
GCCCCTCTCAAAGAGAAGTGGCCTGGTTTACACCAGTTTGAATTAGACCTAGCGGCG
AAGGTCGGGCCTATGAGTATTCAGAAACCGCTCGAAGAGCGTTTCAAGGATATCGA
GGCTTATTACAAAGGTATTCTCCTACCTTCCGAACCAATTCGTGATGAAGCAGTCCG
ATCTGTCATCACTGAATGGAACAGGGCTCGCGGATTGTCAGTTCGCAGTACTTCCAA
AACATGGGACAATATGAAGAAGTCCACTTCTTCAGGCTCTCCATTCTTTACCAAACG
TAAGTTGATTGGTAAATACATAATGGATAGTCAACCATATTTTGACAAAAGAACGCA
AGAGGTACACGATAAGGTGTATCCACATTGGGATCCAATTGCTGTTCTTGGTTGGCG
TGGACAAGAAGGAGGTCCAGAACCAGAGGATGTGAAGCAAAGGGTTGTATGGATGT
TCCCTGCTTCAGTTAACTTGCAAGAATTGCGAGTATATCAACCTCTGATCGAAACAG
CGCAACGTTTCAACTTAGTTCCTGCTTGGGTTAGCATGGATAGTGTGGACGAGCACA
TCACACGTATGTTTGATACTAAAGGCGCAGATGATGTCGTGATATGTACTGATTTCT
CTAAATTTGACCAACATTTTAACGCTGATATGGCTCGCGGCGCATCCGAAATTTTGG
ATGGTCTATTTAACGGGAGTCGAGATTTCGTACAGTGGATGTGGGATATATACCACA
TTAAATACACGATACCTCTATTAGACTCTGAAAACCATGCGTGGTTTGGACGTCATG
GTATGGGTTCCGGTTCAGGCGGAACTAATGCTGATGAGACATTGGCTCATCGCGCGC
TGCAATATGAAGCAGCACTCTCACAAAACCAAACACTAAACCCTTATTCACAATGCT
TGGGTGATGATGGAGTACTAACGTATCCAGGTATTAAAGTGGATGATGTAATGCGAT
CATATACTGCTCATGGTCAAGAAATGAATGAGTCAAAGCAGTACGTGAGCAAACAT
GAATGCATATATCTGAGAAGATGGCATCACAAAGATTATCGTGTGGCAGATATATGT
GTCGGAGTTTATGCAACTACTAGAGCTTTGGGTAGATTGTGTGAACAGGAAAGATAC
TTTGATCCAGAAGTATGGTCAAAGGAAATGGTAGCTTTACGTCAGCTATCGATCCTT
GAGAACGTTAAATACCACCCACTCAAGGAAGAATTCGTGAATTATTGCATGAAAGG
CGACAAGTATAGACTGGGACTAGACTTGCCAGGCTTCTTAGAGAACATTGATGGACT
CGCAAAGCAAGCTACTGATCTTATGCCGGACTTTTTGGGATACGTTAAGTCCCAACA
GAAGGATACTGGAATGAGCGATTGGTGGATCGTGAAGTATCTTAAAAGTTTAAAGT
AGAGATTTGGATGGTGCAGTTAGNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNGACTTAACGTATCCCAAAAAGTCCGGCAT
AAGATCAGTAGCTTGCTTTGCGAGTCCATCAATGTTCTCTAAGAAGCCTGGCAAGTC
TAGTCCCAGTCTATACTNNNNNNNNNNNNNNNNNNNNNNNNNNNNNNNNNNNNNN
NNNNNNNNNNNNNNNNNN
>10-PBV-19-001 RDRP
(SEQ ID NO: 48)
GAATAGAAAAGTAGTCAGTTTAGGTAATTACTTTAAATTACCAAATCCCGGTTGAAG
ACCTATCTATTGAAAACTAAGAGAGGTAACGATGAAGAGTATCGTACTCCATTTTTC
AAAGGTAAATCTTTGTCCGAAGTATTAAAAGGCTGGGAAGTGCACCTTGCCCCTCTC
AAAGAGAAGTGGCCTGGTTTACACCAGTTTGAATTAGACCTAGCGGAAAAGGTCGG
GCCGATGAGTATCCAGAAACCTCTTGATGAGCGTTTCAAGGATATTGAAGCCTATTA
CAAAGGTATTCTCCTACCTTCCACTCCAATTAGTGATGCAGCAATCCAATCTGTACTC
ACTGAATGGAACAGGGCTCGCGGATTGTCAGTTCGCAGTACTTCCAAAACATGGGA
TAAGATGAAGAAGTCTACTTCTTCAGGCTCTCCATTCTTTACCAAACGTAAACTAAT
TGGTAAGTATATTATGGATAGCGAACCATATTTTGACAAAAGAACGCAAGAGGTAC
ATGATAGAAAGTACCGACAATGGGATCCAATTGCTGTTCTTGGTTGGCGAGGACAA
GAAGGAGGTCCAGAACCAGAGGATGTAAAGCAAAGGGTTGTATGGATGTTCCCTGC
TTCGGTGAACCTGCAAGAATTGCGGGTATACCAACCTCTGATCGAAACAGCGCAAC
GTTTCAACTTAGTTCCTGCTTGGGTTAGCATGGATAGTGTGGATCAGCACATCACAC
GTATGTTTGATACTAAAGGCGCAGATGATGTCGTGATTTGTACAGATTTCTCAAAAT
TTGACCAACATTTCAACTCTGATATGGCTCGTGGTGCTTCAGAGATATTAGATGGCT
TGTTTAACGGAAGTCGAGATTTTGTGCAATGGATGTGGGATACATACCACATAAAGT
ACACGATACCTCTATTAGACTCGGAAAACCATGCGTGGTTTGGACGTCATGGTATGG
GTTCCGGTTCAGGCGGAACTAATGCTGATGAGACATTAGCTCATCGCGCGCTGCAAT
ATGAAGCAGCGCTTTCTCAACACCAAACACTTAACCCTTATTCACAATGCCTAGGTG
ATGATGGAGTACTTACGTACCCAGGTATTAAAGTGGATGATGTAATGCGATCATATA
CTGCTCATGGTCAAGAGATGAATGAGTCAAAGCAGTACGTGAGCACACATGAATGC
ATATATCTGAGAAGATGGCATCATAAAGATTATCGTGTGTCAGATGTATGTGTTGGA
GTTTATGCAACTACTCGTGCTTTAGGCAGGTTGTGTGAACAAGAACGCTACTTTGAT
CCAGAAGTATGGTCAAAGGAAATGGTAGCTTTACGTCAGCTATCGATCCTTGAGAAT
GTTAAATTCCACCCACTCAAGGAAGAATTCGTGAATTATTGCATGAAAGGCGACAA
GTATAGACTGGGACTAGACTTGCCAGGCTTCTTGGAGAACATTGATGGACTCGCAAA
GCAAGCTACTGATCTTATGCCGGACTTTTTGGGATACGTTAAGTCCCAACAGNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNCTAACTTCACCATCC
GAATCTTTACTTTAGACTTTTAAGATATTTAACGATCCACCAATTGCTCATTCCCGTA
TCTTTCTGCTGGGACTTAACGTATCCCAAAAAGTCCGGCATAAGATCAGTAGCTTGC
TTTGCGAGTCCATCAATGTTCTCCAAGAAGCNNNNNNNNNNNNNNNNNNNNNNNNN
NNNNNNNNNNNNNNNNNNNNNNNNNNNNNNNNNNNNNNNNNNNNNNNNNNNNNN
NNNN
>11-PBV-19-006_RDRP
(SEQ ID NO: 49)
CGGCAACCACCAAGATCTACACGTTGACCTTCGTCGGCAGCGTCAGATGTTAAATGA
ATAGAAAAGTAGTCAGTTTAGGTAATTACTTTAAATTACCAAATCCCGGTTGAAGAC
CTATCTATTGAAAACTAAGAGAGGTAACGATGAAGAGTATCGTACTCCATTTTTCAA
AGATAAATCTTTGTCCGAAGTGTTAAAAGGCTGGGAAGTGCACCTTGCCCCTCTCAA
AGAGAAGTGGCCTGGTCTACACCAGTTTGAATTAGACCTAGCGGAAAAGGTCGGGC
CGATGAGTATCCAGAAACCTCTTGATGAGCGTTTTAAGGATATTGAAGCCTATTACA
AAGGTATTCTCCTACCTTCCACTCCAATAAGTGATGAAGCAGTCCGATCTGTAATCA
CTGAATGGAATAGGGCTCGCGGATTGTCAGTTCGCAGTACTTCCAAAACATGGGATA
AGATGAAGAAATCAACTTCATCGGGCTCTCCATTCTTTACCAAACGTAAATTGATTG
GTAAATATATTATGGATAGTCAACCATATTTTGACAAGAGAACGCAACAGGTACAT
GATAGAAAGTACCCTCAATGGGATCCGATTGCTGTTCTTGGTTGGCGAGGACAAGA
AGGAGGTCCAGAACCAGAGGATGTGAAGCAAAGGGTTGTATGGATGTTCCCTGCTT
CAGTGAATCTACAAGAATTGCGGGTTTACCAACCTCTGATCGAAACAGCGCAACGTT
TCAACTTAGTTCCTGCTTGGGTTAGTATGGATAGTGTGGATGAGCACATCACACGTA
TGTTTGACACAAAAGGCGCAGATGATGTCGTTGTATGTACTGATTTCTCCAAATTTG
ACCAACATTTCAACTCTGATATGGCTCGCGGTGCGTCAGAGATATTAGATGGCTTGT
TTAACGGAAGTCGAGACTTCGTACAATGGATGTGGGATACATACCACATAAAATAC
ACGATACCTCTATTAGACTCAGAAAACCATGCGTGGTTTGGACGACATGGAATGGGT
TCCGGTTCAGGCGGAACAAATGCTGATGAAACATTAGCACATCGCGCGTTGCAGTAT
GAAGCAGCGCTTTCTCAAAACCAAACACTAAACCCTTATTCACAATGCCTAGGTGAT
GATGGAGTACTTACATACCCAGGTATTAAAGTGGATGATGTAATGCGATCATATACT
GCTCATGGTCAAGAAATGAATGAGTCGAAGCAGTACGTGAGCAAACATGAATGCAT
ATACTTGAGAAGGTGGCATCACAAAGATTATCGTGTGTCAGGTATATGTGTCGGAGT
TTATGCAACTACTCGTGCTTTAGGACGGTTGTGTGAACAAGAAAGATACTTTGACCC
AGAAGTATGGTCAAAGGAAATGGTAGCCTTACGTCAGCTATCGATCCTTGAGAATAT
AAAATACCACCCACTCAAGGAAGAATTCGTGAATTATTGCATGAAAGGGGACAAGT
ATAGACTGGGACTAGACTTGCCAGGCTTCTTAGAGAACATAGATGGACTCGCAAAG
CAAGCTACTGATCTTATGCCGGACTTCTTGGGATACGTTAAGTCCCAACAGAAAGAT
ACAGGAATGAGCGATTGGTGGATCGTTAAGTATCTTAAAAGTCTAAAGTAAAGATTT
GGATGGTGCAGCAAACCATTAGAATTCATATTGAATTCTAACTGCACCATCCAAATC
TTTACTTTAGACTTTTAAGATACTTAACGATCCACCAATCGCTCATTCCTGTATCTTT
CTGTTGGGACTTAACGTATCCCAAGAAGTCCGGCATAAGATCAGTAGCTTGCTTTGC
GAGTCCATCTATGTTCTCTAAGAAGCCTGGCAAGTCTAGTCCCAGTCTATACTTGTCC
CCTTTCATGCAATAATTCACGAATTCTTCCTTGAGTGGGTGGTATTTCATA
>14-PBV-19-015_RDRP
(SEQ ID NO: 50)
CTAAATGAATAGAAAAGTAGTCAAGTTAGGTAATTACTTTAAATTACCAAATCCCGG
TTGAAGACCTATCTATTGAAAACTCAGAGAGGTAACGATGAAGAGTATCGTACTCC
ATTTTTCAAAGATAAATCTTTGTCCGAAGTATTAAATGGCTGGTTAGTGCAACTAGC
GCCTCTCAAAGAGAAGTGGCCTGGTTTACACCAGTTTGAATTAGACCTAGCGGAAA
AGGTCGGGCCTCTAAGCATCCAGAAACCTTTGGAAGAAAGGTTTAAGGATATAGAA
GCTTATTACAAAGGTATTCTCCTACCTTCCAAACCAATTAGTGAGGCGGCAATCCGA
TCCGTCTTAACTGAATGGAATAGGGCACGTGGCTTGTCAGTACGCAGTGTCTCCAAG
ACATGGGATAACATGAAGAAATCGACTTCTTCTGGATCTCCATTCTTTACTAAACGT
AAAGCAGTGGGAAAATATACTATGTATATGGAACCATGTTTTGACAAAAGAACGCA
AGAAGTTCATTTTAAGAACTCAAACCGTTGGGATCCAATTGCGGTCTTAGGTTGGCG
TGGACAAGAAGGTGGACCTGATTTTGAGGATGTGAAGCAAAGGGTAGTATGGATGT
TCCCTGCTTCGGTAAACCTACAAGAGTTACGTGTTTACCAACCTCTAATCGAAACAG
CGCAACGTTTCAACTTAGTTCCTGCTTGGGTTGGCATGGATAGTGTTGATTTGCACAT
CACACGTATGTTTGATACGAAAGGCGCAGATGATGTCGTAATCTGTACAGATTTCTC
GAAATTTGACCAACATTTTAACGCTGATATGGCGCGCGGTGCATCCGAGATATTGGA
TGGCCTCTTTAACGGGCGCAGAGATTTTGTACAATGGATGTGGGATATATATCACAT
CAAATACACGATACCTCTACTCGACTCAGAAGATCATGCCTGGTTTGGCAGACATGG
GATGGGTTCCGGATCTGGTGGAACCAACGCTGATGAAACATTAGCACACAGAGCTT
TGCAGTATGAAGCTGCTTTATCACAGAACCAAACATTAAACCCTTATTCACAATGTC
TAGGTGATGATGGAGTACTAACTTACCCTGGTATTAAGGTGGAGGATGTAATACGA
ACATATACTGCACATGGTCAAGAGATGAATCCCGATAAGCAGTATGTGAGTAAACA
GGAATGCATATATCTGAGAAGATGGCATCACATTGATTATCGTGTTAATGATATATG
TGTCGGAGTTTACGCAACTACTCGAGCTTTAGGTCGTTTGTGTGAACAAGAAAGGTA
TTTTGATCCAGAGATATGGTCAAAAGAAATGGTAGCTCTTCGTCAGCTATCAATACT
TGAGAATGTGAAATACCACCCTCTCAAGGAAGAATTTGTTAAGTATTGCATGAAAG
GGGATAAGTACAGACTGGGACTGGACTTACCAGGCTTTCTCGAGAACATAGATGGA
CTCGCAAAGAAAGCTACCGATCTAATGCCGGACTTTTTAGGTTACGTTAAATCACAA
CAGAAATCTGTCGGTGGTATATCAGATTGGTGGATAGTAAAATATCTACGTAGTCTA
AAGTAATGATTGGGATGGTGCAGTAAACCATTAGAATTCTAACGAATTCTAACTGCA
CCATCCCAATCATTACTTTAGACTACGTAGATATTTTACTATCCACCAATCTGATATA
CCACCGACAGATTTCTGCTGTGATTTAACGTAACCTAAAAAGTCCGGCATAAGATCG
GTAGCTTTCTTTGCGAGTCCATCTATGTTCTCGAGAAAGCCTGGTAAGTCCAGTCCC
AGTCTGTACTTATCCCCTTTCATGCAATACTTAACAAATTCTTCCTTGAGAGGGTGGT
ATTTCAC
>15-PBV-19-016_RDRP_
(SEQ ID NO: 51)
AAAGGAGACGACTTAATGAATAGAAAAGTAGTCAGTTTAGGTAATTACTTTAAATT
ACCAAATCCCGGTTGAAGACCTATCTATTGAAAACTAAGAGAGGTAACGATGAAGA
GTATCGTACTCCATTTTTCAAAGGTAAATCTTTATCCGATGTATTAAAAGGCTGGGA
AGTGCACCTTGCCCCTCTCAAAGAGAAGTGGCCTGGTTTACACCAGTTTGAATTAGA
CCTAGCGGAAAAGGTCGGGCCGATGAGTATTCAGAAACCTCTTGATGAGCGTTTCA
AGGATATTGAAGCCTATTACAAAGGTATTCTCCTACCTTCCACTCCAATAAGTGATG
CAGCAATCCGATCTGTAATCACTGAATGGAATAGGGCTCGCGGATTGTCAGTTCGCA
GTACTTCCAAAACATGGGATAAGATGAAGAAATCAACTTCATCAGGCTCTCCATTCT
TTACCAAACGTAAATTGATTGGTAAGTACATTATGGATAGTCAACCATATTTTGACA
AAAGAACGCAAGAGGTACATGATAAACAGTACCCACAATGGGATCCAATTGCGGTT
CTTGGTTGGCGAGGACAAGAAGGTGGTCCAGAACCAGAGGATGTGAAGCAAAGGGT
TGTATGGATGTTCCCTGCTTCAGTTAACCTGCAAGAATTGCGGGTATACCAACCTCT
GATCGAAACAGCGCAACGTTTCAACTTAGTTCCTGCTTGGGTTAGCATGGATAGTGT
GGATGAGCACATCACACGTATGTTTGATACTAAAGGCGCAGATGATGTCGTGATTTG
TACTGATTTCTCTAAATTTGACCAACATTTCAATTCTGATATGGCTCGAGGCGCATCA
GAGATATTAGATGGCCTATTTAACGGGAGTCGAGATTTCGTACAATGGATGTGGGAT
ACATACCACATAAAGTACACGATACCTCTACTAGACTCAGAAAACCATGCGTGGTTT
GGACGTCATGGAATGGGTTCCGGCTCAGGTGGAACCAATGCTGATGAAACATTAGC
ACATCGCGCGTTGCAATATGAAGCAGCGCTTTCTCAAAACCAAACACTTAACCCTTA
TTCACAATGCCTAGGTGATGATGGAGTACTTACGTACCCAGGTATTAAAGTGGATGA
TGTAATGCGGTCATATACTGCTCATGGTCAAGAGATGAATGAGTCAAAGCAGTACGT
GAGCAAACATGAATGCATATATCTGAGAAGATGGCATCACAAAGATTATCGTGTGT
CAGATGTATGTGTCGGAGTTTATGCAACAACCCGTGCTTTGGGTCGGTTGTGTGAAC
AAGAACGATACTTTGATCCAGAAGTATGGTCAAAGGAAATGGTAGCTCTGCGTCAG
CTATCGATCCTTGAGAATATCAAATACCACCCACTCAAGGAAGAATTCGTGAATTAT
TGCATGAAAGGAGACAAGTATAGACTGGGACTAGACTTGCCAGGCTTCTTGGAGAA
TATTGATGGACTCGCAAAGCAAGCTACTGATCTTATGCCGGACTTCTTGGGATACGT
TAAGTCCCAACAAAAGGATACAGGAATGAGCGATTGGTGGATCGTCAAGTATCTTA
AAAGTCTAAAGTAAAGATTTGGATGGTGCAGNNNNNNNNNNNNTCGTCGGCAGCGT
CAGATGTGTATAAGAGACAGCTAACTGCACCATCCAAATCTTTACTTTAGACTTTTA
AGATACTTGACGATCCACCAATCGCTCATTCCTGTATCCTTTTGTTGGGACTTAACGT
ATCCCAAGAAGTCCGGCATAAGATCAGTAGCTTGCTTTGCGAGTCCATCAATATTCT
CCAAGAAGCCTGGCAAGTCTAGTCCCAGTCTATACTTGTCTCCTTTCATGCAATAATT
CACGAATTCTTCCTTGAGTGGGTGGTATTTGATC
>23-PBV-19-035_RDRP
(SEQ ID NO: 52)
TAGGTAATTACTTTAAATTACCAAATCCCGGTTGAAGACCTATCTATTGAAAACTAA
GAGAGGTAACGATGAAGAGTATCGTACTCCATTTTTCAAAGGTAAATCTTTGTCCGA
TGTATTAAAAGGCTGGGAAGTGCACCTCGCCCCTCTCAAAGAGAAGTGGCCTGGTTT
ACACCAGTTTGAATTAGACCTAGCGGCAAAGGTCGGGCCTATGAGTATTCAGAAAC
CGCTTGATGAGCGATTTAAGGATATTGAGGCTTATTACAAAGGTATTCTCCTACCTT
CCGAACCAATTAGTGATGAAGCAATCCGATCTGTCATCACTGAATGGAACAGGGCT
CGCGGATTGTCAGTTCGCAGTACTTCCAAAACATGGGATAACATGAAGAAGTCAAC
TTCTTCAGGCTCTCCATTCTTTACCAAACGTAAATTGATTGGTAAGTATATAATGGAT
AGTCAACCATATTTTGACAAAAGAACACAAAAGGTACACGATAGAAAGTACCCACA
ATGGGATCCAATTGCTGTTCTTGGTTGGCGTGGACAAGAAGGAGGTCCAGAACCAG
AGGATGTGAAGCAAAGGGTTGTATGGATGTTCCCTGCTTCAGTTAACCTGCAAGAGT
TGCGGGTGTACCAACCTCTGATCGAAACAGCGCAACGTTTCAACTTAGTTCCTGCTT
GGGTTAGCATGGATAGTGTGGACGAGCACATCACACGTATGTTTGATACAAAAGGC
GCAGATGATGTCGTGATTTGTACTGATTTCTCTAAATTTGACCAACACTTTAATTCTG
ATATGGCTCGCGGTGCATCTGAGATATTAGATGGACTATTTAACGGCAGCCGAGATT
TCGTACAATGGATGTGGGATACATACCACATTAAATACACGATACCTCTATTAGACT
CTGAGAACCATGCGTGGTTTGGACGTCATGGTATGGGTTCCGGTTCAGGCGGAACTA
ATGCTGATGAGACATTAGCTCATCGTGCGCTTCAGTATGAAGCAGCACTCTCACAAA
AACAAACACTAAACCCTTATTCACAATGCTTGGGAGATGATGGAGTACTAACGTACC
CAGGTATTAAAGTGGATGATGTAATGCGATCATATACTGCACATGGTCAAGAGATG
AATGAGTCGAAGCAGTACGTGAGCAAACATGAATGCATATATCTGAGAAGATGGCA
TCACAAGGATTATCGTGTGTCAGGTATATGTGTCGGAGTTTATGCAACTACTCGTGC
TTTGGGTAGATTGTGTGAACAAGAAAGGTACTTTGACCCAGAAGTATGGTCAAAGG
AAATGGTAGCTTTACGTCAGCTATCAATCCTTGAGAATATTAAATACCACCCACTCA
AGGAAGAATTCGTGAATTATTGCATGAAAGGCGACAAGTATAGACTGGGACTAGAC
TTGCCAGGCTTCTTGGAGAACATTGATGGACTCGCAAAGCAAGCTACTGATCTTATG
CCAGACTTTTTGGGATACGTTAAATCTCAACAGAAAGATACAGGAATGAGCGATTG
GTGGATCGTGAAGTATCTTAAGAGNNNNNNNNNNNNNNNNNNNNNNNNNTGTGTA
TAAGAGACAGTAACTGCACCATCCAAATCTTTACTTTAGACTCTTAAGATACTTCAC
GATCCACCAATCGCTCATTCCTGTATCTTTCTGTTGAGATTTAACGTATCCCAAAAAG
TCTGGCATAAGATCAGTAGCTTGCTTTGCGAGTCCATCAATGTTCTCCAAGAAGCCT
GGCAAGTCTAGTCCCAGTCCATGCTNNNNN
>25-PBV-19-038 RDRP
(SEQ ID NO: 53)
TACTTATGAATAGAAAAGTAGTCAGTTTAGGTAATTACTTTAAATTACCAAATCCCG
GATTGAAGACCTATCTATTGAAAACTAAGAGAGGTAACGATGAAGAGTATCGTACT
CCATTTTTCAAAGGTAAATTTTATCCGATGTTTTAAAAGGCTGGGAAGTGCACCTCG
CCCCTCTCAAAGAGAAGTGGCCTGGTTTACACCAGTTTGAATTAGACCTAGCGGAAA
AGGTCGGGCCTATGAGTATTCAGAAACCTCTTGAAGAGCGTTTCACGGATATTGAGG
CTTATTACAAAGGTATTCTCCTACCTTCCGAACCAATTAGTGATGAAGCAATCCGAT
CTGTCATCACTGAGTGGAACAGGGCTCGCGGATTGTCAGTTCGCAGTACTTCCAAAA
CATGGGACAATATGAAGAAGTCTACTTCTTCAGGCTCTCCATTCTTTACTAAACGTA
AGTTAATTGGTAAATATATAATGGATAGTCAACCATATTTTGACAAAAGAACGCAA
GAGGTACATGATAAAATGTATCCACATTGGGATCCAATTGCCGTTCTTGGTTGGCGT
GGACAAGAAGGAGGTCCAGAACCAGAGGATGTGAAGCAAAGGGTTGTATGGATGTT
CCCTGCTTCAGTTAACTTGCAAGAATTACGAGTATACCAACCTCTGATCGAAACAGC
GCAACGTTTCAACTTAGTTCCTGCTTGGGTTAGCATGGATAGTGTGGACGAGCACAT
CACACGTATGTTTGATACTAAAGGCGCAGATGATGTCGTGATTTGTACTGATTTCTCT
AAATTTGACCAACACTTTAATGCTGATATGGCTCGCGGCGCATCCGAAATATTGGAT
GGCATATTTAACGGGGGCCGAGACTTCATACAATGGATGTGGGACATATATCACATC
AAATACACGATACCTCTATTAGACTCTGAGAACCATGCGTGGTTTGGACGTCATGGT
ATGGGTTCCGGTTCAGGCGGAACTAATGCTGATGAGACTTTAGCTCATCGTGCGTTG
CAATATGAGGCAGCGCTCTCACAAAACCAAACACTAAACCCTTATTCACAATGCTTG
GGTGATGATGGAGTACTAACATATCCAGGCATCAAAGTGGATGATGTAATGCGATC
ATATACTGCTCATGGTCAAGAAATGAATGAGTCGAAGCAGTACGTGAGCAAACATG
AATGCATATATCTGAGAAGATGGCATCACAAAGATTATCGTGTTGCAGATGTATGTG
TCGGAGTTTATGCAACTACCAGAGCTTTGGGTAGGTTGTGTGAACAAGAAAGATATT
TTGACCCAGAAGTATGGTCAAAAGAAATGGTAGCTTTACGTCAGCTATCGATCCTTG
AGAATGTCAAATACCACCCACTTAAGGAAGAATTCGTGAATTATTGCATGAAAGGC
GACAAGTATAGACTGGGACTAGACTTGCCAGGCTTCTTGGAGAACATTGATGGACTC
GCAAAGCAAGCTACTGATCTGATGCCGGACTTTTTGGGATACGTTAAGTCCCAACAG
AAAGATACAGGAATGAGCGATTGGTGGATCGTCAAGTATCTTAAGAGTCTAAAGTA
AAGATTTGGATGGTGCAGTAAACCATTAGAATTCATTTGAATTCTAACTGCTGCACC
ATCCAAATCTTTACTTTAGACTCTTAAGATACTTGACGATCCACCAATCGCTCATTCC
TGTATCTTTCTGTTGAGACTTAACGTATCCCAAAAAGTCCGGCATCAGATCAGTAGC
TTCCTTTGCGAGTCCATCAATGTTCTCCAAGAAGCCTGGCAAGTCTAGTNNNNNNNN
NNNNNNNNNNNNNNNNNNNNNNNNNNNNNNNNNNNNNNNNNNNNNNNNNNNNNN
NNNNNNN
>27-PBV-19-044_RDRP
(SEQ ID NO: 54)
TCATAAGTAGCCTCCTTTTCTAGTAAACATTTTCGTTAGAATTTATTTACTAGAAAAG
GAGGCTACTTATGAATAGAAAAGTAGTCAGTTTAGGTAATTACTTTAAATTACCAAA
TCCCGGATTGAAGACCTATCTATTGAAAACTAAGAGAGGTAACGATGAAGAGTATC
GTACTCCATTTTTCAAAGGTAAATCTTTATCCGATGTTTTAAAAGGCTGGGAAGTGC
ACCTCGCCCCTCTCAAAGAGAAGTGGCCTGGTTTACACCAGTTTGAATTAGACCTAG
CGGAAAAGGTCGGGCCTATGAGTATTCAGAAACCTCTTGAAGAGCGTTTCACGGAT
ATTGAGGCTTATTACAAAGGTATTCTCCTACCTTCCGAACCAATTAGTGATGAAGCA
ATCCGATCTGTCATCACTGAGTGGAACAGGGCTCGCGGATTGTCAGTTCGCAGTACT
TCCAAAACATGGGACAATATGAAGAAGTCTACTTCTTCAGGCTCTCCATTCTTTACT
AAACGTAAGTTAATTGGTAAATATATAATGGATAGTCAACCATATTTTGACAAAAGA
ACGCAAGAGGTACATGATAAAATGTATCCACATTGGGATCCAATTGCCGTTCTTGGT
TGGCGTGGACAAGAAGGAGGTCCAGAACCAGAGGATGTGAAGCAAAGGGTTGTAT
GGATGTTCCCTGCTTCAGTTAACTTGCAAGAATTACGAGTATACCAACCTCTGATCG
AAACAGCGCAACGTTTCAACTTAGTTCCTGCTTGGGTTAGCATGGATAGTGTGGACG
AGCACATCACACGTATGTTTGATACTAAAGGCGCAGATGATGTCGTGATTTGTACTG
ATTTCTCTAAATTTGACCAACACTTTAATGCTGATATGGCTCGCGGCGCATCCGAAA
TATTGGATGGCATATTTAACGGGGGCCGAGACTTCATACAATGGATGTGGGACATAT
ATCACATCAAATACACGATACCTCTATTAGACTCTGAGAACCATGCGTGGTTTGGAC
GTCATGGTATGGGTTCCGGTTCAGGCGGAACTAATGCTGATGAGACTTTAGCTCATC
GTGCGTTGCAATATGAGGCAGCGCTCTCACAAAACCAAACACTAAACCCTTATTCAC
AATGCTTGGGTGATGATGGAGTACTAACATATCCAGGCATCAAAGTGGATGATGTA
ATGCGATCATATACTGCTCATGGTCAAGAAATGAATGAGTCGAAGCAGTACGTGAG
CAAACATGAATGCATATATCTGAGAAGATGGCATCACAAAGATTATCGTGTTGCAG
ATGTATGTGTCGGAGTTTATGCAACTACCAGAGCTTTGGGTAGGTTGTGTGAACAAG
AAAGATATTTTGACCCAGAAGTATGGTCAAAAGAAATGGTAGCTTTACGTCAGCTAT
CGATCCTTGAGAATGTCAAATACCACCCACTTAAGGAAGAATTCGTGAATTATTGCA
TGAAAGGCGACAAGTATAGACTGGGACTAGACTTGCCAGGCTTCTTGGAGAACATT
GATGGACTCGCAAAGCAAGCTACTGATCTGATGCCGGACTTTTTGGGATACGTTAAG
TCCCAACAGAAAGATACAGGAATGAGCGATTGGTGGATCGTCAAGTATCTTAAGAG
TCTAAAGTAAAGATTTGGATGGTGCAGCAAACCATTAGAATTCATTTGAATTCTAAC
TGCACCATCCAAATCTTTACTTTAGACTCTTAAGATACTTGACGATCCACCAATCGCT
CATTCCTGTATCTTTCTGTTGGGACTTAACGTATCCCAAAAAGTCCGGCATCAGATC
AGTAGCTTGCTTTGCGAGTCCATCAATGTTCTCCAAGAAGCCTGGCAAGTCTAGTCC
CAGTCTATACTTGTCGCCTTTCATGCAATAATTCACGAATTCTTCCTTAAGTGGGTGG
TATTTGACC
>28-PBV-19-046_RDRP
(SEQ ID NO: 55)
TGATACGGCGACCACCGAGATCTACACTTGCGAAGTCGTCGGCAGCGTCAGATGTGT
ATAAGAGACAGCTTATGAATAGAAAAGTAGTCAGTTTAGGTAATTACTTTAAATTAC
CAAATCCCGGTTGAAGACCTATCTATTGAAAACTAAGAGAGGTAACGATGAAGAGT
ATCGTACTCCATTTTTCAAAGGTAAATCTTTATCCGATGTTTTAAAAGGCTGGGAAG
TGCACCTCGCCCCTCTCAAAGAGAAGTGGCCTGGTTTACACCAGTTTGAATTAGACC
TAGCGGAAAAGGTCGGGCCTATGAGTATTCAGAAACCTCTTGAAGAGCGTTTCACG
GATATTGAGGCTTATTACAAAGGTATTCTCCTACCTTCCGAACCAATTAGTGATGAA
GCAATCCGATCTGTCATCACTGAGTGGAACAGGGCTCGCGGATTGTCAGTTCGCAGT
ACTTCCAAAACATGGGACAATATGAAGAAGTCTACTTCTTCAGGCTCTCCATTCTTT
ACTAAACGTAAGTTAATTGGTAAATATATAATGGATAGTCAACCATATTTTGACAAA
AGAACGCAAGAGGTACATGATAAAATGTATCCACATTGGGATCCAATTGCCGTTCTT
GGTTGGCGTGGACAAGAAGGAGGTCCAGAACCAGAGGATGTGAAGCAAAGGGTTG
TATGGATGTTCCCTGCTTCAGTTAACTTGCAAGAATTACGAGTATACCAACCTCTGA
TCGAAACAGCGCAACGTTTCAACTTAGTTCCTGCTTGGGTTAGCATGGATAGTGTGG
ACGAGCACATCACACGTATGTTTGATACTAAAGGCGCAGATGATGTCGTGATTTGTA
CTGATTTCTCTAAATTTGACCAACACTTTAATGCTGATATGGCTCGCGGCGCATCCG
AAATATTGGATGGCATATTTAACGGGGGCCGAGACTTCATACAATGGATGTGGGAC
ATATATCACATCAAATACACGATACCTCTATTAGACTCTGAGAACCATGCGTGGTTT
GGACGTCATGGTATGGGTTCCGGTTCAGGCGGAACTAATGCTGATGAGACTTTAGCT
CATCGTGCGTTGCAATATGAGGCAGCGCTCTCACAAAACCAAACACTAAACCCTTAT
TCACAATGCTTGGGTGATGATGGAGTACTAACATATCCAGGCATCAAAGTGGATGAT
GTAATGCGATCATATACTGCTCATGGTCAAGAAATGAATGAGTCGAAGCAGTACGT
GAGCAAACATGAATGCATATATCTGAGAAGATGGCATCACAAAGATTATCGTGTTG
CAGATGTATGTGTCGGAGTTTATGCAACTACCAGAGCTTTGGGTAGGTTGTGTGAAC
AAGAAAGATATTTTGACCCAGAAGTATGGTCAAAAGAAATGGTAGCTTTACGTCAG
CTATCGATCCTTGAGAATGTCAAATACCACCCACTTAAGGAAGAATTCGTGAATTAT
TGCATGAAAGGCGACAAGTATAGACTGGGACTAGACTTGCCAGGCTTCTTGGAGAA
CATTGATGGACTCGCAAAGCAAGCTACTGATCTGATGCCGGACTTTTTGGGATACGT
TAAGTCCCAACAGAAAGATACAGGAATGAGCGATTGGTGGATCGTCAAGTATCTTA
AGAGTCTAAAGTAAAGATTTGGATGGTGCAGCAAACCATTAGAATTCATTTGAATTC
TAACTGCACCATCCAAATCTTTACTTTAGACTCTTAAGATACTTGACGATCCACCAAT
CGCTCATTCCTGTATCTTTCTGTTGGGACTTAACGTATCCCAAAAAGTCCGGCATCAG
ATCAGTAGCTTGCTTTGCGAGTCCATCAATGTTCTCCAAGAAGCCTGGCAAGTCTAG
TCCCAGTCTATACTTGTCGCCTTTCATGCAATAATTCACGAATTCTTCCTTAAGTGGG
TGGTATTTGACA
>12PBVKM-19-012_RDRP
(SEQ ID NO: 56)
ACTCGTTAACACTAGTTGTAGAGCGCGTACTCCCGCGGTCCGACCAGACCGCTCGCG
ACTTACAGAAAGGAGGTCGATCGTATGCCTAAATACGATAACATCATGGCGGATTA
TTTCGATCTGCCCAATCCAGCGTTGGGGTCATATTTCGGTAGAACCCGACATGGCAA
TCCTGATGTATACAGGACCACATTCTTCAAGAATCGTGAGCCTCAGGATGTTTTGTC
AGAATGGATGAAGTCAGTCCAGGTTCTTAAACAGGATTGGCCTACGCTGTTAACATT
TGAGGAAGACCTTGCTTCCAAAGTAGGTCCACTGTCAGTGCAGAAGCCTTTAGTGGA
TAGGCTCCCTGATATTCAGGCTTACTATGACTGCATTAACCTGGAGTCAAAACCCCT
TGAGAAAGAAGCAGTTCAAGCTTTCTTGAAGGAGTTAAAAGGTTTAAACACCTTATC
GATGCGCGGAATTCCCGCTACGATAGAAAACATGAAGTTGTCCACTTCCAGCGGTTG
TCCATATTTCACCAAACGTAAGAACGATGTTCGCCGTCACAGATACGGGGACGTAA
AGTATGACGGAAATCGTATCACTGCAAACATAGGTGGCAAGGAATTTAAGATGGCC
GCTATTCTTGGATGGAGAGGCCAGGAAGGAGGACCAAATAATTCGGACGTTAAACA
GAGAGTGGTATGGATGTTCCCTTTCACTGTTAACCTCCAAGAACTACGTGTCTACCA
GCCGTTTATGGATATGTTACAAAAGCACAAGATTATACCAGCATGGGTCGGGCTGG
ATGAGGTAGACAATAAGATCACCAAATTGTTTGACACCAAAGGTGAAGATGACGTA
GTTATATGTACCGACTTTTCTAAGTTTGACCAGCACTTTAATGAAGATTGCCAGAAA
GTAGCCCATGATATCTTAGCTTGGTTGTTTATTGGTGATAGCCGTATGGAAGGCTGG
TTGCGTAATGTATTTCCTGTCAAATACAATATTCCTATAATCTGTGATGACAATATTG
TGAAGAATGGTCGTCATGGTATGGGTTCCGGGTCGGGAGGAACAAATCAAGATGAA
ACGCTACTACACAGAGTATTACAACATGAAGCGGCTCTTAGTGTAGGACAGGACCT
CAATCTTAATTCACAATGCTTAGGTGATGATGGTATACTAACTTACCCAGGTATTAA
GGTAGAGGATGTAATACGAACATATACTGCACATGGTCAAGAAATGAATCCCGATA
AGCAGTATGTGAGTAAACAGGACTGCGTATATCTTCGTAGATGGCACCATAAAGAC
TATCGCGAAAACGGCGTATGCGTAGGGGTATATAGTACTGCTCGCGCTTTAGGGCGT
ATGATGTACCAAGAACGCTACTACGACCCTGATGAATGGGGTAAAGAGATGGTTGC
GCTAAGACAACTGTCTATATTAGAGAACTGCAAACACCACCCTCTCAAAGAAAAGT
TTGTGGACTATTGCATTAAAGGGGATAAATATAGGCTTGGTATAGATATCCCAGGTT
TTCTAGACAATCTGGAAACGTTGTCAGAGAAAGCTATCGAAGTAATGCCTGACTTTA
TGGGCTACACACAATCTCTTGGACATAAAGATGAAAGAGTATCCAAAGGTATTAAT
GATTGGTGGATCGTTAAATACTTAAAGTCA
>14PBVKM-19-015_RDRP
(SEQ ID NO: 57)
ACTCGTTAACACTAGTTGTAGAGCGCGTACTCCCGTGGTCCGACCAGACCACACGCG
ACTTACAGAAAGGAGGTCGATCGTATGCCTAAATACGATAACATCATGGCAGACTA
TTTTGATCTGCCCAATCCAGCGTTGGGGTCATATTTCGGTAGAACCCGACATGGCAA
TCCTGATGTATACAGGACCACATTCTTCAAGAATCGTGAGCCTCAGGATGTTCTGTC
AGAATGGATGAAGTCGATCCAGGTTCTTAAACAGGATTGGCCTACGCTGTTAACTTT
TGAGGAAGACCTTGCTTCCAAAGTAGGACCACTGTCCGTTCAGAAGCCTTTAGTGGA
TAGGCTCCCTGACGTTCAAGCCTACTATGACTGCATTAACCTGGAGTCAAAACCCCT
TGCGAAAGAAGCAGTTCAAGCTTTCATCAAGGAGTTAAAAGGTTTAAATACCTTATC
GATGCGTGGAATTCCCGCTACGATAGAAAATATGAAGTTGTCCACTTCCAGTGGCTG
TCCTTATTTCACCAAGCGTAAAAGCGATGTACGCCGTCATAGATACGGGGACGTAAA
ATCTGATGGTAATCGTATAACCGCTGAGATCGGTGGCAAGGAATTTAAGATGGCCG
CTATTCTTGGATGGAGAGGCCAGGAAGGAGGACCAAAGAATTCGGACGTTAAACAG
AGAGTGGTATGGATGTTCCCTTTCACTGTTAACCTCCAAGAACTACGTGTCTACCAG
CCGTTTATGGATATGCTCCAGAAGCATAAAATTGTACCAGCATGGGTCGGACTGGAT
GAGGTAGACAATAAGATCACTAAATTGTTTGACACCAAAGGTGAAGATGACGTAGT
TATATGTACCGACTTTTCTAAGTTTGACCAGCACTTTAATGAAGATTGCCAAAAGGT
AGCCCATGATATCTTAGCTTGGTTGTTTATTGGCGATAGCCGTATGGAAAGCTGGTT
GCGTAATGTATTTCCTGTCAAATACAATATTCCTATAATCTGTGACGACAATATTGTG
AAGAATGGACGTCACGGTATGGGTTCCGGTTCGGGAGGAACAAATCAAGATGAAAC
GCTACTACACAGGGTATTACAACATGAAGCGGCCCTTAGTGTAGGACAGGACCTAA
ACCTTAATTCACAATGCCTTGGTGATGATGGTATACTAACTTACCCTGGTATTAAGG
TGGAGGATGTAATACGAACATATACTGCACATGGTCAAGAGATGAATCCCGATAAG
CAGTATGTGAGTAAACAGGACTGCGTATATCTTCGTAGATGGCACCATAAAGACTAT
CGCGAAAACGGCGTATGCGTAGGGGTATATAGTACTGCCCGCGCTTTAGGGCGTAT
GATGTATCAAGAACGCTACTATGACCCCGATGAATGGGGTAAAGAGATGGTTGCGC
TAAGACAACTGTCTATATTAGAGAACTGCAAACACCACCCTCTCAAAGAAAAGTTTG
TGGACTATTGCATTAAAGGGGATAAATATAGGCTTGGTATAGATATCCCAGGTTTTC
TAGACAATCTGGAAACGTTGTCTGAGAAAGCTATCGAAGTAATGCCTGACTTTATGG
GCTACACACAGTCACTTGGACATAAAGATGAAAAGGTATCCAAAGGTATTAATGAT
TGGTGGATCGTTAAGTACTTAAAGTCA
>18PBVKM-19-023_RDRP
(SEQ ID NO: 58)
ACTCGTTAACACTAGTTGTAGAGCGCGTACTCCCGTGGTCAGACCAGACCACACGCG
ACTTACAGAAAGGAGGTCGATCGTATGCCTAAATACGATAACATCATGGCAGATTA
TTTTGATCTGCCCAATCCAGCGTTGGGGTCATATTTCGGTAGAACCCGACATGGCAA
TCCTGATGTATACAGGACCACATTCTTTAAGGATCGTGAGCCTCAGGATGTTCTGTC
AGAATGGATGAAGTCAGTCCAGGTTCTTAAACAGGATTGGCCTACGCTGTTAACTTT
TGAGGAAGACCTTGCTTCCAAAGTAGGACCATTGTCCGTTCAGAAGCCTTTAGTGGA
TAGGCTCCCTGACGTACAGGCTTACTATGACTGCATTAACCTGGAGTCAAAACCTCT
TCAGAAAGAAGCAGTTCAAGCTTTCTTGAAGGAGTTGAAAGGTTTAAACACCTTATC
GATGCGTGGTATTCCCGCAACGATAGAAAACATGAAGTTGTCCACTTCTAGTGGTTG
TCCATTCTTCACCAGACGTAAGAATGATGTTCGTCGTCATCGCTACGGGGACGTAAG
CTTTGATGGAACTACCATTCATGCTGAAATAGGTGGCAAGGATTACAAGATGGCAG
CCATATTAGGTTGGAGGGGCCAAGAAGGAGGACCAAAGAATTCGGATGTTAAACAG
AGGGTGGTATGGATGTTCCCATTCACTGTTAACCTCCAAGAACTACGTGTCTATCAG
CCGTTTATGGATATGCTACAAAAACACAAAGTAGTACCAGCTTGGGTCGGTCTGGAT
GAGGTAGACAATAAGATTACCAAATTGTTTGACACCAAAGGTAAAGATGACGTAGT
TATTTGTACCGACTTTTCAAAGTTTGACCAGCACTTTAATGAAGATTGCCAAAAGGT
AGCCCATGATGTCTTAGCTTGGTTATTTATTGGTGATAGCCGTATGGAAAGCTGGTT
GCGTAATGTATTTCCTGTCAAATACAATATTCCTATAATCTGTGATGATAATATTGTG
AAGAATGGTCGTCATGGTATGGGTTCCGGTTCGGGAGGAACAAATCAAGATGAAAC
GCTGCTACACAGGGTATTACAACATGAAGCGGCCCTTAGTGTAGGACAGGACCTCA
ACCTTAATTCACAATGCTTGGGTGATGATGGTATACTAACTTATCCAGGTATTAAAG
TTGAGGATGTAATACGAACATATACTGCACATGGTCAAGAAATGAATCCCGATAAG
CAGTATGTGAGTAAACAGGACTGCGTATATCTTCGCAGATGGCACCATAAAGACTAT
CGCGAAAACGGCGTATGCGTAGGGGTATATAGTACAGCTCGCGCTTTAGGGCGTAT
GATGTACCAAGAACGCTACTATGACCCTGATGAATGGGGTAAAGAGATGGTTGCGC
TAAGACAACTGTCTATATTAGAGAACTGCAAACACCACCCTCTTAAGGAAAAGTTTG
TGGACTATTGCATGAAAGGGGATAAATACAGGCTAGGTGTAGATATCCCAGGTTTTC
TGGATAATCTGGAAACGTTATCCGAGAAGGCTATCGAAGTCATGCCCGATTTCATGG
GCTACACACAATCCTTGGGACACAAGGACGAAAAGATATCTAAAGGTATTAATGAC
TGGTGGATCGTTAAATACNNNNNNNNN
>21PBVKM-19-033_RDRP
(SEQ ID NO: 59)
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNATCCAGCGTTGGGGTCATATTTCGGTAGAACCCGACA
TGGCAATCCTGATGTATACAGGACCACATTCATTAAGAATCGTGAGCCTCAGCATGT
TTTGTCAGAATGGATGAAGTCAGTACAGGTTCTTAAACNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNAGCCTATTATGACTGCATTAACCTGGA
GTCAAAACCCCTAGAGAAAGAGGCAGTTCAAGCTTTCTTAAAGGAGTTGAAAGGTT
TAAATACCTNNNNNNNNNNNGGTATTCCCGCTACGATAGAAAACATGAAGTTGTCC
ACTTCCAGTGGCTGTCCTTATTTCACCAAGCGTAAGAACGATGTACGCCGTCACAGA
TACGGGGACGTAAAGTTTGACGGTACACGTGTGACCGCTGATATAGGTGGCAAGGA
ATTTAAGATGGCCGCTATACTTGGATGGAGAGGCCAGGAAGGAGGACCAAAGAATT
CGGACGTTAAACAGAGAGTGGTATGGATGTTCCCTTTCACTGTTAACCTCCAAGAAC
TACGTGTCTACCAGCCGTTTATGGATATGCTACAGAAACATAAAGTAGTACCAGCAT
GGGTCGGACTGGATGAGGTAGACAATAAGATCACCAAATTGTTTGACACCAAAGGT
GAAGATGACGTAGTTATATGTACCGACTTTTCTAAGTTTGACCAGCACTTTAATGAA
GATTGCCAAAAGGTAGCCCATGATATCTTAGCTTGGTTATTTATTGGTGATAGCCGT
ATGNNNNNNNNNNNNNNNNNNNNNNNNNNNNNNNNNNNNNNNNNNNNNNNNNNN
NNGACAGACAATATTGTGAAGTCAGGTCGTCATGGTATGGGTTCCGGTTCGGGAGG
AACAAATCAAGATGAAACGCTACTACACAGGGTATTACAACATGAGGCGGCCCTTA
GTGTAGGACAGGACCTTAATCTTAATTCACAATGCCTAGGTGACGATGGTATACTAA
CTTACCCTGGTATTAAGGTTGAGGATGTAATACGAACATATACTGCACATGGTCAAG
AAATGAATCCCGATAAGCAGTATGTGAGTAAACAGGACTGCGTATATCTTCGTAGAT
GGCACCATAAAGACTATCGCGAAAACGGCGTATGCGTAGGGGTATATAGTACAGCC
CGCGCTTTGGGGCGTATGATGTACCAAGAACGCTACTATGACCCTGATGAATGGGGT
AAAGAGATGGTTGCGCTAAGACAACTGTCTATATTGGAGAACTGCAAACACCATCC
TCTCAAAGAGAAGTTTGTGGACTATTGCATTAAAGGGGATAAATATNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
>22PBVKM-19-034_RDRP
(SEQ ID NO: 60)
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNGTCCGTT
CAGAAACCGTTAGTGGATCGGTTACCAGACGTTCAAGCCTACTATGACTGCATTAAC
CTGGAGTCAAAACCCCTAGAGAAAGAGGCAGTTCAAGCTTTCTTAAAGGAGTTGAA
AGGTTTAAATACCTTATCGATGCGCGGTATTCCCGCTACGATAGAAAACATGAAGTT
GTCCACTTCCAGTGGCTGTCCTTATTTCACCAAGCGTAAGAACGATGTACGCCGTCA
CAGATACGGGGACGTAANNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNTCCAAGAACTACGTGTCTACCAGCCGTTTATGGATATGCTACAGAAACATAAAGT
AGTACCAGCATGGGTCGGACTGGATGAGGTAGACAATAAGATCACCAAATTGTTTG
ACACCAAAGGTGAAGATGACGTAGTTATATGTNNNNNNNNNNNNNNNNNNNNNNN
NNNNNNNNNNNNNNNNNGCCAAAAGGTAGCCCATGATATCTTAGCTTGGTTATTTA
TTGGTGATAGCCGTATGGAAAGCTGGTTGCGTAATGTATTTCCTGTCAAATACAATA
TTCCTATAATCTGTGACGACAATATTGTGAAGTCAGGTCGTCATGGTATGGGTTCCG
GTTCGGGAGGAACAAATCAAGATGAAACGCTACTACACAGGGTATTACAACATGAG
GCGGCCCTTAGTGTAGGACAGGACCTTAATCTTAATTCACAATGCCTAGGTGACGAT
GGTATACTAACTTACCCTGGTATTAAGGTGGAGGATGTAATAAGAACAGATACTGC
ACATGGTCAAGAAATGAATCCCGATAAGCAGTATGTGAGTAAACAGGACTGCGTAT
ATCTTCGTAGATGGCACCATAAAGACTATCGCGAAAACGGCGTATGCGTAGGGGTA
TATAGTACAGCCCGCGCTTTGGGGCGTATGATGTACCAAGAACGCTACTATGACCCT
GATGAATGGGGTAAAGAGATGGTTGCGCTAAGACAACTGTCTATATTGGAGAACTG
CAAACACCATCCCTNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNN
>26PBVKM-19-039_RDRP
(SEQ ID NO: 61)
NNNNNNNNNNNNNNGTTGTAGAGCGCGTACTCCCGCGGCCCGACCAGGTCGCACGC
GACTTACAGAAAGGAGGTCGATCGTATGCCTAAATACGATAACATCATGGCAGATT
ATTTCGATCTGCCCAATCCAGCGTTGGGGTCATATTTCGGTAGAACCCGACATGGCA
ATCCTGATGTATACAGGACCACATTCTTTAAGAATCGTGAGCCTCAGGATGTTTTGT
CAGAATGGATGAAGTCAGTCCAGGTTCTTAAACAGGATTGGCCTACGCTGTTAACTT
TTGAGGAAGACCTTGCTTCCAAAGTAGGACCTCTGTCCGTTCAGAAACCGTTAGTGG
ATCGGTTACCAGACGTTCAAGCCTACTATGACTGCATTAACCTGGAGTCAAAACCCC
TAGAGAAAGAGGCAGTTCAAGCTTTCTTAAAGGAGTTGAAAGGTTTAAATACCTTAT
CGATGCGCGGTATTCCCGCTACGATAGAAAACATGAAGTTGTCCACTTCCAGTGGCT
GTCCTTATTTCACCAAGCGTAAGAACGATGTACGCCGTCACAGATACGGGGACGTA
AAGTTTGACGGTACACGTGTGACCGCTGATATAGGTGGCAAGGAATTTAAGATGGC
CGCTATACTTGGATGGAGAGGCCAGGAAGGAGGACCAAAGAATTCGGACGTTAAAC
AGAGAGTGGTATGGATGTTCCCTTTCACTGTTAACCTCCAAGAACTACGTGTCTACC
AGCCGTTTATGGATATGCTACAGAAACATAAAGTAGTACCAGCATGGGTCGGACTG
GATGAGGTAGACAATAAGATCACCAAATTGTTTGACACCAAAGGTGAAGATGACGT
AGTTATATGTACCGACTTTTCTAAGTTTGACCAGCACTTTAATGAAGATTGCCAAAA
GGTAGCCCATGATATCTTAGCTTGGTTATTTATTGGTGATAGCCGTATGGAAAGCTG
GTTGCGTAATGTATTTCCTGTCAAATACAATATTCCTATAATCTGTGACGACAATATT
GTGAAGTCAGGTCGTCATGGTATGGGTTCCGGTTCGGGAGGAACAAATCAAGATGA
AACGCTACTACACAGGGTATTACAACATGAGGCGGCCCTTAGTGTAGGACAGGACC
TTAATCTTAATTCACAATGCCTAGGTGACGATGGTATACTAACTTACCCTGGTATTA
AGGTTGAGGATGTAATACGAACATATACTGCACATGGTCAAGAAATGAATCCCGAT
AAGCAGTATGTGAGTAAACAGGACTGCGTATATCTTCGTAGATGGCACCATAAAGA
CTATCGCGAAAACGGCGTATGCGTAGGGGTATATAGTACAGCCCGCGCTTTGGGGC
GTATGATGTACCAAGAACGCTACTATGACCCTGATGAATGGGGTAAAGAGATGGTT
GCGCTAAGACAACTGTCTATATTGGAGAACTGCAAACACCATCCTCTCAAAGAGAA
GTTTGTGGACTATTGCATTAAAGGGGATAAATATAGGCTTGGTATAGATATCCCAGG
TTTTCTAGATAATCTGGAAACGTTATCTGAGAAAGCTATCGAAGTAATGCCAGACTT
TATGGGCTACACACAATCACTTGGACACCATGAAGATAAGGTGTCAAAAGGTATTA
ATGATTGGTGGATCGTTAAATACCTGAAGTCN
>27PBVKM-19-044_RDRP
(SEQ ID NO: 62)
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNGTTTAAATACCTTATCGATGCGCGGTATTCCCGCTACGATAGAAAACATG
AAGTTGTCCACTTCCAGTGGCTGTCCTTATTTCACCAAGCGTAAGAACGATGTACGC
CGTCACAGATACGGGGACGTAAAGTTTGACGGTACACGTGTGACCGCTGATATAGG
TGGCAAGGAATTTAAGATGGCCGCTATACTTGGATGGAGAGGCCAGGAAGGAGGAC
CAAAGAATTCGGACGTTAAACAGAGAGTGGTATGGATGTTCCCTTTCACTGTTAACC
TCCAAGAACTACGTGTCTACCAGCCGTTTATGGATATGCTACAGAAACATAAAGTAG
TACCAGCATGGGTCGGACTGGATGAGGTAGACAATAAGATAACCAAATTGTTTGAC
ACCAAAGGTGAAGATGACGTAGTTATATGTACCGACTTTTCTAAGTTTGACCAGCAC
TTTAATGAAGATTGCCAAAAAGTAGCCCATGATATCTTAGCTTGGTTATTTATTGGT
GATAGCCGTATCGAAAGCTGGTTGCGTAATGTATTTCCTGTCAAATACAATATTCCT
ATAATCTGTGACGACAATATTGTGAAGTCAGGTCGTCATGGTATGGGTTCCGGTTCG
GGAGGAACAAATCAAGATGAAACGCTACTACACAGGGTATTACAACATGAGGCGGC
CCTTAGTGTAGGACAGGACCTTAATCTTANTTCACAATGCTTGGGTGATGATGGAGT
ACTAACATATCCAGGCATCAAAGTGGATGATGTAATGCGATCATATACTGCTCATGG
TCAAGAAATGAATGAGTCGAAGCAGTACGTGAGCAAACATGAATGCATATATCTGA
GAAGATGGCATCACAAAGATTATCGCGAAAACGGCGTATGCGTAGGGGTATATAGT
ACAGCCCGCGCTTTGGGGCGTATGATGTACCAAGAACGCTACTATGACCCTGATGAA
TGGGGTAAAGAGATGGTTGCGCTAAGACAACTGTCTATATTGGAGAACTGCAAACA
CCATCCTCTCAAAGAGAAGTTTGTGGACTATTGCATTAAAGGGGATAAATATAGGCT
TGGTATAGATATCCCAGGTTTTCTAGATAATCTGGAAACGTTATCTGAGAAAGCTAT
CGAAGTAATGCCAGACTTTATGGGCTACACACAATCACTTGGACACCATGAAGATA
AGGTGTCAAAAGGTATTAATGNNNNNNNNNNNNNNNNNNNNNNNNNNNNN
>28PBVKM-19-046_RDRP
(SEQ ID NO: 63)
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNGGATATGCTA
CAGAAACATAAAGTAGTACCAGCATGGGTCGGACTGGATGAGGTAGACAATAAGAT
CACCAAATTGTTTGACACCAAAGGTGAAGATGACGTAGTTANNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNCTGCACATGGTCAAGAAATGAATCCCGATAA
GCAGTATGTGAGTAAACAGGACTGCGTATATCTTCGTAGATGGCACCATAAAGACT
ATCGCGAAAACGGCGTATGCGTAGGGGTATATAGTACAGCCCGCGCTTTGGGGCGT
ATGATGTACCAAGAACGCTACTATGACCCTGATGAATGGGGTAAAGAGATGGTTGC
GCTAAGACAACTGTCTATATTGGAGAACTGCAAACACCATCCTCTCAAAGAGAAGTT
TGTGGACTATTGCATTAAAGGGGATAAATNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNNNNNNNNNNNNNNNNNNNNNNNNNNNNNNNNNNN.

For Capsid, 14 strains had sufficient sequence to analyze (gaps were stripped in the nucleotide alignment). New strains bear 39-94% nucleotide identity relative to the index and 39-100% to each other. The qPCR capsid FAM+/RDRP Cy5+ group are all very similar to the index (93.1-94.7%) as expected, while the qPCR capsid FAM−/RDRP Cy3+ strains are only 38-41% similar to the index, but 74.6-90.3% to each other. Note that in individuals like 18-PBVKM-19-023 having the Cy3+ profile, these are mono-infected according to mNGS, and thus it appears that the single capsid sequence belongs to the single RDRP sequence compiled. The US strains and 15-PBV-19-016 which are capsid FAM−/RDRP Cy5± have capsid sequences that are far more similar to MRN3406 (79-88%) than they are to the capsid FAM−/RDRP Cy3+ strains (˜40%). This suggest that these RDRP and capsid sequences co-segregate.

| | 2_PBV- | | 3-PBV- | 10-PBV- | | 14_PBV- | | |
|---|---|---|---|---|---|---|---|---|
| | MRN3406 | 1-PBV- | 4138_CG | 19-001 | 11-PBV-19- | 19-015 | 15-PBV-19- | 23-PBV-19- |
| | Capsid | 4466_CG_Capsid | Capsid | Capsid | 006_Capsid | Capsid | 016_Capsid | 035_Capsid |
| 2_PBV-MRN3406 Capsid | ID | 0.789 | 0.857 | 0.931 | 0.93 | 0.939 | 0.881 | 0.935 |
| 1-PBV-4466_CG_Capsid | 0.789 | ID | 0.78 | 0.789 | 0.792 | 0.792 | 0.788 | 0.798 |
| 3-PBV-4138_CG Capsid | 0.857 | 0.78 | ID | 0.854 | 0.859 | 0.855 | 0.916 | 0.859 |
| 10-PBV-19-001 Capsid | 0.931 | 0.789 | 0.854 | ID | 0.923 | 0.936 | 0.867 | 0.933 |
| 11-PBV-19-006_Capsid | 0.93 | 0.792 | 0.859 | 0.923 | ID | 0.933 | 0.875 | 0.935 |
| 14_PBV-19-015 Capsid | 0.939 | 0.792 | 0.855 | 0.936 | 0.933 | ID | 0.876 | 0.933 |
| 15-PBV-19-016_Capsid | 0.881 | 0.788 | 0.916 | 0.867 | 0.875 | 0.876 | ID | 0.878 |
| 23-PBV-19-035_Capsid | 0.935 | 0.798 | 0.859 | 0.933 | 0.935 | 0.933 | 0.878 | ID |
| 25-PBV-19-038 Capsid | 0.947 | 0.793 | 0.86 | 0.932 | 0.935 | 0.935 | 0.879 | 0.944 |
| 27-PBV-19-044_Capsid | 0.947 | 0.793 | 0.86 | 0.932 | 0.935 | 0.935 | 0.879 | 0.944 |
| 28-PBV-19-046_Capsid | 0.947 | 0.793 | 0.862 | 0.932 | 0.935 | 0.934 | 0.879 | 0.944 |
| 12PBVKM-19-012_Capsid | 0.414 | 0.405 | 0.328 | 0.416 | 0.416 | 0.411 | 0.423 | 0.424 |
| 14PBVKM-19-015_Capsid | 0.389 | 0.386 | 0.381 | 0.391 | 0.395 | 0.39 | 0.391 | 0.396 |
| 18PBVKM-19-023_Capsid | 0.407 | 0.386 | 0.397 | 0.414 | 0.411 | 0.408 | 0.398 | 0.415 |
| 26PBVKM-19-039_Capsid | 0.402 | 0.393 | 0.399 | 0.412 | 0.406 | 0.402 | 0.407 | 0.414 |

| | 25-PBV- | | | | | | |
|---|---|---|---|---|---|---|---|
| | 19-038 | 27-PBV-19- | 28-PBV-19- | 12PBVKM-19- | 14PBVKM-19- | 18PBVKM-19- | 26PBVKM-19- |
| | Capsid | 044_Capsid | 046_Capsid | 012_Capsid | 015_Capsid | 023_Capsid | 039_Capsid |
| 2_PBV-MRN3406 Capsid | 0.947 | 0.947 | 0.947 | 0.414 | 0.389 | 0.407 | 0.402 |
| 1-PBV-4466_CG_Capsid | 0.793 | 0.793 | 0.793 | 0.405 | 0.386 | 0.386 | 0.393 |
| 3-PBV-4138_CG Capsid | 0.86 | 0.86 | 0.862 | 0.328 | 0.381 | 0.397 | 0.399 |
| 10-PBV-19-001 Capsid | 0.932 | 0.932 | 0.932 | 0.416 | 0.391 | 0.414 | 0.412 |
| 11-PBV-19-006_Capsid | 0.935 | 0.935 | 0.935 | 0.416 | 0.395 | 0.411 | 0.406 |
| 14_PBV-19-015 Capsid | 0.935 | 0.935 | 0.934 | 0.411 | 0.39 | 0.408 | 0.402 |
| 15-PBV-19-016_Capsid | 0.879 | 0.879 | 0.879 | 0.423 | 0.391 | 0.398 | 0.407 |
| 23-PBV-19-035_Capsid | 0.944 | 0.944 | 0.944 | 0.424 | 0.396 | 0.415 | 0.414 |
| 25-PBV-19-038 Capsid | ID | 1 | 0.998 | 0.422 | 0.395 | 0.414 | 0.407 |
| 27-PBV-19-044_Capsid | 1 | ID | 0.998 | 0.422 | 0.395 | 0.414 | 0.407 |
| 28-PBV-19-046_Capsid | 0.998 | 0.998 | ID | 0.422 | 0.395 | 0.413 | 0.406 |
| 12PBVKM-19-012_Capsid | 0.422 | 0.422 | 0.422 | ID | 0.771 | 0.77 | 0.903 |
| 14PBVKM-19-015_Capsid | 0.395 | 0.395 | 0.395 | 0.771 | ID | 0.838 | 0.746 |
| 18PBVKM-19-023_Capsid | 0.414 | 0.414 | 0.413 | 0.77 | 0.838 | ID | 0.753 |
| 26PBVKM-19-039_Capsid | 0.407 | 0.407 | 0.406 | 0.903 | 0.746 | 0.753 | ID |

### Nucleotide Identity Matrix for the Capsid Open Reading Frame.

For capsid proteins, gaps were not stripped. New strains bear 20-97% amino acid identity relative to the index and 18-100% to each other. The qPCR capsid FAM+/RDRP Cy5+ strains are all very similar to the index (96-97%) and average 91.9% when including FAM−/RDRP Cy5±. The qPCR capsid FAM−/RDRP Cy3+ strains are only 20-22% similar to the index and US strains, but 76-93% (average 82.5%) to each other.

| | 2_PBV- | | 3-PBV- | 10-PBV- | | 14_PBV- | | |
|---|---|---|---|---|---|---|---|---|
| | MRN3406 | 1-PBV- | 4138_CG | 19-001 | 11-PBV-19- | 19-015 | 15-PBV-19- | 23-PBV-19- |
| | Capsid | 4466_CG_Capsid | Capsid | Capsid | 006_Capsid | Capsid | 016_Capsid | 035_Capsid |
| 2_PBV-MRN3406 Capsid | ID | 0.786 | 0.905 | 0.963 | 0.943 | 0.965 | 0.907 | 0.965 |
| 1-PBV-4466_CG_Capsid | 0.786 | ID | 0.806 | 0.786 | 0.784 | 0.786 | 0.803 | 0.789 |
| 3-PBV-4138_CG_Capsid | 0.905 | 0.806 | ID | 0.911 | 0.907 | 0.917 | 0.95 | 0.919 |
| 10-PBV-19-001 Capsid | 0.963 | 0.786 | 0.911 | ID | 0.95 | 0.963 | 0.905 | 0.965 |
| 11-PBV-19-006_Capsid | 0.943 | 0.784 | 0.907 | 0.95 | ID | 0.948 | 0.893 | 0.96 |
| 14_PBV-19-015 Capsid | 0.965 | 0.786 | 0.917 | 0.963 | 0.948 | ID | 0.912 | 0.963 |
| 15-PBV-19-016_Capsid | 0.907 | 0.803 | 0.95 | 0.905 | 0.893 | 0.912 | ID | 0.91 |
| 23-PBV-19-035_Capsid | 0.965 | 0.789 | 0.919 | 0.965 | 0.96 | 0.963 | 0.91 | ID |
| 25-PBV-19-038 Capsid | 0.971 | 0.789 | 0.917 | 0.969 | 0.963 | 0.967 | 0.91 | 0.982 |
| 27-PBV-19-044_Capsid | 0.971 | 0.789 | 0.917 | 0.969 | 0.963 | 0.967 | 0.91 | 0.982 |
| 28-PBV-19-046_Capsid | 0.971 | 0.789 | 0.917 | 0.969 | 0.962 | 0.967 | 0.909 | 0.982 |
| 12PBVKM-19-012_Capsid | 0.223 | 0.232 | 0.187 | 0.227 | 0.229 | 0.225 | 0.236 | 0.227 |
| 14PBVKM-19-015_Capsid | 0.204 | 0.209 | 0.209 | 0.198 | 0.209 | 0.205 | 0.209 | 0.202 |
| 18PBVKM-19-023_Capsid | 0.204 | 0.214 | 0.205 | 0.202 | 0.209 | 0.209 | 0.211 | 0.209 |
| 26PBVKM-19-039_Capsid | 0.209 | 0.212 | 0.216 | 0.207 | 0.218 | 0.212 | 0.218 | 0.214 |

| | 25-PBV- | | | | | | |
|---|---|---|---|---|---|---|---|
| | 19-038 | 27-PBV-19- | 28-PBV-19- | 12PBVKM-19- | 14PBVKM-19- | 18PBVKM-19- | 26PBVKM-19- |
| | Capsid | 044_Capsid | 046_Capsid | 012_Capsid | 015_Capsid | 023_Capsid | 039_Capsid |
| 2_PBV-MRN3406 Capsid | 0.971 | 0.971 | 0.971 | 0.223 | 0.204 | 0.204 | 0.209 |
| 1-PBV-4466_CG_Capsid | 0.789 | 0.789 | 0.789 | 0.232 | 0.209 | 0.214 | 0.212 |
| 3-PBV-4138_CG_Capsid | 0.917 | 0.917 | 0.917 | 0.225 | 0.209 | 0.205 | 0.216 |
| 10-PBV-19-001 Capsid | 0.969 | 0.969 | 0.969 | 0.227 | 0.198 | 0.202 | 0.207 |
| 11-PBV-19-006_Capsid | 0.963 | 0.963 | 0.962 | 0.229 | 0.209 | 0.209 | 0.218 |
| 14_PBV-19-015 Capsid | 0.967 | 0.967 | 0.967 | 0.225 | 0.205 | 0.209 | 0.212 |
| 15-PBV-19-016_Capsid | 0.91 | 0.91 | 0.909 | 0.236 | 0.209 | 0.211 | 0.218 |
| 23-PBV-19-035_Capsid | 0.982 | 0.982 | 0.982 | 0.227 | 0.202 | 0.209 | 0.214 |
| 25-PBV-19-038 Capsid | ID | 1 | 0.996 | 0.225 | 0.205 | 0.209 | 0.212 |
| 27-PBV-19-044_Capsid | 1 | ID | 0.996 | 0.225 | 0.205 | 0.209 | 0.212 |
| 28-PBV-19-046_Capsid | 0.996 | 0.996 | ID | 0.227 | 0.205 | 0.209 | 0.214 |
| 12PBVKM-19-012_Capsid | 0.225 | 0.225 | 0.227 | ID | 0.781 | 0.786 | 0.933 |
| 14PBVKM-19-015_Capsid | 0.205 | 0.205 | 0.205 | 0.781 | ID | 0.916 | 0.766 |
| 18PBVKM-19-023_Capsid | 0.209 | 0.209 | 0.209 | 0.786 | 0.916 | ID | 0.768 |
| 26PBVKM-19-039_Capsid | 0.212 | 0.212 | 0.214 | 0.933 | 0.766 | 0.768 | ID |

### Amino Acid Identity Matrix for the Capsid Open Reading Frame.

For RDRP, 14 strains had sufficient sequence to analyze (gaps were not stripped in the nucleotide alignment). New strains bear 59-93.6% nucleotide identity relative to the index and 59-100% to each other. The qPCR capsid FAM+/RDRP Cy5+ are all very similar to the index (84-93.6%), while the qPCR capsid FAM−/RDRP Cy3+ strains are only 59-61% similar to the index, but 87-95% to each other. The US strains and 15-PBV-19-016 which are capsid FAM−/RDRP Cy5± have RDRP sequences that are far more similar to MRN3406 (85%) than they are to the capsid FAM−/RDRP Cy3+ strains (59%).

| | 2-PBV | 1-PBV- | 3-PBV- | 10-PBV- | 11-PBV- | 14-PBV- | 15-PBV- | 23-PBV- | 25-PBV- | 27-PBV- |
|---|---|---|---|---|---|---|---|---|---|---|
| Seq−> | MRN3406 | 4466 | 4138 | 19-001 | 19-006 | 19-015 | 19-016 | 19-035 | 19-038 | 19-044 |
| 2-PBV MRN3406 | ID | 0.852 | 0.853 | 0.866 | 0.853 | 0.936 | 0.856 | 0.843 | 0.862 | 0.863 |
| 1-PBV-4466 | 0.852 | ID | 0.837 | 0.843 | 0.832 | 0.84 | 0.834 | 0.817 | 0.836 | 0.837 |
| 3-PBV-4138 | 0.853 | 0.837 | ID | 0.934 | 0.926 | 0.844 | 0.937 | 0.936 | 0.957 | 0.957 |
| 10-PBV-19-001 | 0.866 | 0.843 | 0.934 | ID | 0.947 | 0.857 | 0.956 | 0.928 | 0.928 | 0.928 |
| 11-PBV-19-006 | 0.853 | 0.832 | 0.926 | 0.947 | ID | 0.851 | 0.953 | 0.919 | 0.922 | 0.923 |
| 14-PBV-19-015 | 0.936 | 0.84 | 0.844 | 0.857 | 0.851 | ID | 0.852 | 0.835 | 0.85 | 0.851 |
| 15-PBV-19-016 | 0.856 | 0.834 | 0.937 | 0.956 | 0.953 | 0.852 | ID | 0.923 | 0.935 | 0.936 |
| 23-PBV-19-035 | 0.843 | 0.817 | 0.936 | 0.928 | 0.919 | 0.835 | 0.923 | ID | 0.929 | 0.93 |
| 25-PBV-19-038 | 0.862 | 0.836 | 0.957 | 0.928 | 0.922 | 0.85 | 0.935 | 0.929 | ID | 0.999 |
| 27-PBV-19-044 | 0.863 | 0.837 | 0.957 | 0.928 | 0.923 | 0.851 | 0.936 | 0.93 | 0.999 | ID |
| 28-PBV-19-046 | 0.863 | 0.837 | 0.958 | 0.929 | 0.924 | 0.851 | 0.936 | 0.931 | 0.998 | 0.999 |
| KM285233.1 | 0.598 | 0.588 | 0.592 | 0.599 | 0.598 | 0.608 | 0.594 | 0.588 | 0.596 | 0.596 |
| 12PBVKM-19-012 | 0.595 | 0.586 | 0.596 | 0.602 | 0.598 | 0.61 | 0.592 | 0.595 | 0.6 | 0.6 |
| 14PBVKM-19-015 | 0.588 | 0.581 | 0.591 | 0.601 | 0.597 | 0.605 | 0.593 | 0.591 | 0.596 | 0.596 |
| 18PBVKM-19-023 | 0.592 | 0.583 | 0.593 | 0.597 | 0.586 | 0.602 | 0.587 | 0.588 | 0.595 | 0.595 |
| 26PBVKM-19-039 | 0.592 | 0.583 | 0.59 | 0.595 | 0.591 | 0.605 | 0.592 | 0.589 | 0.595 | 0.595 |
| 27PBVKM-19-044 | 0.611 | 0.6 | 0.623 | 0.631 | 0.621 | 0.61 | 0.622 | 0.63 | 0.631 | 0.631 |

| | 28-PBV- | | 12PBVKM- | 14PBVKM- | 18PBVKM- | 26PBVKM- | 27PBVKM- |
|---|---|---|---|---|---|---|---|
| Seq−> | 19-046 | KM285233.1 | 19-01 [symbol] | 19-01 [symbol] | 19-02 [symbol] | 19-03 [symbol] | 19-04 [symbol] |
| 2-PBV MRN3406 | 0.863 | 0.598 | 0.595 | 0.588 | 0.592 | 0.592 | 0.611 |
| 1-PBV-4466 | 0.837 | 0.588 | 0.586 | 0.581 | 0.583 | 0.583 | 0.6 |
| 3-PBV-4138 | 0.958 | 0.592 | 0.596 | 0.591 | 0.593 | 0.59 | 0.623 |
| 10-PBV-19-001 | 0.929 | 0.599 | 0.602 | 0.601 | 0.597 | 0.595 | 0.631 |
| 11-PBV-19-006 | 0.924 | 0.598 | 0.598 | 0.597 | 0.586 | 0.591 | 0.621 |
| 14-PBV-19-015 | 0.851 | 0.608 | 0.61 | 0.605 | 0.602 | 0.605 | 0.61 |
| 15-PBV-19-016 | 0.936 | 0.594 | 0.592 | 0.593 | 0.587 | 0.592 | 0.622 |
| 23-PBV-19-035 | 0.931 | 0.588 | 0.595 | 0.591 | 0.588 | 0.589 | 0.63 |
| 25-PBV-19-038 | 0.998 | 0.596 | 0.6 | 0.596 | 0.595 | 0.595 | 0.631 |
| 27-PBV-19-044 | 0.999 | 0.596 | 0.6 | 0.596 | 0.595 | 0.595 | 0.631 |
| 28-PBV-19-046 | ID | 0.596 | 0.6 | 0.596 | 0.595 | 0.595 | 0.631 |
| KM285233.1 | 0.596 | ID | 0.942 | 0.94 | 0.924 | 0.94 | 0.875 |
| 12PBVKM-19-012 | 0.6 | 0.942 | ID | 0.953 | 0.922 | 0.943 | 0.887 |
| 14PBVKM-19-015 | 0.596 | 0.94 | 0.953 | ID | 0.917 | 0.952 | 0.893 |
| 18PBVKM-19-023 | 0.595 | 0.924 | 0.922 | 0.917 | ID | 0.921 | 0.872 |
| 26PBVKM-19-039 | 0.595 | 0.94 | 0.943 | 0.952 | 0.921 | ID | 0.931 |
| 27PBVKM-19-044 | 0.631 | 0.875 | 0.887 | 0.893 | 0.872 | 0.931 | ID |

[symbol]

indicates data missing or illegible when filed

### Nucleotide Identity Matrix for the RDRP Open Reading Frame.

For the RDRP protein alignment, gaps were not stripped. New strains bear 57-96% amino acid identity relative to the index and 56-100% to each other. The qPCR capsid FAM+/RDRP Cy5+ are all similar to the index (88-96%; avg 92.6%), while the qPCR capsid FAM−/RDRP Cy3+ strains are only 57-61% similar to the index and US strains, but 91-98% (avg 94.8%) to each other.

| | 2-PBV | 1-PBV- | 3-PBV- | 10-PBV- | 11-PBV- | 14-PBV- | 15-PBV- | 23-PBV- | 25-PBV- | 27-PBV- |
|---|---|---|---|---|---|---|---|---|---|---|
| Seq−> | MRN3406 | 4466 | 4138 | 19-001 | 19-006 | 19-015 | 19-016 | 19-035 | 19-038 | 19-044 |
| 2-PBV MRN3406 | ID | 0.867 | 0.907 | 0.915 | 0.9 | 0.96 | 0.911 | 0.883 | 0.905 | 0.907 |
| 1-PBV-4466 | 0.867 | ID | 0.85 | 0.866 | 0.85 | 0.856 | 0.854 | 0.83 | 0.849 | 0.85 |
| 3-PBV-4138 | 0.907 | 0.85 | ID | 0.954 | 0.966 | 0.9 | 0.971 | 0.954 | 0.977 | 0.979 |
| 10-PBV-19-001 | 0.915 | 0.866 | 0.954 | ID | 0.966 | 0.903 | 0.974 | 0.95 | 0.949 | 0.95 |
| 11-PBV-19-006 | 0.9 | 0.85 | 0.966 | 0.966 | ID | 0.892 | 0.981 | 0.962 | 0.956 | 0.958 |
| 14-PBV-19-015 | 0.96 | 0.856 | 0.9 | 0.903 | 0.892 | ID | 0.898 | 0.871 | 0.896 | 0.898 |
| 15-PBV-19-016 | 0.911 | 0.854 | 0.971 | 0.974 | 0.981 | 0.898 | ID | 0.96 | 0.969 | 0.971 |
| 23-PBV-19-035 | 0.883 | 0.83 | 0.954 | 0.95 | 0.962 | 0.871 | 0.96 | ID | 0.943 | 0.945 |
| 25-PBV-19-038 | 0.905 | 0.849 | 0.977 | 0.949 | 0.956 | 0.896 | 0.969 | 0.943 | ID | 0.998 |
| 27-PBV-19-044 | 0.907 | 0.85 | 0.979 | 0.95 | 0.958 | 0.898 | 0.971 | 0.945 | 0.998 | ID |
| 28-PBV-19-046 | 0.909 | 0.852 | 0.981 | 0.952 | 0.96 | 0.9 | 0.973 | 0.947 | 0.996 | 0.998 |
| KM285233.1 | 0.573 | 0.567 | 0.567 | 0.564 | 0.565 | 0.586 | 0.565 | 0.565 | 0.567 | 0.567 |
| 12PBVKM-19-012 | 0.569 | 0.565 | 0.563 | 0.562 | 0.561 | 0.582 | 0.561 | 0.561 | 0.563 | 0.563 |
| 14PBVKM-19-015 | 0.569 | 0.567 | 0.567 | 0.56 | 0.565 | 0.582 | 0.565 | 0.565 | 0.567 | 0.567 |
| 18PBVKM-19-023 | 0.571 | 0.563 | 0.563 | 0.566 | 0.563 | 0.586 | 0.561 | 0.565 | 0.563 | 0.563 |
| 26PBVKM-19-039 | 0.573 | 0.571 | 0.569 | 0.568 | 0.567 | 0.586 | 0.567 | 0.571 | 0.569 | 0.569 |
| 27PBVKM-19-044 | 0.611 | 0.603 | 0.606 | 0.62 | 0.603 | 0.599 | 0.606 | 0.608 | 0.606 | 0.606 |

| | 28-PBV- | | 12PBVKM- | 14PBVKM- | 18PBVKM- | 26PBVKM- | 27PBVKM- |
|---|---|---|---|---|---|---|---|
| Seq−> | 19-046 | KM285233.1 | 19-01 [symbol] | 19-01 [symbol] | 19-02 [symbol] | 19-03 [symbol] | 19-04 [symbol] |
| 2-PBV MRN3406 | 0.909 | 0.573 | 0.569 | 0.569 | 0.571 | 0.573 | 0.611 |
| 1-PBV-4466 | 0.852 | 0.567 | 0.565 | 0.567 | 0.563 | 0.571 | 0.603 |
| 3-PBV-4138 | 0.981 | 0.567 | 0.563 | 0.567 | 0.563 | 0.569 | 0.606 |
| 10-PBV-19-001 | 0.952 | 0.564 | 0.562 | 0.56 | 0.566 | 0.568 | 0.62 |
| 11-PBV-19-006 | 0.96 | 0.565 | 0.561 | 0.565 | 0.563 | 0.567 | 0.603 |
| 14-PBV-19-015 | 0.9 | 0.586 | 0.582 | 0.582 | 0.586 | 0.586 | 0.599 |
| 15-PBV-19-016 | 0.973 | 0.565 | 0.561 | 0.565 | 0.561 | 0.567 | 0.606 |
| 23-PBV-19-035 | 0.947 | 0.565 | 0.561 | 0.565 | 0.565 | 0.571 | 0.608 |
| 25-PBV-19-038 | 0.996 | 0.567 | 0.563 | 0.567 | 0.563 | 0.569 | 0.606 |
| 27-PBV-19-044 | 0.998 | 0.567 | 0.563 | 0.567 | 0.563 | 0.569 | 0.606 |
| 28-PBV-19-046 | ID | 0.567 | 0.563 | 0.567 | 0.563 | 0.569 | 0.606 |
| KM285233.1 | 0.567 | ID | 0.973 | 0.975 | 0.964 | 0.979 | 0.926 |
| 12PBVKM-19-012 | 0.563 | 0.973 | ID | 0.979 | 0.95 | 0.971 | 0.917 |
| 14PBVKM-19-015 | 0.567 | 0.975 | 0.979 | ID | 0.956 | 0.973 | 0.924 |
| 18PBVKM-19-023 | 0.563 | 0.964 | 0.95 | 0.956 | ID | 0.958 | 0.907 |
| 26PBVKM-19-039 | 0.569 | 0.979 | 0.971 | 0.973 | 0.958 | ID | 0.948 |
| 27PBVKM-19-044 | 0.606 | 0.926 | 0.917 | 0.924 | 0.907 | 0.948 | ID |

[symbol]

indicates data missing or illegible when filed

### Protein Identity Matrix for the RDRP Open Reading Frame.

While the RDRP and capsid consensus sequences are virtually identical for 19-038, 19-044, and 19-046, these are from different individuals. The Cts for each qPCR were different, as were the number of PBV and total NGS reads obtained. The compostion of other viral and bacterial reads determined by mNGS illustrates they are distinct samples and 19-044 is co-infected with the KM285233 strain, whereas the others are mono-infected. It is possible given their ages that 19-044 and 19-046 are spouses.

| | | | | | | TIME | TIME TO | |
|---|---|---|---|---|---|---|---|---|
| | | | | | DATE | COLLECTED | FREEZER | |
| SAMPLE ID | AGE | GENDER | ETH | RACE | COLLECTED | (MILITARY) | (MILITARY) | COUNTRY |
| Sptm19-0 [symbol] | [symbol] | Male | Hisp | Me [symbol] | Jul. 2, 2019 | 11:15 | 11:30 | Colombia |
| Sptm19-0 [symbol] 4 | 72 | Male | Hisp | Me [symbol] | Jul. 2, 2019 | 14: [symbol] | 14: [symbol] 0 | Colombia |
| Sptm19-04 [symbol] | 72 | Female | Hisp | [symbol] | Jul. 2, 2019 | 15:30 | 15:45 | Colombia |

| | | METHODS OF | SYSMPTOMS THAT REQUIRED | # OF |
|---|---|---|---|---|
| SAMPLE ID | DIAGNOSIS | DIAGNOSIS | HOSPTALIZATOIN | TUBES |
| Sptm19-0 [symbol] | EPOC [symbol] ( [symbol] ) | UNK | Fatigue, Night [symbol] | 1 |
| Sptm19-0 [symbol] 4 | EPOC [symbol] (—) | UNK | Night sweats, Chills, Fatigue | 1 |
| Sptm19-04 [symbol] | [symbol] ( [symbol] ) | UNK | Chest pains when coughing, Weight loss, Chills | 1 |

[symbol]

indicates data missing or illegible when filed

### Phylogenetic Analysis of New Strains

Protein sequences from new genomes were merged into alignments to generate new trees of capsid (aa 91-333; expanded from aa 110-250), shown previously in FIG. 5B, and RDRP (aa 126-473; as before) shown previously in FIG. 7B.

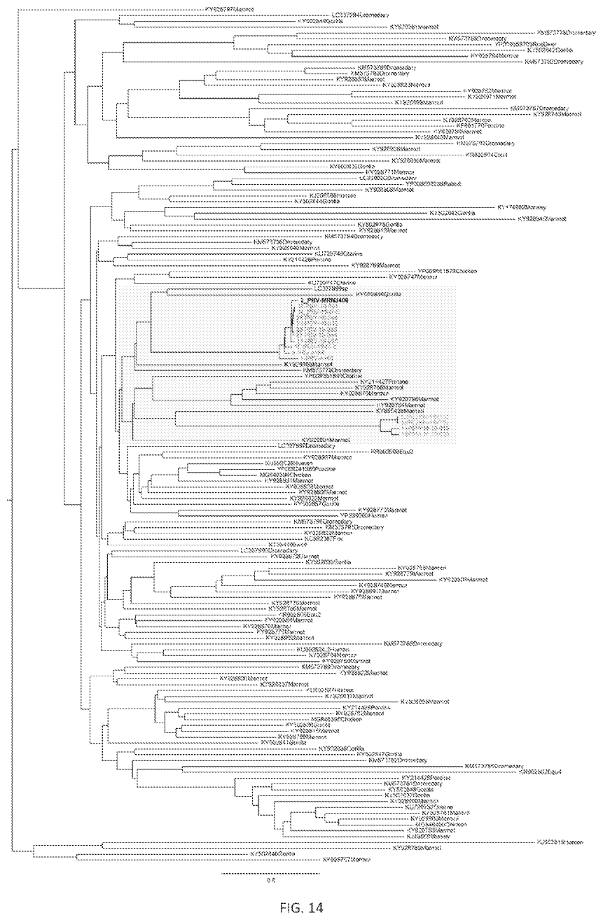

The capsid phylogenetic tree (FIG. 14) shows that new capsid FAM+ strains from Colombia all cluster together tightly with the MRN3406 index case and with short branch lengths, consistent with their ˜97% identity. Slightly basal to these are capsid FAM−/RDRP Cy5+ (in green) and FAM−/RDRP Cy5− strains (blue). These share a branch with camel and gorilla sequences. The capsid FAM−/RDRP Cy3+ (in orange) strains all cluster with one another on a separate long branch indicative of their significant genetic distance from other, with marmot sequences being the most closely related. Interestingly these capsids share a very distant common ancestor with the other new sequences, but they only have ˜20% amino acid identity.

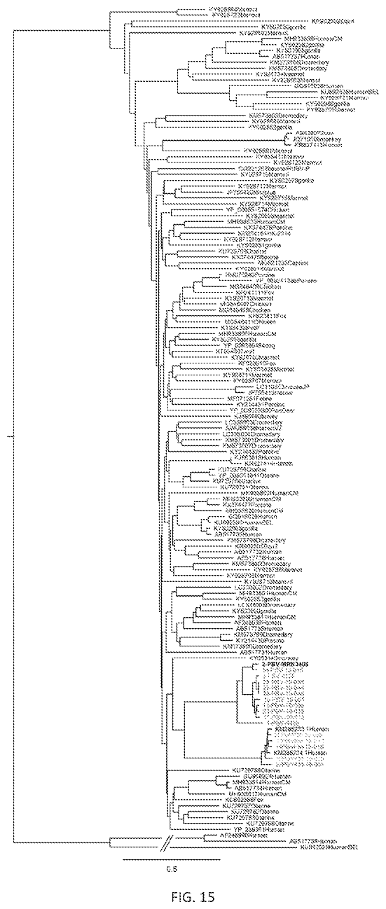

The phylogenetic tree (FIG. 15) of RDRP shows a very similar pattern as for capsid. RDRP proteins of the capsid FAM+/RDRP Cy5+ strains cluster together with the index case with short branch lengths, although not in exactly the same manner as capsid. While 14PBV 015 is highly similar to MRN3406, the others are slightly more distant, reflective of the 86-90% identity. Also unlike capsid, US and Colombian RDRP sequences branch independent of geography. As expected, the RDRP proteins of capsid FAM−/RDRP Cy3+ strains branched closely with the Cambodian respiratory strains, KM285233 and KM285234. These 2 types of respiratory viruses share a recent common ancestor and unlike capsid, there are no stool-derived sequences on this branch.

To summarize, qPCR profiles and subsequent full genome sequencing of 17 individuals confirmed that two groups of strains resembling either MRN3406 or another respiratory PBV originally found in Cambodia are in circulation. Capsid (91.9%/82.5%) and RDRP (92.6%/94.8%) amino acid sequences are highly similar within each group, respectively, and these segregate with the same pattern for individuals, demonstrating the capsid and RDRP sequences are linked. However, the large genetic distance separating these capsids (20% identity) that branch together along with GI tract-derived PBV strains is contrasted by the monophyletic relationship of RDRP sequences (60% identity) to indicate that the RDRP protein determines respiratory tropism.

### Metagenomics

It was further addressed whether picobirnavirus is simply a non-pathogenic bystander (e.g. like TTV or GBV-C), an opportunistic infection that is always secondary to a primary viral, bacterial, or fungal respiratory infection but perhaps exacerbates disease, or is it the sole pathogen present in sputum samples and the cause of illness.

For all 25 PBV+ hits sequenced, the approximate numbers of reads from co-infecting pathogens are tabulated below:

| | Capsid [symbol] | RDRP [symbol] | RDRP [symbol] | RDRP [symbol] | | | | | Fungal | |
|---|---|---|---|---|---|---|---|---|---|---|
| | FAM | FAM | CY3 | CY5 | Viral coinfection | | Bacterial coinfection | | coinfection | |
| Sample ID | Ct | Ct | Ct | Ct | virus | reads | bacteria | reads | fungus | reads |
| 564466 | −1 | 29.41 | −1 | −1 | HHV-4, Strep phage | 54,901 | Stenotrophomonas, Strep | 89,000 | none | — |
| 564468 | −1 | 33.36 | −1 | 30.34 | influenza, Strep phage | 251 | Strep pneumonia | 340,000 | none | — |
| 564138 | −1 | 26.4 | −1 | 23.87 | Klebsiella pneumonia | 3,000,000 | Klebsiella pneumonia | 76,340 | none | — |
| 564477 | −1 | 36.26 | −1 | −1 | Strep phage | 3,500 | none | — | none | — |
| 564470 | −1 | 34. [symbol] | −1 | 32.28 | HHV−1 | 1,674 | none | — | none | — |
| D000046722 | −1 | 35.04 | −1 | 33.75 | none | — | none | — | none | — |
| Sptm19-001 | 26.53 | 30.7 [symbol] | −1 | 24.06 | none | — | Prevotella, (TB+) | 40,000 | none | — |
| Sptm19-006 | 22.18 | 28.33 | −1 | 20.42 | none | — | none, (TB+) | — | none | — |
| Sptm19-012 | −1 | 23.3 [symbol] | 22.78 | −1 | Respirovirus 3, Strep | 16,299 | Strep, (TB+) | 25,000 | none | — |
| | | | | | phage | | | | | |
| Sputum Control | 35.26 | 36.34 | −1 | 33.6 | none | — | none | — | none | — |
| Sptm19-015 | 24.69 | 23.1 | 24.15 | 25.5 | none | — | none (TB+) | — | none | — |
| Sptm19-016 | −1 | 28.71 | −1 | 25.56 | Pseudomonas phage | 5,700 | Pseudomonas aeruginosa, | 100,000 | none | — |
| | | | | | | | (TB+) | | | |
| Sptm19-020 | −1 | 35.51 | −1 | −1 | none | — | Strep | 80,000 | none | — |
| Sptm19-021 | 35.59 | 35.25 | −1 | 31.49 | Enterovirus D | 72 | none | — | none | — |
| Sptm19-023 | −1 | 25.12 | 24.22 | −1 | Strep phage | 1,500 | Strep, Haemophilus | 100,000 | none | — |
| | | | | | | | parainflu | | | |
| Sptm19-031 | −1 | 32.26 | −1 | 30.16 | Rhinovirus A, Strep | 600 | Strep pneumonia | 75,000 | none | — |
| | | | | | phage | | | | | |
| Sptm19-032 | −1 | 27.69 | −1 | 25.94 | Rhinovirus A, | 333 | Haemophilus influenzae | 100,000 | none | — |
| | | | | | Haemophilus viru [symbol] | | | | | |
| Sptm19-033 | −1 | 25.35 | 26.91 | 34.56 | | | | | | |
| Sptm19-034 | −1 | 26.41 | 27.39 | 35.75 | | | | | | |
| Sptm19-035 | 35.84 | 29.3 | −1 | 26.64 | Strep phage | 1,107 | (Porphyromonas gingivalis, | 500,000 | none | — |
| | | | | | | | Tanner | | | |
| Sptm19-036 | −1 | 34.19 | −1 | 30.89 | none | — | (Strep m [symbol] ri) | 55,000 | none | — |
| Sptm19-038 | 26.57 | 30.67 | −1 | 29.08 | none | — | none | — | none | — |
| Sptm19-039 | 34.43 | 21.56 | 25.45 | 29.43 | Strep phage | 1,000 | Aggregatibacter segnis, | 7,500 | none | — |
| | | | | | | | Porphyrom | | | |
| Sptm19-044 | 22.58 | 31.48 | −1 | 26.95 | none | — | none | — | none | — |
| Sptm19-046 | 19.61 | 27.14 | −1 | 23.29 | none | — | none | — | none | — |

[symbol]

indicates data missing or illegible when filed

Indeed, mNGS for most samples did include considerable viral (HHV-4, Rhinovirus A, Respirovirus 3) and bacterial (Streptococcus, Haemophilus, Klebsiella, TB) reads (but not fungal), suggesting PBV may be an opportunistic infection of the respiratory tract. However, 3 high viral load PBV infections (Cts<26; ≥10⁵ cp/ml) did not show enrichment for any addtional microbes which argue it may be the sole pathogen causing symptoms.

It as also worth pointing out that dual PBV infections have thus far been detected in samples 14-PBV-19-015 and 26-PBV-19-039, as the qPCR would indicate. Note that the Cy5 and Cy3 probes are mutually exclusive, meaning they bind to very different sequences present at the same location in RDRP, so a sample that is positive for both is in fact co-infected with these two PBV strains.

### Discussion

In total, 25 samples (19.2%) were positive for PBV. It was entirely conceivable from the outset that despite having a reliable qPCR assay, no new strains among the samples screened would be detected. On the contrary, it is demonstrated herein that picobirnavirus infections are quite prevalent in individuals with severe respiratory symptoms. This confirms and extends the observations that PBV are not simply restricted to the GI tract, but can also be found in respiratory secretions/fluids.

There are several key points regarding the data. First, technically, the qPCR assay performed well. Ct values for each positive sample ranged from as low as 22 (≥10⁶ copies/ml) to as high as 38 (≤10² copies/ml). If all the viral loads had similar values, it might suggest a contaminant or issue with the assay. The variability among samples here suggests they are real: either it is reflection of the true titers or there are delays in Ct due to mismatches in the probe. Also, for the multiplex RDRP assay, any sample that was positive in the Cy5 or Cy3 channel was also dually positive for the ‘universal PBV’ probe in the FAM channel, as expected. Similarly, there were no instances where capsid positives were RDRP negative, although this could certainly have been possible. It was noted that samples can be triple positive for RDRP, in which case it indicates a dual infection.

Second, the capsid results are consistent with geography and the extreme genetic variability of PBV, but show that capsid and RDRP segments co-segregate. None of the samples (n=80) from the US were positive for capsid; the only capsid positives (n=10) were from the original site in Colombia. Of course, all PBV have a capsid encoding segment, but the tests herein seeks only to detect those similar to the ABT capsid. In the US, PBV strains were found with (n=4) and without (n=2) the ABT RDRP sequence (e.g. Cy5 reactivity). Despite the negative reactivity for FAM, the capsid for this group of US sequences were actually quite similar 91.9% to each other and the index. By contrast, the second group (Cy3+) resembling the Cambodian strain also with negative reactivity for FAM was very different from the index case (only 20% identity), but again highly similar to each other (82.5%). RDRP (92.6%/94.8%) amino acid sequences were likewise highly identical within each group, respectively. Thus, capsid and RDRP sequences branch with the same pattern by individual, demonstrating these are linked.

Third, the qPCR is able to detect a wide range of genetic diversity. If it were only restricted to primers and probes amplifying and detecting one genome segment with similarity to the index case (capsid FAM+ or RDRP Cy5+), the assay would have only demonstrated detection of strains with 7% or 15% dissimilarity. However, a set of primers with conserved RDRP probes were used, which in practice can amplify a large range of PBV sequences. Indeed, full genome sequencing of the hits obtained show that capsid and RDRP nucleotide sequences can have as little as 39% and 59% overall identity to the index case, respectively, and still be readily detected. This level of divergence from the index case is what is observed for all PBV, regardless of whether it comes from stool or sputum (FIG. 3 & FIG. 4). This broad tolerance for sequence diversity, coupled to its high sensitivity, make the qPCR assay a very useful discovery and diagnostic tool.

Fourth, is the prevalence of PBV and the role that this particular RDRP sequence may play in respiratory tract tropism and disease. While 3 hits with potentially altogether new RDRP sequences (FAM+ only; 2 of which had Ct>35) were found, the majority (22/25) were RDRP dual positives, either FAM+Cy5+ or FAM+Cy3+, and fell into 2 distinct groups. This striking result confirms that PBV strains bearing these RDRP sequences are either involved or possibly implicated in severe respiratory symptoms. It also says that when a PBV is detected in respiratory samples, it will likely have a sequence phylogenetically close to the ABT Colombian or the Cambodian sequences. Thus, a large genetic distance separates the capsids (20% identity) of these groups and they branch together with GI tract-derived PBV strains. By contrast, the RDRP sequences (60% identity) of the groups branch together monophyletically to indicate that it is the RDRP protein that determines respiratory tropism.

Fifth, by using unbiased mNGS and analyzing in SURPI, it was possible to assess whether other pathogens are present that might also provide plausible explanations for the respiratory symptoms exhibited. Clinical information on the US samples was not available, but for the Colombian patients, 27/50 were positive for tuberculosis (TB), which is very difficult to detect by NGS. Of the 19 PBV+ hits from this cohort, only 6 were TB positive. A majority of the other samples did show evidence of another respiratory pathogen, suggesting it could be an opportunistic infection or only found in immunocompromised individuals, which is often the case for PBV infecting the GI tract. However, in a handful of strains thus far, PBV appears to be the only pathogen present. Given these have high titers, PBV may actually be the primary acute infection, and what is observed in other patients is the progression to secondary viral or bacterial co-infections.

A new picobirnavirus strain in the sputum of a patient from Colombia was discovered. While PBV are involved in gastroenteritis and diarrhea, recent isolated reports of PBV in respiratory secretions are known. Phylogenetic analysis of the new strain indicated that out of hundreds of deposited RDRP sequences, the strain resembled those found in Cambodian patients with respiratory illness, this despite only 58% identity overall at the amino acid level. A novel quantitative PCR assay was developed to detect the capsid and RDRP segments of this strain. This assay also serves as a discovery tool, to find related and altogether new PBV sequences by virtue of sequence conservation. Active PBV infection was observed in nearly 20% of sputum samples from patients with severe respiratory illness. PBV strains similar to the novel strain (e.g. the index case) and the Cambodian strain appear to be circulating in Colombia, while related strains have spread to the United States. The high prevalence observed, coupled with its ability to rapidly evolve, reassort its segmented genome, and crossover to other species, indicates a need for greater public health awareness and future studies of picobirnaviruses.

### REFERENCES

Banyai, K. et al. Genome sequencing identifies genetic and antigenic divergence of porcine picobirnaviruses. J Gen Virol 95, 2233-2239, doi:10.1099/vir.0.057984-0 vir.0.057984-0 [pii] (2014).

2 Ganesh, B., Masachessi, G. & Mladenova, Z. Animal picobirnavirus. Virusdisease 25, 223-238, doi:10.1007/s13337-014-0207-y 207 [pii] (2014).

3 Malik, Y. S. et al. Epidemiology, phylogeny, and evolution of emerging enteric Picobirnaviruses of animal origin and their relationship to human strains. Biomed Res Int 2014, 780752, doi:10.1155/2014/780752 (2014). 4 Ribeiro Silva, R. et al. Genogroup I avian picobirnavirus detected in Brazilian broiler chickens: a molecular epidemiology study. J Gen Virol 95, 117-122, doi:10.1099/vir.0.054783-0 vir.0.054783-0 [pii] (2014).

Banyai, K. et al. Genome sequencing identifies genetic and antigenic divergence of porcine picobirnaviruses. J Gen Virol 95, 2233-2239, doi:10.1099/vir.0.057984-0 vir.0.057984-0 [pii] (2014).

2 Ganesh, B., Masachessi, G. & Mladenova, Z. Animal picobirnavirus. Virusdisease 25, 223-238, doi:10.1007/s13337-014-0207-y 207 [pii] (2014).

3 Malik, Y. S. et al. Epidemiology, phylogeny, and evolution of emerging enteric Picobirnaviruses of animal origin and their relationship to human strains. Biomed Res Int 2014, 780752, doi:10.1155/2014/780752 (2014).

4 Ribeiro Silva, R. et al. Genogroup I avian picobirnavirus detected in Brazilian broiler chickens: a molecular epidemiology study. J Gen Virol 95, 117-122, doi:10.1099/vir.0.054783-0 vir.0.054783-0 [pii] (2014).

5 Smits, S. L. et al. Genogroup I and II picobirnaviruses in respiratory tracts of pigs. Emerg Infect Dis 17, 2328-2330, doi:10.3201/eid1712.110934 (2011). 6 Cummings, M. J. et al. Precision surveillance for viral respiratory pathogens: virome capture sequencing for the detection and genomic characterization of severe acute respiratory infection in Uganda. Clin Infect Dis, doi:10.1093/cid/ciy656 5067586 [pii] (2018).

7 Rosen, B. I., Fang, Z. Y., Glass, R. I. & Monroe, S. S. Cloning of human picobirnavirus genomic segments and development of an RT-PCR detection assay. Virology 277, 316-329, doi:10.1006/viro.2000.0594 S00426822(00)90594-4 [pii] (2000).

8 Woo, P. C. et al. High Diversity of Genogroup I Picobirnaviruses in Mammals. Front Microbiol 7, 1886, doi:10.3389/fmicb.2016.01886 (2016). 9 Wakuda, M., Pongsuwanna, Y. & Taniguchi, K. Complete nucleotide sequences of two RNA segments of human picobirnavirus. J Virol Methods 126, 165-169, doi:S0166-0934(05)00063-7 [pii] 10.1016/j.jviromet.2005.02.010 (2005).

10 Da Costa, B., Duquerroy, S., Tarus, B. & Delmas, B. Picobirnaviruses encode a protein with repeats of the ExxRxNxxxE motif. Virus Res 158, 251-256, doi:10.1016/j.virusres.2011.02.018 S0168-1702(11)00070-0 [pii] (2011).

11 Knox, M. A., Gedye, K. R. & Hayman, D. T. S. The Challenges of Analysing Highly Diverse Picobirnavirus Sequence Data. Viruses 10, doi:E685 [pii] 10.3390/v10120685 v10120685 [pii] (2018).

12 van Leeuwen, M. et al. Human picobirnaviruses identified by molecular screening of diarrhea samples. J Clin Microbiol 48, 1787-1794, doi:10.1128/JCM.02452-09 JCM.02452-09 [pii] (2010).

13 Banyai, K. et al. Sequence heterogeneity among human picobirnaviruses detected in a gastroenteritis outbreak. Arch Virol 148, 2281-2291, doi:10.1007/s00705-003-0200-z (2003).

What is claimed is:

1. A primer for amplifying human picobirnavirus (PBV) in a sample, wherein the primer comprises a sequence with 80% or more sequence identity to SEQ ID NO: 4, SEQ ID NO: 5, SEQ ID NO: 7, SEQ ID NO: 8, or complements thereof.

2. A probe for detecting PBV in a sample, wherein the probe comprises a sequence with 80% or more sequence identity to SEQ ID NO: 6, SEQ ID NO: 9, or complements thereof.

3. A composition for amplifying PBV in a sample, comprising:
a) at least one forward primer comprising a sequence with 80% or more sequence identity to SEQ ID NO: 4 or a complement thereof and at least one reverse primer comprising a sequence with 80% or more sequence identity to SEQ ID NO: 5 or a complement thereof; or
b) at least one forward primer comprising a sequence with 80% or more sequence identity to SEQ ID NO: 7 or a complement thereof and at least one reverse primer comprising a sequence with 80% or more sequence identity to SEQ ID NO: 8 or a complement thereof.

4. The composition of claim 3, further comprising a probe having a sequence with 80% or more sequence identity to SEQ ID NO: 6, SEQ ID NO: 9, or complements thereof.

5. A composition for detecting PBV in a sample, comprising
a) at least one forward primer comprising a sequence with 80% or more sequence identity to SEQ ID NO: 4 or a complement thereof, at least one reverse primer comprising a sequence with 80% or more sequence identity to SEQ ID NO: 5 or a complement thereof, and a probe comprising a sequence with 80% or more sequence identity to SEQ ID NO: 6 or a complement thereof; or
b) at least one forward primer comprising a sequence with 80% or more sequence identity to SEQ ID NO: 7 or a complement thereof, at least one reverse primer comprising a sequence with 80% or more sequence identity to SEQ ID NO: 8 or a complement thereof, and a probe comprising a sequence with 80% or more sequence identity to SEQ ID NO: 9 or a complement thereof.

6. A method of detecting PBV in a sample, comprising contacting the sample with at least one primer and/or at least one probe, wherein the PBV comprises at least one sequence selected from SEQ ID NO: 1, SEQ ID NO: 6, SEQ ID NO: 7, SEQ ID NO: 9, SEQ ID NO: 10, and SEQ ID NO: 11.

7. (canceled)

8. The method of claim 6, wherein the PBV is detected by PCR or FISH.

9. The method of claim 6, comprising contacting the sample with at least one forward primer, at least one reverse primer, and at least one probe.

10. The method of claim 6, comprising contacting the sample with the composition of claim 5.

11. The method of claim 9, comprising contacting the sample with the at least one forward primer and the at least one reverse primer under amplification conditions to generate a first target sequence, and detecting hybridization between the first target sequence and the at least one probe as an indication of the presence of PBV in the sample.

12. The method of claim 11, wherein the amplification conditions comprise submitting the sample to an amplification reaction carried out in the presence of suitable amplification reagents.

13. The method of claim 12, wherein the amplification reaction comprises PCR, real-time PCR, or reverse-transcriptase PCR.

14. The method of claim 11, wherein the at least one probe is labeled with a detectable label.

15. The method of claim 14, wherein the detectable label is: (a) directly attached to the at least one probe; (b) indirectly attached to the at least one probe; (d) directly detectable; or (e) indirectly detectable.

16. (canceled)

17. (canceled)

18. (canceled)

19. The method of claim 14, wherein the detectable label comprises a fluorescent moiety attached at a 5′ end of the at least one probe.

20. The method of claim 14, wherein the at least one probe further comprises a quencher moiety attached at a 3′ end of the at least one probe.

21. A kit for detecting PBV in a sample, comprising:
a) at least one forward primer comprising a sequence with 80% or more sequence identity to SEQ ID NO: 4 or a complement thereof, at least one reverse primer comprising a sequence with 80% or more sequence identity to SEQ ID NO: 5 or a complement thereof, and a probe comprising a sequence with 80% or more sequence identity to SEQ ID NO: 6 or a complement thereof;
b) at least one forward primer comprising a sequence with 80% or more sequence identity to SEQ ID NO: 7 or a complement thereof, at least one reverse primer comprising a sequence with 80% or more sequence identity to SEQ ID NO: 8 or a complement thereof, and a probe comprising a sequence with 80% or more sequence identity to SEQ ID NO: 9 or a complement thereof; or
c) at least one forward primer comprising a sequence with 80% or more sequence identity to SEQ ID NO: 4 or a complement thereof, at least one reverse primer comprising a sequence with 80% or more sequence identity to SEQ ID NO: 5 or a complement thereof, and a probe comprising a sequence with 80% or more sequence identity to SEQ ID NO: 6 or a complement thereof, at least one forward primer comprising a sequence with 80% or more sequence identity to SEQ ID NO: 7 or a complement thereof, at least one reverse primer comprising a sequence with 80% or more sequence identity to SEQ ID NO: 8 or a complement thereof, and a probe comprising a sequence with 80% or more sequence identity to SEQ ID NO: 9 or a complement thereof.

22. An isolated polynucleotide having:
(a) 50% or more sequence identity to SEQ ID NO: 1, SEQ ID NO: 6, SEQ ID NO: 9, SEQ ID NO: 10, or fragments thereof;
(b) 80% or more sequence identity to SEQ ID NO: 1, SEQ ID NO: 6, SEQ ID NO: 9, SEQ ID NO: 10, or fragments thereof;
(c) 90% or more sequence identity to SEQ ID NO: 1, SEQ ID NO: 6, SEQ ID NO: 9, SEQ ID NO: 10, or fragments thereof; or
(d) 95% or more sequence identity to SEQ ID NO: 1, SEQ ID NO: 6, SEQ ID NO: 9, SEQ ID NO: 10, or fragments thereof.

23. (canceled)

24. (canceled)

25. (canceled)

26. A vector comprising the isolated polynucleotide of claim 22.

27. An isolated polypeptide having:
(a) 80% or more sequence identity to SEQ ID NO: 7, SEQ ID NO: 11, or fragments thereof;
(b) 90% or more sequence identity to SEQ ID NO: 7, SEQ ID NO: 11, or fragments thereof; or
(c) 95% or more sequence identity to SEQ ID NO: 7, SEQ ID NO: 11, or fragments thereof.

28. (canceled)

29. (canceled)

30. A host cell comprising the vector of claim 26.

31. A host cell comprising the isolated polypeptide of claim 27.


SEQUENCE LISTING (65 sequences)

SEQ ID NO: 1 - DNA, 2251 bp, Artificial sequence
  Synthetic
aatttgtactttaatggtttacaagagtttaaaaccatacaacactttctacactctaagaacaccagctaccgcacatagtttagtgcaaatagctaggatcagagatagtaaagtgggattatctgaaaggaggttaaattaatgacaggtaatcaaattaaatatggtgaattacaagaaaatattcgccataacactacaacagaagttgaaaccaatagacacaacgtcgtgactgaaggtgaaaccaacagacataacgttgttacagaggttgaaactaatcgacacaatactgtgactgaaagtattggatggtacgatgctgtatcaaaacgaatctcagcaaatgcttcaatgagtcaagcgggtgcagcttgggctaatgttgcaattaatcaacaaaatgcagatacaaagcgatttgaagctgaacgcaatgctgaaataaatcagcaaaatgcggacactagaacatttagtgcacgtagtgaggatgcagctagatatgctcattcttacaatgaagatcgtaaaactacagctgaaattgagcgaatgaacacacaaaattcgcaaggatgggtgaaatcaatcactgatgcaatcagctcacctatcaaagcattaccattattaggaggataaattttatggtaaagaataacaacaaaaagcgttttcaggataaaagtgataagtattctagaaaacctaagttcaaggttgaaaagaaagatatcttggacgatgacaaattggaaggatctaagtttggcaaagttaatgacatatcctggtatcagaagaatgctgatttactcagagctgctggtaacttgtcttttgctaatgcgttgggatctggaattgatctatctaacgcaaactttaacgttaagcttgctgctgatgagcaacgtgttcctggtattgcaactatacatactattacaggacctggactcagtcgcgacgcacactctggtgtcaacgtggcaatgcgtaacttatattcttttgttcgtcatgcaaatagtggtcatagtaactatgatcctgtagatctaatgttatatctacctgctatggatgcagcatacatgctctactaccgtgctgttcgtgcatatggcgcaatgttcacatttaatactgtgaatcgctatgctccaaaagctcttgtggaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatttgatgagaagtattctaaaggtggacgccttgtggctaaagcctggaaaacaaagttaactgttaaagattggattacagtagcaaatgaggttgctgatccaattacagtttcagaagatttaggtattatctcaggtgacttaattaaagcatttggtaaggaaaacttacacatgttagctaccttggctgataactacgttgtattaccaacatatgtacctgaagttatggatcaaattcataacttgcaagcagtaggtcagattgatctagaaagtaacaatattgaacaagatccaaacattggtaagggtaacttgatttacaacccagttgtaactgtcaataataatccaatggcttacgcaaatcgtattatggatttcaaaattgatacacctactccagatgatgtcgttgtagcttcacgattagctgtggcattagaaccaggcgctacaaccggtaaggcagtattcactgctatgggtacagaatttgtgactaaagttggtattcacacattctacaagggaaataatggattacttaagtctattgaacagactttcaatacttttgattctactgaaggtggtctcactgacgccgcatcagttagtttgcacatgtctgcctacacaaaggcctctaagtttgtacactttccaattcaatatatgtgtatgggtagccctactcaacctgacaaacgtgaagtcagaatctttggcgaattgggcacgtacactattattaatggggtcactcttaataagttacacgacgtgtgtgtattaagtttatttgatgtacctattaagctttagatgcattaggg

SEQ ID NO: 2 - DNA, 144 bp, Artificial sequence
  Synthetic
aatttgtactttaatggtttacaagagtttaaaaccatacaacactttctacactctaagaacaccagctaccgcacatagtttagtgcaaatagctaggatcagagatagtaaagtgggattatctgaaaggaggttaaatta

SEQ ID NO: 3 - protein, 43 aa, Artificial sequence
  Synthetic
MVYKSLKPYNTFYTLRTPATAHSLVQIARIRDSKVGLSERRLN

SEQ ID NO: 4 - DNA, 507 bp, Artificial sequence
  Synthetic
atgacaggtaatcaaattaaatatggtgaattacaagaaaatattcgccataacactacaacagaagttgaaaccaatagacacaacgtcgtgactgaaggtgaaaccaacagacataacgttgttacagaggttgaaactaatcgacacaatactgtgactgaaagtattggatggtacgatgctgtatcaaaacgaatctcagcaaatgcttcaatgagtcaagcgggtgcagcttgggctaatgttgcaattaatcaacaaaatgcagatacaaagcgatttgaagctgaacgcaatgctgaaataaatcagcaaaatgcggacactagaacatttagtgcacgtagtgaggatgcagctagatatgctcattcttacaatgaagatcgtaaaactacagctgaaattgagcgaatgaacacacaaaattcgcaaggatgggtgaaatcaatcactgatgcaatcagctcacctatcaaagcattaccattattaggaggataa

SEQ ID NO: 5 - protein, 168 aa, Artificial sequence
  Synthetic
MTGNQIKYGELQENIRHNTTTEVETNRHNVVTEGETNRHNVVTEVETNRHNTVTESIGWYDAVSKRISANASMSQAGAAWANVAINQQNADTKRFEAERNAEINQQNADTRTFSARSEDAARYAHSYNEDRKTTAEIERMNTQNSQGWVKSITDAISSPIKALPLLGG

SEQ ID NO: 6 - DNA, 1581 bp, Artificial sequence
  Synthetic
atggtaaagaataacaacaaaaagcgttttcaggataaaagtgataagtattctagaaaacctaagttcaaggttgaaaagaaagatatcttggacgatgacaaattggaaggatctaagtttggcaaagttaatgacatatcctggtatcagaagaatgctgatttactcagagctgctggtaacttgtcttttgctaatgcgttgggatctggaattgatctatctaacgcaaactttaacgttaagcttgctgctgatgagcaacgtgttcctggtattgcaactatacatactattacaggacctggactcagtcgcgacgcacactctggtgtcaacgtggcaatgcgtaacttatattcttttgttcgtcatgcaaatagtggtcatagtaactatgatcctgtagatctaatgttatatctacctgctatggatgcagcatacatgctctactaccgtgctgttcgtgcatatggcgcaatgttcacatttaatactgtgaatcgctatgctccaaaagctcttgtggaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatttgatgagaagtattctaaaggtggacgccttgtggctaaagcctggaaaacaaagttaactgttaaagattggattacagtagcaaatgaggttgctgatccaattacagtttcagaagatttaggtattatctcaggtgacttaattaaagcatttggtaaggaaaacttacacatgttagctaccttggctgataactacgttgtattaccaacatatgtacctgaagttatggatcaaattcataacttgcaagcagtaggtcagattgatctagaaagtaacaatattgaacaagatccaaacattggtaagggtaacttgatttacaacccagttgtaactgtcaataataatccaatggcttacgcaaatcgtattatggatttcaaaattgatacacctactccagatgatgtcgttgtagcttcacgattagctgtggcattagaaccaggcgctacaaccggtaaggcagtattcactgctatgggtacagaatttgtgactaaagttggtattcacacattctacaagggaaataatggattacttaagtctattgaacagactttcaatacttttgattctactgaaggtggtctcactgacgccgcatcagttagtttgcacatgtctgcctacacaaaggcctctaagtttgtacactttccaattcaatatatgtgtatgggtagccctactcaacctgacaaacgtgaagtcagaatctttggcgaattgggcacgtacactattattaatggggtcactcttaataagttacacgacgtgtgtgtattaagtttatttgatgtacctattaagctt

SEQ ID NO: 7 - protein, 524 aa, Artificial sequence
  Synthetic
MVKNNKKRFQDKSKYSRKPKSREKKDILDDDKLEGSKFGKVNDISWYQKNADLLRAAGNLSFANALGSGIDLSNANFNVKLAADEQRVPGIATIHTITGPGLSRDAHSGVNVAMRNLYSFVRHANSGHSNYDPVDLMLYLPAMDAAYMLYYRAVRAYGAMFTFNTVNRYAPKALVEALGFDYEDVNSNLATFRYAINAYAARINAYAVPTNMPIFKRHAWLFSSIYTDENVSKAQIYAFTSDHYRVFDEKYSKGGRLVAKAWKTKLTVKDWITVANEVADPITVSEDLGIISGDLIKAFGKENLHMLATLADNYVVLPTYVPEVMDQIHNLQAVGQIDLESNNIEQDPNIGKGNLIYNPVVTVNNNPMAYANRIMDFKIDTPTPDDVVVASRLAVALEPGATTGKAVFTAMGTEFVTKVGIHTFYKGNNGLLKSIEQTFNTFDSTEGGLTDAASVSLHMSAYTKASKFVHFPIQYMCMGSPTQPDKREVRIFGELGTYTIINGVTLNKLHDVCVLSLFDVPIKL

SEQ ID NO: 8 - DNA, 8 bp, Artificial sequence
  Synthetic
tgatgcgg

SEQ ID NO: 9 - DNA, 1892 bp, Artificial sequence
  Synthetic
ctaaatgaatagaaaagtagtcaagttaggtaattattttaaattaccgaatcccggattgaagacctatctattgaaaactaagagaggtaacgatgaagagtatcgtactccatttttcaaagataaatctttgtccgatgtattacaaggctggttagtgcacctagcccctctcaagagtgagtggcctggtttacaccagtttgaattagacctagcggaaaaggtcgggcctttaagcatccagaaacctttagatgagcggtttaaggatattgaggcttattacaaaggtattctcctaccttccaaaccaatcagtgaaacagcaatccgatctgttttaactgaatggaatagggcacgtggcttgtcggtacgcagtgtctccaaaacgtgggataacatgaagaaatctacatcttcaggttctccattctttactaaacgtaaagcagtcggaaaatatacgatgtatatggagccatgttttgacaaaagaacgcaagaagttcattttaagaactcaaaccgttgggatccaattgcggtcttaggttggcgtggacaagaaggtggacctgattttgaggatgtaaagcaaagggttgtatggatgttccctgcttcggtaaacctacaagagttacgtgtttaccaacctctaatcgaaacagcgcaacgtttcaacttagttcctgcttgggttggcatggatagtgttgatttgcacatcacacgtatgtttgatacgaaaggcgaagacgatgtcgtaatatgtacagatttctcaaaatttgaccaacattttaatgctgatatggctcgcggtgcatccgaaatattggatggcctctttaacgggagcagagattttgtacaatggatgtgggatatatatcacatcaaatacacgatacctctattagactcagaagatcatgcctggtttggcagacatggtatgggctctggttcaggtggaaccaatgccgatgaaacattagctcatagagctttgcagtacgaagctgctttatcacagaaccaaacattaaacccttattcacaatgtctaggtgatgatggagtactaacatatcctggaattaaagtggatgatgtaatgcgatcatatactgcacatggtcaagagatgaatgagtcaaaacagtatgtgagcaaacatgaatgcatatatcttcgtagatggcatcatattaattatcgtgtcgatgatgtatgtgtcggagtttacgcaacaactcgtgctttgggtagattgtgtgaacaagagagatattttgacccagagatatggtcaaaagaaatggtagctttacgtcagctatcgatacttgagaatgtgaaataccaccctctcaaggaagaatttgttaaatattgcatgaaaggggataagtacagactgggactggacttaccaggcttcttggagaacatagatggactcgcaaagcaagctactgatctaatgccggactttttaggttacgttaaatcacaacagaaatctgtcggtggtatatcagaatggtggatagtaaaatatctacgtagtctaaagtaaagattgggatggtgcagtaaaccattagaattctaacgaattctaactgcaccatcccaatctttactttagactacgtagatattttactatccaccactctgatataccaccgacagatttctgttgtgatttaacgtaacctaaaaagtccggcatcagatcagtagcttgctttgcgagtccatctatgttctccaagaagcctggtaagtccagtcccagtctgtacttatcccctttcatgcaatatttaacaaattcttccttgagagggtggtatttcacattctcaagt

SEQ ID NO: 10 - DNA, 1587 bp, Artificial sequence
  Synthetic
atgaatagaaaagtagtcaagttaggtaattattttaaattaccgaatcccggattgaagacctatctattgaaaactaagagaggtaacgatgaagagtatcgtactccatttttcaaagataaatctttgtccgatgtattacaaggctggttagtgcacctagcccctctcaagagtgagtggcctggtttacaccagtttgaattagacctagcggaaaaggtcgggcctttaagcatccagaaacctttagatgagcggtttaaggatattgaggcttattacaaaggtattctcctaccttccaaaccaatcagtgaaacagcaatccgatctgttttaactgaatggaatagggcacgtggcttgtcggtacgcagtgtctccaaaacgtgggataacatgaagaaatctacatcttcaggttctccattctttactaaacgtaaagcagtcggaaaatatacgatgtatatggagccatgttttgacaaaagaacgcaagaagttcattttaagaactcaaaccgttgggatccaattgcggtcttaggttggcgtggacaagaaggtggacctgattttgaggatgtaaagcaaagggttgtatggatgttccctgcttcggtaaacctacaagagttacgtgtttaccaacctctaatcgaaacagcgcaacgtttcaacttagttcctgcttgggttggcatggatagtgttgatttgcacatcacacgtatgtttgatacgaaaggcgaagacgatgtcgtaatatgtacagatttctcaaaatttgaccaacattttaatgctgatatggctcgcggtgcatccgaaatattggatggcctctttaacgggagcagagattttgtacaatggatgtgggatatatatcacatcaaatacacgatacctctattagactcagaagatcatgcctggtttggcagacatggtatgggctctggttcaggtggaaccaatgccgatgaaacattagctcatagagctttgcagtacgaagctgctttatcacagaaccaaacattaaacccttattcacaatgtctaggtgatgatggagtactaacatatcctggaattaaagtggatgatgtaatgcgatcatatactgcacatggtcaagagatgaatgagtcaaaacagtatgtgagcaaacatgaatgcatatatcttcgtagatggcatcatattaattatcgtgtcgatgatgtatgtgtcggagtttacgcaacaactcgtgctttgggtagattgtgtgaacaagagagatattttgacccagagatatggtcaaaagaaatggtagctttacgtcagctatcgatacttgagaatgtgaaataccaccctctcaaggaagaatttgttaaatattgcatgaaaggggataagtacagactgggactggacttaccaggcttcttggagaacatagatggactcgcaaagcaagctactgatctaatgccggactttttaggttacgttaaatcacaacagaaatctgtcggtggtatatcagaatggtggatagtaaaatatctacgtagtctaaag

SEQ ID NO: 11 - protein, 529 aa, Artificial sequence
  Synthetic
MNRKVVKLGNYFKLPNPGLKTYLLKTKRGNDEEYRTPFFKDKSLSDVLQGWLVHLAPLKSEWPGLHQFELDLAEKVGPLSIQKPLDERFKDIEAYYKGILLPSKPISETAIRSVLTEWNRARGLSVRSVSKTWDNMKKSTSSGSPFFTKRKAVGKYTMYMEPCFDKRTQEVHFKNSNRWDPIAVLGWRGQEGGPDFEDVKQRVVWMFPASVNLQELRVYQPLIETAQRFNLVPAWVGMDSVDLHITRMFDTKGEDDVVICTDFSKFDQHFNADMARGASEILDGLFNGSRDFVQWMWDIYHIKYTIPLLDSEDHAWFGRHGMGSGSGGTNADETLAHRALQYEAALSQNQTLNPYSQCLGDDGVLTYPGIKVDDVMRSYTAHGQEMNESKQYVSKHECIYLRRWHHINYRVDDVCVGVYATTRALGRLCEQERYFDPEIWSKEMVALRQLSILENVKYHPLKEEFVKYCMKGDKYRLGLDLPGFLENIDGLAKQATDLMPDFLGYVKSQQKSVGGISEWWIVKYLRSLK

SEQ ID NO: 12 - DNA, 301 bp, Artificial sequence
  Synthetic
taaagattgggatggtgcagtaaaccattagaattctaacgaattctaactgcaccatcccaatctttactttagactacgtagatattttactatccaccactctgatataccaccgacagatttctgttgtgatttaacgtaacctaaaaagtccggcatcagatcagtagcttgctttgcgagtccatctatgttctccaagaagcctggtaagtccagtcccagtctgtacttatcccctttcatgcaatatttaacaaattcttccttgagagggtggtatttcacattctcaagt

SEQ ID NO: 13 - DNA, 20 bp, Artificial sequence
  Synthetic
cacctactccagatgatgtc

SEQ ID NO: 14 - DNA, 21 bp, Artificial sequence
  Synthetic
ctgtacccatagcagtgaata

SEQ ID NO: 15 - DNA, 26 bp, Artificial sequence
  Synthetic
ttagctgtggcattagaaccaggcgc

SEQ ID NO: 16 - protein, 17 aa, Artificial sequence
  Synthetic
TGGCGIGGICARGAAGG

SEQ ID NO: 17 - protein, 17 aa, Artificial sequence
  Synthetic
TGGAGAGGICAIGARGG

SEQ ID NO: 18 - protein, 17 aa, Artificial sequence
  Synthetic
TGGCGIGGICARGAGGG

SEQ ID NO: 19 - protein, 19 aa, Artificial sequence
  Synthetic
CCATICIAAYCCAIGCAGG

SEQ ID NO: 20 - protein, 19 aa, Artificial sequence
  Synthetic
CIAWGCIAACCCAIGCTGG

SEQ ID NO: 21 - protein, 18 aa, Artificial sequence
  Synthetic
CAIICCGACCCAWGCTGG

SEQ ID NO: 22 - DNA, 22 bp, Artificial sequence
  Synthetic
ataaaccaatccatggcgctat

SEQ ID NO: 23 - protein, 20 aa, Artificial sequence
  Synthetic
ACCICGTCATTRCIIWCCCA

SEQ ID NO: 24 - protein, 30 aa, Artificial sequence
  Synthetic
CGTIAARCARIGIGTIGTITGGATGTTYCC

SEQ ID NO: 25 - protein, 30 aa, Artificial sequence
  Synthetic
CGTIAARCARAGIGTIGTITGGATGTTCCC

SEQ ID NO: 26 - protein, 30 aa, Artificial sequence
  Synthetic
CGTIAARCAGCGIGTIGTITGGATGTTYCC

SEQ ID NO: 27 - DNA, 27 bp, Artificial sequence
  Synthetic
cgttgcgctgtttcgattagaggttgg

SEQ ID NO: 28 - protein, 29 aa, Artificial sequence
  Synthetic
TGTAGCATATCCATAAACGGCTGRTAGAC

SEQ ID NO: 29 - DNA, 2309 bp, Artificial sequence
  Synthetic
tctttaaataagtcattactagaaaggagaaatttgtactttaatggtttacaagagtttaaaaccatacttcactttctgcactctaagaacaccagctaccgcacatagtttagtgcaaatagctaggatcagagacagatttgtgaaattacctgaaaggaggataaaatatgacaggtaatcaaattaaatatggcgaattacaagaaaacattcgtcataatcaaactaccgaagtcgaaaccaatcgacataacgttgtgactgagggtgaaacaaaccgacataacgttgtcactgagaatgagacgaatcggcacaatgtagtgacagagagtattggatggtatgatgcagtatcaaagagaatatctgctaatgcctcaatgagtcaagctggtgcagcttgggctaatgtcgctataaatcagcagaatgcggatactcgtagatatgaagctgaaagaaatgctgagattaatcagcaaaatgccgacactaagagatttagtgctgaaagtgaggatgctgctcgctatgcgcattcttacaatgaagatcgtaaaactactgctgaaattgagagaatgcagaatcaaaattctcagggatgggtgaaagctattactgatagtattagcgcaccaattaaagctttaccattattaggaggataagataaaatggcaaaatttaaagataaagaaagtttccagaaaagaaacaaaacaaagaaatgggataaaaaggatcctaagaagaatcctaaacatgatgaaccaactgaaaagttggacgacgacaaattggaaggatctaagtttggcaaagttaatgacatatcctggtatcagaagaaccctgatttactcagagctgctggtaacttgtcttttgctaatgcgttgggatctggaattaacctatctaacgctaactgtaaacttagtcttgctgctgatgagcaacgtattcctggcattgcaactatacatactattacaggacctggacttagccgatcagctaattctggagtcaatattgctatgcgtaatttatattcatttgttcgtcatgctaatagcggtcatagtaactatgatcccgtagatttaatgttatatctcttagctatggatgaggcttatatggcctatttccgtgccgtacgtgcttatggcgctatgtttacttttaatacattaaatcgatacgcacctaaggctcttgttgaagcattaggattcgattatgaagatatcaacaagaatcttgctacattcagatatgcaattaacgcatatgctgcaagaatcaatgcttacgctgtccctacgaatatgcctttgtttaagagacatgcgtggctattctcatctatttatacagatgaaaatgtatctaaagctcagatttatgcatttactactgatcattatagaacatatgatgaaaagtattctaaaggtggacgacttgtggctaaagcctggaagcctaaactaaaggtagaggattggatttcagttgctaatgaaattgcggacccaattactacttctgaagatctgggtattatatcgggcgacttaattaaagcgttcggtaaagaaaatttacacacacttgcaacattagctgataactatgttgtgttaccaacttacgtacctgaagttatggaccaaattcacaatttacaggcagttggcgatgttgaattagcgagcaacaacatcgaacaggaccctcaaattggaaagggcaacctaatctatgatccaattcttaaatcgggtaagaatccggtattatatggggatcgtattatggatttcaagattgacacaccaacaccagaagatgtaattgttgcatcacgattggctgtatcactagaaccatcaccggatggtaataaggctcattttgtagccatgggtacagagtttgtgacacatgttggaattcatacactttatcagacaacctctggtaatgttaaatgtcttgaacagacttttgatactattgcagctgttgagggtggtcttgctgatgccgcatcagttagtttgttcctatctggatacacaaaggcctctaagtttgtacattttcctattcaatatgcttgtctgggtaacgctagtgaccctaatggacaatcaatcagaatctttggtgaattggggacgtacagtactattaacagcactactcttaataaattacacgatgtgtgtgtattaagtttgttagatgtacctatcaaattatagatacatgggggaagtgaggag

SEQ ID NO: 30 - DNA, 2270 bp, Artificial sequence
  Synthetic
  misc_feature (896)..(928): n is a, c, g, or t
  misc_feature (1762)..(1779): n is a, c, g, or t
ggagaaatttgtactttaatggtttacaagagtttaaaaccatacaatgcttttctcactctaagaacaccagctaacgcacatagtctagtgcaaatagctaggatcagagatggaagagcaagattatctgaaaggaggttaaatatgaaatgacaggtaatcaaattaagtatggcgaattacaagagaacatacgccataacactactaccgaagttgagaccaaccgtcataacgttgttacagaaggcgaaacaaatcgtcacaatgttgtgactgaggctgaaactaatcggcacaatactgtaactgaaagtattggatggtacgatgcagtatcaaagagaatctcagctaacgcgtccatgagccaagcaggtgcagcttgggctaatgttgctatcaatcaacagaacgcagacacacgtaaatatgaagttgagaagaacgttgaaatcaatcaacaaaatgcagatactaaagcatttagtgccagaagtgaagatgctgctagatatgctcattcatataatgaagatcgcaaaactacagctgaaattgagcgaatgaagactcaaaattcacaaggatgggtgaaatcaattactgatgctatcagtgcgcctatcaaagcattaccattattaggaggataaattatatggtaaagaaaaatgataacaacaaacgttttcagaataaaagtgagaaatattctagaaaacctaaattcaagattgaaaagaaagatatcttggatgatgacaagcttgaaggatctaagtttggaaaagttaatgacatcagctggtatcagaagaatcctgatttactcagagctgctggtaacttgtcttttgctaacgctttgggatctggaattaacttatctnnnnnnnnnnnnnnnnnnnnnnnnnnnnnnnnngaacaacgtattcctggtattgcaactatacatactatcacaggacctgggcttagcagagacgcgcactctggtgttaacgtcgcaatgcgtaacctttattcttatgttcgtcatgcaaatagcggtcatagtaattatgaccctgtagatttgatgctttatctattggcaatggatgaggcttatatggtctactatcgcgctgtccgtgcatatggagcaatgtttacatttaatacagtaaatagatatgcgcctaaagctcttgttgaagcattaggttttgattatgaagatgtcaacgcaaaccttgctacattcagatatgcaattaacgcatatgctgcaagaatcaacgcatacgctgttcctacgaatatgcctatcttcaaacgacacgcatggctcttttcatctatctatacagatgaaaacgtatctaaggctcagatttatgcatttacttctgatcattatagaatatatgatgagaagtattctaaaggtggacgccttgtggctaaagcctggaaatcaaagttaactgtacaagactggatcaatgtagcaaatcaagtggcagatccaattactgtctcagaggatttgggtattatatctggtgatataattaaagcatttggtaaagaaaatctacatatgctggctactttagctgataactacatagttttaccaacttatgttcctgaagtcatggatcaaattcataacttacaggctgttggtaacattactcttgagagtaataatattgaacaagatccagcaattggtaaaggtaacttaatctataatccaattgtaacnnnnnnnnnnnnnnnnnnagcatatgctgatcgtattatggatttcaaaattgacactccgaccccagacgatgtagtcatagcttcacgtttagctgtggcacttgaacctggatcaacaaccgataaagcagtatttactgcaatgggtacagagtttgtaacaaaagttggaattcacacattataccgcacatctgcgggatctattaagtgtcttgaacaggacttcaatacttttgagtctactgaaggtggtcttgttgacgcctcatcagttagtttgcacttatctgcatacacgaaggcctctaaatttgtacactttccaattcaatatatgtgtttgggtagccctactactcctgacaaacgtgaagtcagaatttttggtgagttgggcacgtacactgttattaatggggtcactcttagtaagcttcacgatgtgtgtgtactgagtctatttgatgtacctatcaaattatagatacatggaaagtgaggag

SEQ ID NO: 31 - DNA, 2218 bp, Artificial sequence
  Synthetic
acactttctacactctaagaacaccagctaccgcacatagtttagtgcaaatagctaggatcagagatagtaaagtgggattatctgaaaggaggttaaatttaaatgacaggtaatcagattaagtatggcgaattacaagaaaatattcgtcataatacaacaacagaagttgagactaacagacacaacgttgttacggaaggtgaaacaaatcgtcataatgttgtaactgaagtcgagactaatcgacacaatactgttactgaaagtattggatggtacgatgctgtatcaaaacgtatctcagcgaatgcttcaatgagtcaagcaggtgcagcttgggctaacgtggctattaatcagcaaaacgctgacactaagcgctttgaagccgaacgcaatgctgaaattaatcagcagaatgcagacactaaaacatttagtgcacgcagtgaggatgccgctagatatgcacattcttacaatgaagatcgtaaaactacagcagaaattgagcgaatgaacacacaaaattcgcaaggatgggtgaaatcaataactgattcaatcagtgcacctatcagagcattaccattattaggaggataaattatatggtaaagaatactaataagaagcgttttcaggataaaagtgagaaatattctagaaaacctaagttcaaggttgaaaagaaagatatcttggacgatgacaaacttgaaggatctaagtttggaaaagttaatgacatttcctggtaccagaagaaccctgatttgctcagagctgctggtaacttgtcttttgctaatgcgttgggatctggaattgatctatctaacgcaaactttaacgttaagcttgctgctgatgagcaacgtgttcctggtattgcaactatacatactattacaggacctggactcagtcgcgacgcacactctggtgttaacgtggcaatgcgtaacttatattcttttgttcgtcatgcaaatagtggtcatagtaactatgatcctgtagacctgatgctatatctactagccatggatgaagcgtatatggtctactaccgtgctgttcgtgcatatggcgcaatgttcactttcaacacagtgaatcgctatgctccgaaagctcttgtggaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatttgatgagaagtattctaaaggtggacgccttgtggctaaagcctggaaatcaaaattaactgttaaggattggattacggtagcgaatgaagttgcagatcctattacagtgtctgaggatttaggtattatatcaggtgacttaattaaagcatttggtaaggaaaacttacacatgctggctactttagccgataattatgtcgtattaccaacatatgtacctgaggttatggaccagattcataacttacaagctgttggaacaattgacttagaaagtaataatattgaacaggatccaaacattggtaagggtaatttaatttacaatcctattgttactgtcaataataatccaatagcttacgcaaatcgtattatggatttcaagatcgagacacctactcctgaagatgtagttgttgcatcgagattagccgtagcattagaaccaggcgcgacaaccggtaaagcggtattcactgctatgggtacagaatttgtgacaaaagttggtattcatacgttctataaaggaaacaatggactacttacgtctattgaacagactttcaatacttttgattctactgaaggtggtcttgctgacgcctcatcagttagtttgcacatgtctgcctacacaaaggcctctaagttcttacactttcctattcaatatatgtgtatgggtagccctactcaacctgacaaacgtgcagtcagaatctttggcgaattgggcacttacactattgttaatggggtcactcttagtaagcttcacgatgtgtgtgtattaagtctatttgatgtacctattaaactttagatgcattaggggaaaca

SEQ ID NO: 32 - DNA, 2266 bp, Artificial sequence
  Synthetic
agtggagaaatttgtactttaatggtttacaagagtttaaaaccatacaacactttctacactctaagaacaccagctacagcacatagtttagtgcaaatagctaggatcagagatagtaaagtgggattatctgaaaggaggttaaattaaatgacaggtaatcagattaaatatggcgagttacaagaaaatattcgtcataacacaacaacagaagtcgaaactaacagacacaatgttgttacggaaggtgaaactaaccgacacaatgttgctactgaagttgagacaaatcgacacaatactgtgactgaaagtattggatggtacgatgctgtatcaaaacgaatctcagcaaatgcttcaatgagtcaagcaggtgcagcttgggcaaatgttgctattaatcagcaaaatgctgatacaaaacgatttgaagctgagcgtaatgctgaaattaatcagcaaaacgctgacaccaaaagatttagtgcacgtagtgaggatgccgctagatatgcgcactcctacaacgaagatcgtaaaactacagcagaaattgagcgaatgcacacacagaattcgcaaggatgggtgaaatcaattactgatgcaatcagtgcacctatcaaagcattaccattattaggaggataaattatatggtaaagaataacaacaaaaagcgttttcagaataaaagtgagaaatattctcgaaaacctaagttcaaggttgaaaagaaagatatcttggacgatgacaaacttgaaggatctaaatttggcaaagttaatgacatatcgtggtatcagaagaatcctgatttactcagagctgctggtaacttgtcttttgctaatgcgttgggatctggaattgatctatctaacgcaaactttaacgttaagcttgctgctgatgagcaacgtattcctggtattgcaactatacatactattacaggacctggactcagtagagacgctcactctggtgtcaacgtggcaatgcgtaacttatattcttttgttcgtcatgcaaatagcggtcatagtaattatgatcctgtagatttaatgctttatctattagctatggatgaagcgtacatggtctactaccgtgctgttcgtgcatatggcgcaatgttcacatttaatacggtgaaccgctatgctccaaaggctcttgttgaagcgttaggtttcgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatcgagtatatgatgagaagtattctaaaggtggacgccttgtggctaaagcctggaaagcaaaattaacagtacaagattggataactgtagctaatgaagttgcagatcctattacagtttctgaggatttaggcatcatatctggtgacttaattaaagcgtttggtaaggaaaacttgcatatgttagctaatttagctgataactacgttgtattaccaacttatgtacctgaagttatggatcaaattcataacttacaatcagtaggaacaatcgatctagagagtaacaatattgaacaagatccaagtattggtaagggtaatttaatttataacccaattgttactgtagataataatccaatggcattcgctaatcgtattatggattttaagatcgatacacctactcctgatgatgtagttgtagcatcacgattggctgtagcattagaaccaggcgccacgaccggtaaagcagtgttcactgctatgggtacagaatttgtgaccaaaattggtattcacacattctgcaaaggaagtaatggattacttaagtctattgaacagactttcaatacttttgattctgttgaaggtggtcttgctgacgcctcatcagttagtttgcacatgtctgcctacacaaaggcctctaagtttgtacactttcctattcaatatctgtgtatgggtagctctgctcaacctgacaagcgtgaagtcagagtctttggcgaattgggcacttacactattgttagtggggtcactcttagtaagttacacgatgtgtgtgtattaagtctatttgatgtgcctattaaactttagatgcattaggggaagtg

SEQ ID NO: 33 - DNA, 2265 bp, Artificial sequence
  Synthetic
agaaatttgtactttaatggtttacaagagtttaaaaccatacaacactttctacactctaagaacaccagctaccgcacatagtttagtgcaaatagctaggatcagagatagtaaagtgggattatctgaaaggaggttaaattgaataaatgacaggtaatcagattaagtatggcgaattacaagagagtattcgtcataattcaacgacagaagtcgaaaccaatagacacaacgttgttactgagaatgaaacgaatcgtcacaatgttgtaactgaggtggagactaatcgacacaatactgttactgaaagtattggatggtacgatgctgtatcaaaacgtatctcagctaatgcttcaatgagtcaagcaggtgcagcttgggcgaatgtcgctatcaatcagcaaaatgctgataccaaacaatttgaagctgagcgcaatgctgaaattaatcagcaaaatgcagacactaaagcgtttagtgcacgtagtgaagatgctgcgagatatgcgcattcctacaatgaagatcgtaaaactacagcagaaatcgagcgaatgaacgcacaaaattcgcaaggatgggtgaaatcaattactgatgcaatcagcgcacctatcagagcattaccattattaggaggataaattatatggtaaagaataacaacaaaaagcgttttcaggataaaagtgataagtattctagaaaacctaagttcaaggttgaaaagaaagatatcttggacgatgacaaatttgaaggatctaagtttggaaaagttaatgacattagttggtaccagaagaatcctgatttactcagagctgctggtaacttgtcttttgctaatgcgttgggatctggaattgatctatctaacgcaaactttaacgttaagcttgctgctgatgagcaacgtgttcctggtattgcaactatacatactattacaggacctggactcagtcgcgacgcacactctggtgtcaacgtggcaatgcgtaacttatattcttttgttcgtcatgcaaatagcggtcatagtaactatgatcctgtagacttaatgctatatctattagccatggatgaagcgtacatggtctactaccgtgctgttcgtgcatatggcgcaatgttcacatttaatacagtgaatcgctatgctccaaaagctcttgttgaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatttgatgagaagtattctaaaggtggacgccttgtggctaaagcctggaagtcaaaattgactgttaaggattggattactgtagctaatgaagttgcagatcctattacagtgtctgaggacctaggtattatatcaggtgacttaattaaagcattcggtaaggaaaacttacatatgttagctacattagctgacaattatgttgtgttaccaacttatgtacctgaggttatggatcaaatccataatttacaagcagttggaacaatcgatttggaaagtaacaacattgaacaggatccgactatcggtaagggtaatttaatttataacccaattgcaactgtcaataataatccattggcgtacgcaaatcgtatcatggatttcaagatcgatacacctactccagatgatgtggttgtggcatcacgattagctgtggcattagaaccaggcgctacgaccggtaaagcagtatttactgctatgggtacagaatttgtgacaaagattggtattcacacattctacaaaggaagtaatggacttattaagtctattgaacagactttcaatacttttgattctactgaaggtggtctcactgacgccacatcagttagtttgcacatgtctgcctacacaaaggcctctaagtttgtacactttcctcttcaatatatgtgtctgggtagccctactcaacctgacaaacgtgaagtcagaatctttggtgaattgggcacttacactattattaatggggtcactcttagtaagttacacgacgtgtgtgtattaagcttatttgatgtacctattaaactttagatgcattaggggaagtg

SEQ ID NO: 34 - DNA, 2292 bp, Artificial sequence
  Synthetic
ttcagtcgtcggcagcgtcagatgtgtataatttgtactttaatggtttacaagagtttaaaaccatacaatgcttttctcactctaagaacaccagctaccgcacatagtttagtgcaaatagctaggatcagagacagagaagcaagattatctgaaaggaggttaaatatgaaatgacaggtaatcaaattaagtatggcgaattacaagagaacatacgccataacactactactgaggttgaaaccaatcgtcacaatgttgttactgaaggtgaaactaatcgccataacgttgtaactgaggttgagactaatcgacacaatactgtaactgagagtattggatggtacgatgccgtatcgaaaagaatttctgcgaatgcatcaatgagtcaagcaggtgcagcttgggctaatgttgcaattaatcagcaaaatgcggatacacgcagatatgaagctgagagcaatgttgcaattaatcaacagaacgcagatacaaaggcatttagtgccagaagtgaagatgctgctagatatgctcattcatataacgaagatcgcaaaactacagctgaaattgagcgaatgaacactcaaaattcacagggatgggtgaaatcaattactgatgcaatcagtgcacctatcaaagcattaccattattaggaggataaattatatggtaaagaagaatgacaacaacaaacgttttcagaataaaagtgagaaatattctagaaaacctagattcaagattgagaagaaagatatcttggatgatgacaagcttgagggatctaagtttggaaaagttaatgacatcagctggtatcagaagaaccctgatttactcagagctgctggtaacttgtcttttgctaacgctttgggatctggaattaacttatctaactcaaactttaatattaagcttgctgctgatgaacaacgtgttcctggtattgcaactatacatactattacaggacctgggcttagcagagacgcacactctggtgttaacgtcgcaatgcgtaacctttattcttatgttcgtcatgcaaatagtggtcatagtaattatgatcctgtagatctaatgctttatctcttagccatggatgaagcttatatggtctactatcgtgccgttcgtgcatatggagcaatgtttacatttaacacagtgaatagatatgcgcctaaagctcttgttgaagcattaggttttgattatgaagatgtcaacgcaaaccttgctacattcagatatgcaattaacgcatacgctgcaagaatcaacgcatacgctgttcctacgaatatgcctatcttcaaacgacacgcatggctcttttcatctatctatacagatgaaaacgtatctaaggctcagatttatgcatttacttctgatcattatagaacatatgatgagaagtatactaaaggtggacgccttgtggctaaagcctggaaaccaaagttaactgtacgagattggatcgcagtatcaaatgaagttgcggatccaattactgtttctgaagatttgggtattatatctggtgatataattaaggcttttggtaaagaaaatctgcatatgttagctacactggctgacaattatgttgtattaccaagctatgtgcctgaagttatggatcaaattcataacctacaagcagtaggtgatgtagctcttgagagcaataacatcgaacaagatccaacaattggtaagggcaatttaatctataacccaattgtaacagttaacaataatcctttagcgtacgctgatcgcattatggatttcaaaattgacactccaactccggatgatgtagtcgtagcttctcgtttagctgtggctcttgaacccgggtcaacaaccggtaaagcagtattcactgctatgggtacagaatttgtaacaaaagttggaattcacacattataccgcacaactgagggatctattaagtgtattgaacagattttcaatacttttgagtctactgaaggcggtcttgctgacgccgcatcagttagtctgcacctatctacatacacgaaggcctctaagtttgtacactttccaattcaatatatgtgtctgggtagccctactactcctgacaaacgtgaagtcagaatctttggtgaattgggcacgtacactgttattaatggggtcactcttaataagttacacgatgtgtgtgtattgagtctatttgatgtacctattaaactttagatgcattaggggaagtg

SEQ ID NO: 35 - DNA, 2267 bp, Artificial sequence
  Synthetic
ttaggagaaaatttgtactttaatggtttacaagagtttaaaaccatacaacactttctacactctaagaacaccagctacagcacatagtttagtgcaaatagctaggatcagagatagtaaagtgggattatctgaaaggaggttaaattaaatgacaggtaatcaaattaagtatggcgaattacaagaaaatattcgtcataacactacaacagaagttgaaactaataggcacaacgttgttacagaaggcgagattaaccgacacaacattgtgactgaagtggaaacgaatcgacacaatactgttactgaaagtattggatggtacgatgctgtatcaaagcgaatctcagcaaacgcttcaatgagccaggcaggtgcagcttgggctaatgttgctattaatcagcagaatgctgatactaagcgatttgaagcagaacgcaatgctgaaattaatcagcaaaatgcagacactaaaacatttagtgcacgtagtgaggacgccgctagatatgcgcactcctacaatgaggatcggaaaactacagcagaaattgagcgaatgaacacacagaattcgcaaggatgggtgaaatctatcactgatgcaatcggtgcacctatcaaagcattaccattattaggaggataaattatatggtaaagaataataacaagaagcgttttcaggataaaagtgagaaatattctagaaaacctaagttcaaggttgaaaagaaagatatcttggacgatgacaaattggaaggatctaagtttggcaaagttaatgacatatcatggtaccagaagaatcctgatttactcagagctgctggtaacttgtcttttgctaatgcgttgggatctggaattgatctatctaacgcaaactttaacattaagcttgctgctgatgagcaacgtattcctggtattgcaactatacatactattacaggacctggactcagtagagacgcacactctggtgtcaatgtggcaatgcgtaacttatattcttttgttcgtcatgcaaatagtggtcatagtaactatgatcctgtagacttaatgctttatctattagctatggatgaagcgtatatggtttactaccgtgctgttcgtgcatatggcgcaatgttcacgtttaatacagtgaatcgctatgctccaaaagctcttgttgaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatatgatgagaagtattctaaaggtggacgccttgtggctaaagcctggaaatcaaaattgactgtaaaagattggattactgtagcaaatgaagttgcagatcctattacagtttctgaagatttaggtatcatttcaggtgacttaattaaggcatttggtaaagagaatttacacatgctagctacactagctgataactatgtagtgctaccaacatatgtacctgaagttatggaccaaattcataacttacaagctgtaggagcgattgacctagaaagtaacaatatcgaacaggatccaaatattggtaagggtaatttaatctataacccgattgtcactgttaataataatcctatggcatacgcgaatcgtattatggatttcaagattgatacacctacccctgatgatgtagttgtagcatccagattagctgtagcattggaaccaggcgcaactaccggtaaagcagtatttactgccatgggtacggaatttgtgacaaaagttggtattcacacattcttcaaaggaagtaatggattacttaaaactattgaacagactttcaacacttttgattctactgaaggtggtctcactgacgccgcatcagttagtttgcacatgtctgcctacacaaaggcctctaagtttgtacactttcctattcaatatatgtgtatgggcagccctactcaacctgacaaacgtgaagtcagagtctttggcgaattgggcacgtacactattgttaatgggatcactcttagtaagttacacgacgtgtgtgtattaagtctatttgatgtacctattaaactttagatgcattaggggaagtg

SEQ ID NO: 36 - DNA, 2257 bp, Artificial sequence
  Synthetic
aatttgtactttaatggtttacaagagtttaaaaccatacaacactttctacactctaagaacaccagctactgcacatagtatagtgcaaatagctaggatcagagatagtgaagtgggattatctgaaaggaggttaaattaatgacaggtaatcaaattaaatatggtgaattacaagagaatattcgccataacacaacaacagaagttgaaaccaatagacataacgttgtaactgaaggtgaaactaacagacataacgttgtcacagaggttgagactaatcgacacaatactgtgactgaaagtattggatggtacgatgctgtatcaaaacgtatctcagcaaatgcttcaatgagccaagccggtgcagcttgggctaacgttgcaatcaatcaacaaaatgcagacacgaaacgatttgaggctgaacgtaatgctgaaataaatcagcaaaatgcggatactaaagcatttagtgcacgcagtgaggatgcagctagatatgctcattcttacaatgaggatcgtaagactacagcagaaattgagcgaatgaacacacaaaattcgcaaggatgggtgaagtcaatcactgatgcaattagcgcacctatcaaagcattaccattattaggaggataaattttatggtaaagaataacaacaagaagcgttttcaggataaaagtgagaaatattctagaaaacctaagttcaaggttgagaagaaagatatcttggacgatgacaaattggaaggatctaagtttggcaaagttaatgacatatcctggtatcagaagaatcctgatttactcagagctgctggtaacttgtcttttgctaatgcgttgggatctggaattgatctatctaacgcaaactttaacgttaagcttgctgctgatgagcaacgtgttcctggtattgcaactatacatactattacaggacctggactcagtcgcgacgcacactctggtgtcaacgtggcaatgcgtaacttatattcttttgttcgtcatgcaaatagtggtcatagtaactatgatcctgtagatttaatgctatatctacttgctatggatgaagcatatatggtctactaccgtgctgttcgtgcatatggcgcaatgttcacatttaataccgtgaaccgctatgctccaaaagctcttgtggaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatatgatgagaagtattctaaaggtggacgccttgtggctaaagcctggaaatcaaagttaactgtcaatgattggataactgtagctaatgaagttgcggatccaattacagtctctgaggatctaggaatcatctcgggtgacttaatcaaggcatttggtaaagagaatttacatatgctagctactttagctgacaattatgttgtattaccaacatatgtacctgaagttatggatcagattcataacttacaagcagtaggtcaaattgatctagaaagtaataatattgaacaggatccaaatattggaaagggtaatttaatttacaatcctattgtaactgtcaataataatccaatggcatatgctaaccgcatcatggatttcaagattgatacacctacaccagatgatgttgttgtagcttcacgattagctgtggcattagaaccaggcgctacaaccggtaaagcagtattcactgccatgggtacagagtttgtgacaaatgttggtattcacacattctacaaaggaagtaatggattgcttaaatctattgaacaaactttcaacacttttgattctactgaaggtggtcttactgacgccgcatcagttagtttgcacatgtctgcctacacaaaggcctctaagtttgtacactttcctattcaatatatgtgtatgggtagctctactcaacctgacaagcgtgaagtcagagtctttggcgaattgggcacgtacactattgttaatggggtcactcttagtaagttacacgatgtgtgtgtattaagtctatttgatgtacctattaaactttagatgcattaggggaagtg

SEQ ID NO: 37 - DNA, 2297 bp, Artificial sequence
  Synthetic
tttctagtaagaacttaaaagttatttactagaaaggagaaatttgtactttaatggtttacaagagtttaaaaccatacaacactttctacactctaagaacaccagctactgcacatagtatagtgcaaatagctaggatcagagatagtgaagtgggattatctgaaaggaggttaaattaatgacaggtaatcaaattaaatatggtgaattacaagagaatattcgccataacacaacaacagaagttgaaaccaatagacataacgttgtaactgaaggtgaaactaacagacataacgttgtcacagaggttgagactaatcgacacaatactgtgactgaaagtattggatggtacgatgctgtatcaaaacgtatctcagcaaatgcttcaatgagccaagccggtgcagcttgggctaacgttgcaatcaatcaacaaaatgcagacacgaaacgatttgaggctgaacgtaatgctgaaataaatcagcaaaatgcggatactaaagcatttagtgcacgcagtgaggatgcagctagatatgctcattcttacaatgaggatcgtaagactacagcagaaattgagcgaatgaacacacaaaattcgcaaggatgggtgaagtcaatcactgatgcaattagcgcacctatcaaagcattaccattattaggaggataaattttatggtaaagaataacaacaagaagcgttttcaggataaaagtgagaaatattctagaaaacctaagttcaaggttgagaagaaagatatcttggacgatgacaaattggaaggatctaagtttggcaaagttaatgacatatcctggtatcagaagaatcctgatttactcagagctgctggtaacttgtcttttgctaatgcgttgggatctggaattgatctatctaacgcaaactttaacgttaagcttgctgctgatgagcaacgtgttcctggtattgcaactatacatactattacaggacctggactcagtcgcgacgcacactctggtgtcaacgtggcaatgcgtaacttatattcttttgttcgtcatgcaaatagtggtcatagtaactatgatcctgtagatttaatgctatatctacttgctatggatgaagcatatatggtctactaccgtgctgttcgtgcatatggcgcaatgttcacatttaataccgtgaaccgctatgctccaaaagctcttgtggaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatatgatgagaagtattctaaaggtggacgccttgtggctaaagcctggaaatcaaagttaactgtcaatgattggataactgtagctaatgaagttgcggatccaattacagtctctgaggatctaggaatcatctcgggtgacttaatcaaggcatttggtaaagagaatttacatatgctagctactttagctgacaattatgttgtattaccaacatatgtacctgaagttatggatcagattcataacttacaagcagtaggtcaaattgatctagaaagtaataatattgaacaggatccaaatattggaaagggtaatttaatttacaatcctattgtaactgtcaataataatccaatggcatatgctaaccgcatcatggatttcaagattgatacacctacaccagatgatgttgttgtagcttcacgattagctgtggcattagaaccaggcgctacaaccggtaaagcagtattcactgccatgggtacagagtttgtgacaaatgttggtattcacacattctacaaaggaagtaatggattgcttaaatctattgaacaaactttcaacacttttgattctactgaaggtggtcttactgacgccgcatcagttagtttgcacatgtctgcctacacaaaggcctctaagtttgtacactttcctattcaatatatgtgtatgggtagctctactcaacctgacaagcgtgaagtcagagtctttggcgaattgggcacgtacactattgttaatggggtcactcttagtaagttacacgatgtgtgtgtattaagtctatttgatgtacctattaaactttagatgcattaggggaagtg

SEQ ID NO: 38 - DNA, 2292 bp, Artificial sequence
  Synthetic
  misc_feature (1795)..(1795): n is a, c, g, or t
tgtaaataacttttaagttcttactagaaaggagaaatttgtactttaatggtttacaagagtttaaaaccatacaacactttctacactctaagaacaccagctactgcacatagtatagtgcaaatagctaggatcagagatagtgaagtgggattatctgaaaggaggttaaattaatgacaggtaatcaaattaaatatggtgaattacaagagaatattcgccataacacaacaacagaagttgaaaccaatagacataacgttgtaactgaaggtgaaactaacagacataacgttgtcacagaggttgagactaatcgacacaatactgtgactgaaagtattggatggtacgatgctgtatcaaaacgtatctcagcaaatgcttcaatgagccaagccggtgcagcttgggctaacgttgcaatcaatcaacaaaatgcagacacgaaacgatttgaggctgaacgtaatgctgaaataaatcagcaaaatgcggatactaaagcatttagtgcacgcagtgaggatgcagctagatatgctcattcttacaatgaggatcgtaagactacagcagaaattgagcgaatgaacacacaaaattcgcaaggatgggtgaagtcaatcactgatgcaatcagcgcacctatcaaagcattaccattattaggaggataaattttatggtaaagaataacaacaagaagcgttttcaggataaaagtgagaaatattctagaaaacctaagttcaaggttgagaagaaagatatcttggacgatgacaaattggaaggatctaagtttggcaaagttaatgacatatcctggtatcagaagaatcctgatttactcagagctgctggtaacttgtcttttgctaatgcgttgggatctggaattgatctatctaacgcaaactttaacgttaagcttgctgctgatgagcaacgtgttcctggtattgcaactatacatactattacaggacctggactcagtcgcgacgcacactctggtgtcaacgtggcaatgcgtaacttatattcttttgttcgtcatgcaaatagtggtcatagtaactatgatcctgtagatttaatgctatatctacttgctatggatgaagcatatatggtctactaccgtgctgttcgtgcatatggcgcaatgttcacatttaataccgtgaaccgctatgctccaaaagctcttgtggaagcgttaggttttgattatgaagatgtcaactcaaaccttgctacattcagatatgcaattaacgcatacgctgcaagaatcaacgcatacgctgtgcctacgaatatgcctatcttcaaacgacatgcatggctcttttcatctatctatacagatgaaaacgtatctaaagctcagatttatgcatttacttctgatcattatagagtatatgatgagaagtattctaaaggtggacgccttgtggctaaagcctggaaatcaaagttaactgtcaaagattggataactgtagctaatgaagttgcggatccaattacagtctctgaggatctaggaatcatctcgggtgacttaatcaaggcatttggtaaagagaatttacatatgctagctactttagctgacaattatgttgtattaccaacatatgtacctgaagttatggatcagattcataacttacaagcagtaggtcaaattgatctagaaagtaataatattgaacaggatccaaatattggaaagggtaatttaatttacaatcctattgtaactgtcaataanaatccaatggcatatgctaaccgcatcatggatttcaagattgatacacctacaccagatgatgttgttgtagcttcacgattagctgtggcattagaaccaggcgctacaaccggtaaagcagtattcactgccatgggtacagagtttgtgacaaatgttggtattcacacattctacaaaggaagtaatggattgcttaaatctattgaacaaactttcaacacttttgattctactgaaggtggtcttactgacgccgcatcagttagtttgcacatgtctgcctacacaaaggcctctaagtttgtacactttcctattcaatatatgtgtatgggtagctctactcaacctgacaagcgtgaagtcagagtctttggcgaattgggcacgtacactattgttaatggggtcactcttagtaagttacacgatgtgtgtgtattaagtctatttgatgtacctattaaactttagatgcattagggaatgtt

SEQ ID NO: 39 - DNA, 2639 bp, Artificial sequence
  Synthetic
  misc_feature (1)..(11): n is a, c, g, or t
  misc_feature (942)..(970): n is a, c, g, or t
  misc_feature (2117)..(2452): n is a, c, g, or t
  misc_feature (2564)..(2639): n is a, c, g, or t
nnnnnnnnnnntttatttttcttttgagcattcgctcatctaatccactattttaaaatctttaataagttagattctaaacaaacttacacatctagaacatttgtatgatttaaccacagaaaggaggttaacgcatatgttttatgtgatttactttactcggaactgtggcacagattggtcattacatttgtgtcctttgtccgggtagtaacatccagaaaggaggtcgatatgaccgaaaaccaattaaagtattgggatttgcaagaaacaaaacggcataacttgcgaacagaagaacttgatcagtatagaactgataaacaatttgaaggtactaagtatagtgccgacagaaactatgaaggcgtagtttattcagcaaataagaattatgaaggagtaaagtactccgctgatagacattacgcagcagcaattggatcagctaaaattcacgctggtgctacggttgcagcagctcgtattggagcaggcgctgcaatagcttcagcaagaattggagctaacgctgcaataagttcttcacaaatcaacgcagcagctaatatgtttaattcaaatcgaatggcagctgcacaaacttatagtgctgacagacattaccaagctcagataacgacaaccaagatgaacaattaccaatcttggaagaatacgcgtgatactaataacgcaagtaatttcaatgcattaataggtgcatttggtaaagttggtgctgcagcagtaggaaacggcctgagaggtcgcagataagaaaggaggccacaaatggctaagacaaacaaaactaacaaaagatccaagtttaagggagactctaaatttgacagcagaggaggaaatagaaatggtaaaggtaacagaaacaagcgaccagatagtagaggcgctagcgaactacctaaagannnnnnnnnnnnnnnnnnnnnnnnnnnnnacagtgacggaaccaacaatgtcaattggtatgctcctactgaacgcatacttaatgacacagcaacgatcccgttcaaccacgcgattggtaacgtatttaacgatagtattcctaacgttcgtcttgttaatgctattcctggtattatggaagtcaagtacattcccacaattggcataagtactgactactcctcacctataaacattgcagctaagaatatttactcaacgattcgtcacagcatatcaggcacaagaaaatatgatgcgagtgacgttatgacttacttaattccaattacatctatcttcaattattggaattggtgctgcaggttatatggtattcataagtattttgttcttagaaacagatacgttcctgaaggtatattccgtgcaatgcacgttgactatgatgattttgttaacaatctagcagactttagagctaagctgaatcagattgccttcaggttgtcaaactacaaattacctaaggatataccgctaattgatagacagacactcttaaatgaagcggtgtttaaagatggtgatagtgatctcgatcagatctacttcttcaatccaattggacactacaaatatcagcctgtacttacacagacaggtggcgcgtgccagctggttccatctatcacagaactgttcacttataacaaacctgcaaaggttaaagatctaatatcctattttgatacattattctttgatatcaatactgacaatgatttcgataagattggagctgatatcgaaactgcatactcggacaatgcacttatgcatattgctgagcttactgaggattatatgatcgagccattcttctcaatcgaaatggcagaacagctaaataatgcagatattgtgcctattaaaccattccctaaggatatcacaactaagggtttcacacgtaaaacagctacagactttgatattattcaggatgtagacagaaacattctgtacagtgatccaaatacttggctacactcatctgtagattgggataaagtattctacttaccaactctaagactaattaatactagcgtggttaatccaaatccagctgtagtcatggcttcaactagattaaaagtggctattgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcaagagcgaggtttactacacttcagaatctagcaaatttcactccaatcagtaacttcaagttagctgatttgcattctgttgcaataatgggtgaattcaatataccnnnnnnnnnnnnnnnnnnnnnnnnnnnnnnnnnnnnnnnnnnnnnnnnnnnnnnnnnnnnnnnnnnnnnnnnnnnn

SEQ ID NO: 40 - DNA, 2641 bp, Artificial sequence
  Synthetic
  misc_feature (1)..(8): n is a, c, g, or t
nnnnnnnntttatttatttctttagagcattcgctcttcaaaacaatccacttttaaatctttagaaagttagattctaaacaaactttcatatatctagaaagcatttgtatgatttaaccacagaaaggaggttaaacacttatgttttatgtgatttacaatactcgaaactgtggcacagactggtcattacgtttgtgtcctttaatcgagtagtaacatccagaaaggaggtcgatatgaccgagaaccaattaaaatattgggatttgcaggaattaaaacggcataacctgcgaacagaagaattggatcagtacagaactgacaaacaattcgaaggaactaagtatagtgcggataagaactacgaaggtgtagtttattcagctaacaagaattatgaaggagtgaaatactccgctgatcgacactatgctgcagcgattggatcagcaagaatacatgctggtgctacagtagcagcagcgagaattggagcaggcgcagcagtcgcttctgctcgtattaatgctaatgctgcaattagttctgcacaaattggagctgcgtcaaacatgttcaattcacagagaatggcagctgcacaacagtatagtgctgatagacattatcaagcacaggtaacaacaacgcgtatgaataattaccagtcgtggaagaacacacgtgacactaacgacgctagcaacttcaatgcacttattggtgcatttggtaaagttggcgctgcagcggttggatctggtatgagacgcggaagatagaaaggaggccaataatggctaagaaatcaaacactaattcaagatccaagtttaagggagaaccagactttaagtcaaaaggaggtaaattcaatggtaaaggtaacagaaacaggagatcagatggtagaagcgctaacggcatacctgaagaatcaggagacaaattcgaaaagcagcgcaacagtgacagaaccaactctgtcaattggtatgctcctactgagcgcatacttacagatacagcaacgatcccgttcaaccacgcgattggtaacgtatttgacgatagtatttctaacgttcgtcttgttaatgctattcctggtattatggaagtcaagtacatacccacaattggcgtaagcactgactattcatcaccaattaatattgcagctaagaatatatattctaatattaggcacagtatctcaggtactagaaagtatgatgcaagtgacgtcatgacttatctaataccaattacatcaatattcaattactgggcttggtgctgcagactgtacggaattcacaaatatttcgtactcaggaatagatatgtacctgaaggcatattccgtgctatgcacgtggattatgatgattttgtcaatcatctagctgactttagggctaaactaaatcagatagcgttccgcctatctaattacaagttaccaaaagatattccactgatcgatagacagatgttacttaacgaaggaatatttgcagacggcatgagtgatctagaccagatctacttctataatactataggccactataaatatcagccagccatgactgaaactggtggttcatgccaactaattccatctatcacggaattatttacttacaataaaccagcaaaagtttctgatttaatcagctactttgatcagttattctttgatattaatacggataatgatttcgataagatcggtgctgatattgaaactgcatattccgatggagcacttatgcatatagctgaacttacagaagattacagcattgctccaatctactcgttggaaatgaatgaacagcttaacaacgctgacattctaccaatcaatccattccctaaggatatcacaactaagggattcactcgtaaaacagcaagcgtatacgacatagtgcaggatgttgatagaaacattctataccatgatccagcttcgtggctatacaatatcaacggagatggagaaacgtttgatttgcctactttacgtattttaaatactaaagtatctgaccctaatcctagtattattatggcagccactcgactaaaagtagcgattgatgaaactgggaaaatactaggttgcggaactgaaattgtaacaggtatcactgtgcataatatgtcacaagatattgatactaagggtaagtggtacaccgtaccacaggagtggtccattaaatccaatattgtatacactattaacggtgagtttaaagtcatttactcacgtgacgaaaatagtggagaaattggaaaccttatgggtctgaagtatctactagaatatttcagcaagtgggagtatgctcctatgatatacacatatgatgtagcacctctattagataacgaggaaacagtagctcaatttaagagcaagaataaggaagcaagaggtagatttactacgctccagaatttggccaatttcacaccaattagtaacttcaaactaagtgatttacactcagttgctatcatgggtgaatttaacatacctggcacaattagttataaaggtactaaataacatttagtgtaaaggtatgcagtagggagaaccctactgcatacc

SEQ ID NO: 41 - DNA, 2639 bp, Artificial sequence
  Synthetic
  misc_feature (1)..(12): n is a, c, g, or t
nnnnnnnnnnnntttattttctttagagcattcgctcttcaaaacaatccactaataatcttttagaagttagattcaaaacaaactttctatatcttaagaacatttgtatgatttaaccacagaaaggaggttaaacacttatgttttatgtgatttacaatactcgaaactgtggcacagactggtcattacgtttgtgtcctttagtcgagtagcaacatccagaaaggaggtcgatatgaccgaaaaccaattaaagtattgggatttgcaggaaacaaaacggcataacctgcgaacagaggaattggatcagtatcgaactgataaacaattcgaaggtactaagtatagtgcagataagaactacgaaggagtagtttattcagcaaacaagaattacgaaggagtcaagtactccgctgacagacactatgcagcagcaattggatcagcaagaatacatgctggtgctaccgtagcagcagcaagaattggagcaggcgcagcagtcgcttctgcccgtattagtgctaacgctgcaattagttccgcacaaattggagcagcttcaaatatgtttaattcacaaagaatggctgccgcacaacagtatagtgctgatagacattatcaagcacagataacaactacgcgtatgaataatgcacaatcctggaagaacaccagagatactaacgatgcaagtaactttaatgccctcattggagcatttggaaagataggagcatcagcagttagttcaggcatgagacgcggaagatagaaaggaggccaaatatggctaagaaaacaaacactaaatcaagatccaagtttaagggagaaccagactttaagtcaaaaggaggtaaatttaatggtaaaagtgacagaaacaggagatcaaatggtagaggcgctaacagcttacctgaagaatcaggagacaaattcgaaaagcagcgcaacagtgacagaaccaactctgtcaattggtatgctcctactgagcgcatacttgcagatacagcaacgatcccgttcaaccacgcgattggtaacgtatttgacgatagtatttctaacgttcgtcttgttaatgctattcctggtattatggaagtcaagtacatacccacagttggcgtaagcactgactattcatcaccaattaatattgcagcaaagaatatttattctacaattagacacgaaatttcaggtacaaggaaatatgacgctagtgacgtcatgacgtacctaattccaataacatcaatctttaattattgggcttggtgctgcagactatatggaatacataaatacttcgtccttaggaatagatatgttcctgaaggcattttccgtgctatgcacgttgattatgatgattttgtaaatcatctagctgattttagagccaaactaaatcagatcgcgttcagactatcaaactataaattaccaaaggatattccattaattgatcgtcagatgcttctaaacgaggcaatctttgctgacggaatgagtgatcttgaccagatctatttttataatactataggacattataagtatcagcctgctatgacagatacaggtggtgcttgccagcttattccgtccattactgaactgttcacctacaataaaccagctaaggtatccgacttaattgaatattttgataaattgtttttcgatattaatacagataatgattttgacaagatcggagccgatattgaaactgcttattctgagaatgctctaatgcatatagctgaattaacggaggactataacatagctccaatttattctttagaaatgaatgagcagctgaataacgctgatattctaccaattaaaccatttcctaaagatattacaactaagggatttaatcgtaaaacagctacagatttcgacatcatacaggatgtaaatcgcaatatactttatcatgatccaaatgcttggctcaagaacatcaatactgaggaagaaatatttgatgtgcctacattaagaatcttgaatacaaaggtttctgatcctaatcctagcatcgtaatggcagctacaagattgaaagtagccattgatgaaaagggtagaattctaggctgtggctcagagatagttacaggcattactgtgcataatatgtcacaggatctggatacaaatggtaaatggtacacagtaccacaggaatggtccattaaatctaacatcgtgtacacgattaatggtgagttcaaagtactttactcaactgatgaaaacagtggagaaattggtaatttaatgggactcaaatatttactagagtactttagtaaatgggaatatgccccaatgatctatacatatgatgttacacctcttctggagaatgaggaaacaattgcacaatttaagagtaaacataaagaggcaagaggtaggtttacaacactccagaatttagctaacttcacaccaattagtaactttaagctaagtgatctacactcagttgctataatgggtgagtttaatatacctggtaccattacatacaagggtactaaataatttatctataaaggtatgcagtagggagaaccctactgcatacc

SEQ ID NO: 42 - DNA, 2641 bp, Artificial sequence
  Synthetic
  misc_feature (1)..(434): n is a, c, g, or t
  misc_feature (568)..(978): n is a, c, g, or t
  misc_feature (1273)..(1285): n is a, c, g, or t
  misc_feature (1435)..(1785): n is a, c, g, or t
  misc_feature (1933)..(2455): n is a, c, g, or t
  misc_feature (2600)..(2641): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacactacgctgcagctattggatctgctaaaattcatgctggtgcaacagtagcagctgctcgcatcggagctggcgctgcaatcgcttcagcaagaattggagctaacgctgcaattagttcttcacaaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgacggaaccaacaatgtcaattggtatgctcctactgaacgcatacttaatgacacagcaacgatcccgttcaaccacgcgattggtaacgtatttaacgatagtattcctaacgttcgtcttgttaatgctattcctggtattatggaagtcaagtacattcccacaattggcataagtacagactactcctcaccaataaacattgcagctaagaatatttattcaactatacgtcacagtatctcaggcacaagaaaatatgatgctagtgatgttatgacttatctgatnnnnnnnnnnnnnatctttaattattggaactggtgctgcaggttatatggtattcacaagtattttgttttaaggaacagatacgtgcctgaaggtatattccgtgctatgcacgttgattacgatgatttcgttaataacctagctgatttcagagctaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatgcatatcgctgagctcactgaggattatatgattgagcctttcttctcaattgagatggctgaacagttaaataacgcagatattgtgcctattaaaccattccctaaggatatcacaactaagggattcacacgtaaaactgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcaagagcaaggtttaccacacttcagaatctagcgaatttcacaccaatcagtaattttaaattagctgatttgcattctgttgcaattatgggtgaatttaacatacctggtacaattacttataagggtacaaagtaatatnnnnnnnnnnnnnnnnnnnnnnnnnnnnnnnnnnnnnnnnnn

SEQ ID NO: 43 - DNA, 2641 bp, Artificial sequence
  Synthetic
  misc_feature (1)..(978): n is a, c, g, or t
  misc_feature (1296)..(1642): n is a, c, g, or t
  misc_feature (1774)..(2641): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgacggaaccaacaatgtcaattggtatgctcctactgaacgcatacttaatgacacagcaacgatcccgttcaaccacgcgattggtaacgtatttaacgatagtattcctaacgttcgtcttgttaatgctattcctggtattatggaagtcaagtacattcccacaattggcataagtacagactactcctcaccaataaacattgcagctaagaatatttattcaactatacgtcacagtatctcaggcacaagaaaatatgatgctagtgatgttatgacttatctgattccaattacatcaatctttaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactgaactgttcacttacaataagccagctaaggttaaggacctaatatcctattttgatactctattctttgatatcaatacagataatgatttcgataagattggagctgatatcgaaactgcgtactcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 44 - DNA, 2637 bp, Artificial sequence
  Synthetic
  misc_feature (1)..(72): n is a, c, g, or t
nnnnnnnnnnnnnnnnnnnnnnnnnnnnnnnnnnnnnnnnnnnnnnnnnnnnnnnnnnnnnnnnnnnnnnnnagattctaaacaaacttacacacctagaacatttgtatgatttaaccacagaaaggaggttaacgcatatgttttatgtgatttacttaactcggaactgtggcacagattggtcattacatttgtgtcctttatccgggtagtaacatccagaaaggaggtcgatatgaccgaaaaccaattaaagtattgggatttgcaagaaactaaacggcataacttgcgaacagaagaactagatcagtacagaactgacaaacaattcgaaggcacaaaatacagtgctgaccgaaattatgaaggcgtagtctattcagctaataagaattatgaaggagtgaaatattccgctgatcgacactacgctgcagctattggatctgctaaaattcatgctggtgcaacagtagcagctgctcgcatcggagctggcgctgcaatcgcttcagcaagaattggagctaacgctgcaattagttcttcacaaattaacgcagcggctaatatgtttaattctaatagaatggcggccgcacagacttatagcgctgacagacattatcaggctcaaattactactaccaagatgaataattaccaatcttggaagaatacgcgtgatactaatgacgcgagcaatttcaatgcactgataggtgcatttggaaaggtcggcgctgcagcagttgggagcggcttgagaggtcgcagataagaaaggaggcctactatggctaagacaaacaaaactaataaaagatccaggtctaagggagacttcaagaatgacactagaggaggaaacagaaatggtaaaggtaacagaaacaagcgaccagatagtagaggcgctagcgaactacctaaagacgcccagggaagaggggacgagcaatttaagaatgacggaaccaacaatgtcaattggtatgctcctactgaacgcatacttaatgacacagcaacgatcccgttcaaccacgcgattggtaacgtatttaacgatagtattcctaacgttcgtcttgttaatgctattcctggtattatggaagtcaagtacattcccacaattggcataagtacagactactcctcaccaataaacattgcagctaagaatatttattcaactatacgtcacagtatctcaggcacaagaaaatatgatgctagtgatgttatgacttatctgattccaattacatcaatctttaattattggaactggtgctgcaggttatatggtattcacaagtattttgttttaaggaacagatacgtgcctgaaggtatattccgtgctatgcacgttgattacgatgatttcgttaataacctagctgatttcagagctaagctaaatcagattgctttcaggttgtcaaattataaactacctaaggatataccgctaatagatagacagactctattaaatgaagcggtattcaaagatggtgatagtgatcttgaccagatttacttcttcaatccaattggccactataagtatcagcctgtacttactcaaacaggtggtgcttgccagctggttccatctattactgaactgttcacttacaataagccagctaaggttaaggacctaatatcctattttgatactctattctttgatatcaatacagataatgatttcgataagattggagctgatatcgaaactgcgtactcagataatgcacttatgcatatcgctgagctcactgaggattatatgattgagcctttcttctcaattgagatggctgaacagttaaataacgcagatattgtgcctattaaaccattccctaaggatatcacaactaagggattcacacgtaaaactgctacggatttcgacattattcaggatgtagatagaaatattctatatagcgatccaaatacttggctacattctgatgttaactgggatcaggtattttatttacctacactgagattaatcaacacaagcgtggttaacccaaacccagctgtagtaatggcttcgacaagattaaaagtagctattgatgaagttggtaaaatagcaggttgtggaactgagattgtaactggaattaccattcataatatttctcaggaagttgatgataaagggaaatgggctacacttccacaagagtggagtatcagatcaaacatggtgtatacattaaatggtgtatatcagcatctatactcatctgacgcagatagcggtgaaatagttgatactatgacaatgaagtatttgctagagtactttagtaagtgggagtatgctccaatggtgtatacatatgatgtaacacctcttttgaatgaagacgaagatatttcaacgtttaaacgtggtaatcgtgttgcaagagcaaggtttaccacacttcagaatctagcgaatttcacaccaatcagtaattttaaattagctgatttgcattctgttgcaattatgggtgaatttaacatacctggtacaattacttataagggtacaaagtaatatataccacatttaggtagcagtagggagaccctactgctacc

SEQ ID NO: 45 - DNA, 2641 bp, Artificial sequence
  Synthetic
  misc_feature (1)..(189): n is a, c, g, or t
  misc_feature (890)..(980): n is a, c, g, or t
  misc_feature (1167)..(1692): n is a, c, g, or t
  misc_feature (1816)..(2641): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtcattacatttgtgtcctttatccgggtagtaacatccagaaaggaggtcgatatgaccgaaaaccaattaaagtattgggatttgcaagaaactaaacggcataacttgcgaacagaagaactagatcagtacagaactgacaaacaattcgaaggcacaaaatacagtgctgggcctatgagtattcagaaacctcttgaagagcgtttcacggatattgaggcttattacaaaggtattctcctaccttccgaaccaattagtgatgaagcaatccgatctgtcatcactgagtggaacagggctcgcggattgtcagttcgcagtacttccaaaacatgggacaatatatgtaaattctctttaccaaatgccttgattaagtcacccgagatgattcctagatcctcagagactgtaattggatccgcaacttcattagctacagttatccaatcattgacagttaactttgatttccaggctttagccacaaggcgtccacctttagaatacttctcaattgaaaactaagagaggtaacgatgaagagtatcgtactccatttttcaaaggtaaatctttatccgatgttttaaaaggctgggaagtgcacctcgcccctctcaaagagaagtggcctggtttacaccagtttgaattagacctagcggaaaaggtcgggcctatgagtattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacggaaccaacaatgtcaattggtatgctcctactgaacgcatacttaatgacacagcaacgatcccgttcaaccacgcgattggtaacgtatttaacgatagtattcctaacgttcgtcttgttaatgctattcctggtattatggaagtcaagtacattcccacaattggcataagtacagactnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctattttgatactctattctttgatatcaatacagataatgatttcgataagattggagctgatatcgaaactgcgtactcagataatgcacttatgcatatcgctgagctcactgaggattannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 46 - DNA, 1849 bp, Artificial sequence
  Synthetic
  misc_feature (1849)..(1849): n is a, c, g, or t
ctagaaaaggaggctactaatgaatagaaaagtagtcaagttaggtaattactttaaattaccaaatcccggttgaagacctatctattgaaaacagagagaggtaacgatgaagagtatcgtactccatttttcaaaggtaaatctttgtccgaaatattagaaggctggaaagtgcacctagcccctctcgaagttgagtggcctggtttacaccagtttgaattagacctagcggaaaaggtcgggccattaagtatccaaaagccattaaaagatagacttaaggatattgaggcctattacaaaggtattctcctaccttccaaacccattgactcagacgcaatccaagcggttcttgatgaatgggaaaaggcacgcggtttgtcacttcgatctactcccaaaacgtgggaaaagatgaagaaatcaacttcatctggtagtccattatttacaaagagacgcagtgtaggtcaatttacaatggactcacaaccgtgttttgacttagttacgcgagaagtacatgacgcaaaatatcgtcagtgggatccaatcgctatactaggttggcgaggacaagaaggcggtcctgactttgaggatgtaaaacagagggttgtatggatgttccctgctgcagtgaacttgcaagaattgcgagtgtatcaacctctagtcgaagtagctcaacggttcaacttagttcctgcttgggttagcatggatagtgttgattggcacatcacacgaatgtttgataccaaaggagcaaatgatgtcgtgatttgtactgatttctccaaatttgaccaacattttaatgtagatatggcgcgcggcgcatccgaaatattggatggcctctttaacgggcgcggagattttattcagtggatgtgggcaatattccacattaaatacacgatacctctattagactctgaaaatcatgcctggtttggcagacatggtatgggttccggatctggtggaactaacgctgatgaaacattagcacatagagcgttacaacatgaagtagcgctatcccataaccaaacacttaacccttattcacaatgtctaggtgatgatggagtacttacttaccctggaattaaagtgaatgatgtaatgcgatcatatactgcacatggtcaagaaatgaatgagtctaaacagtatgtgagcaaacatgagtgcatatatcttcgtagatggcatcatgaaaattatcgtgtcgacgatatatgtgttggagtttacgcaaccactagagctttgggtagattgtgtgaacaagaaagatactttgacccaggagtttggtcaaaggaaatggtagctttacgtcagctatcgatccttgagaatgtgaaataccacccgctcaaggaagaatttgttaagtattgcatgaaaggagacaagtatagactgggactagacttgccaggcttcctcgagaacatagatggaatcgttaaggaagctactgatcttatgccggacttcttaggttacgttaaatcacaacagaaaaaggttggtggtgcatcagaatggtggattgtaaaatatctgcttagtctaaagtaacagatcgggatggtgcagtaaaccattagaattcttaatgaattctaactgcaccatcccgatctgttactttagactaagcagatattttacaatccaccattctgatgcaccaccaacctttttctgttgtgatttaacgtaacctaagaagtccggcataagatcagtagcttccttaacgattccatctatgttctcgaggaagcctggcaagtctagtcccagtctatacttgtcn

SEQ ID NO: 47 - DNA, 1940 bp, Artificial sequence
  Synthetic
  misc_feature (1670)..(1781): n is a, c, g, or t
  misc_feature (1885)..(1940): n is a, c, g, or t
gcagaagacggcatacgagatgagcaatcgtctcgtgggctcggagatgtgtataagacttaatgaatagaaaagtagtcagtttaggtaattactttaaattaccaaatcccggttgaagacctatctattgaaaactaagagaggtaacgatgaagagtatcgtactccatttttcaaaggtaaatctttatccgatgtgttaaaaggctgggaagtgcaccttgcccctctcaaagagaagtggcctggtttacaccagtttgaattagacctagcggcgaaggtcgggcctatgagtattcagaaaccgctcgaagagcgtttcaaggatatcgaggcttattacaaaggtattctcctaccttccgaaccaattcgtgatgaagcagtccgatctgtcatcactgaatggaacagggctcgcggattgtcagttcgcagtacttccaaaacatgggacaatatgaagaagtccacttcttcaggctctccattctttaccaaacgtaagttgattggtaaatacataatggatagtcaaccatattttgacaaaagaacgcaagaggtacacgataaggtgtatccacattgggatccaattgctgttcttggttggcgtggacaagaaggaggtccagaaccagaggatgtgaagcaaagggttgtatggatgttccctgcttcagttaacttgcaagaattgcgagtatatcaacctctgatcgaaacagcgcaacgtttcaacttagttcctgcttgggttagcatggatagtgtggacgagcacatcacacgtatgtttgatactaaaggcgcagatgatgtcgtgatatgtactgatttctctaaatttgaccaacattttaacgctgatatggctcgcggcgcatccgaaattttggatggtctatttaacgggagtcgagatttcgtacagtggatgtgggatatataccacattaaatacacgatacctctattagactctgaaaaccatgcgtggtttggacgtcatggtatgggttccggttcaggcggaactaatgctgatgagacattggctcatcgcgcgctgcaatatgaagcagcactctcacaaaaccaaacactaaacccttattcacaatgcttgggtgatgatggagtactaacgtatccaggtattaaagtggatgatgtaatgcgatcatatactgctcatggtcaagaaatgaatgagtcaaagcagtacgtgagcaaacatgaatgcatatatctgagaagatggcatcacaaagattatcgtgtggcagatatatgtgtcggagtttatgcaactactagagctttgggtagattgtgtgaacaggaaagatactttgatccagaagtatggtcaaaggaaatggtagctttacgtcagctatcgatccttgagaacgttaaataccacccactcaaggaagaattcgtgaattattgcatgaaaggcgacaagtatagactgggactagacttgccaggcttcttagagaacattgatggactcgcaaagcaagctactgatcttatgccggactttttgggatacgttaagtcccaacagaaggatactggaatgagcgattggtggatcgtgaagtatcttaaaagtttaaagtagagatttggatggtgcagttagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacttaacgtatcccaaaaagtccggcataagatcagtagcttgctttgcgagtccatcaatgttctctaagaagcctggcaagtctagtcccagtctatactnnnnnnnnnnnnnnnnnnnnnnnnnnnnnnnnnnnnnnnnnnnnnnnnnnnnnnnn

SEQ ID NO: 48 - DNA, 1870 bp, Artificial sequence
  Synthetic
  misc_feature (1528)..(1626): n is a, c, g, or t
  misc_feature (1788)..(1870): n is a, c, g, or t
gaatagaaaagtagtcagtttaggtaattactttaaattaccaaatcccggttgaagacctatctattgaaaactaagagaggtaacgatgaagagtatcgtactccatttttcaaaggtaaatctttgtccgaagtattaaaaggctgggaagtgcaccttgcccctctcaaagagaagtggcctggtttacaccagtttgaattagacctagcggaaaaggtcgggccgatgagtatccagaaacctcttgatgagcgtttcaaggatattgaagcctattacaaaggtattctcctaccttccactccaattagtgatgcagcaatccaatctgtactcactgaatggaacagggctcgcggattgtcagttcgcagtacttccaaaacatgggataagatgaagaagtctacttcttcaggctctccattctttaccaaacgtaaactaattggtaagtatattatggatagcgaaccatattttgacaaaagaacgcaagaggtacatgatagaaagtaccgacaatgggatccaattgctgttcttggttggcgaggacaagaaggaggtccagaaccagaggatgtaaagcaaagggttgtatggatgttccctgcttcggtgaacctgcaagaattgcgggtataccaacctctgatcgaaacagcgcaacgtttcaacttagttcctgcttgggttagcatggatagtgtggatcagcacatcacacgtatgtttgatactaaaggcgcagatgatgtcgtgatttgtacagatttctcaaaatttgaccaacatttcaactctgatatggctcgtggtgcttcagagatattagatggcttgtttaacggaagtcgagattttgtgcaatggatgtgggatacataccacataaagtacacgatacctctattagactcggaaaaccatgcgtggtttggacgtcatggtatgggttccggttcaggcggaactaatgctgatgagacattagctcatcgcgcgctgcaatatgaagcagcgctttctcaacaccaaacacttaacccttattcacaatgcctaggtgatgatggagtacttacgtacccaggtattaaagtggatgatgtaatgcgatcatatactgctcatggtcaagagatgaatgagtcaaagcagtacgtgagcacacatgaatgcatatatctgagaagatggcatcataaagattatcgtgtgtcagatgtatgtgttggagtttatgcaactactcgtgctttaggcaggttgtgtgaacaagaacgctactttgatccagaagtatggtcaaaggaaatggtagctttacgtcagctatcgatccttgagaatgttaaattccacccactcaaggaagaattcgtgaattattgcatgaaaggcgacaagtatagactgggactagacttgccaggcttcttggagaacattgatggactcgcaaagcaagctactgatcttatgccggactttttgggatacgttaagtcccaacagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctaacttcaccatccgaatctttactttagacttttaagatatttaacgatccaccaattgctcattcccgtatctttctgctgggacttaacgtatcccaaaaagtccggcataagatcagtagcttgctttgcgagtccatcaatgttctccaagaagcnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 49 - DNA, 1926 bp, Artificial sequence
  Synthetic
cggcaaccaccaagatctacacgttgaccttcgtcggcagcgtcagatgttaaatgaatagaaaagtagtcagtttaggtaattactttaaattaccaaatcccggttgaagacctatctattgaaaactaagagaggtaacgatgaagagtatcgtactccatttttcaaagataaatctttgtccgaagtgttaaaaggctgggaagtgcaccttgcccctctcaaagagaagtggcctggtctacaccagtttgaattagacctagcggaaaaggtcgggccgatgagtatccagaaacctcttgatgagcgttttaaggatattgaagcctattacaaaggtattctcctaccttccactccaataagtgatgaagcagtccgatctgtaatcactgaatggaatagggctcgcggattgtcagttcgcagtacttccaaaacatgggataagatgaagaaatcaacttcatcgggctctccattctttaccaaacgtaaattgattggtaaatatattatggatagtcaaccatattttgacaagagaacgcaacaggtacatgatagaaagtaccctcaatgggatccgattgctgttcttggttggcgaggacaagaaggaggtccagaaccagaggatgtgaagcaaagggttgtatggatgttccctgcttcagtgaatctacaagaattgcgggtttaccaacctctgatcgaaacagcgcaacgtttcaacttagttcctgcttgggttagtatggatagtgtggatgagcacatcacacgtatgtttgacacaaaaggcgcagatgatgtcgttgtatgtactgatttctccaaatttgaccaacatttcaactctgatatggctcgcggtgcgtcagagatattagatggcttgtttaacggaagtcgagacttcgtacaatggatgtgggatacataccacataaaatacacgatacctctattagactcagaaaaccatgcgtggtttggacgacatggaatgggttccggttcaggcggaacaaatgctgatgaaacattagcacatcgcgcgttgcagtatgaagcagcgctttctcaaaaccaaacactaaacccttattcacaatgcctaggtgatgatggagtacttacatacccaggtattaaagtggatgatgtaatgcgatcatatactgctcatggtcaagaaatgaatgagtcgaagcagtacgtgagcaaacatgaatgcatatacttgagaaggtggcatcacaaagattatcgtgtgtcaggtatatgtgtcggagtttatgcaactactcgtgctttaggacggttgtgtgaacaagaaagatactttgacccagaagtatggtcaaaggaaatggtagccttacgtcagctatcgatccttgagaatataaaataccacccactcaaggaagaattcgtgaattattgcatgaaaggggacaagtatagactgggactagacttgccaggcttcttagagaacatagatggactcgcaaagcaagctactgatcttatgccggacttcttgggatacgttaagtcccaacagaaagatacaggaatgagcgattggtggatcgttaagtatcttaaaagtctaaagtaaagatttggatggtgcagcaaaccattagaattcatattgaattctaactgcaccatccaaatctttactttagacttttaagatacttaacgatccaccaatcgctcattcctgtatctttctgttgggacttaacgtatcccaagaagtccggcataagatcagtagcttgctttgcgagtccatctatgttctctaagaagcctggcaagtctagtcccagtctatacttgtcccctttcatgcaataattcacgaattcttccttgagtgggtggtatttcata

SEQ ID NO: 50 - DNA, 1881 bp, Artificial sequence
  Synthetic
ctaaatgaatagaaaagtagtcaagttaggtaattactttaaattaccaaatcccggttgaagacctatctattgaaaactcagagaggtaacgatgaagagtatcgtactccatttttcaaagataaatctttgtccgaagtattaaatggctggttagtgcaactagcgcctctcaaagagaagtggcctggtttacaccagtttgaattagacctagcggaaaaggtcgggcctctaagcatccagaaacctttggaagaaaggtttaaggatatagaagcttattacaaaggtattctcctaccttccaaaccaattagtgaggcggcaatccgatccgtcttaactgaatggaatagggcacgtggcttgtcagtacgcagtgtctccaagacatgggataacatgaagaaatcgacttcttctggatctccattctttactaaacgtaaagcagtgggaaaatatactatgtatatggaaccatgttttgacaaaagaacgcaagaagttcattttaagaactcaaaccgttgggatccaattgcggtcttaggttggcgtggacaagaaggtggacctgattttgaggatgtgaagcaaagggtagtatggatgttccctgcttcggtaaacctacaagagttacgtgtttaccaacctctaatcgaaacagcgcaacgtttcaacttagttcctgcttgggttggcatggatagtgttgatttgcacatcacacgtatgtttgatacgaaaggcgcagatgatgtcgtaatctgtacagatttctcgaaatttgaccaacattttaacgctgatatggcgcgcggtgcatccgagatattggatggcctctttaacgggcgcagagattttgtacaatggatgtgggatatatatcacatcaaatacacgatacctctactcgactcagaagatcatgcctggtttggcagacatgggatgggttccggatctggtggaaccaacgctgatgaaacattagcacacagagctttgcagtatgaagctgctttatcacagaaccaaacattaaacccttattcacaatgtctaggtgatgatggagtactaacttaccctggtattaaggtggaggatgtaatacgaacatatactgcacatggtcaagagatgaatcccgataagcagtatgtgagtaaacaggaatgcatatatctgagaagatggcatcacattgattatcgtgttaatgatatatgtgtcggagtttacgcaactactcgagctttaggtcgtttgtgtgaacaagaaaggtattttgatccagagatatggtcaaaagaaatggtagctcttcgtcagctatcaatacttgagaatgtgaaataccaccctctcaaggaagaatttgttaagtattgcatgaaaggggataagtacagactgggactggacttaccaggctttctcgagaacatagatggactcgcaaagaaagctaccgatctaatgccggactttttaggttacgttaaatcacaacagaaatctgtcggtggtatatcagattggtggatagtaaaatatctacgtagtctaaagtaatgattgggatggtgcagtaaaccattagaattctaacgaattctaactgcaccatcccaatcattactttagactacgtagatattttactatccaccaatctgatataccaccgacagatttctgctgtgatttaacgtaacctaaaaagtccggcataagatcggtagctttctttgcgagtccatctatgttctcgagaaagcctggtaagtccagtcccagtctgtacttatcccctttcatgcaatacttaacaaattcttccttgagagggtggtatttcac

SEQ ID NO: 51 - DNA, 1907 bp, Artificial sequence
  Synthetic
  misc_feature (1619)..(1630): n is a, c, g, or t
aaaggagacgacttaatgaatagaaaagtagtcagtttaggtaattactttaaattaccaaatcccggttgaagacctatctattgaaaactaagagaggtaacgatgaagagtatcgtactccatttttcaaaggtaaatctttatccgatgtattaaaaggctgggaagtgcaccttgcccctctcaaagagaagtggcctggtttacaccagtttgaattagacctagcggaaaaggtcgggccgatgagtattcagaaacctcttgatgagcgtttcaaggatattgaagcctattacaaaggtattctcctaccttccactccaataagtgatgcagcaatccgatctgtaatcactgaatggaatagggctcgcggattgtcagttcgcagtacttccaaaacatgggataagatgaagaaatcaacttcatcaggctctccattctttaccaaacgtaaattgattggtaagtacattatggatagtcaaccatattttgacaaaagaacgcaagaggtacatgataaacagtacccacaatgggatccaattgcggttcttggttggcgaggacaagaaggtggtccagaaccagaggatgtgaagcaaagggttgtatggatgttccctgcttcagttaacctgcaagaattgcgggtataccaacctctgatcgaaacagcgcaacgtttcaacttagttcctgcttgggttagcatggatagtgtggatgagcacatcacacgtatgtttgatactaaaggcgcagatgatgtcgtgatttgtactgatttctctaaatttgaccaacatttcaattctgatatggctcgaggcgcatcagagatattagatggcctatttaacgggagtcgagatttcgtacaatggatgtgggatacataccacataaagtacacgatacctctactagactcagaaaaccatgcgtggtttggacgtcatggaatgggttccggctcaggtggaaccaatgctgatgaaacattagcacatcgcgcgttgcaatatgaagcagcgctttctcaaaaccaaacacttaacccttattcacaatgcctaggtgatgatggagtacttacgtacccaggtattaaagtggatgatgtaatgcggtcatatactgctcatggtcaagagatgaatgagtcaaagcagtacgtgagcaaacatgaatgcatatatctgagaagatggcatcacaaagattatcgtgtgtcagatgtatgtgtcggagtttatgcaacaacccgtgctttgggtcggttgtgtgaacaagaacgatactttgatccagaagtatggtcaaaggaaatggtagctctgcgtcagctatcgatccttgagaatatcaaataccacccactcaaggaagaattcgtgaattattgcatgaaaggagacaagtatagactgggactagacttgccaggcttcttggagaatattgatggactcgcaaagcaagctactgatcttatgccggacttcttgggatacgttaagtcccaacaaaaggatacaggaatgagcgattggtggatcgtcaagtatcttaaaagtctaaagtaaagatttggatggtgcagnnnnnnnnnnnntcgtcggcagcgtcagatgtgtataagagacagctaactgcaccatccaaatctttactttagacttttaagatacttgacgatccaccaatcgctcattcctgtatccttttgttgggacttaacgtatcccaagaagtccggcataagatcagtagcttgctttgcgagtccatcaatattctccaagaagcctggcaagtctagtcccagtctatacttgtctcctttcatgcaataattcacgaattcttccttgagtgggtggtatttgatc

SEQ ID NO: 52 - DNA, 1787 bp, Artificial sequence
  Synthetic
  misc_feature (1555)..(1579): n is a, c, g, or t
  misc_feature (1783)..(1787): n is a, c, g, or t
taggtaattactttaaattaccaaatcccggttgaagacctatctattgaaaactaagagaggtaacgatgaagagtatcgtactccatttttcaaaggtaaatctttgtccgatgtattaaaaggctgggaagtgcacctcgcccctctcaaagagaagtggcctggtttacaccagtttgaattagacctagcggcaaaggtcgggcctatgagtattcagaaaccgcttgatgagcgatttaaggatattgaggcttattacaaaggtattctcctaccttccgaaccaattagtgatgaagcaatccgatctgtcatcactgaatggaacagggctcgcggattgtcagttcgcagtacttccaaaacatgggataacatgaagaagtcaacttcttcaggctctccattctttaccaaacgtaaattgattggtaagtatataatggatagtcaaccatattttgacaaaagaacacaaaaggtacacgatagaaagtacccacaatgggatccaattgctgttcttggttggcgtggacaagaaggaggtccagaaccagaggatgtgaagcaaagggttgtatggatgttccctgcttcagttaacctgcaagagttgcgggtgtaccaacctctgatcgaaacagcgcaacgtttcaacttagttcctgcttgggttagcatggatagtgtggacgagcacatcacacgtatgtttgatacaaaaggcgcagatgatgtcgtgatttgtactgatttctctaaatttgaccaacactttaattctgatatggctcgcggtgcatctgagatattagatggactatttaacggcagccgagatttcgtacaatggatgtgggatacataccacattaaatacacgatacctctattagactctgagaaccatgcgtggtttggacgtcatggtatgggttccggttcaggcggaactaatgctgatgagacattagctcatcgtgcgcttcagtatgaagcagcactctcacaaaaacaaacactaaacccttattcacaatgcttgggagatgatggagtactaacgtacccaggtattaaagtggatgatgtaatgcgatcatatactgcacatggtcaagagatgaatgagtcgaagcagtacgtgagcaaacatgaatgcatatatctgagaagatggcatcacaaggattatcgtgtgtcaggtatatgtgtcggagtttatgcaactactcgtgctttgggtagattgtgtgaacaagaaaggtactttgacccagaagtatggtcaaaggaaatggtagctttacgtcagctatcaatccttgagaatattaaataccacccactcaaggaagaattcgtgaattattgcatgaaaggcgacaagtatagactgggactagacttgccaggcttcttggagaacattgatggactcgcaaagcaagctactgatcttatgccagactttttgggatacgttaaatctcaacagaaagatacaggaatgagcgattggtggatcgtgaagtatcttaagagnnnnnnnnnnnnnnnnnnnnnnnnntgtgtataagagacagtaactgcaccatccaaatctttactttagactcttaagatacttcacgatccaccaatcgctcattcctgtatctttctgttgagatttaacgtatcccaaaaagtctggcataagatcagtagcttgctttgcgagtccatcaatgttctccaagaagcctggcaagtctagtcccagtccatgctnnnnn

SEQ ID NO: 53 - DNA, 1880 bp, Artificial sequence
  Synthetic
  misc_feature (1812)..(1880): n is a, c, g, or t
tacttatgaatagaaaagtagtcagtttaggtaattactttaaattaccaaatcccggattgaagacctatctattgaaaactaagagaggtaacgatgaagagtatcgtactccatttttcaaaggtaaattttatccgatgttttaaaaggctgggaagtgcacctcgcccctctcaaagagaagtggcctggtttacaccagtttgaattagacctagcggaaaaggtcgggcctatgagtattcagaaacctcttgaagagcgtttcacggatattgaggcttattacaaaggtattctcctaccttccgaaccaattagtgatgaagcaatccgatctgtcatcactgagtggaacagggctcgcggattgtcagttcgcagtacttccaaaacatgggacaatatgaagaagtctacttcttcaggctctccattctttactaaacgtaagttaattggtaaatatataatggatagtcaaccatattttgacaaaagaacgcaagaggtacatgataaaatgtatccacattgggatccaattgccgttcttggttggcgtggacaagaaggaggtccagaaccagaggatgtgaagcaaagggttgtatggatgttccctgcttcagttaacttgcaagaattacgagtataccaacctctgatcgaaacagcgcaacgtttcaacttagttcctgcttgggttagcatggatagtgtggacgagcacatcacacgtatgtttgatactaaaggcgcagatgatgtcgtgatttgtactgatttctctaaatttgaccaacactttaatgctgatatggctcgcggcgcatccgaaatattggatggcatatttaacgggggccgagacttcatacaatggatgtgggacatatatcacatcaaatacacgatacctctattagactctgagaaccatgcgtggtttggacgtcatggtatgggttccggttcaggcggaactaatgctgatgagactttagctcatcgtgcgttgcaatatgaggcagcgctctcacaaaaccaaacactaaacccttattcacaatgcttgggtgatgatggagtactaacatatccaggcatcaaagtggatgatgtaatgcgatcatatactgctcatggtcaagaaatgaatgagtcgaagcagtacgtgagcaaacatgaatgcatatatctgagaagatggcatcacaaagattatcgtgttgcagatgtatgtgtcggagtttatgcaactaccagagctttgggtaggttgtgtgaacaagaaagatattttgacccagaagtatggtcaaaagaaatggtagctttacgtcagctatcgatccttgagaatgtcaaataccacccacttaaggaagaattcgtgaattattgcatgaaaggcgacaagtatagactgggactagacttgccaggcttcttggagaacattgatggactcgcaaagcaagctactgatctgatgccggactttttgggatacgttaagtcccaacagaaagatacaggaatgagcgattggtggatcgtcaagtatcttaagagtctaaagtaaagatttggatggtgcagtaaaccattagaattcatttgaattctaactgctgcaccatccaaatctttactttagactcttaagatacttgacgatccaccaatcgctcattcctgtatctttctgttgagacttaacgtatcccaaaaagtccggcatcagatcagtagcttcctttgcgagtccatcaatgttctccaagaagcctggcaagtctagtnnnnnnnnnnnnnnnnnnnnnnnnnnnnnnnnnnnnnnnnnnnnnnnnnnnnnnnnnnnnnnnnnnnnn

SEQ ID NO: 54 - DNA, 1941 bp, Artificial sequence
  Synthetic
tcataagtagcctccttttctagtaaacattttcgttagaatttatttactagaaaaggaggctacttatgaatagaaaagtagtcagtttaggtaattactttaaattaccaaatcccggattgaagacctatctattgaaaactaagagaggtaacgatgaagagtatcgtactccatttttcaaaggtaaatctttatccgatgttttaaaaggctgggaagtgcacctcgcccctctcaaagagaagtggcctggtttacaccagtttgaattagacctagcggaaaaggtcgggcctatgagtattcagaaacctcttgaagagcgtttcacggatattgaggcttattacaaaggtattctcctaccttccgaaccaattagtgatgaagcaatccgatctgtcatcactgagtggaacagggctcgcggattgtcagttcgcagtacttccaaaacatgggacaatatgaagaagtctacttcttcaggctctccattctttactaaacgtaagttaattggtaaatatataatggatagtcaaccatattttgacaaaagaacgcaagaggtacatgataaaatgtatccacattgggatccaattgccgttcttggttggcgtggacaagaaggaggtccagaaccagaggatgtgaagcaaagggttgtatggatgttccctgcttcagttaacttgcaagaattacgagtataccaacctctgatcgaaacagcgcaacgtttcaacttagttcctgcttgggttagcatggatagtgtggacgagcacatcacacgtatgtttgatactaaaggcgcagatgatgtcgtgatttgtactgatttctctaaatttgaccaacactttaatgctgatatggctcgcggcgcatccgaaatattggatggcatatttaacgggggccgagacttcatacaatggatgtgggacatatatcacatcaaatacacgatacctctattagactctgagaaccatgcgtggtttggacgtcatggtatgggttccggttcaggcggaactaatgctgatgagactttagctcatcgtgcgttgcaatatgaggcagcgctctcacaaaaccaaacactaaacccttattcacaatgcttgggtgatgatggagtactaacatatccaggcatcaaagtggatgatgtaatgcgatcatatactgctcatggtcaagaaatgaatgagtcgaagcagtacgtgagcaaacatgaatgcatatatctgagaagatggcatcacaaagattatcgtgttgcagatgtatgtgtcggagtttatgcaactaccagagctttgggtaggttgtgtgaacaagaaagatattttgacccagaagtatggtcaaaagaaatggtagctttacgtcagctatcgatccttgagaatgtcaaataccacccacttaaggaagaattcgtgaattattgcatgaaaggcgacaagtatagactgggactagacttgccaggcttcttggagaacattgatggactcgcaaagcaagctactgatctgatgccggactttttgggatacgttaagtcccaacagaaagatacaggaatgagcgattggtggatcgtcaagtatcttaagagtctaaagtaaagatttggatggtgcagcaaaccattagaattcatttgaattctaactgcaccatccaaatctttactttagactcttaagatacttgacgatccaccaatcgctcattcctgtatctttctgttgggacttaacgtatcccaaaaagtccggcatcagatcagtagcttgctttgcgagtccatcaatgttctccaagaagcctggcaagtctagtcccagtctatacttgtcgcctttcatgcaataattcacgaattcttccttaagtgggtggtatttgacc

SEQ ID NO: 55 - DNA, 1943 bp, Artificial sequence
  Synthetic
tgatacggcgaccaccgagatctacacttgcgaagtcgtcggcagcgtcagatgtgtataagagacagcttatgaatagaaaagtagtcagtttaggtaattactttaaattaccaaatcccggttgaagacctatctattgaaaactaagagaggtaacgatgaagagtatcgtactccatttttcaaaggtaaatctttatccgatgttttaaaaggctgggaagtgcacctcgcccctctcaaagagaagtggcctggtttacaccagtttgaattagacctagcggaaaaggtcgggcctatgagtattcagaaacctcttgaagagcgtttcacggatattgaggcttattacaaaggtattctcctaccttccgaaccaattagtgatgaagcaatccgatctgtcatcactgagtggaacagggctcgcggattgtcagttcgcagtacttccaaaacatgggacaatatgaagaagtctacttcttcaggctctccattctttactaaacgtaagttaattggtaaatatataatggatagtcaaccatattttgacaaaagaacgcaagaggtacatgataaaatgtatccacattgggatccaattgccgttcttggttggcgtggacaagaaggaggtccagaaccagaggatgtgaagcaaagggttgtatggatgttccctgcttcagttaacttgcaagaattacgagtataccaacctctgatcgaaacagcgcaacgtttcaacttagttcctgcttgggttagcatggatagtgtggacgagcacatcacacgtatgtttgatactaaaggcgcagatgatgtcgtgatttgtactgatttctctaaatttgaccaacactttaatgctgatatggctcgcggcgcatccgaaatattggatggcatatttaacgggggccgagacttcatacaatggatgtgggacatatatcacatcaaatacacgatacctctattagactctgagaaccatgcgtggtttggacgtcatggtatgggttccggttcaggcggaactaatgctgatgagactttagctcatcgtgcgttgcaatatgaggcagcgctctcacaaaaccaaacactaaacccttattcacaatgcttgggtgatgatggagtactaacatatccaggcatcaaagtggatgatgtaatgcgatcatatactgctcatggtcaagaaatgaatgagtcgaagcagtacgtgagcaaacatgaatgcatatatctgagaagatggcatcacaaagattatcgtgttgcagatgtatgtgtcggagtttatgcaactaccagagctttgggtaggttgtgtgaacaagaaagatattttgacccagaagtatggtcaaaagaaatggtagctttacgtcagctatcgatccttgagaatgtcaaataccacccacttaaggaagaattcgtgaattattgcatgaaaggcgacaagtatagactgggactagacttgccaggcttcttggagaacattgatggactcgcaaagcaagctactgatctgatgccggactttttgggatacgttaagtcccaacagaaagatacaggaatgagcgattggtggatcgtcaagtatcttaagagtctaaagtaaagatttggatggtgcagcaaaccattagaattcatttgaattctaactgcaccatccaaatctttactttagactcttaagatacttgacgatccaccaatcgctcattcctgtatctttctgttgggacttaacgtatcccaaaaagtccggcatcagatcagtagcttgctttgcgagtccatcaatgttctccaagaagcctggcaagtctagtcccagtctatacttgtcgcctttcatgcaataattcacgaattcttccttaagtgggtggtatttgaca

SEQ ID NO: 56 - DNA, 1671 bp, Artificial sequence
  Synthetic
actcgttaacactagttgtagagcgcgtactcccgcggtccgaccagaccgctcgcgacttacagaaaggaggtcgatcgtatgcctaaatacgataacatcatggcggattatttcgatctgcccaatccagcgttggggtcatatttcggtagaacccgacatggcaatcctgatgtatacaggaccacattcttcaagaatcgtgagcctcaggatgttttgtcagaatggatgaagtcagtccaggttcttaaacaggattggcctacgctgttaacatttgaggaagaccttgcttccaaagtaggtccactgtcagtgcagaagcctttagtggataggctccctgatattcaggcttactatgactgcattaacctggagtcaaaaccccttgagaaagaagcagttcaagctttcttgaaggagttaaaaggtttaaacaccttatcgatgcgcggaattcccgctacgatagaaaacatgaagttgtccacttccagcggttgtccatatttcaccaaacgtaagaacgatgttcgccgtcacagatacggggacgtaaagtatgacggaaatcgtatcactgcaaacataggtggcaaggaatttaagatggccgctattcttggatggagaggccaggaaggaggaccaaataattcggacgttaaacagagagtggtatggatgttccctttcactgttaacctccaagaactacgtgtctaccagccgtttatggatatgttacaaaagcacaagattataccagcatgggtcgggctggatgaggtagacaataagatcaccaaattgtttgacaccaaaggtgaagatgacgtagttatatgtaccgacttttctaagtttgaccagcactttaatgaagattgccagaaagtagcccatgatatcttagcttggttgtttattggtgatagccgtatggaaggctggttgcgtaatgtatttcctgtcaaatacaatattcctataatctgtgatgacaatattgtgaagaatggtcgtcatggtatgggttccgggtcgggaggaacaaatcaagatgaaacgctactacacagagtattacaacatgaagcggctcttagtgtaggacaggacctcaatcttaattcacaatgcttaggtgatgatggtatactaacttacccaggtattaaggtagaggatgtaatacgaacatatactgcacatggtcaagaaatgaatcccgataagcagtatgtgagtaaacaggactgcgtatatcttcgtagatggcaccataaagactatcgcgaaaacggcgtatgcgtaggggtatatagtactgctcgcgctttagggcgtatgatgtaccaagaacgctactacgaccctgatgaatggggtaaagagatggttgcgctaagacaactgtctatattagagaactgcaaacaccaccctctcaaagaaaagtttgtggactattgcattaaaggggataaatataggcttggtatagatatcccaggttttctagacaatctggaaacgttgtcagagaaagctatcgaagtaatgcctgactttatgggctacacacaatctcttggacataaagatgaaagagtatccaaaggtattaatgattggtggatcgttaaatacttaaagtca

SEQ ID NO: 57 - DNA, 1671 bp, Artificial sequence
  Synthetic
actcgttaacactagttgtagagcgcgtactcccgtggtccgaccagaccacacgcgacttacagaaaggaggtcgatcgtatgcctaaatacgataacatcatggcagactattttgatctgcccaatccagcgttggggtcatatttcggtagaacccgacatggcaatcctgatgtatacaggaccacattcttcaagaatcgtgagcctcaggatgttctgtcagaatggatgaagtcgatccaggttcttaaacaggattggcctacgctgttaacttttgaggaagaccttgcttccaaagtaggaccactgtccgttcagaagcctttagtggataggctccctgacgttcaagcctactatgactgcattaacctggagtcaaaaccccttgcgaaagaagcagttcaagctttcatcaaggagttaaaaggtttaaataccttatcgatgcgtggaattcccgctacgatagaaaatatgaagttgtccacttccagtggctgtccttatttcaccaagcgtaaaagcgatgtacgccgtcatagatacggggacgtaaaatctgatggtaatcgtataaccgctgagatcggtggcaaggaatttaagatggccgctattcttggatggagaggccaggaaggaggaccaaagaattcggacgttaaacagagagtggtatggatgttccctttcactgttaacctccaagaactacgtgtctaccagccgtttatggatatgctccagaagcataaaattgtaccagcatgggtcggactggatgaggtagacaataagatcactaaattgtttgacaccaaaggtgaagatgacgtagttatatgtaccgacttttctaagtttgaccagcactttaatgaagattgccaaaaggtagcccatgatatcttagcttggttgtttattggcgatagccgtatggaaagctggttgcgtaatgtatttcctgtcaaatacaatattcctataatctgtgacgacaatattgtgaagaatggacgtcacggtatgggttccggttcgggaggaacaaatcaagatgaaacgctactacacagggtattacaacatgaagcggcccttagtgtaggacaggacctaaaccttaattcacaatgccttggtgatgatggtatactaacttaccctggtattaaggtggaggatgtaatacgaacatatactgcacatggtcaagagatgaatcccgataagcagtatgtgagtaaacaggactgcgtatatcttcgtagatggcaccataaagactatcgcgaaaacggcgtatgcgtaggggtatatagtactgcccgcgctttagggcgtatgatgtatcaagaacgctactatgaccccgatgaatggggtaaagagatggttgcgctaagacaactgtctatattagagaactgcaaacaccaccctctcaaagaaaagtttgtggactattgcattaaaggggataaatataggcttggtatagatatcccaggttttctagacaatctggaaacgttgtctgagaaagctatcgaagtaatgcctgactttatgggctacacacagtcacttggacataaagatgaaaaggtatccaaaggtattaatgattggtggatcgttaagtacttaaagtca

SEQ ID NO: 58 - DNA, 1671 bp, Artificial sequence
  Synthetic
  misc_feature (1663)..(1671): n is a, c, g, or t
actcgttaacactagttgtagagcgcgtactcccgtggtcagaccagaccacacgcgacttacagaaaggaggtcgatcgtatgcctaaatacgataacatcatggcagattattttgatctgcccaatccagcgttggggtcatatttcggtagaacccgacatggcaatcctgatgtatacaggaccacattctttaaggatcgtgagcctcaggatgttctgtcagaatggatgaagtcagtccaggttcttaaacaggattggcctacgctgttaacttttgaggaagaccttgcttccaaagtaggaccattgtccgttcagaagcctttagtggataggctccctgacgtacaggcttactatgactgcattaacctggagtcaaaacctcttcagaaagaagcagttcaagctttcttgaaggagttgaaaggtttaaacaccttatcgatgcgtggtattcccgcaacgatagaaaacatgaagttgtccacttctagtggttgtccattcttcaccagacgtaagaatgatgttcgtcgtcatcgctacggggacgtaagctttgatggaactaccattcatgctgaaataggtggcaaggattacaagatggcagccatattaggttggaggggccaagaaggaggaccaaagaattcggatgttaaacagagggtggtatggatgttcccattcactgttaacctccaagaactacgtgtctatcagccgtttatggatatgctacaaaaacacaaagtagtaccagcttgggtcggtctggatgaggtagacaataagattaccaaattgtttgacaccaaaggtaaagatgacgtagttatttgtaccgacttttcaaagtttgaccagcactttaatgaagattgccaaaaggtagcccatgatgtcttagcttggttatttattggtgatagccgtatggaaagctggttgcgtaatgtatttcctgtcaaatacaatattcctataatctgtgatgataatattgtgaagaatggtcgtcatggtatgggttccggttcgggaggaacaaatcaagatgaaacgctgctacacagggtattacaacatgaagcggcccttagtgtaggacaggacctcaaccttaattcacaatgcttgggtgatgatggtatactaacttatccaggtattaaagttgaggatgtaatacgaacatatactgcacatggtcaagaaatgaatcccgataagcagtatgtgagtaaacaggactgcgtatatcttcgcagatggcaccataaagactatcgcgaaaacggcgtatgcgtaggggtatatagtacagctcgcgctttagggcgtatgatgtaccaagaacgctactatgaccctgatgaatggggtaaagagatggttgcgctaagacaactgtctatattagagaactgcaaacaccaccctcttaaggaaaagtttgtggactattgcatgaaaggggataaatacaggctaggtgtagatatcccaggttttctggataatctggaaacgttatccgagaaggctatcgaagtcatgcccgatttcatgggctacacacaatccttgggacacaaggacgaaaagatatctaaaggtattaatgactggtggatcgttaaatacnnnnnnnnn

SEQ ID NO: 59 - DNA, 1671 bp, Artificial sequence
  Synthetic
  misc_feature (1)..(127): n is a, c, g, or t
  misc_feature (260)..(359): n is a, c, g, or t
  misc_feature (452)..(462): n is a, c, g, or t
  misc_feature (955)..(1007): n is a, c, g, or t
  misc_feature (1504)..(1671): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatccagcgttggggtcatatttcggtagaacccgacatggcaatcctgatgtatacaggaccacattcattaagaatcgtgagcctcagcatgttttgtcagaatggatgaagtcagtacaggttcttaaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcctattatgactgcattaacctggagtcaaaacccctagagaaagaggcagttcaagctttcttaaaggagttgaaaggtttaaatacctnnnnnnnnnnnggtattcccgctacgatagaaaacatgaagttgtccacttccagtggctgtccttatttcaccaagcgtaagaacgatgtacgccgtcacagatacggggacgtaaagtttgacggtacacgtgtgaccgctgatataggtggcaaggaatttaagatggccgctatacttggatggagaggccaggaaggaggaccaaagaattcggacgttaaacagagagtggtatggatgttccctttcactgttaacctccaagaactacgtgtctaccagccgtttatggatatgctacagaaacataaagtagtaccagcatgggtcggactggatgaggtagacaataagatcaccaaattgtttgacaccaaaggtgaagatgacgtagttatatgtaccgacttttctaagtttgaccagcactttaatgaagattgccaaaaggtagcccatgatatcttagcttggttatttattggtgatagccgtatgnnnnnnnnnnnnnnnnnnnnnnnnnnnnnnnnnnnnnnnnnnnnnnnnnnnnngacagacaatattgtgaagtcaggtcgtcatggtatgggttccggttcgggaggaacaaatcaagatgaaacgctactacacagggtattacaacatgaggcggcccttagtgtaggacaggaccttaatcttaattcacaatgcctaggtgacgatggtatactaacttaccctggtattaaggttgaggatgtaatacgaacatatactgcacatggtcaagaaatgaatcccgataagcagtatgtgagtaaacaggactgcgtatatcttcgtagatggcaccataaagactatcgcgaaaacggcgtatgcgtaggggtatatagtacagcccgcgctttggggcgtatgatgtaccaagaacgctactatgaccctgatgaatggggtaaagagatggttgcgctaagacaactgtctatattggagaactgcaaacaccatcctctcaaagagaagtttgtggactattgcattaaaggggataaatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 60 - DNA, 1671 bp, Artificial sequence
  Synthetic
  misc_feature (1)..(317): n is a, c, g, or t
  misc_feature (569)..(715): n is a, c, g, or t
  misc_feature (859)..(898): n is a, c, g, or t
  misc_feature (1460)..(1671): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtccgttcagaaaccgttagtggatcggttaccagacgttcaagcctactatgactgcattaacctggagtcaaaacccctagagaaagaggcagttcaagctttcttaaaggagttgaaaggtttaaataccttatcgatgcgcggtattcccgctacgatagaaaacatgaagttgtccacttccagtggctgtccttatttcaccaagcgtaagaacgatgtacgccgtcacagatacggggacgtaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccaagaactacgtgtctaccagccgtttatggatatgctacagaaacataaagtagtaccagcatgggtcggactggatgaggtagacaataagatcaccaaattgtttgacaccaaaggtgaagatgacgtagttatatgtnnnnnnnnnnnnnnnnnnnnnnnnnnnnnnnnnnnnnnnngccaaaaggtagcccatgatatcttagcttggttatttattggtgatagccgtatggaaagctggttgcgtaatgtatttcctgtcaaatacaatattcctataatctgtgacgacaatattgtgaagtcaggtcgtcatggtatgggttccggttcgggaggaacaaatcaagatgaaacgctactacacagggtattacaacatgaggcggcccttagtgtaggacaggaccttaatcttaattcacaatgcctaggtgacgatggtatactaacttaccctggtattaaggtggaggatgtaataagaacagatactgcacatggtcaagaaatgaatcccgataagcagtatgtgagtaaacaggactgcgtatatcttcgtagatggcaccataaagactatcgcgaaaacggcgtatgcgtaggggtatatagtacagcccgcgctttggggcgtatgatgtaccaagaacgctactatgaccctgatgaatggggtaaagagatggttgcgctaagacaactgtctatattggagaactgcaaacaccatccctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 61 - DNA, 1671 bp, Artificial sequence
  Synthetic
  misc_feature (1)..(14): n is a, c, g, or t
  misc_feature (1671)..(1671): n is a, c, g, or t
nnnnnnnnnnnnnngttgtagagcgcgtactcccgcggcccgaccaggtcgcacgcgacttacagaaaggaggtcgatcgtatgcctaaatacgataacatcatggcagattatttcgatctgcccaatccagcgttggggtcatatttcggtagaacccgacatggcaatcctgatgtatacaggaccacattctttaagaatcgtgagcctcaggatgttttgtcagaatggatgaagtcagtccaggttcttaaacaggattggcctacgctgttaacttttgaggaagaccttgcttccaaagtaggacctctgtccgttcagaaaccgttagtggatcggttaccagacgttcaagcctactatgactgcattaacctggagtcaaaacccctagagaaagaggcagttcaagctttcttaaaggagttgaaaggtttaaataccttatcgatgcgcggtattcccgctacgatagaaaacatgaagttgtccacttccagtggctgtccttatttcaccaagcgtaagaacgatgtacgccgtcacagatacggggacgtaaagtttgacggtacacgtgtgaccgctgatataggtggcaaggaatttaagatggccgctatacttggatggagaggccaggaaggaggaccaaagaattcggacgttaaacagagagtggtatggatgttccctttcactgttaacctccaagaactacgtgtctaccagccgtttatggatatgctacagaaacataaagtagtaccagcatgggtcggactggatgaggtagacaataagatcaccaaattgtttgacaccaaaggtgaagatgacgtagttatatgtaccgacttttctaagtttgaccagcactttaatgaagattgccaaaaggtagcccatgatatcttagcttggttatttattggtgatagccgtatggaaagctggttgcgtaatgtatttcctgtcaaatacaatattcctataatctgtgacgacaatattgtgaagtcaggtcgtcatggtatgggttccggttcgggaggaacaaatcaagatgaaacgctactacacagggtattacaacatgaggcggcccttagtgtaggacaggaccttaatcttaattcacaatgcctaggtgacgatggtatactaacttaccctggtattaaggttgaggatgtaatacgaacatatactgcacatggtcaagaaatgaatcccgataagcagtatgtgagtaaacaggactgcgtatatcttcgtagatggcaccataaagactatcgcgaaaacggcgtatgcgtaggggtatatagtacagcccgcgctttggggcgtatgatgtaccaagaacgctactatgaccctgatgaatggggtaaagagatggttgcgctaagacaactgtctatattggagaactgcaaacaccatcctctcaaagagaagtttgtggactattgcattaaaggggataaatataggcttggtatagatatcccaggttttctagataatctggaaacgttatctgagaaagctatcgaagtaatgccagactttatgggctacacacaatcacttggacaccatgaagataaggtgtcaaaaggtattaatgattggtggatcgttaaatacctgaagtcn

SEQ ID NO: 62 - DNA, 1671 bp, Artificial sequence
  Synthetic
  misc_feature (1)..(439): n is a, c, g, or t
  misc_feature (1142)..(1142): n is a, c, g, or t
  misc_feature (1643)..(1671): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtttaaataccttatcgatgcgcggtattcccgctacgatagaaaacatgaagttgtccacttccagtggctgtccttatttcaccaagcgtaagaacgatgtacgccgtcacagatacggggacgtaaagtttgacggtacacgtgtgaccgctgatataggtggcaaggaatttaagatggccgctatacttggatggagaggccaggaaggaggaccaaagaattcggacgttaaacagagagtggtatggatgttccctttcactgttaacctccaagaactacgtgtctaccagccgtttatggatatgctacagaaacataaagtagtaccagcatgggtcggactggatgaggtagacaataagataaccaaattgtttgacaccaaaggtgaagatgacgtagttatatgtaccgacttttctaagtttgaccagcactttaatgaagattgccaaaaagtagcccatgatatcttagcttggttatttattggtgatagccgtatcgaaagctggttgcgtaatgtatttcctgtcaaatacaatattcctataatctgtgacgacaatattgtgaagtcaggtcgtcatggtatgggttccggttcgggaggaacaaatcaagatgaaacgctactacacagggtattacaacatgaggcggcccttagtgtaggacaggaccttaatcttanttcacaatgcttgggtgatgatggagtactaacatatccaggcatcaaagtggatgatgtaatgcgatcatatactgctcatggtcaagaaatgaatgagtcgaagcagtacgtgagcaaacatgaatgcatatatctgagaagatggcatcacaaagattatcgcgaaaacggcgtatgcgtaggggtatatagtacagcccgcgctttggggcgtatgatgtaccaagaacgctactatgaccctgatgaatggggtaaagagatggttgcgctaagacaactgtctatattggagaactgcaaacaccatcctctcaaagagaagtttgtggactattgcattaaaggggataaatataggcttggtatagatatcccaggttttctagataatctggaaacgttatctgagaaagctatcgaagtaatgccagactttatgggctacacacaatcacttggacaccatgaagataaggtgtcaaaaggtattaatgnnnnnnnnnnnnnnnnnnnnnnnnnnnnn

SEQ ID NO: 63 - DNA, 1671 bp, Artificial sequence
  Synthetic
  misc_feature (1)..(746): n is a, c, g, or t
  misc_feature (854)..(1216): n is a, c, g, or t
  misc_feature (1502)..(1671): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatatgctacagaaacataaagtagtaccagcatgggtcggactggatgaggtagacaataagatcaccaaattgtttgacaccaaaggtgaagatgacgtagttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgcacatggtcaagaaatgaatcccgataagcagtatgtgagtaaacaggactgcgtatatcttcgtagatggcaccataaagactatcgcgaaaacggcgtatgcgtaggggtatatagtacagcccgcgctttggggcgtatgatgtaccaagaacgctactatgaccctgatgaatggggtaaagagatggttgcgctaagacaactgtctatattggagaactgcaaacaccatcctctcaaagagaagtttgtggactattgcattaaaggggataaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 64 - DNA, 2251 bp, Artificial sequence; identical to SEQ ID NO: 1
  Synthetic

SEQ ID NO: 65 - DNA, 1882 bp, Artificial sequence
  Synthetic
ctaaatgaatagaaaagtagtcaagttaggtaattattttaaattaccgaatcccggttgaagacctatctattgaaaactaagagaggtaacgatgaagagtatcgtactccatttttcaaagataaatctttgtccgatgtattacaaggctggttagtgcacctagcccctctcaagagtgagtggcctggtttacaccagtttgaattagacctagcggaaaaggtcgggcctttaagcatccagaaacctttagatgagcggtttaaggatattgaggcttattacaaaggtattctcctaccttccaaaccaatcagtgaaacagcaatccgatctgttttaactgaatggaatagggcacgtggcttgtcggtacgcagtgtctccaaaacgtgggataacatgaagaaatctacatcttcaggttctccattctttactaaacgtaaagcagtcggaaaatatacgatgtatatggagccatgttttgacaaaagaacgcaagaagttcattttaagaactcaaaccgttgggatccaattgcggtcttaggttggcgtggacaagaaggtggacctgattttgaggatgtaaagcaaagggttgtatggatgttccctgcttcggtaaacctacaagagttacgtgtttaccaacctctaatcgaaacagcgcaacgtttcaacttagttcctgcttgggttggcatggatagtgttgatttgcacatcacacgtatgtttgatacgaaaggcgaagacgatgtcgtaatatgtacagatttctcaaaatttgaccaacattttaatgctgatatggctcgcggtgcatccgaaatattggatggcctctttaacgggagcagagattttgtacaatggatgtgggatatatatcacatcaaatacacgatacctctattagactcagaagatcatgcctggtttggcagacatggtatgggctctggttcaggtggaaccaatgccgatgaaacattagctcatagagctttgcagtacgaagctgctttatcacagaaccaaacattaaacccttattcacaatgtctaggtgatgatggagtactaacatatcctggaattaaagtggatgatgtaatgcgatcatatactgcacatggtcaagagatgaatgagtcaaaacagtatgtgagcaaacatgaatgcatatatcttcgtagatggcatcatattaattatcgtgtcgatgatgtatgtgtcggagtttacgcaacaactcgtgctttgggtagattgtgtgaacaagagagatattttgacccagagatatggtcaaaagaaatggtagctttacgtcagctatcgatacttgagaatgtgaaataccaccctctcaaggaagaatttgttaaatattgcatgaaaggggataagtacagactgggactggacttaccaggcttcttggagaacatagatggactcgcaaagcaagctactgatctaatgccggactttttaggttacgttaaatcacaacagaaatctgtcggtggtatatcagaatggtggatagtaaaatatctacgtagtctaaagtaaagattgggatggtgcagtaaaccattagaattctaacgaattctaactgcaccatcccaatctttactttagactacgtagatattttactatccaccactctgatataccaccgacagatttctgttgtgatttaacgtaacctaaaaagtccggcatcagatcagtagcttgctttgcgagtccatctatgttctccaagaagcctggtaagtccagtcccagtctgtacttatcccctttcatgcaatatttaacaaattcttccttgagagggtggtatttcaca